SEQUENCE LISTING (173 sequences)

SEQ ID NO: 1 - protein, 437 aa, Saccharomyces cervisiae [sequence omitted]

SEQ ID NO: 2 - protein, 900 aa, Saccharomyces cervisiae
MPGNLSFKDRVVVITGAGGGLGKVYALAYASRGAKVVVNDLGGTLGGSGHNSKAADLVVDEIKKAGGIAVANYDSVNENGEKIIETAIKEFGRVDVLINNAGILRDVSFAKMTEREFASVVDVHLTGGYKLSRAAWPYMRSQKFGRIINTASPAGLFGNFGQANYSAAKMGLVGLAETLAKEGAKYNINVNSIAPLARSRMTENVLPPHILKQLGPEKIVPLVLYLTHESTKVSNSIFELAAGFFGQLRWERSSGQIFNPDPKTYTPEAILNKWKEITDYRDKPFNKTQHPYQLSDYNDLITKAKKLPPNEQGSVKIKSLCNKVVVVTGAGGGLGKSHAIWFARYGAKVVVNDIKDPFSVVEEINKLYGEGTAIPDSHDVVTEAPLIIQTAISKFQRVDILVNNAGILRDKSFLKMKDEEWFAVLKVHLFSTFSLSKAVWPIFTKQKSGFIINTTSTSGIYGNFGQANYAAAKAAILGFSKTIALEGAKRGIIVNVIAPHAETAMTKTIFSEKELSNHFDASQVSPLVVLLASEELQKYSGRRVIGQLFEVGGGWCGQTRWQRSSGYVSIKETIEPEEIKENWNHITDFSRNTINPSSTEESSMATLQAVQKAHSSKELDDGLFKYTTKDCILYNLGLGCTSKELKYTYENDPDFQVLPTFAVIPFMQATATLAMDNLVDNFNYAMLLHGEQYFKLCTPTMPSNGTLKTLAKPLQVLDKNGKAALVVGGFETYDIKTKKLIAYNEGSFFIRGAHVPPEKEVRDGKRAKFAVQNFEVPHGKVPDFEAEISTNKDQAALYRLSGDFNPLHIDPTLAKAVKFPTPILHGLCTLGISAKALFEHYGPYEELKVRFTNVVFPGDTLKVKAWKQGSVVVFQTIDTTRNVIVLDNAAVKLSQAKSKL

SEQ ID NO: 3 - protein, 373 aa, Saccharomyces cervisiae
MAAVTLHLRAETKPLEARAALTPTTVKKLIAKGFKIYVEDSPQSTFNINEYRQAGAIIVPAGSWKTAPRDRIIIGLKEMPETDTFPLVHEHIQFAHCYKDQAGWQNVLMRFIKGHGTLYDLEFLENDQGRRVAAFGFYAGFAGAALGVRDWAFKQTHSDDEDLPAVSPYPNEKALVKDVTKDYKEALATGARKPTVLIIGALGRCGSGAIDLLHKVGIPDANILKWDIKETSRGGPFDEIPQADIFINCIYLSKPIAPFTNMEKLNNPNRRLRTVVDVSADTTNPHNPIPIYTVATVFNKPTVLVPTTAGPKLSVISIDHLPSLLPREASEFFSHDLLPSLELLPQRKTAPVWVRAKKLFDRHCARVKRSSRL

SEQ ID NO: 4 - protein, 593 aa, Saccharomyces cervisiae
MVRDLVTLPSSLPLITAGFATDQVHLLIGTGSTDSVSVCKNRIHSILNAGGNPIVVNPSSPSHTKQLQLEFGKFAKFEIVEREFRLSDLTTLGRVLVCKVVDRVFVDLPITQSRLCEEIFWQCQKLRIPINTFHKPEFSTFNMIPTWVDPKGSGLQISVTTNGNGYILANRIKRDIISHLPPNISEVVINMGYLKDRIINEDHKALLEEKYYQTDMSLPGFGYGLDEDGWESHKFNKLIREFEMTSREQRLKRTRWLSQIMEYYPMNKLSDIKLEDFETSSSPNKKTKQETVTEGVVPPTDENIENGTKQLQLSEVKKEEGPKKLGKISLVGSGPGSVSMLTIGALQEIKSADIILADKLVPQAILDLIPPKTETFIAKKFPGNAERAQQELLAKGLESLDNGLKVVRLKQGDPYIFGRGGEEFNFFKDHGYIPVVLPGISSSLACTVLAQIPATQRDIADQVLICTGTGRKGALPIIPEFVESRTTVFLMALHRANVLITGLLKHGWDGDVPAAIVERGSCPDQRVTRTLLKWVPEVVEEIGSRPPGVLVVGKAVNALVEKDLINFDESRKFVIDEGFREFEVDVDSLFKLY

SEQ ID NO: 5 - protein, 382 aa, Saccharomyces cervisiae
MPKKQTNFPVDNENRPFRCDTCHRGFHRLEHKKRHLRTHTGEKPHHCAFPGCGKSFSRSDELKRHMRTHTGQSQRRLKKASVQKQEFLTVSGIPTIASGVMIHQPIPQVLPANMAINVQAVNGGNIIHAPNAVHPMVIPIMAQPAPIHASAASFQPATSPMPISTYTPVPSQSFTSFQSSIGSIQSNSDVSSIFSNMNVRVNTPRSVPNSPNDGYLHQQHIPQQYQHQTASPSVAKQQKTFAHSLASALSTLQKRTPVSAPSTTIESPSSPSDSSHTSASSSAISLPFSNAPSQLAVAKELESVYLDSNRYTTKTRRERAKFEIPEEQEEDTNNSSSGSNEEEHESLDHESSKSRKKLSGVKLPPVRNLLKQIDVFNGPKRV

SEQ ID NO: 6 - protein, 231 aa, Saccharomyces cervisiae
MSACPCNIVILPVEILKNSSKDTKYSLYTTINRGYDVPRLKYGIIVSPRVHSLETLFSDLGFDKNIEKSSLYLLLNDPTLAYPNFHEHFEQLKGETNKDLSLPTYYIPKVQFLTEAFDSEHTLATIGYKPNNKESYEITGFTSMGNGYGIKLFNYSVIHMMRSHKCKRVVADIIMEHDLLGYYEKKLGFVEVQRFKVLKEQHQVKVFDDKVDFTKDFHVIKMIKELGNHRL

SEQ ID NO: 7 - protein, 300 aa, Saccharomyces cervisiae
MSPCYGQNSAIAKGSWNREVLQEVQPIYHWHDFGQNMKEYSASPLEGDSSLPSSLPSSTEDCLLLSLENTITVIAGNQRQAYDSTSSTEEGTAPQLRPDEIADSTHCITSLVDPEFRDLINYGRQKGANPVFIESNTTEQSHSQCILGYPQKSHVAQLYHDPKVLSTISEGQTKRGSYHCSHCSEKFATLVEFAAHLDEFNLERPCKCPIEQCPWKILGFQQATGLRRHCASQHIGELDIEMEKSLNLKVEKYPGLNCPFPICQKTFRRKDAYKRHVAMVHNNADSRFNKRLKKILNNTK

SEQ ID NO: 8 - protein, 815 aa, Saccharomyces cervisiae
MGNSPSTQDPSHSTKKEHGHHFHDAFNKDRQGSITSQLFNNRKSTHKRRASHTSEHNGAIPPRMQLLASHDPSTDCDGRMSSDTTIDKGPSHLFKKDYSLSSAADVNDTTLANLTLSDDHDVGAPEEQVKSPSFLSPGPSMATVKRTKSDLDDLSTLNYTMVDETTENERNDKPHHERHRSSIIALKKNLLESSATASPSPTRSSSVHSASLPALTKTDSIDIPVRQPYSKKPSIHAYQYQYLNNDETFSENSQMDKEGNSDSVDAEAGVLQSEDMVLNQSLLQNALKKDMQRLSRVNSSNSMYTAERISHANNNGNIENNTRNKGNAGGSNDDFTAPISATAKMMMKLYGDKTLMERDLNKHHNKTKKAQNKKIRSVSNSRRSSFASLHSLQSRKSILTNGLNLQPLHPLHPIINDNESQYSAPQHREISHHSNSMSSMSSISSTNSTENTLVVLKWKDDGTVAATTEVFIVSTDIASALKEQRELTLDENASLDSEKQLNPRIRMVYDDVHKEWFVPDLFLPAGIYRLQFSINGILTHSNFLPTATDSEGNFVNWFEVLPGYHTIEPFRNEADIDSQVEPTLDEELPKRPELKRFPSSSRKSSYYSAKGVERPSTPFSDYRGLSRSSSINMRDSFVRLKASSLDLMAEVKPERLVYSNEIPNLFNIGDGSTISVKGDSDDVHPQEPPSFTHRVVDCNQDDLFATLQQGGNIDAETAEAVFLSRYPVPDLPIYLNSSYLNRILNQSNQNSESHERDEGAINHIIPHVNLNHLLTSSIRDEIISVACTTRYEGKFITQVVYAPCYYKTQKSQISN

SEQ ID NO: 9 - protein, 300 aa, Saccharomyces cervisiae
MNYNLSKYPDDVSRLFKPRPPLSYKRPTDYPYAKRQTNPNITGVANLLSTSLKHYMEEFPEGSPNNHLQRYEDIKLSKIKNAQLLDRRLQNWNPNVDPHIKDTDPYRTIFIGRLPYDLDEIELQKYFVKFGEIEKIRIVKDKITQKSKGYAFIVFKDPISSKMAFKEIGVHRGIQIKDRICIVDIERGRTVKYFKPRRLGGGLGGRGYSNRDSRLPGRFASASTSNPAERNYAPRLPRRETSSSAYSADRYGSSTLDARYRGNRPLLSAATPTAAVTSVYKSRNSRTRESQPAPKEAPDY

SEQ ID NO: 10 - protein, 332 aa, Saccharomyces cervisiae
MIRIAINGFGRIGRLVLRLALQRKDIEVVAVNDPFISNDYAAYMVKYDSTHGRYKGTVSHDDKHIIIDGVKIATYQERDPANLPWGSLKIDVAVDSTGVFKELDTAQKHIDAGAKKVVITAPSSSAPMFVVGVNHTKYTPDKKIVSNASCTTNCLAPLAKVINDAFGIEEGLMTTVHSMTATQKTVDGPSHKDWRGGRTASGNIIPSSTGAAKAVGKVLPELQGKLTGMAFRVPTVDVSVVDLTVKLEKEATYDQIKKAVKAAAEGPMKGVLGYTEDAVVSSDFLGDTHASIFDASAGIQLSPKFVKLISWYDNEYGYSARVVDLIEYVAKA

SEQ ID NO: 11 - protein, 391 aa, Saccharomyces cervisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 12 - protein, 1380 aa, Saccharomyces cervisiae
MEPFAFGRGAPALCILTAAARINLDNFVPCCWALFRLSFFFPLDPAYIRNENKETRTSWISIEFFFFVKHCLSQHTFFSKTLAPKRNFRAKKLKDIGDTRIDRADKDFLLVPEPSMFVNGNQSNFAKPAGQGILPIPKKSRIIKTDKPRPFLCPTCTRGFVRQEHLKRHQHSHTREKPYLCIFCGRCFARRDLVLRHQQKLHAALVGTGDPRRMTPAPNSTSSFASKRRHSVAADDPTDLHIIKIAGNKETILPTPKNLAGKTSEELKEAVVALAKSNNVELPVSAPVMNDKREKTPPSKAGSLGFREFKFSTKGVPVHSASSDAVIDRANTPSSMHKTKRHASFSASSAMTYMSSSNSPHHSITNFELVEDAPHQVGFSTPQMTAKQLMESVSELDLPPLTLDEPPQAIKFNLNLFNNDPSGQQQQQQQQQQNSTSSTIVNSNNGSTVATPGVYLLSSGPSLTDLLTMNSAHAGAGGYMSSHHSPFDLGCFSHDKPTVSEFNLPSSFPNTIPSNSTTASNSYSNLANQTYRQMSNEQPLMSLSPKNPPTTVSDSSSTINFNPGTNNLLEPSMEPNDKDSNIDPAAIDDKWLSEFINNSDPKSTFKINFNHFNDIGFIYSPPSSRSSIPNKSPPNHSATSLNHEKASLSPRLNLSLNGSTDLPSTPQNQLKEPSYSDPISHSSHKRRRDSVMMDYDLSNFFSSRQLDISKVLNGTEQNNSHVNDDVLTLSFPGETDSNATQKQLPVLTPSDLLSPFSVPSVSQVLFTNELRSMMLADNNIDSGAFPTTSQLNDYVTYYKEEFHPFFSFIHLPSIIPNMDSYPLLLSISMVGALYGFHSTHAKVLANAASTQIRKSLKVSEKNPETTELWVIQTLVLLTFYCIFNKNTAVIKGMHGQLTTIIRLLKASRLNLPLESLCQPPIESDHIMEYENSPHMFSKIREQYNAPNQMNKNYQYFVLAQSRIRTCHAVLLISNLFSSLVGADCCFHSVDLKCGVPCYKEELYQCRNSDEWSDLLCQYKITLDSKFSLIELSNGNEAYENCLRFLSTGDSFFYGNARVSLSTCLSLLISIHEKILIERNNARISNNNTNSNNIELDDIEWKMTSRQRIDTMLKYWENLYLKNGGILTPTENSMSTINANPAMRLIIPVYLFAKMRRCLDLAHVIEKIWLKDWSNMNKALEEVCYDMGSLREATEYALNMVDAWTSFFTYIKQGKRRIFNTPVFATTCMFTAVLVISEYMKCVEDWARGYNANNPNSALLDFSDRVLWLKAERILRRLQMNLIPKECDVLKSYTDFLRWQDKDALDLSALNEEQAQRAMDPNTDINETIQLIVAASLSSKCLYLGVQILGDAPIWPIILSFAHGLQSRAIYSVTKKRNTRI

SEQ ID NO: 13 - protein, 492 aa, Saccharomyces cervisiae
MSKAVGDLGLVGLAVMGQNLILNAADHGFTVVAYNRTQSKVDRFLANEAKGKSIIGATSIEDLVAKLKKPRKIMLLIKAGAPVDTLIKELVPHLDKGDIIIDGGNSHFPDTNRRYEELTKQGILFVGSGVSGGEDGARFGPSLMPGGSAEAWPHIKNIFQSIAAKSNGEPCCEWVGPAGSGHYVKMVHNGIEYGDMQLICEAYDIMKRIGRFTDKEISEVFDKWNTGVLDSFLIEITRDILKFDDVDGKPLVEKIMDTAGQKGTGKWTAINALDLGMPVTLIGEAVFARCLSAIKDERKRASKLLAGPTVPKDAIHDREQFVYDLEQALYASKIISYAQGFMLIREAARSYGWKLNNPAIALMWRGGCIIRSVFLAEITKAYRDDPDLENLLFNEFFASAVTKAQSGWRRTIALAATYGIPTPAFSTALAFYDGYRSERLPANLLQAQRDYFGAHTFRILPECASAHLPVDKDIHINWTGHGGNISSSTYQA

SEQ ID NO: 14 - protein, 1235 aa, Saccharomyces cervisiae
MHFRRTMSRVPTLASLEIRYKKSFGHKFRDFIALCGHYFAPVKKYIFPSFIAVHYFYTISLTLITSILLYPIKNTRYIDTLFLAAGAVTQGGLNTVDINNLSLYQQIVLYIVCCISTPIAVHSCLAFVRLYWFERYFDGIRDSSRRNFKMRRTKTILERELTARTMTKNRTGTQRTSYPRKQAKTDDFQEKLFSGEMVNRDEQDSVHSDQNSHDISRDSSNNNTNHNGSSGSLDDFVKEDETDDNGEYQENNSYSTVGSSSNTVADESLNQKPKPSSLRFDEPHSKQRPARVPSEKFAKRRGSRDISPADMYRSIMMLQGKHEATAEDEGPPLVIGSPADGTRYKSNVNKLKKATGINGNKIKIRDKGNESNTDQNSVSSEANSTASVSDESSLHTNFGNKVPSLRTNTHRSNSGPIAITDNAETDKKHGPSIQFDITKPPRKISKRVSTFDDLNPKSSVLYRKKASKKYLMKHFPKARRIRQQIKRRLSTGSIEKNSSNNVSDRKPITDMDDDDDDDDNDGDNNEEYFADNESGDEDERVQQSEPHSDSELKSHQQQQEKHQLQQNLHRMYKTKSFDDNRSRAVPMERSRTIDMAEAKDLNELARTPDFQKMVYQNWKAHHRKKPNFRKRGWNNKIFEHGPYASDSDRNYPDNSNTGNSILHYAESILHHDGSHKNGSEEASSDSNENIYSTNGGSDHNGLNNYPTYNDDEEGYYGLHFDTDYDLDPRHDLSKGSGKTYLSWQPTIGRNSNFLGLTRAQKDELGGVEYRAIKLLCTILVVYYVGWHIVAFVMLVPWIILKKHYSEVVRDDGVSPTWWGFWTAMSAFNDLGLTLTPNSMMSFNKAVYPLIVMIWFIIIGNTGFPILLRCIIWIMFKISPDLSQMRESLGFLLDHPRRCFTLLFPKAATWWLLLTLAGLNITDWILFIILDFGSTVVKSLSKGYRVLVGLFQSVSTRTAGFSVVDLSQLHPSIQVSYMLMMYVSVLPLAISIRRTNVYEEQSLGLYGDMGGEPEDTDTEDDGNDEDDDEENESHEGQSSQRSSSNNNNNNNRKKKKKKKTENPNEISTKSFIGAHLRKQLSFDLWFLFLGLFIICICEGDKIKDVQEPNFNIFAILFEIVSAYGTVGLSLGYPDTNQSFSRQFTTLSKLVIIAMLIRGKNRGLPYSLDRAIILPSDRLEHIDHLEGMKLKRQARTNTEDPMTEHFKRSFTDVKHRWGALKRKTTHSRNPKRSSTTL

SEQ ID NO: 15 - protein, 237 aa, Saccharomyces cervisiae
MAPKKVLLALTSYNDVFYSDGAKTGVFVVEALHPFNTFRKEGFEVDFVSETGKFGWDEHSLAKDFLNGQDETDFKNKDSDFNKTLAKIKTPKEVNADDYQIFFASAGHGTLFDYPKAKDLQDIASEIYANGGVVAAVCHGPAIFDGLTDKKTGRPLIEGKSITGFTDVGETILGVDSILKAKNLATVEDVAKKYGAKYLAPVGPWDDYSITDGRLVTGVNPASAHSTAVRSIDALKN

SEQ ID NO: 16 - protein, 237 aa, Saccharomyces cervisiae
MTPKRALISLTSYHGPFYKDGAKTGVFVVEILRSFDTFEKHGFEVDFVSETGGFGWDEHYLPKSFIGGEDKMNFETKNSAFNKALARIKTANEVNASDYKVFFASAGHGALFDYPKAKNLQDIASKIYANGGVIAAICHGPLLFDGLIDIKTTRPLIEGKAITGFPLEGEIALGVDDILRSRKLTTVERVANKNGAKYLAPIHPWDDYSITDGKLVTGVNANSSYSTTIRAINALYS

SEQ ID NO: 17 - protein, 332 aa, Saccharomyces cervisiae
MNDTLSSFLNRNEALGLNPPHGLDMHITKRGSDWLWAVFAVFGFILLCYVVMFFIAENKGSRLTRYALAPAFLITFFEFFAFFTYASDLGWTGVQAEFNHVKVSKSITGEVPGIRQIFYSKYIAWFLSWPCLLFLIELAASTTGENDDISALDMVHSLLIQIVGTLFWVVSLLVGSLIKSTYKWGYYTIGAVAMLVTQGVICQRQFFNLKTRGFNALMLCTCMVIVWLYFICWGLSDGGNRIQPDGEAIFYGVLDLCVFAIYPCYLLIAVSRDGKLPRLSLTGGFSHHHATDDVEDAAPETKEAVPESPRASGETAIHEPEPEAEQAVEDTA

SEQ ID NO: 18 - protein, 237 aa, Saccharomyces cervisiae
MTPKRALISLTSYHGPFYKDGAKTGVFVVEILRSFDTFEKHGFEVDFVSETGGFGWDEHYLPKSFIGGEDKMNFETKNSAFNKALARIKTANEVNASDYKIFFASAGHGALFDYPKAKNLQDIASKIYANGGVIAAICHGPLLFDGLIDIKTTRPLIEGKAITGFPLEGEIALGVDDILRSRKLTTVERVANKNGAKYLAPIHPWDDYSITDGKLVTGVNANSSYSTTIRAINALYS

SEQ ID NO: 19 - protein, 350 aa, Saccharomyces cervisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFT

SEQ ID NO: 20 - protein, 1483 aa, Saccharomyces cervisiae
MSENNSHNLDEHESHSENSDYMMDTQVEDDYDEDGHVQGEYSYYPDEDEDEHMLSSVGSFEADDGEDDDNDYHHEDDSGLLYGYHRTQNGSDEDRNEEEDGLERSHDNNEFGSNPLHLPDILETFAQRLEQRRQTSEGLGQHPVGRTLPEILSMIGGRMERSAESSARNERISKLIENTGNASEDPYIAMESLKELSENILMMNQMVVDRIIPMETLIGNIAAILSDKILREELELQMQACRCMYNLFEVCPESISIAVDEHVIPILQGKLVEISYIDLAEQVLETVEYISRVHGRDILKTGQLSIYVQFFDFLTIHAQRKAIAIVSNACSSIRTDDFKTIVEVLPTLKPIFSNATDQPILTRLVNAMYGICGALHGVDKFETLFSLDLIERIVQLVSIQDTPLENKLKCLDILTVLAMSSDVLSRELREKTDIVDMATRSFQHYSKSPNAGLHETLIYVPNSLLISISRFIVVLFPPEDERILSADKYTGNSDRGVISNQEKFDSLVQCLIPILVEIYTNAADFDVRRYVLIALLRVVSCINNSTAKAINDQLIKLIGSILAQKETASNANGTYSSEAGTLLVGGLSLLDLICKKFSELFFPSIKREGIFDLVKDLSVDFNNIDLKEDGNENISLSDEEGDLHSSIEECDEGDEEYDYEFTDMEIPDSVKPKKISIHIFRTLSLAYIKNKGVNLVNRVLSQMNVEQEAITEELHQIEGVVSILENPSTPDKTEEDWKGIWSVLKKCIFHEDFDVSGFEFTSTGLASSITKRITSSTVSHFILAKSFLEVFEDCIDRFLEILQSALTRLENFSIVDCGLHDGGGVSSLAKEIKIKLVYDGDASKDNIGTDLSSTIVSVHCIASFTSLNEFLRHRMVRMRFLNSLIPNLTSSSTEADREEEENCLDHMRKKNFDFFYDNEKVDMESTVFGVIFNTFVRRNRDLKTLWDDTHTIKFCKSLEGNNRESEAAEEANEGKKLRDFYKKREFAQVDTGSSADILTLLDFLHSCGVKSDSFINSKLSAKLARQLDEPLVVASGALPDWSLFLTRRFPFLFPFDTRMLFLQCTSFGYGRLIQLWKNKSKGSKDLRNDEALQQLGRITRRKLRISRKTIFATGLKILSKYGSSPDVLEIEYQEEAGTGLGPTLEFYSVVSKYFARKSLNMWRCNSYSYRSEMDVDTTDDYITTLLFPEPLNPFSNNEKVIELFGYLGTFVARSLLDNRILDFRFSKVFFELLHRMSTPNVTTVPSDVETCLLMIELVDPLLAKSLKYIVANKDDNMTLESLSLTFTVPGNDDIELIPGGCNKSLNSSNVEEYIHGVIDQILGKGIEKQLKAFIEGFSKVFSYERMLILFPDELVDIFGRVEEDWSMATLYTNLNAEHGYTMDSSIIHDFISIISAFGKHERRLFLQFLTGSPKLPIGGFKSLNPKFTVVLKHAEDGLTADEYLPSVMTCANYLKLPKYTSKDIMRSRLCQAIEEGAGAFLLS

SEQ ID NO: 21 - protein, 428 aa, Saccharomyces cervisiae
MKDANESKSYTIVIKLGSSSLVDEKTKEPKLAIMSLIVETVVKLRRMGHKVIIVSSGGIAVGLRTMRMNKRPKHLAEVQAIAAIGQGRLIGRWDLLFSQFDQRIAQILLTRNDILDWTQYKNAQNTINELLNMGVIPIVNENDTLSVREIKFGDNDTLSAITSALIHADYLFLLTDVDCLYTDNPRTNPDAMPILVVPDLSKGLPGVNTAGGSGSDVGTGGMETKLVAADLATNAGVHTLIMKSDTPANIGRIVEYMQTLELDDENKVKQAYNGDLTDLQKREFEKLKALNVPLHTKFIANDNKHHLKNREFWILHGLVSKGAVVIDQGAYAALTRKNKAGLLPAGVIDVQGTFHELECVDIKVGKKLPDGTLDPDFPLQTVGKARCNYTSSELTKIKGLHSDQIEEELGYNDSEYVAHRENLAFPPR

SEQ ID NO: 22 - protein, 622 aa, Saccharomyces cervisiae
MRLHYRRRFNFLRRILFILCITSLYLSRDSLKLHAKNVLMDHNVAEYHGGMIDDIQILRCYHWYRQCSSLYAPKLHPSNTAKKIKDKNSILWTRVSKNITVETLYSLQSGPFYNSYLYVHLKDFQSNPKNTIKELAIARDSALIPLQVLRDINKLVKSSDSSVFHNHVYLREKPTSSWWKLLFGISVDTDNIAVFGEEWVYKGSGIWCKYILNDDDNDAPITNLEIYLGSSFIESRPSWKEVIHEFHRNNIPSLPISITRKLETKNHHHKFSNGLLGSLRTPSKDINIQVDADYKITSPHIQFSRGQRSFKILQITDFHFKCTDNSMTVINEIKTVNFIDRVLASENPDLVVITGDLLDSHNTIDYQTCIMKVVQPMISNKIPYAISLGVSDESNLATSAQIRDFIRNLPYTFNNVASEEGHMAIEVSFKKKLTKNTLLERDIDTEDETNPSEALFFVFDSFAPVNNFLQDYNDLIGKIDFGLAFQYFPLSEYRPHGLFPIIGQYNERSTLTVDTPRSRGQVSMTINGKHYKSFLDILSLWNIKGVSCGHEHNNDCCLQSKNEMWLCYGGSAGIGLPRIQGIYPTVRLFNLDDILDEITSWKRNSNLVDEVYDYQYIYKGKQ

SEQ ID NO: 23 - protein, 347 aa, Saccharomyces cervisiae
MTTDTTVFVSGATGFIALHIMNDLLKAGYTVIGSGRSQEKNDGLLKKFNNNPKLSMEIVEDIAAPNAFDEVFKKHGKEIKIVLHTASPFHFETTNFEKDLLTPAVNGTKSILEAIKKYAADTVEKVIVTSSTAALVTPTDMNKGDLVITEESWNKDTWDSCQANAVAAYCGSKKFAEKTAWEFLKENKSSVKFTLSTINPGFVFGPQMFADSLKHGINTSSGIVSELIHSKVGGEFYNYCGPFIDVRDVSKAHLVAIEKPECTGQRLVLSEGLFCCQEIVDILNEEFPQLKGKIATGEPATGPSFLEKNSCKFDNSKTKKLLGFQFYNLKDCIVDTAAQMLEVQNEA

SEQ ID NO: 24 - protein, 274 aa, Saccharomyces cervisiae
MISVMADEKHKEYFKLYYFQYMIIGLCTILFLYSEISLVPRGQNIEFSLDDPSISKRYVPNELVGPLECLILSVGLSNMVVFWTCMFDKDLLKKNRVKRLRERPDGISNDFHFMHTSILCLMLIISINAALTGALKLIIGNLRPDFVDRCIPDLQKMSDSDSLVFGLDICKQTNKWILYEGLKSTPSGHSSFIVSTMGFTYLWQRVFTTRNTRSCIWCPLLALVVMVSRVIDHRHHWYDVVSGAVLAFLVIYCCWKWTFTNLAKRDILPSPVSV

SEQ ID NO: 25 - protein, 947 aa, Saccharomyces cervisiae
MSSTEAKQYKEKPSKEYLHASDGDDPANNSAASSSSSSSTSTSASSSAAAVPRKAAAASAADDSDSDEDIDQLIDELQSNYGEGDESGEEEVRTDGVHAGQRVVPEKDLSTDPAYGLTSDEVARRRKKYGLNQMAEENESLIVKFLMFFVGPIQFVMEAAAILAAGLSDWVDVGVICALLLLNASVGFIQEFQAGSIVDELKKTLANTATVIRDGQLIEIPANEVVPGEILQLESGTIAPADGRIVTEDCFLQIDQSAITGESLAAEKHYGDEVFSSSTVKTGEAFMVVTATGDNTFVGRAAALVGQASGVEGHFTEVLNGIGIILLVLVIATLLLVWTACFYRTVGIVSILRYTLGITIIGVPVGLPAVVTTTMAVGAAYLAKKQAIVQKLSAIESLAGVEILCSDKTGTLTKNKLSLHEPYTVEGVSPDDLMLTACLAASRKKKGLDAIDKAFLKSLIEYPKAKDALTKYKVLEFHPFDPVSKKVTAVVESPEGERIVCVKGAPLFVLKTVEEDHPIPEDVHENYENKVAELASRGFRALGVARKRGEGHWEILGVMPCMDPPRDDTAQTINEARNLGLRIKMLTGDAVGIAKETCRQLGLGTNIYNAERLGLGGGGDMPGSELADFVENADGFAEVFPQHKYRVVEILQNRGYLVAMTGDGVNDAPSLKKADTGIAVEGATDAARSAADIVFLAPGLSAIIDALKTSRQIFHRMYSYVVYRIALSLHLEIFLGLWIAILNNSLDINLIVFIAIFADVATLTIAYDNAPYAPEPVKWNLPRLWGMSIILGIVLAIGSWITLTTMFLPNGGIIQNFGAMNGVMFLQISLTENWLIFVTRAAGPFWSSIPSWQLAGAVFAVDIIATMFTLFGWWSENWTDIVSVVRVWIWSIGIFCVLGGFYYIMSTSQAFDRLMNGKSLKEKKSTRSVEDFMAAMQRVSTQHEKSS

SEQ ID NO: 26 - protein, 1511 aa, Saccharomyces cervisiae
MSSTDEHIEKDISSRSNHDDDYANSVQSYAASEGQVDNEDLAATSQLSRHLSNILSNEEGIERLESMARVISHKTKKEMDSFEINDLDFDLRSLLHYLRSRQLEQGIEPGDSGIAFKNLTAVGVDASAAYGPSVEEMFRNIASIPAHLISKFTKKSDVPLRNIIQNCTGVVESGEMLFVVGRPGAGCSTFLKCLSGETSELVDVQGEFSYDGLDQSEMMSKYKGYVIYCPELDFHFPKITVKETIDFALKCKTPRVRIDKMTRKQYVDNIRDMWCTVFGLRHTYATKVGNDFVRGVSGGERKRVSLVEAQAMNASIYSWDNATRGLDASTALEFAQAIRTATNMVNNSAIVAIYQAGENIYELFDKTTVLYNGRQIYFGPADKAVGYFQRMGWVKPNRMTSAEFLTSVTVDFENRTLDIKPGYEDKVPKSSSEFEEYWLNSEDYQELLRTYDDYQSRHPVNETRDRLDVAKKQRLQQGQRENSQYVVNYWTQVYYCMIRGFQRVKGDSTYTKVYLSSFLIKALIIGSMFHKIDDKSQSTTAGAYSRGGMLFYVLLFASVTSLAEIGNSFSSRPVIVKHKSYSMYHLSAESLQEIITEFPTKFVAIVILCLITYWIPFMKYEAGAFFQYILYLLTVQQCTSFIFKFVATMSKSGVDAHAVGGLWVLMLCVYAGFVLPIGEMHHWIRWLHFINPLTYAFESLVSTEFHHREMLCSALVPSGPGYEGISIANQVCDAAGAVKGNLYVSGDSYILHQYHFAYKHAWRNWGVNIVWTFGYIVFNVILSEYLKPVEGGGDLLLYKRGHMPELGTENADARTASREEMMEALNGPNVDLEKVIAEKDVFTWNHLDYTIPYDGATRKLLSDVFGYVKPGKMTALMGESGAGKTTLLNVLAQRINMGVITGDMLVNAKPLPASFNRSCGYVAQADNHMAELSVRESLRFAAELRQQSSVPLEEKYEYVEKIITLLGMQNYAEALVGKTGRGLNVEQRKKLSIGVELVAKPSLLLFLDEPTSGLDSQSAWSIVQFMRALADSGQSILCTIHQPSATLFEQFDRLLLLKKGGKMVYFGDIGPNSETLLKYFERQSGMKCGVSENPAEYILNCIGAGATASVNSDWHDLWLASPECAAARAEVEELHRTLPGRAVNDDPELATRFAASYMTQIKCVLRRTALQFWRSPVYIRAKFFECVACALFVGLSYVGVNHSVGGAIEAFSSIFMLLLIALAMINQLHVFAYDSRELYEVREAASNTFHWSVLLLCHAAVENFWSTLCQFMCFICYYWPAQFSGRASHAGFFFFFYVLIFPLYFVTYGLWILYMSPDVPSASMINSNLFAAMLLFCGILQPREKMPAFWRRLMYNVSPFTYVVQALVTPLVHNKKVVCNPHEYNIMDPPSGKTCGEFLSTYMDNNTGYLVNPTATENCQYCPYTVQDQVVAKYNVKWDHRWRNFGFMWAYICFNIAAMLICYYVVRVKVWSLKSVLNFKKWFNGPRKERHEKDTNIFQTVPGDENKITKK

SEQ ID NO: 27 - protein, 505 aa, Saccharomyces cervisiae
MSEGPVKFEKNTVISVFGASGDLAKKKTFPALFGLFREGYLDPSTKIFGYARSKLSMEEDLKSRVLPHLKKPHGEADDSKVEQFFKMVSYISGNYDTDEGFDELRTQIEKFEKSANVDVPHRLFYLALPPSVFLTVAKQIKSRVYAENGITRVIVEKPFGHDLASARELQKNLGPLFKEEELYRIDHYLGKELVKNLLVLRFGNQFLNASWNRDNIQSVQISFKERFGTEGRGGYFDSIGIIRDVMQNHLLQIMTLLTMERPVSFDPESIRDEKVKVLKAVAPIDTDDVLLGQYGKSEDGSKPAYVDDDTVDKDSKCVTFAAMTFNIENERWEGVPIMMRAGKALNESKVEIRLQYKAVASGVFKDIPNNELVIRVQPDAAVYLKFNAKTPGLSNATQVTDLNLTYASRYQDFWIPEAYEVLIRDALLGDHSNFVRDDELDISWGIFTPLLKHIERPDGPTPEIYPYGSRGPKGLKEYMQKHKYVMPEKHPYAWPVTKPEDTKDN

SEQ ID NO: 28 - DNA, 1314 bp, Saccharomyces cervisiae
atgtccatcacgaaggtacatgctagaacggtgtatgattctcgcggtaatccgactgttgaggttgaaattacaacagagaatggtctcttcagagcgatcgtcccatctggtgcctccaccggcattcacgaagctgttgaacttagagacgggaacaagtccgaatggatgggaaaaggggtgaccaaggcagtcagtaacgtcaatagtatcatagggcctgctttaatcaagtccgacttatgtgtaaccaatcagaagggcatagacgagctcatgatatcgttagacggaacttctaacaagtcaaggttgggcgccaatgctatccttggtgtttccttgtgcgttgctcgagctgctgccgcacaaaagggaattactctctacaagtatatagccgagttagcggatgctagacaggacccctttgttattcctgttccttttttcaatgttttgaatggtggagcccacgccggtggctctttagctatgcaagaattcaagatcgcgccagtcggggctcagagctttgcagaagccatgaggatgggttcggaggtttaccatcatttgaagatattggcgaaggagcaatatggaccttccgctggaaatgttggtgacgagggtggagtcgcccccgatatcgacactgccgaggacgccttggacatgattgtgaaagccattaacatatgcggttacgagggtagagtgaaagtaggaatcgatagtgctccttctgttttttataaggacgggaaatacgacctaaatttcaaggaaccgaactctgacccatctcactggctcagtccagctcagttagcagaatattatcattcattgctaaagaaatacccaatcatttccctggaagacccctacgccgaagatgattggtcctcgtggtctgccttcctaaagactgtcaatgttcagataattgcagatgacctgacatgcaccaacaagaccaggatcgcccgtgctatagaggagaaatgtgcgaatactctgttgctgaaactcaaccagatcggtactctgactgagtctattgaagccgccaatcaggctttcgatgctggatggggtgtaatgatatcacatagatcaggtgaaaccgaagatccgtttatcgctgatttggtcgttggtttaagatgtggtcaaattaaatcgggcgctttgtcgagatcagaaagactggccaagtataatgaacttttgcgtatcgaagaggaactgggggacgattgtatatatgctggtcataggtttcatgatggaaacaaactataa

SEQ ID NO: 29 - DNA, 2703 bp, Saccharomyces cervisiae
atgcctggaaatttatccttcaaagatagagttgttgtaatcacgggcgctggagggggcttaggtaaggtgtatgcactagcttacgcaagcagaggtgcaaaagtggtcgtcaatgatctaggtggcactttgggtggttcaggacataactccaaagctgcagacttagtggtggatgagataaaaaaagccggaggtatagctgtggcaaattacgactctgttaatgaaaatggagagaaaataattgaaacggctataaaagaattcggcagggttgatgtactaattaacaacgctggaatattaagggatgtttcatttgcaaagatgacagaacgtgagtttgcatctgtggtagatgttcatttgacaggtggctataagctatcgcgtgctgcttggccttatatgcgctctcagaaatttggtagaatcattaacaccgcttcccctgccggtctatttggaaattttggtcaagctaattattcagcagctaaaatgggcttagttggtttggcggaaaccctcgcgaaggagggtgccaaatacaacattaatgttaattcaattgcgccattggctagatcacgtatgacagaaaacgtgttaccaccacatatcttgaaacagttaggaccggaaaaaattgttcccttagtactctatttgacacacgaaagtacgaaagtgtcaaactccatttttgaactcgctgctggattctttggacagctcagatgggagaggtcttctggacaaattttcaatccagaccccaagacatatactcctgaagcaattttaaataagtggaaggaaatcacagactatagggacaagccatttaacaaaactcagcatccatatcaactctcggattataatgatttaatcaccaaagcaaaaaaattacctcccaatgaacaaggctcagtgaaaatcaagtcgctttgcaacaaagtcgtagtagttacgggtgcaggaggtggtcttgggaagtctcatgcaatctggtttgcacggtacggtgcgaaggtagttgtaaatgacatcaaggatcctttttcagttgttgaagaaataaataaactatatggtgaaggcacagccattccagattcccatgatgtggtcaccgaagctcctctcattatccaaactgcaataagtaagtttcagagagtagacatcttggtcaataacgctggtattttgcgtgacaaatcttttttaaaaatgaaagatgaggaatggtttgctgtcctgaaagtccaccttttttccacattttcattgtcaaaagcagtatggccaatatttaccaaacaaaagtctggatttattatcaatactacttctacctcaggaatttatggtaattttggacaggccaattatgccgctgcaaaagccgccattttaggattcagtaaaactattgcactggaaggtgccaagagaggaattattgttaatgttatcgctcctcatgcagaaacggctatgacaaagactatattctcggagaaggaattatcaaaccactttgatgcatctcaagtctccccacttgttgttttgttggcatctgaagaactacaaaagtattctggaagaagggttattggccaattattcgaagttggcggtggttggtgtgggcaaaccagatggcaaagaagttccggttatgtttctattaaagagactattgaaccggaagaaattaaagaaaattggaaccacatcactgatttcagtcgcaacactatcaacccgagctccacagaggagtcttctatggcaaccttgcaagccgtgcaaaaagcgcactcttcaaaggagttggatgatggattattcaagtacactaccaaggattgtatcttgtacaatttaggacttggatgcacaagcaaagagcttaagtacacctacgagaatgatccagacttccaagttttgcccacgttcgccgtcattccatttatgcaagctactgccacactagctatggacaatttagtcgataacttcaattatgcaatgttactgcatggagaacaatattttaagctctgcacgccgacaatgccaagtaatggaactctaaagacacttgctaaacctttacaagtacttgacaagaatggtaaagccgctttagttgttggtggcttcgaaacttatgacattaaaactaagaaactcatagcttataacgaaggatcgttcttcatcaggggcgcacatgtacctccagaaaaggaagtgagggatgggaaaagagccaagtttgctgtccaaaattttgaagtgccacatggaaaggtaccagattttgaggcggagatttctacgaataaagatcaagccgcattgtacaggttatctggcgatttcaatcctttacatatcgatcccacgctagccaaagcagttaaatttcctacgccaattctgcatgggctttgtacattaggtattagtgcgaaagcattgtttgaacattatggtccatatgaggagttgaaagtgagatttaccaatgttgttttcccaggtgatactctaaaggttaaagcttggaagcaaggctcggttgtcgtttttcaaacaattgatacgaccagaaacgtcattgtattggataacgccgctgtaaaactatcgcaggcaaaatctaaactataa

SEQ ID NO: 30 - DNA, 1122 bp, Saccharomyces cervisiae
atggctgccgtcacattacatctaagagctgaaactaaacccctagaggcacgtgctgccttaacacctaccacggttaaaaaactgatagctaagggcttcaaaatatatgtagaggacagtccacaatctactttcaatattaacgaatatcgtcaagcaggtgccattatagtgcctgcaggttcatggaaaaccgctccacgcgacagaatcattataggtttgaaggaaatgcctgaaaccgatactttccctctagtccacgaacacatccagtttgctcactgctacaaagaccaagctgggtggcaaaatgtccttatgagatttattaagggacacggtactctatatgatttggaatttttggaaaatgaccaaggtagaagagttgctgcctttggattttacgctgggttcgcaggtgcagcccttggtgtaagagactgggcattcaagcaaacgcattctgacgatgaagacttgcctgcagtgtcgccttaccccaatgaaaaggcattggttaaagatgttaccaaagattataaagaagccttagccaccggagccagaaagccaaccgtgttaatcattggtgcgctaggaagatgtggttccggtgccatcgatctgttgcacaaagttggtattccagatgctaacatattaaaatgggatatcaaagaaacttcccgtggtggtccctttgacgaaattccacaagctgatatttttatcaattgtatatatctatcgaagccaattgctcctttcactaacatggagaaactgaataatcctaacagaagactaaggaccgtggtggacgtatcagcagacactaccaaccctcacaaccccatcccaatatacactgtggctactgtgtttaacaaacctaccgttctggtacctaccactgccgggcctaaattatctgtcatctctattgatcacttgccttctttgctgccaagagaagcttcagaatttttctctcatgatctcttaccatctttagagctcctacctcaaagaaaaactgctcctgtctgggttagagccaagaaattgttcgatagacattgcgctcgtgttaaaagatcttcaagattgtag

SEQ ID NO: 31 - DNA, 1782 bp, Saccharomyces cervisiae
atggtacgagacttagtgacattgccttcatcactgcccttgattactgctggttttgctactgatcaggttcatttgcttattggtacagggtccacggactcagtaagcgtttgtaagaatagaatccactccattttgaatgctggtggtaatcccatagtagtgaatccctcgtcaccaagccatactaaacaattacaattggaatttggtaagtttgcaaagttcgaaatagtagaaagggagtttaggttatctgatttaactactttggggagagttctggtatgcaaggtagtggatagagtattcgtagatctacccataacacaaagtcgcctatgcgaggagatcttttggcaatgccaaaaactgagaattcccataaatacattccacaaaccagagttttctaccttcaatatgattcctacgtgggtcgacccaaaaggaagtggtttacaaatctcagttactacgaatgggaatggatacatcttggcaaacaggataaaaagagatataatatcacacttacctccaaacatatctgaggtggtgataaacatggggtatttgaaagaccgtattataaacgaagaccataaggccttgttagaggaaaagtactaccagactgacatgtcattacctggatttggctacggcttagatgaggacggttgggagagccataagtttaataagctaattcgtgaatttgaaatgaccagtagagaacagagacttaagagaaccagatggttatctcagataatggagtattacccgatgaacaagctgagtgacatcaagttggaagatttcgagacttcatcttctccaaataaaaagacaaagcaggaaactgtcacagagggtgtagtacctcctaccgatgaaaatattgaaaacggtacaaaacaactacaattatcggaagtgaaaaaagaggagggacctaaaaaactagggaagatttctttagtcggaagtggtccaggctcggtatctatgctaacgataggtgcattacaagaaataaagtctgcagatataatactggcagataaactggtaccgcaagccattttagatttaatacctccaaaaactgaaaccttcatagccaaaaaatttcccggtaatgcagaacgagcacaacaggaattactagctaaaggtttagaatcgttggataatggattgaaagtagtccgtttgaagcaaggtgatccgtatatttttggccgtggtggcgaggaatttaatttcttcaaagatcacggatatattcctgtggttttaccgggcataagctcatccctagcttgtactgtattggctcagatacccgctactcaacgtgatatagcagaccaagtgctcatatgtactgggactgggagaaagggcgctctgcctataattcctgaatttgttgaaagcagaaccaccgtctttctaatggcactgcatcgcgccaacgttctgatcacgggattattgaagcatggctgggatggtgatgtccccgctgcaattgtcgagagaggatcgtgccctgaccagcgtgttactagaactcttcttaaatgggtaccagaagtcgtggaggagattggttcaaggccccccggtgtcttggttgtaggcaaggctgtgaatgcattggttgaaaaagatctgataaattttgacgaatcaagaaaatttgtcattgatgaaggttttagagaatttgaggttgatgtagatagtctatttaagttatactaa

SEQ ID NO: 32 - DNA, 1149 bp, Saccharomyces cervisiae
atgcctaaaaagcaaacgaatttcccagtagataacgaaaacagaccttttagatgtgatacctgtcaccgtggtttccatcggttagaacataaaaagagacacttgagaacacacactggggaaaaacctcatcattgcgcatttcctggttgtgggaaaagtttcagtagaagcgatgaactgaaaaggcacatgagaacgcatacagggcaatctcaaaggagattgaagaaagctagcgtacagaaacaggagtttttgacagtaagcggaattcctaccattgcatcgggcgtgatgatacaccaaccaataccgcaagtcctaccagcaaatatggccataaatgttcaggcagtaaatggaggtaacattatacacgctcccaatgcggtgcacccaatggtgataccaatcatggcccaaccagcccccattcatgcctccgctgcatctttccagcctgcaacttctcctatgccaatttctacatacactccagttccatcgcaatcattcacctctttccagagctctattggctccatacagtcaaatagtgatgtttcatctatcttctcgaacatgaatgttcgcgtaaacactccacgctctgtgccaaactctccgaatgatggatatttacaccagcaacatatcccacagcagtatcagcatcaaactgccagcccttctgttgccaagcagcagaaaacttttgcacattctcttgcatctgcattatctaccttacaaaaaagaacgcctgtaagtgccccttccaccactatagaatcaccatcctcaccaagtgattccagtcatacctctgcatccagcagcgctatctctttgcctttcagcaatgctccttctcagctcgccgtggccaaagaacttgagtccgtctatttagattccaatagatacaccaccaagactaggagggaaagagcaaaattcgaaattcctgaagaacaagaagaagataccaataacagcagcagtggtagtaatgaggaggagcacgagtcgctagatcatgaatctagcaaaagccgaaagaaattgtcaggcgtaaaattgccgcctgtacgtaacctactgaaacaaattgatgttttcaacggtcccaaaagagtttaa

SEQ ID NO: 33 - DNA, 696 bp, Saccharomyces cervisiae
atgtcagcttgcccttgcaacatcgttatactcccagtcgagattttgaagaattcatctaaagatactaagtatagcttgtatacaacaattaatcgaggatatgatgtcccaagactcaaatatggcatcatagttagccctcgagtgcacagccttgagactttattcagtgatctgggctttgacaagaatatagagaaatcctcgctttacttattattaaatgatcctaccttagcataccctaatttccatgaacattttgaacagcttaaaggtgaaacaaacaaagatttatctctaccgacatattatattccgaaggtccagtttttgacagaggcattcgattcagaacataccctagcaaccatcggctacaaaccaaataataaggagagttatgagatcacaggttttacatccatgggtaatggttatggtataaaactattcaattacagtgtaattcatatgatgcggtctcataagtgtaaaagagtggttgcagatattatcatggagcatgacctattgggttactatgaaaagaagcttggctttgtagaggtgcaaaggttcaaagttctcaaagaacagcaccaagtaaaggtatttgacgataaagttgactttaccaaagactttcatgtgatcaaaatgattaaagagttgggaaatcatagattgtag

SEQ ID NO: 34 - DNA, 903 bp, Saccharomyces cervisiae
atgtcaccgtgttatggacaaaacagtgccatcgccaaggggtcttggaacagagaggttttacaagaggtgcaaccgatttatcattggcacgatttcgggcaaaacatgaaagaatattcggcatcacccttagagggggattccagcctgccttccagcctgccttccagcactgaggactgtttactactatcattagaaaacacaatcacagttatagccggaaatcagagacaggcttatgactctacgtcgtctactgaggaaggtacagcacctcaattacggccggatgaaatagcggacagtacacactgtatcacgtcattagttgatccggagttcagagatcttattaattatggacgtcaaaaaggagcaaatcctgtatttattgagagcaatacaacagaacaatcccattcacagtgtattctaggctatccccaaaaatcgcacgtggcacagctatatcacgaccccaaagtactcagcacaatttccgaagggcaaacaaaaagaggaagttaccactgttctcattgttctgaaaagttcgcaacgttagttgagtttgccgcgcacttagacgaattcaaccttgaaagaccgtgtaagtgtcccatagagcaatgtccctggaaaatattgggtttccaacaagcaactggtctgagaagacattgtgcttcccaacatataggagagcttgatatagagatggagaaatcattaaatctaaaagtagaaaaatatccaggactgaattgcccatttcctatctgtcagaaaacgtttaggcgcaaagacgcctataagagacatgtggccatggtgcataacaacgctgattcaagatttaacaagcgtttgaagaaaattttgaacaataccaaatag

SEQ ID NO: 35 - DNA, 2448 bp, Saccharomyces cervisiae
atgggaaacagtccttctactcaggatccatcgcattcaaccaaaaaggagcatggacatcattttcatgatgcattcaataaagatcgtcagggaagcataacctctcaactgtttaacaataggaagagtactcataagagacgcgccagtcatactagcgaacataatggtgccattccccctagaatgcaattacttgcatctcacgatccatcgacagattgtgatgggcgcatgagcagtgatactactatcgataagggcccgtcccatctattcaaaaaggattattctttgtcctctgccgcagacgtaaatgacactacgttggccaatttgactttaagtgatgatcacgatgtgggtgcacccgaagagcaggtgaaatctccgtcgtttttgagcccaggtccatcaatggccactgtcaaacgaactaaaagtgatctggatgatctgtctactttgaattacaccatggttgacgaaacaacagaaaatgaaagaaacgacaaaccacaccatgaaaggcatcgctcaagcatcatagctttgaaaaaaaatcttttagaaagttcagctactgcttccccttctccaacgaggtcttcatcggtgcattcagcatcacttcccgccttgaccaagacggattccattgatattcctgtaagacaaccctattcaaagaaaccatctatccatgcataccaatatcaatatctcaataacgacgaaacattttctgagaactctcaaatggataaagagggaaacagtgatagtgtagatgcagaagcaggcgtacttcaaagtgaagatatggttttgaaccagtctcttttacaaaatgctttaaaaaaggatatgcaacgtctttcaagggtgaattcctctaattcaatgtatactgcagaaaggataagccacgctaataacaatggaaatattgaaaataacacccgtaacaagggaaacgcgggaggctccaacgacgattttaccgcacctatatctgctactgcaaaaatgatgatgaaactgtacggtgataagaccttgatggaaagagatttaaataagcaccataataagacaaagaaagcccagaataaaaaaataaggtcggtttcaaattcgagaagatcctcatttgcttctttgcattctttacaatcaagaaagagtatcttaacaaacggtttgaatttgcagcctttgcatccactacatccaattattaacgacaatgaaagtcaatactctgcaccgcagcatagagaaatatcgcatcactctaattccatgtcgagtatgtcttcaatatcatcaacaaactccaccgaaaatactttagttgttctaaaatggaaagacgatggtaccgtggctgcaaccacggaagtctttatagtaagcacggatattgcttctgctcttaaagagcaaagagagcttaccttggatgagaatgcaagtttagactcagaaaaacagttaaatcctaggatccgcatggtttatgatgatgtgcataaggaatggtttgttccagatctttttttgcctgcaggaatttatagactgcaattctccatcaatggtatattgactcactcaaattttcttcctacagctacggattcagaaggcaattttgtcaactggtttgaagtattgcccggttatcatacgatcgagccatttagaaacgaagcagacatcgactcgcaagtagagccaacactggatgaagaattgcccaagagaccggaacttaaaagatttccttcttcctctcgaaaatcttcctactattctgctaagggtgttgaaaggccaagtacaccgttttcagattatagaggcttaagtaggtccagttcaataaatatgcgtgactcatttgtacgtctgaaggctagcagtttggatttgatggccgaggtcaagcctgagaggttggtgtattcgaacgaaataccaaatttatttaatataggtgacggctctacgatttctgtaaaaggagattctgatgacgtgcatccccaagaacctcccagctttacacatagagttgttgactgtaatcaagatgatttatttgctactttacagcaaggcggtaatattgatgcagaaacagcagaggcggtttttctaagtagatacccagttcctgacttgcccatatacctgaattcatcctatctgaacagaatactaaaccaaagcaatcagaattcagaatcacatgagagggatgaaggtgcgataaatcatattataccccacgtgaatttgaaccatttactgacaagcagtattagagatgaaataatcagcgtagcttgcactactagatatgaggggaaatttatcactcaagtagtttatgcaccttgttattataaaacacaaaagtctcagatcagtaattag

SEQ ID NO: 36 - DNA, 903 bp, Saccharomyces cervisiae
atgaattataatctatccaagtatccagacgacgtgtcgagacttttcaagccaaggccacctttatcttacaaaagaccaaccgattacccatatgcgaagagacaaacaaatccaaatatcactggcgttgcaaacttactatcaacctctttgaagcactatatggaggagtttcctgaaggatctccaaacaaccatctccaaagatacgaagacatcaaactttccaagatcaaaaatgctcaattgttagaccggagactacaaaattggaatcctaacgttgaccctcatatcaaggacacagatccctacagaacgatatttattgggaggctaccatacgatcttgacgaaattgaactgcaaaagtattttgttaagtttggcgagatcgaaaaaattaggatagtcaaggacaagataacccagaagagtaaaggctacgccttcatagttttcaaagacccaataagtagtaaaatggcattcaaggagattggagtacacagaggtatccaaatcaaagacagaatctgcatagtcgacatagaaagaggcagaaccgttaaatatttcaagccaagaagattgggcggcggcctaggaggcagaggctattccaacagagacagcaggcttccaggaaggtttgcaagcgcaagtacatcaaatcccgccgaaagaaattatgctcccaggctgccacgcagggaaacttcttcctccgcatatagcgctgatagatacggcagttccacattggacgcgaggtaccgtggaaacaggccattgctctccgccgccactcctactgctgctgttacttctgtatataaatctagaaactcacggactcgagagtctcaaccagctcccaaagaagcgcccgactattga

SEQ ID NO: 37 - DNA, 999 bp, Saccharomyces cervisiae
atgatcagaattgctattaacggtttcggtagaatcggtagattggtcttgagattggctttgcaaagaaaagacattgaggttgttgctgtcaacgatccatttatctctaacgattatgctgcttacatggtcaagtacgattctactcatggtagatacaagggtactgtttcccatgacgacaagcacatcatcattgatggtgtcaagatcgctacctaccaagaaagagacccagctaacttgccatggggttctctaaagatcgatgtcgctgttgactccactggtgttttcaaggaattggacaccgctcaaaagcacattgacgctggtgccaagaaggttgtcatcactgctccatcttcttctgctccaatgtttgttgttggtgttaaccacactaaatacactccagacaagaagattgtctccaacgcttcttgtaccaccaactgtttggctccattggccaaggttatcaacgatgctttcggtattgaagaaggtttgatgaccactgttcactccatgaccgccactcaaaagactgttgatggtccatcccacaaggactggagaggtggtagaaccgcttccggtaacattatcccatcctctaccggtgctgctaaggctgtcggtaaggtcttgccagaattgcaaggtaagttgaccggtatggctttcagagtcccaaccgtcgatgtttccgttgttgacttgactgtcaagttggaaaaggaagctacttacgaccaaatcaagaaggctgttaaggctgccgctgaaggtccaatgaagggtgttttgggttacaccgaagatgccgttgtctcctctgatttcttgggtgacactcacgcttccatcttcgatgcctccgctggtatccaattgtctccaaagttcgtcaagttgatttcctggtacgataacgaatacggttactccgccagagttgttgacttgatcgaatatgttgccaaggcttaa

SEQ ID NO: 38 - DNA, 1176 bp, Saccharomyces cervisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 39 - DNA, 4143 bp, Saccharomyces cervisiae
atggaaccgttcgcatttggacgaggggcgcctgcattatgcatactaaccgcggccgctcgaataaatctggacaattttgttccgtgttgctgggcacttttccgtctgtctttctttttcccgcttgaccctgcatatattagaaacgaaaacaaagaaacaaggacttcttggatttccatagagttttttttcttcgttaaacattgcctctctcaacacacgtttttctcgaagactcttgcaccaaaaagaaactttagggcgaagaagctaaaagacattggcgatactagaatagatagggcagataaagattttttattagtgccggagccaagtatgtttgtgaacggtaatcaatctaatttcgctaagcccgctggtcaaggtattctgcccattcctaaaaaatctcgaattattaagactgataagccaagaccgttcttgtgtcccacatgcactaggggttttgtcaggcaggagcatttgaagagacatcagcattcgcatacccgtgagaaaccgtatctttgtatcttttgcggtaggtgttttgctcgtagagatttagtgctcaggcatcagcaaaaacttcatgctgctcttgtaggtacgggggatccacggcgaatgacgccagcaccaaattcgacttcttcttttgcctccaagcggcgccattccgtggcggcggatgatccaaccgaccttcatatcattaaaatagccggaaataaagagactattctacccaccccgaagaaccttgctggtaagacatctgaagaattgaaagaggccgtggttgccttggccaaatcaaataatgtagaacttcccgtctcggccccagtaatgaacgataagcgagagaaaactcctcctagtaaggcaggctccctaggatttcgagagttcaagttcagcacgaaaggcgtgccagttcactctgcatcaagcgatgctgttatcgacagggcgaacactccctcttccatgcataagacgaaaagacatgcgtctttctctgcatccagtgcaatgacttacatgtctagtagcaatagcccccaccattcaattaccaatttcgagctcgttgaagacgctccgcatcaagtcggcttttctactccacaaatgaccgcgaagcagctcatggaaagcgtgtcagaattggatttacctccgttaaccctggacgaaccaccgcaagctatcaagtttaacttaaatctatttaacaatgacccctccggacagcaacaacaacaacaacaacaacagcaaaattccacctctagtaccatagtgaacagcaacaatggaagtacagttgctacacctggagtgtatctcttaagtagcggtccatctttaaccgatcttttgacaatgaactctgcacatgcaggtgcgggaggatacatgtctagccaccattcgccatttgatttgggctgcttcagtcatgataaaccgacagtttctgaatttaaccttccgtcaagcttcccgaatactataccgtctaattctactacggcttctaatagttacagtaatttggcaaatcaaacttataggcaaatgagcaatgagcagccgcttatgtcactatctcctaaaaacccaccaacaactgtttcagattcctcttccacgatcaatttcaatccaggcacaaataatttactggaaccatcaatggagcccaatgataaggatagtaatatcgatcctgctgccatagatgacaagtggttatcagagtttattaacaactctgatccaaaatctaccttcaagatcaacttcaatcatttcaatgacattgggtttatttattctccaccttcatcaaggtcatctataccaaacaagtcacctccaaaccattctgctacctcattaaatcatgaaaaagcttctttatcacctcgcttaaacttgagtttgaatggaagcacagatttaccaagtacaccacaaaaccaactaaaggagccttcctattctgaccctatttcccatagttctcataagaggcgtcgtgatagcgtcatgatggactacgatctatccaattttttcagctcaaggcaattggatatttccaaggtattaaacgggacagagcaaaataattctcatgtgaacgacgatgttctcactttgtctttccccggcgaaactgattctaatgcaacacagaaacagctgcctgttcttactccttcggatttgttatctccgttttctgtcccttcagtatctcaagtgctttttaccaatgagctaaggagtatgatgctagccgacaataatatcgattcaggagccttccccacaactagtcaattgaacgattatgtgacttactataaggaagaattccatccatttttttcatttattcatcttccttctatcatacctaatatggacagttatcccttgttattatctatctccatggtcggagcattgtatgggtttcattcgacgcatgcaaaagtgttagctaatgcagctagcacccaaattaggaaaagcttgaaagttagtgagaaaaacccggagacgacagagttatgggttatacagacattagtattgctaacgttctactgtattttcaataaaaatacagccgtgatcaaggggatgcatggtcagttgacgactattattcgtctcttgaaggcctctcgtttaaatttgcccctagagtccctatgccagccgcctattgagagtgatcatattatggaatatgaaaacagtcctcatatgttttcaaaaataagagagcaatacaacgcgccgaatcaaatgaacaaaaactaccaatattttgtattggcgcagtcacgtatcaggacttgccatgcggtattacttatatctaacttattttcttcactggtaggtgctgattgctgttttcattcagtcgatttaaaatgtggtgttccatgctataaagaagaattatatcagtgccgaaattccgatgaatggtcggacctattatgtcaatacaaaataacgttagattcgaaattttcgttgattgaattgtctaatggtaacgaggcatatgaaaattgtttgaggtttctttctacaggcgatagttttttttacggaaatgctagggtttcgttaagtacatgtctatcattgttgatatctatccatgagaaaatacttattgaaagaaataacgcaaggatcagtaataacaacaccaatagcaataacattgagttggacgatattgagtggaagatgacttccagacaacggatcgatacaatgttaaaatactgggaaaacctttatttgaaaaatggtggcatcttgacacctaccgagaatagcatgtcaacaataaacgccaatccagcaatgaggttaataattccggtatatttgtttgccaaaatgagacggtgtttggacctggcacatgttattgagaaaatctggttgaaagattggtccaatatgaataaagctttggaggaagtttgctatgacatgggttcattgagggaagctaccgagtatgcactgaatatggtggatgcgtggacttcattttttacgtacattaaacagggcaagcgcagaattttcaatactcctgtatttgcgaccacatgtatgttcactgcagtattagtgatttcggaatacatgaaatgtgtagaggattgggcacgcgggtacaatgccaacaaccctaactcagcattattggatttttcggaccgtgtcttatggctaaaagcagaaaggattttgagaagattacaaatgaacttgataccgaaggagtgtgatgtgttgaaatcgtacactgatttcttaagatggcaggacaaggatgccctagatttgtcagcactaaatgaagaacaagcacaaagggccatggacccgaataccgatataaatgagacaattcaactaattgtagcggcaagtctatcctccaaatgtttatatttgggtgttcaaatattgggtgatgcgccaatttggcctataatattatcgttcgctcatggtttgcaatcaagagctatctatagtgttacgaaaaaaagaaacactagaatataa

SEQ ID NO: 40 - DNA, 1479 bp, Saccharomyces cervisiae
atgtcaaaggcagtaggtgatttaggcttagttggtttagccgtgatgggtcaaaatttgatcttaaacgcagcggatcacggatttaccgtggttgcttataataggacgcaatcaaaggtagataggtttctagctaatgaggcaaaaggaaaatcaataattggtgcaacttcaattgaggacttggttgcgaaactaaagaaacctagaaagattatgcttttaatcaaagccggtgctccggtcgacactttaataaaggaacttgtaccacatcttgataaaggcgacattattatcgacggtggtaactcacatttcccggacactaacagacgctacgaagagctaacaaagcaaggaattctttttgtgggctctggtgtctcaggcggtgaagatggtgcacgttttggtccatctttaatgcctggtgggtcagcagaagcatggccgcacatcaagaacatctttcaatctattgccgccaaatcaaacggtgagccatgctgcgaatgggtggggcctgccggttctggtcactatgtgaagatggtacacaacggtatcgagtacggtgatatgcagttgatttgcgaggcttacgatatcatgaaacgaattggccggtttacggataaagagatcagtgaagtatttgacaagtggaacactggagttttggattctttcttgattgaaatcacgagggacattttaaaattcgatgacgtcgacggtaagccattggtggaaaaaattatggatactgccggtcaaaagggtactggtaaatggactgcaatcaacgccttggatttaggaatgccagtcactttaattggggaggctgttttcgctcgttgtttgtcagccataaaggacgaacgtaaaagagcttcgaaacttctggcaggaccaacagtaccaaaggatgcaatacatgatagagaacaatttgtgtatgatttggaacaagcattatacgcttcaaagattatttcatatgctcaaggtttcatgctgatccgcgaagctgccagatcatacggctggaaattaaacaacccagctattgctctaatgtggagaggtggctgtataatcagatctgtgttcttagctgagattacgaaggcttatagggacgatccagatttggaaaatttattattcaacgagttcttcgcttctgcagttactaaggcccaatccggttggagaagaactattgcccttgctgctacttacggtattccaactccagctttctctactgctttagcgttttacgacggctatagatctgagaggctaccagcaaacttgttacaagcgcaacgtgattattttggcgctcatacatttagaattttacctgaatgtgcttctgcccatttgccagtagacaaggatattcatatcaattggactgggcacggaggtaatatatcttcctcaacctaccaagcttaa

SEQ ID NO: 41 - DNA, 3708 bp, Saccharomyces cervisiae
atgcattttagaagaacgatgagtagagtgcccacattggcatctcttgaaatacgatataaaaaatctttcggccataaatttcgtgattttattgctctatgtggtcactattttgctccagttaaaaaatatatcttccccagttttatcgcggttcactacttctacacgatatccctgacattaataacttcaatcctgctatatcccattaagaataccagatacattgatacattgtttttagcagcgggcgcagttacacaaggtggcttaaatactgtggatatcaacaatctaagcttataccaacaaattgttctgtatatcgtatgctgcatatcaacaccaattgcagttcatagttgcttggcatttgtacggctttactggtttgagcgctacttcgatggtattagagactcttctagacgaaattttaagatgagaagaacgaaaacaatcttagaaagggaactaacagcaagaaccatgaccaagaatagaacaggtacccaaagaacgtcttatcctaggaaacaagctaaaacagatgatttccaagaaaaattgttcagcggagaaatggttaatagagatgagcaggactcagttcacagcgaccagaattctcatgacattagtagggacagcagcaataataatacgaatcacaatggtagcagtggcagtttagatgatttcgttaaggaagacgaaacggatgacaatggagaatatcaggagaacaactcctactcgacggtaggtagttcgtctaacacagttgcagacgaaagtttaaatcagaagcccaagccaagcagtcttcggtttgatgagccacacagcaaacaaagacccgcaagagttccctcagagaaatttgcaaaaagaaggggttcaagagatattagcccagccgatatgtatcgatccattatgatgctacaaggtaagcatgaagcaactgctgaagatgaaggtccccctttagtcatcgggtcccctgcggatggcacaagatataaaagtaatgtcaataagctaaagaaggccaccggcataaatggtaacaaaatcaagattcgagataagggaaatgaaagtaacactgatcaaaattccgtgtcaagtgaagcaaacagtacggcgagcgtttcggacgaaagctcgttacacacaaattttggtaacaaagtaccttcattaagaacaaatactcatagatcaaattcgggcccgatagccattactgataacgcagaaacagacaaaaagcatgggccatcaattcaattcgatataactaaacctcctagaaaaatttcaaaaagagtttcaaccttcgatgatttgaacccaaaatcttccgttctttatcgaaaaaaagcatcgaagaagtacctcatgaaacattttcctaaagcgcggcgaatacggcaacaaattaagagaaggctttctactggttcaattgagaaaaacagcagtaacaatgtttcagatagaaaacctattactgatatggatgatgatgatgatgacgatgacaacgacggcgataacaacgaagaatactttgctgacaacgaaagcggcgatgaagatgaacgagtacagcagtctgaaccacattctgattcagaactcaaatcgcaccaacaacagcaagaaaaacaccaactgcagcagaacctgcaccgcatgtataaaaccaaatcatttgatgataatcgttcaagagcagttcctatggaacgttccaggaccatcgatatggcagaggctaaggatctaaatgagctcgcaaggacgcctgattttcaaaaaatggtctatcaaaattggaaagcccatcatagaaaaaaaccgaactttaggaagaggggatggaataacaagatatttgaacatggtccctatgcatctgacagcgatcgcaattatcctgataatagtaatactggaaacagtattcttcattacgcagagtctattttacatcatgatggctctcataaaaatggaagcgaagaagcctcttccgactctaatgagaatatctattccacgaatggaggaagcgaccacaatggtcttaacaactatcctacttacaacgacgatgaagaaggctattatggtttacatttcgataccgattatgacctagatcctcgtcatgatttatctaaaggcagtggtaaaacgtatctatcatggcaaccaactattggacgtaactcaaacttccttggattaacaagagcccagaaagatgaattaggtggtgtcgagtacagagcaatcaaacttttatgcaccatattggttgtctactacgttggatggcatattgttgcttttgttatgttagtaccttggattattttgaaaaagcattatagtgaagttgttagagatgatggtgtttcacctacatggtggggattttggacagcaatgagtgcatttaatgatttaggtttgacattaactccaaattcaatgatgtcgtttaacaaagctgtatacccattgatcgttatgatttggtttatcattatcggaaatacagggtttcccatccttcttagatgcatcatttggataatgtttaaaatttctcctgatttatcacagatgagagaaagtttaggttttctcttagaccatccacgtcgttgtttcaccttgctatttcctaaggcagctacatggtggctacttttaacgcttgcaggattgaatataactgattggattttatttattattctagattttggctcaacagttgtgaaatcattatcgaaaggctatagagtccttgtcggcctgtttcaatctgttagcacaagaactgctggattcagcgttgtcgatttaagtcaactgcatccttctatccaagtctcctatatgctaatgatgtatgtctccgtattaccattggccatctctattcgacggacaaatgtttacgaggagcaatctttaggactatatggagatatggggggagaaccagaagatacggatactgaagacgatggtaacgatgaagatgacgacgaggaaaacgagagtcacgaaggtcaaagtagtcaaagaagtagttcgaacaacaacaacaataacaacaggaaaaagaaaaagaaaaagaaaactgaaaatccaaatgaaatatctacaaaatcctttatcggtgcccatttaaggaaacagctttcatttgacttgtggtttctatttttagggttatttatcatttgcatttgtgaaggggacaagataaaggacgtacaagaaccaaactttaatatatttgcaattctttttgaaattgttagcgcttacggtacagttgggctatcgctaggttatccggacaccaaccaatcgttttcaagacagtttactacattatctaagttggtgatcatagctatgctgatcagaggcaagaatagaggtctaccatactcactggatcgtgcaattatcttgcctagtgatagacttgaacatattgaccaccttgagggcatgaaattgaagagacaggctagaaccaatacagaagacccaatgacggaacatttcaagagaagtttcactgatgtgaaacatcgttggggagctcttaagcgtaagaccacacattcccgaaatcctaaaaggagcagcacaacgctctaa

SEQ ID NO: 42 - DNA, 714 bp, Saccharomyces cervisiae
atggccccaaaaaaagttttactcgctcttacctcatataacgatgtattctacagtgacggcgccaagaccggtgtttttgttgtagaagccttacatcccttcaacacattccgaaaagaaggttttgaagtcgattttgtatctgaaaccggaaaatttggttgggatgagcattccttagccaaagattttctaaatggtcaagacgaaacggattttaaaaataaagactcagatttcaacaagacattggctaaaattaagacaccaaaagaggtgaatgccgatgattaccaaattttttttgcatctgcaggccacggtaccttatttgactatcctaaggctaaagacttgcaggacattgcttccgaaatttatgctaacggtggtgttgtcgcagctgtttgtcacggtcctgctatttttgatgggttaacagacaaaaaaacaggaagaccattgatcgaaggtaaatctatcaccggttttactgatgttggtgaaaccattttgggtgttgatagtattttgaaagccaagaatttggcaaccgttgaagatgttgctaaaaaatatggcgctaagtatttagctccggttgggccctgggatgattactctattactgacggaagactggtaacaggtgtgaatcctgcttctgcgcactccactgccgtaagatccatcgacgctttaaaaaactga

SEQ ID NO: 43 - DNA, 714 bp, Saccharomyces cervisiae
atgactccaaaaagagcgctaatatctcttacttcataccacggtcccttctacaaagatggtgcgaaaacaggcgtttttgtagttgagattttgcgatcgttcgatacattcgaaaagcatggtttcgaagtggacttcgtttctgagactggtggatttggctgggatgaacattacttgccaaagagctttattggtggcgaagataagatgaactttgaaacgaaaaattccgccttcaataaggcgttagcgaggatcaagaccgcaaatgaagtcaacgccagcgactataaagtattctttgcatctgctggacatggtgctctatttgactatcccaaagctaaaaatctgcaagatattgcatccaagatatatgccaatgggggtgtgatcgctgccatctgtcatggaccgctccttttcgatggattaatagatatcaaaacaacaagaccattaatcgaaggcaaagctataacaggtttcccactcgagggtgaaatcgccctgggagttgacgacatcttgaggagcagaaaattgacaacggttgaacgcgttgcaaacaagaatggagccaagtacttggcgccaatccatccctgggatgactactctattacagatggaaagctagttacgggtgttaacgcaaattcttcctattcgaccacaattagagctataaacgcattatatagctga

SEQ ID NO: 44 - DNA, 999 bp, Saccharomyces cervisiae
atgaacgatacgctatcaagctttttaaatcgtaacgaggctttagggcttaatccaccacatggcctggatatgcacattaccaagagaggttcggattggttatgggcagtgtttgcagtctttggctttatattgctatgctatgttgtgatgttcttcattgcggagaacaagggctccagattgactagatatgccttagctcctgcatttttgatcactttctttgaattttttgctttcttcacttatgcttctgatttaggttggactggtgttcaagctgaatttaaccacgtcaaggttagcaagtctatcacaggtgaagttcccggtattagacaaatcttttactcgaaatatattgcctggttcttgtcctggccatgccttttatttttaatcgagttagccgctagtactactggtgagaatgacgacatttccgccttggatatggtacattcgctgttaattcaaatcgtgggtaccttattctgggttgtttcgctattagttggttcattgatcaagtccacctacaagtggggttattacaccattggtgctgtcgctatgttggttacccaaggtgtgatatgccaacgtcaattcttcaatttgaaaactagagggttcaatgcacttatgctgtgtacctgcatggtaatcgtttggttgtactttatctgttggggtctaagtgatggtggtaaccgtattcaaccagacggtgaggctatcttttatggtgttttggatttatgtgtatttgccatttatccatgttacttgctaattgcagtcagccgtgatggcaaattgccaaggctatctttgacaggaggattctctcatcaccatgctacggacgatgtggaagatgcggctcctgaaacaaaagaagctgttccagagagcccaagagcatctggagagactgcaatccacgaacccgaacctgaagcagagcaagctgtcgaagatactgcttag

SEQ ID NO: 45 - DNA, 714 bp, Saccharomyces cervisiae
atgactccaaaaagagcgctaatatctcttacttcataccacggtcccttctataaagatggtgcgaaaacaggcgtttttgtagttgagattttgcggtcgttcgatactttcgaaaagcatggtttcgaagtggacttcgtttctgagactggtggatttggctgggatgaacattacttgccaaagagctttattggtggcgaagataagatgaactttgaaacgaaaaattccgccttcaataaggcgttagcgaggatcaagaccgcaaatgaagtcaacgccagcgactataaaatattctttgcatctgctggacatggtgctctatttgactatcccaaagctaaaaatctgcaagatattgcatccaagatatatgccaatgggggtgtgatcgctgccatctgtcatggaccgctccttttcgatggattaatagatatcaaaacaacaagaccattaatcgaaggcaaagctataacaggtttcccactcgagggtgaaatcgccctgggagttgacgacatcttgaggagcagaaaattgacaacggttgaacgcgttgcaaacaagaatggagccaagtacttggcgccaatccatccctgggatgactactctattacagatggaaagctagttacgggtgttaacgcaaattcttcctattcgaccacaattagagctataaacgcattatatagctga

SEQ ID NO: 46 - DNA, 1083 bp, Saccharomyces cervisiae
atgtcttatcctgagaaatttgaaggtatcgctattcaatcacacgaagattggaaaaacccaaagaagacaaagtatgacccaaaaccattttacgatcatgacattgacattaagatcgaagcatgtggtgtctgcggtagtgatattcattgtgcagctggtcattggggcaatatgaagatgccgctagtcgttggtcatgaaatcgttggtaaagttgtcaagctagggcccaagtcaaacagtgggttgaaagtcggtcaacgtgttggtgtaggtgctcaagtcttttcatgcttggaatgtgaccgttgtaagaatgataatgaaccatactgcaccaagtttgttaccacatacagtcagccttatgaagacggctatgtgtcgcagggtggctatgcaaactacgtcagagttcatgaacattttgtggtgcctatcccagagaatattccatcacatttggctgctccactattatgtggtggtttgactgtgtactctccattggttcgtaacggttgcggtccaggtaaaaaagttggtatagttggtcttggtggtatcggcagtatgggtacattgatttccaaagccatgggggcagagacgtatgttatttctcgttcttcgagaaaaagagaagatgcaatgaagatgggcgccgatcactacattgctacattagaagaaggtgattggggtgaaaagtactttgacaccttcgacctgattgtagtctgtgcttcctcccttaccgacattgacttcaacattatgccaaaggctatgaaggttggtggtagaattgtctcaatctctataccagaacaacacgaaatgttatcgctaaagccatatggcttaaaggctgtctccatttcttacagtgctttaggttccatcaaagaattgaaccaactcttgaaattagtctctgaaaaagatatcaaaatttgggtggaaacattacctgttggtgaagccggcgtccatgaagccttcgaaaggatggaaaagggtgacgttagatatagatttaccttagtcggctacgacaaagaattttcagactag

SEQ ID NO: 47 - DNA, 4452 bp, Saccharomyces cervisiae
atgtctgaaaataattcgcacaaccttgatgaacatgagtcccatagcgaaaacagtgattatatgatggatacgcaggtagaagatgactatgatgaggatggccatgtacagggtgagtactcttattatcctgatgaagatgaagatgaacatatgctttctagcgtcggaagttttgaggcagatgatggtgaagatgacgataacgattaccatcatgaagatgattctggacttttatatggatatcatagaactcagaatggcagtgacgaagacagaaatgaagaagaagatggacttgaacgttctcacgataataatgaatttggcagcaaccccctacatttacctgacattttggaaacattcgcacaaagactagaacaaagaagacaaacaagtgaaggacttgggcaacacccggttggaagaacactacccgagattttatcgatgattggaggaaggatggagaggagcgcagagagttcggcaaggaatgagcggatttctaaattgatagagaatactgggaatgcctccgaggatccttatattgcaatggagagtttaaaagaactttctgaaaacatattaatgatgaatcaaatggttgtcgatagaattataccgatggaaaccttaataggtaatatagctgccatactctctgataaaattttacgggaagaattagaattacaaatgcaagcttgtagatgcatgtataatctttttgaggtctgccctgaatctatttcaatagctgttgatgaacacgttataccaattttacaaggaaaattggtagagatcagttacattgacctcgcagaacaagttttagaaacggtggaatatatttctagagtacatgggagagacattttaaaaacgggccaattatcaatctacgtccaattcttcgattttttaactatacatgcgcagaggaaggctatcgcaattgtttcgaacgcctgtagcagtatccgaacggatgactttaagaccattgttgaagtacttccaacgctgaagccaattttctcgaatgcgacagaccaaccaatattaaccaggcttgtaaatgccatgtacggtatttgcggggcgttgcatggggttgacaaatttgagactttgttttcgttggatttaatcgaaagaatagttcagctagtttctattcaggatacccccttggagaataaactgaaatgtttggatattttaaccgtattagcgatgagtagtgatgtactttcaagagaactgagagagaaaactgacattgtcgacatggcaacacgatcattccagcattatagtaaaagtcctaacgcagggttacatgaaacactgatttatgtcccaaacagtttattgattagcatttctagatttatagttgtattgtttcctcccgaggatgaaagaatactgtcagcggataaatataccggaaatagcgaccgtggcgtaatttctaaccaggaaaagtttgattccctagttcaatgtctaattccaattctcgttgaaatttatacaaatgctgctgactttgacgtaagaagatacgtacttattgctttactgagggttgtatcatgcataaataattccacagcaaaggcaatcaatgatcaacttattaagttaatcggatctatcctggcccaaaaagaaacagcgtctaacgctaatggtacttactcatcagaagctggtacactgttggttggtggtctctcgttgcttgacttaatttgtaaaaagttttccgaactgttctttccttccatcaaaagagagggcatttttgatttggttaaggatttgtctgtggatttcaataacattgatttaaaggaagacgggaatgaaaatatttcactttctgacgaagaaggggatttgcatagcagtattgaggaatgtgatgagggtgatgaagaatatgattacgaatttactgatatggaaattcctgattcagtaaaaccaaagaaaatttcaatacacattttcagaactctatctctagcttatattaaaaacaagggtgtgaacctagttaatagagtactttctcagatgaacgttgagcaagaagcaataacagaggagctccatcaaatcgaaggcgttgtttctattttagaaaatccttccactccggacaaaactgaagaggattggaagggaatttggtctgttttaaaaaaatgtattttccatgaagatttcgacgtgtcaggtttcgaatttacttctacagggctagcttcctccataactaaaagaattacatcctcaacggtatcccatttcattcttgctaaatcatttttagaggtatttgaggattgtattgacagatttttagaaatcctacaatctgctctcacaaggctggagaatttctctatagttgattgcggtttacacgatggtggtggtgtatcttcactggctaaagagataaaaattaagttggtttatgatggcgatgcaagcaaagataatattggtactgatttatcatctactatcgtttcggtccattgcatagcttcttttacctcacttaatgagtttttgagacacagaatggtaagaatgcgttttttgaattcattaatcccaaaccttacatcttccagtaccgaagctgatagggaagaagaagaaaattgcttggatcatatgagaaaaaagaactttgactttttttatgataatgaaaaagttgacatggagtctacagtatttggtgtgatatttaatacattcgtcaggcgaaatcgtgacttaaaaactttatgggatgatacacatacaatcaaattttgcaaaagtttagaaggtaacaatagagagagtgaggcagccgaggaagctaatgaggggaaaaagttaagagatttttataaaaaaagagaattcgcacaggttgatactggatcttcagcggatattctgacattgctggattttctacatagctgcggtgttaaaagcgacagttttatcaactcaaaactaagcgctaagctcgctagacaactagatgaaccattggtagtagcaagtggagctttgccggattggtcactatttttgaccaggagattcccatttttgtttccgtttgataccaggatgcttttcctacaatgtacttcatttggttacggaagattgattcaactttggaagaataagagtaaaggctcgaaagatttaaggaatgacgaagctttacaacaacttgggagaattactaggcgtaagctgcggatttcaagaaaaacaatattcgctaccggtctcaagattttatccaagtacggaagtagccctgacgtactggaaattgaatatcaagaagaagcaggaacaggtttaggaccgactttggaattttactccgtagtttccaagtattttgcaagaaagtcgttaaatatgtggcgttgtaactcttatagttacagaagcgaaatggatgttgatactactgacgattatattaccactttattgttcccagagcccctcaacccgttttccaataatgaaaaagttattgaactttttggatatttggggacatttgttgccagatcgttgcttgataatagaattcttgactttagatttagcaaagtcttttttgagttattgcacagaatgtctacgcccaatgtgacgacagtgccgagcgacgttgaaacctgtctgttaatgatcgaattggtagatccgttactcgcaaaatcccttaaatacatagtagcgaataaggatgacaatatgaccctagaatcgttgtccttgacatttaccgttcctggaaatgatgacattgagttgattccggggggttgtaataaatccttgaactcttctaatgttgaagaatatatccatggcgttatcgaccaaatattaggtaagggcattgaaaaacagttaaaagcatttattgaaggtttttcaaaggtgttttcctatgagaggatgctaatactttttccggatgaattagtggatattttcggacgagttgaggaagactggtctatggcaactttatacacaaacttgaacgctgaacatggctatacaatggattcttcaatcattcatgattttatatcaataatatccgcgtttggtaagcatgaaagaagattatttttgcaatttttaacgggatctcccaagcttccaattgggggatttaaaagtttgaacccaaagtttacagttgtgttaaagcatgctgaagatggcctaacagcagacgaatatctaccaagtgtaatgacatgtgctaattatttgaaattgccgaagtatactagcaaagatattatgcggtctcgtctttgtcaagccattgaagagggtgcaggagcttttctactttcctaa

SEQ ID NO: 48 - DNA, 1287 bp, Saccharomyces cervisiae
atgaaggatgctaatgagagtaaatcgtatactatagtgatcaaattaggctcttcatcgctagtagatgaaaaaaccaaagaacctaagttagctatcatgtcgcttattgtcgaaactgtagtcaaattgagaagaatgggacacaaagttatcatcgtgtccagtggtggtattgctgttggtttgaggactatgcgtatgaataaaagaccaaaacatttagcagaagttcaggccatcgcagctattgggcagggtagattgatcgggagatgggatcttctgttttcgcaatttgatcaacgtatcgctcaaattctattgaccagaaatgatattctggactggacccaatataagaacgctcaaaacacaattaatgaattgttgaacatgggcgttattcccattgtgaatgaaaacgacacactatctgttagagaaatcaaatttggtgacaatgacactttatcagcaattacttctgctttaatccatgcagattatcttttcttactgacagatgttgactgtttgtatactgataatccaaggacaaacccagatgccatgccgatcttagttgtcccagatctctcaaagggtttgcccggtgtgaatactgctggtggttcaggttctgacgttgggaccggtggtatggaaactaaattggttgctgcagatttggcaacgaatgccggtgttcatacgttgatcatgaaaagcgatacacctgcgaatataggtagaattgtcgagtatatgcaaactctagaacttgacgatgaaaataaagttaaacaagcatataatggcgatttaacggatttgcaaaaaagagaatttgagaaattaaaggctcttaacgttccactacatacgaagttcattgctaatgataataaacaccatctaaagaatagagagttttggattttacacggtcttgtctctaaaggcgctgttgttatagaccaaggtgcgtacgcagccttaacaaggaaaaataaggcgggattattgccagcaggtgttattgatgttcagggcactttccatgagttagaatgtgttgacataaaagttggtaaaaagttaccagatggcacgttagatccagattttcccttgcaaacagtaggcaaggcaagatgcaattacacgagttctgaattaactaaaattaaaggtttgcacagtgaccaaatcgaagaggaattgggctataatgacagcgaatatgtcgctcatagagaaaatttggcattcccacctcgttga

SEQ ID NO: 49 - DNA, 1869 bp, Saccharomyces cervisiae
atgagattacattatagaagaagatttaattttttaaggagaatactttttatattatgcattacttcattgtatttatcgagagattcactaaagctacatgcaaaaaatgtattaatggatcataatgtagcagaatatcatggcggaatgatagacgatattcaaatcctgcggtgctaccattggtacaggcaatgtagttctttgtatgccccgaaattacacccctccaatactgctaaaaagatcaaagacaaaaacagcattctgtggaccagagtttctaagaatattactgtagagacattgtattcacttcagtctggaccattctacaacagttacttatatgttcatctgaaagatttccaaagtaatccaaaaaacacaataaaagaactagccatagcaagggactcagcactaatacccctacaagtgctgagagacattaataagttggtgaaatcgagcgacagttctgtctttcacaatcatgtgtatctacgagaaaagcctacttcgtcatggtggaagctgcttttcggcatatccgttgatacagataacatagctgtgttcggtgaggagtgggtatacaaggggagcggcatatggtgtaagtatatccttaatgatgatgataatgacgctcctataactaatttggaaatatatctaggatcatcgtttattgaatcgaggccttcttggaaagaagttatccatgaatttcatagaaataacataccttctctgcccatatcaattacaagaaagcttgaaaccaaaaaccatcatcacaaattttctaatggattgctaggttctttgagaacacccagcaaagacattaatatccaagtcgatgcagattacaaaataacatctccccatatacaattttcgaggggacaaagatcattcaaaattctccaaataactgattttcatttcaaatgtacggataatagcatgaccgtaatcaatgaaataaaaacagtaaattttattgatagggtactcgcatcagaaaaccctgatttagttgtgatcacaggtgatttgttagactcacataatactatcgactatcaaacgtgcattatgaaagttgtccaaccaatgatttctaataaaataccctacgcaatttcattgggtgtttctgacgaatccaatttggccacatcggcacaaattagagactttatcaggaatttaccttacacatttaacaacgttgcatcagaagagggtcatatggccatagaagtctcatttaaaaagaagctcacgaagaatactcttttggaaagagacattgataccgaagacgaaacaaacccatcagaggctttatttttcgtctttgattcatttgcgcccgtcaataatttcctacaagattataacgacctgattgggaaaatagactttggcttggcatttcaatattttccattatcggaatataggcctcatggtttatttcctattattgggcagtataatgagaggtctaccttaacagtagatacgccaaggtctagaggacaagtttcaatgacgatcaatggcaaacattacaaaagcttccttgatatcctgagtctttggaatataaagggtgtcagttgcggacatgaacataataatgactgttgcttacagtcaaaaaatgagatgtggttatgttacggtgggtccgctggtataggcttgccgagaatccaaggtatatatccaaccgttagattatttaacttggatgatattttggacgaaataacttcgtggaagaggaatagcaatcttgttgacgaggtttacgattatcagtacatctataaggggaagcaataa

SEQ ID NO: 50 - DNA, 1044 bp, Saccharomyces cervisiae
atgactactgataccactgttttcgtttctggcgcaaccggtttcattgctctacacattatgaacgatctgttgaaagctggctatacagtcatcggctcaggtagatctcaagaaaaaaatgatggcttgctcaaaaaatttaataacaatcccaaactatcgatggaaattgtggaagatattgctgctccaaacgcctttgatgaagttttcaaaaaacatggtaaggaaattaagattgtgctacacactgcctccccattccattttgaaactaccaattttgaaaaggatttactaacccctgcagtgaacggtacaaaatctatcttggaagcgattaaaaaatatgctgcagacactgttgaaaaagttattgttacttcgtctactgctgctctggtgacacctacagacatgaacaaaggagatttggtgatcacggaggagagttggaataaggatacatgggacagttgtcaagccaacgccgttgccgcatattgtggctcgaaaaagtttgctgaaaaaactgcttgggaatttcttaaagaaaacaagtctagtgtcaaattcacactatccactatcaatccgggattcgtttttggtcctcaaatgtttgcagattcgctaaaacatggcataaatacctcctcagggatcgtatctgagttaattcattccaaggtaggtggagaattttataattactgtggcccatttattgacgtgcgtgacgtttctaaagcccacctagttgcaattgaaaaaccagaatgtaccggccaaagattagtattgagtgaaggtttattctgctgtcaagaaatcgttgacatcttgaacgaggaattccctcaattaaagggcaagatagctacaggtgaacctgcgaccggtccaagctttttagaaaaaaactcttgcaagtttgacaattctaagacaaaaaaactactgggattccagttttacaatttaaaggattgcatagttgacaccgcggcgcaaatgttagaagttcaaaatgaagcctaa

SEQ ID NO: 51 - DNA, 825 bp, Saccharomyces cervisiae
atgatctctgtcatggcggatgagaaacataaggagtattttaagctatactactttcagtacatgataattggtctatgtacgatattattcctctattcggagatatccctggtacctaggggccaaaacatcgaatttagtcttgatgaccccagtatatcaaaacgttatgtacctaacgaactcgtgggcccactagaatgtttgattttgagtgttggactgagtaacatggtcgtcttctggacctgcatgtttgacaaggacttactgaagaagaatagagtaaagagactaagagagaggccggacggaatctcgaacgattttcacttcatgcatactagcattctatgtctgatgctgattataagcataaatgctgccctaacaggcgccttaaagttgattataggaaacttgaggcctgactttgttgatagatgtatacctgacctccaaaagatgagtgattcagattctttggtttttggcttggacatttgcaagcagactaacaaatggattctatacgaaggcttaaaaagcactccaagcggacattcaagtttcatagtcagtaccatgggctttacatatctttggcaaagggttttcaccacacgcaatacaagaagttgcatttggtgccctttattagctctagtagtaatggtttcaagggttatcgatcacagacatcattggtacgatgttgtctctggagctgttctagcatttttagtcatttattgttgctggaaatggacatttacaaacttggcgaaaagagacatacttccttcaccggttagtgtttag

SEQ ID NO: 52 - DNA, 2844 bp, Saccharomyces cervisiae
atgtcttccactgaagcaaagcaatacaaggagaaaccctcgaaagagtacctccatgccagtgatggcgatgaccctgcaaataattctgccgcttcttcgtcatcttcgtcttctacatcaacttccgcctcgtcatcggctgcagccgttccacggaaggccgcagccgcttctgccgctgatgattctgactcagatgaagatatagaccaattgattgatgaactacaatctaactacggtgagggtgatgaatctggtgaagaagaagtacgtactgatggggtgcacgctggccaaagggttgttcctgaaaaggacctttctacggaccctgcgtatggtttgacttcggatgaagtcgccaggagaagaaagaaatatgggttaaatcaaatggctgaggagaatgaatcgttgattgtgaagtttttgatgttcttcgtagggcctattcaattcgttatggaggctgctgctattttggctgccggtttgtctgattgggttgatgtcggtgtcatctgtgctttactgctattaaacgcatctgtcggatttattcaagaattccaggcaggttccatcgtagacgagctgaaaaagacgttggccaatactgcaacagttattagagatggccaattgatcgaaattccggctaatgaggtagttcctggtgagattttgcaattggaaagtggcacaattgctcccgcagatggtcgtattgtcactgaagactgttttttgcagatcgatcaatcggccatcactggtgaatccttagccgctgaaaagcattacggtgatgaggtgttctcctcatccactgtgaaaaccggcgaggcttttatggttgttactgccactggtgacaataccttcgtcggtagggctgccgccttagtggggcaggcttccggtgtagagggccatttcactgaagtattgaatggaattggtattatcttacttgttctagttatcgctactttgttgttggtctggaccgcatgtttctatagaacggtcggtattgtaagcattttgagatatactttgggtataaccatcattggtgtcccagtcggtttgccagcagttgttaccacgaccatggctgtcggtgcagcttacttggctaagaagcaagccattgttcaaaagttatctgctattgaatcccttgctggtgttgagattttatgttctgacaagactggtactttaaccaaaaacaagttatctttacacgaaccctacactgtcgaaggcgtttctccggacgacttgatgttgaccgcttgtttagctgcctctagaaagaagaaaggtttggatgctattgataaggcttttttgaagtcattgattgagtatccaaaagctaaagacgccctgaccaagtacaaagttttggaattccatccgttcgaccctgtctcaaaaaaggttaccgctgttgtagaatccccagaaggtgaaagaattgtttgtgtcaagggagccccattgtttgtcttgaagactgttgaagaagatcacccaattccggaagatgtgcatgaaaactacgaaaataaggttgctgaactagcttctagaggtttccgtgctttaggtgttgctagaaagagaggggaaggtcactgggaaatcttgggtgttatgccatgtatggacccccctagagatgacaccgctcaaacaatcaatgaggccagaaaccttggtttgagaatcaagatgttaaccggtgacgctgttggtatcgcgaaagaaacgtgtaggcaattaggacttggtacaaacatttataacgcagaaaggttaggtctgggaggtggaggtgatatgcctggttcagagttggctgattttgttgaaaatgccgatggtttcgcagaagttttcccacagcataaatacagagtcgttgaaatcttgcaaaacagaggttacttggttgctatgactggtgatggtgttaacgatgccccatctttgaagaaggctgatactggtattgctgtcgaaggtgctaccgatgctgccagatcagccgctgatattgttttcttggcccctggtctctctgctattattgatgccttaaagacttctagacagattttccacagaatgtactcctatgttgtttatcgtattgccctatccttacatttggagattttcctgggtttatggattgctattttaaacaactctttggatatcaatttgatcgtttttattgctattttcgcagacgttgccactttaactattgcttatgacaatgctccttatgctcctgaacctgtgaaatggaacctaccaagattatggggtatgtctattattttgggcatagttttagctataggttcttggattactttaaccaccatgttcttgcctaatggtggtattatccaaaattttggtgccatgaatggtgtcatgttcctgcagatttcactaactgaaaattggttaatttttgtcactagagctgctggcccattctggtcttccattccatcgtggcagttagccggtgccgttttcgccgttgatattattgctaccatgtttaccttatttggctggtggtctgaaaactggactgatattgtgtcagtcgttcgtgtctggatttggtccattggtattttttgtgtattgggaggattttactatattatgtccacgtctcaagcctttgataggttgatgaatggtaagtcattaaaggaaaagaagtctacaagaagtgtcgaagatttcatggctgctatgcaaagagtttctactcaacacgaaaaaagcagttag

SEQ ID NO: 53 - DNA, 4536 bp, Saccharomyces cervisiae
atgtcttcgactgacgaacatattgagaaagacatttcgtcgagatcgaaccatgacgatgattatgctaattcggtacaatcctacgctgcctccgaaggccaagttgataatgaggatttggcagccacttctcagctatcccgtcacctttcaaacattctttccaatgaagaaggtattgaaaggttggagtctatggcgagagtcatttcacataagacaaagaaggaaatggactcttttgaaattaatgacttagattttgatttgcgctcactattacattatttgaggtctcgtcaattggaacagggaattgaacctggtgattctggtattgcctttaaaaacctaacagcagtcggtgttgatgcctctgctgcatatgggcctagtgttgaagagatgtttagaaatattgctagtataccggcacatctcataagtaaatttaccaagaaatctgatgtcccattaaggaatattattcaaaattgtacgggtgtcgttgaatctggtgaaatgttatttgtcgtcggtaggccaggtgcaggttgctccactttcctaaagtgtctatctggtgaaacttcagaattagttgatgtacaaggtgaattctcctatgatggtctggaccaaagcgaaatgatgtctaagtataaaggttacgttatttactgtcccgagcttgatttccatttcccaaaaattactgtgaaggaaacaatcgattttgccctaaaatgtaagactcctcgtgttagaattgacaaaatgacgagaaagcaatacgttgataacatcagagatatgtggtgtaccgtttttggtttaagacacacatatgccaccaaagtcggtaacgatttcgtaagaggtgtttctggtggtgaacgtaagcgtgtttccttggttgaagctcaggcaatgaatgcctccatctactcttgggataacgccacaagaggtttggatgcctctactgctttagagtttgcccaagccattagaacggctacaaatatggtaaacaactctgctattgttgctatttaccaagctggtgaaaatatttatgaattatttgataaaactactgttctatataacggtagacagatttacttcggtcctgctgataaagctgttggatatttccaaagaatgggttgggttaaaccaaacagaatgacctctgcggaatttttaacatccgtcacggtcgattttgaaaataggacattggatattaaacctggctatgaagataaagttccaaaatctagttcagagtttgaggaatactggttgaactctgaggattatcaggaacttttaagaacttatgatgattatcaaagtagacaccctgttaatgaaacgagagatagactggatgtggccaagaagcaaagactgcaacaaggccaaagagaaaattctcaatatgttgtcaattattggacacaagtttattattgtatgattcgtggttttcaaagggttaagggtgattcaacgtatactaaggtctacttaagttcttttttgatcaaagctttgattatcggttctatgttccacaaaattgatgacaaaagtcaatccaccacggcaggtgcttattctcgtggtggtatgttattctatgttttattgttcgcttctgttacttccttggccgaaattggtaactctttttctagtagacctgttattgtcaaacacaaatcatattccatgtaccatttgtctgcggaatcgttacaagagattatcactgagttccctactaaatttgtcgctattgtgatactatgtttgattacttactggattccatttatgaaatatgaagctggtgctttcttccagtatattttatatctactgactgtgcaacaatgtacttctttcattttcaagtttgttgctactatgagtaaatctggtgtggatgcccatgccgtcggtggtttatgggtcctgatgctttgtgtttatgctggttttgtcttgccaattggtgaaatgcatcattggattagatggcttcatttcattaatcctttaacttatgcttttgaaagtttagtttccactgaatttcaccacagggaaatgttgtgtagcgccttagtcccatctggtcctggttatgaaggtatttctattgctaaccaagtctgtgatgctgctggtgcggttaagggtaacttgtatgttagcggtgactcttacatcttacaccaatatcatttcgcatataagcatgcttggagaaattggggtgtgaacattgtgtggacttttggttatattgtgttcaatgtcatcttatcagaatatttgaaacctgttgagggaggaggtgacttgctgttatataagagaggtcatatgccggagttaggtaccgaaaatgcagatgcaagaaccgcttccagagaggaaatgatggaggctctgaatggtccaaatgtcgatttagaaaaggtcattgcagaaaaggacgttttcacctggaaccatctggactacaccattccatacgacggagctacaagaaaattattatcggatgtctttggttacgttaagcctggtaagatgaccgccttgatgggtgaatccggtgctggtaaaactaccttgttaaatgttttagcacaaagaatcaatatgggtgtcatcactggtgatatgttagtcaatgccaagcccttgcctgcttctttcaacagatcatgtggttatgttgcgcaagccgataatcatatggccgaattatctgttagggaatccctgagatttgcagccgagttaagacagcaaagttccgttccgttagaggagaaatatgaatatgttgaaaaaattatcacattgctaggtatgcaaaattacgctgaagccttagttggtaagactggtagaggtttgaacgttgaacagagaaagaagttatctattggtgttgaactggttgctaaaccatcattattattgtttttggatgagcctacctctggtctggactctcagtctgcttggtcaattgttcaattcatgagagccttagctgattctggtcaatccattttgtgtacgattcatcaaccctctgctaccttgtttgaacagtttgacagattgttgttgttaaagaaaggtggtaagatggtttactttggtgacattggtccaaattctgaaactttgttgaagtattttgaacgtcaatctggtatgaagtgtggtgtttctgaaaatccagctgaatatattttgaattgtattggtgccggtgccactgctagtgttaactctgattggcacgacttatggcttgcttccccagaatgtgccgctgcaagggctgaagttgaagaattacatcgtactttacctggtagagcagttaatgatgatcctgagttagctacaagatttgctgccagttacatgactcaaatcaaatgtgttttacgtagaacagctcttcaattttggagatcgcctgtctatatcagggccaaattctttgaatgtgtcgcatgtgctttgttcgtcggtttatcatatgttggtgtaaatcactctgttggtggtgccattgaggccttttcgtctattttcatgctattattgattgctctggctatgatcaatcaactgcacgtcttcgcttatgatagtagggaattatatgaggttagagaagccgcttctaacactttccattggagtgtcttgttattatgtcatgctgctgttgaaaacttttggtccacactttgtcagtttatgtgtttcatttgctactactggccagctcaattcagtggacgtgcatctcatgcaggtttcttcttcttcttctatgttttaattttcccattatattttgtcacatatggtctatggatcctgtacatgtctcctgatgttccctcagcttctatgattaattccaatttgtttgctgctatgttactgttctgtggtattttacaaccaagagagaaaatgcctgccttctggagaagattgatgtataatgtatcaccatttacctacgtggttcaagctttggttacaccattagttcacaataaaaaggtcgtttgtaatcctcatgaatacaacatcatggacccaccaagcggaaaaacttgtggtgagtttttatctacctatatggacaataataccggttatttggtaaatccaactgccaccgaaaactgtcaatattgcccatacactgttcaagatcaagttgtggctaaatacaatgtcaaatgggatcacagatggagaaactttggtttcatgtgggcttatatttgcttcaatattgccgctatgttgatttgttactatgttgtaagagttaaggtgtggtctttgaagtctgttttgaatttcaagaaatggtttaatgggccaagaaaggaaagacatgaaaaagataccaacattttccaaacagttccaggtgacgaaaataaaatcacgaagaaataa

SEQ ID NO: 54 - DNA, 1518 bp, Saccharomyces cervisiae
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 55 - protein, 561 aa, Saccharomyces cervisiae
MSTPANYTRVPLCEPEELPDDIQKENEYGTLDSPGHLYQVKSRHGKPLPEPVVDTPPYYISLLTYLNYLILIILGHVHDFLGMTFQKNKHLDLLEHDGLAPWFSNFESFYVRRIKMRIDDCFSRPTTGVPGRFIRCIDRISHNINEYFTYSGAVYPCMNLSSYNYLGFAQSKGQCTDAALESVDKYSIQSGGPRAQIGTTDLHIKAEKLVARFIGKEDALVFSMGYGTNANLFNAFLDKKCLVISDELNHTSIRTGVRLSGAAVRTFKHGDMVGLEKLIREQIVLGQPKTNRPWKKILICAEGLFSMEGTLCNLPKLVELKKKYKCYLFIDEAHSIGAMGPTGRGVCEIFGVDPKDVDILMGTFTKSFGAAGGYIAADQWIIDRLRLDLTTVSYSESMPAPVLAQTISSLQTISGEICPGQGTERLQRIAFNSRYLRLALQRLGFIVYGVADSPVIPLLLYCPSKMPAFSRMMLQRRIAVVVVAYPATPLIESRVRFCMSASLTKEDIDYLLRHVSEVGDKLNLKSNSGKSSYDGKRQRWDIEEVIRRTPEDCKDDKYFVN

SEQ ID NO: 56 - protein, 360 aa, Saccharomyces cervisiae
MSIVYNKTPLLRQFFPGKASAQFFLKYECLQPSGSFKSRGIGNLIMKSAIRIQKDGKRSPQVFASSGGNAGFAAATACQRLSLPCTVVVPTATKKRMVDKIRNTGAQVIVSGAYWKEADTFLKTNVMNKIDSQVIEPIYVHPFDNPDIWEGHSSMIDEIVQDLKSQHISVNKVKGIVCSVGGGGLYNGIIQGLERYGLADRIPIVGVETNGCHVFNTSLKIGQPVQFKKITSIATSLGTAVISNQTFEYARKYNTRSVVIEDKDVIETCLKYTHQFNMVIEPACGAALHLGYNTKILENALGSKLAADDIVIIIACGGSSNTIKDLEEALDSMRKKDTPVIEVADNFIFPEKNIVNLKSA

SEQ ID NO: 57 - protein, 592 aa, Saccharomyces cervisiae
MSELENAHQGPLEGSATVSTNSNSYNEKSGNSTAPGTAGYNDNLAQAKPVSSYISHEGPPKDELEELQKEVDKQLEKKSKSDLLFVSVCCLMVAFGGFVFGWDTGTISGFVRQTDFIRRFGSTRANGTTYLSDVRTGLMVSIFNIGCAIGGIVLSKLGDMYGRKIGLMTVVVIYSIGIIIQIASIDKWYQYFIGRIISGLGVGGITVLAPMLISEVSPKQLRGTLVSCYQLMITFGIFLGYCTNFGTKNYSNSVQWRVPLGLCFAWSIFMIVGMTFVPESPRYLVEVGKIEEAKRSLARANKTTEDSPLVTLEMENYQSSIEAERLAGSASWGELVTGKPQMFRRTLMGMMIQSLQQLTGDNYFFYYGTTIFQAVGLEDSFETAIVLGVVNFVSTFFSLYTVDRFGRRNCLLWGCVGMICCYVVYASVGVTRLWPNGQDQPSSKGAGNCMIVFACFYIFCFATTWAPVAYVLISESYPLRVRGKAMSIASACNWIWGFLISFFTPFITSAINFYYGYVFMGCMVFAYFYVFFFVPETKGLTLEEVNEMYEENVLPWKSTKWIPPSRRTTDYDLDATRNDPRPFYKRMFTKEK

SEQ ID NO: 58 - protein, 320 aa, Saccharomyces cervisiae
MSKPGRTILASKVAETFNTEIINNVEEYKKTHNGQGPLLVGFLANNDPAAKMYATWTQKTSESMGFRYDLRVIEDKDFLEEAIIQANGDDSVNGIMVYFPVFGNAQDQYLQQVVCKEKDVEGLNHVYYQNLYHNVRYLDKENRLKSILPCTPLAIVKILEFLKIYNNLLPEGNRLYGKKCIVINRSEIVGRPLAALLANDGATVYSVDVNNIQKFTRGESLKLNKHHVEDLGEYSEDLLKKCSLDSDVVITGVPSENYKFPTEYIKEGAVCINFACTKNFSDDVKEKASLYVPMTGKVTIAMLLRNMLRLVRNVELSKEK

SEQ ID NO: 59 - protein, 692 aa, Saccharomyces cervisiae
MLSHNALRAFDCSKVIISRRCLTSSTSIYQQSSVHLQETDDGHSGNREKHVSPFERVQNLAADLKNELKAPDSDINEVFNDFKDKIESLKQKLRNPSPMERSHLLANFSSDLLQELSYRSKNMTLDPYQVLNTLCQYKLARSQHFTIVLKYLLYNQSPQDVIALWVKYLETISENPVILLQNSSSRAHMQNIAITTIAYLSLPENTVDINILYKILQIDRKMGQVLPFNMIRRMLSTEFSSLERRDVIIKNLNTLYYQYTVQDSDHFLSQIENAPRWIDLRDLYGQYNKLEGEKNVEIISKFMDKFIDLDKPDQVVTIYNQYSKVFPNSTSLKDCLLRAVSHLRAKSSKEKLDRILAVWNSVIKPGDNIKNTSYATLVNALTDSGNFNHLKEFWEEELPKKFKKDPIVKEAFLLALCQTSPLKYDQVKGELAETVKTKKLFNKVLLLMLDDEKVSEEQFNTFYYNHYPSDGVLPPTLDTLSIKMYANYKFQAEDTRPQFDLLQSVSINPTDYEKVEKITKAFISVCPTVEPIRQLYKQLGTHLNARNYADFISAEFNKPDGTVAEAKNLFSDFLSYQKTRKRNVDNTPLNALLLGFCDKLYKSKHSEYVPYIEKYYNLAKDSSIRVSNLAVSKILFNLATFARNTQQLSDKEVAFINQFMRDLGTNEGFRPNPKDIQILKECDGITVPEKLT

SEQ ID NO: 60 - protein, 389 aa, Saccharomyces cervisiae
MRFSNFLTVSALLTGALGAPAVRHKHEKRDVVTATVHAQVTVVVSGNSGETIVPVNENAVVATTSSTAVASQATTSTLEPTTSANVVTSQQQTSTLQSSEAASTVGSSTSSSPSSSSSTSSSASSSASSSISASGAKGITYSPYNDDGSCKSTAQVASDLEQLTGFDNIRLYGVDCSQVENVLQAKTSSQKLFLGIYYVDKIQDAVDTIKSAVESYGSWDDITTVSVGNELVNGGSATTTQVGEYVSTAKSALTSAGYTGSVVSVDTFIAVINNPDLCNYSDYMAVNAHAYFDENTAAQDAGPWVLEQIERVYTACGGKKDVVITETGWPSKGDTYGEAVPSKANQEAAISSIKSSCGSSAYLFTAFNDLWKDDGQYGVEKYWGILSSD

SEQ ID NO: 61 - protein, 128 aa, Saccharomyces cervisiae
MNSATSETTTNTGAAETTTSTGAAETKTVVTSSISRFNHAETQTASATDVIGHSSSVVSVSETGNTKSLITSGLSTMSQQPRSTPASSIIGSSTASLEISTYVGIANGLLTNNGISVFISTVLLAIVW

SEQ ID NO: 62 - protein, 387 aa, Saccharomyces cervisiae
MDGRNEKPTTPVSDFRVGSSEQSQAGVNLEDSSDHRTSNSAESKKGNLSGKSISDLGISNNDNKNVRFTADTDALENDLSSRSTETSDNSKGTDGQDEEDRPARHKRKPKVSFTHLRNNGKDGDDETFIKKIINNLTGNQGGLVPGLAPIPSENENGKNDIEKNNRNEEIPLSDLADASKIVDVHEGDDKEKLEALKLEGDVNCTSDGETLGSSSKNSFLAPAVDHFDDYAENNSSDDNEGFIETSTYVPPPSQVKSGVLGSLLKLYQNEDQNSSSIFSDSQAVTTDDEGISSTAGNKDVPVAKRSRLQNLKGKAKKGRMPRLKKRLKTEAKITVHIADILQRHRFILRMCRALMMYGAPTHRLEEYMVMTSRVLEIDGQFCIFQVV

SEQ ID NO: 63 - protein, 317 aa, Saccharomyces cervisiae
MDQDFDSLLLGFNDSDSVQKDPTVPNGLDGSVVDPTIADPTAITARKRRPQVKLTAEKLLSDKGLPYVLKNAHKRIRISSKKNSYDNLSNIIQFYQLWAHELFPKAKFKDFMKICQTVGKTDPVLREYRVSLFRDEMGMSFDVGTRETGQDLERQSPMVEEHVTSAEERPIVADSFAQDKRNVNNVDYDNDEDDDIYHLSYRNRRGRVLDERGNNETVLNNVVPPKEDLDALLKTFRVQGPVGLEENEKKLLLGWLDAHRKMEKGSMTEEDVQLIQSLEEWEMNDIEGQHTHYDLLPGGDEFGVDQDELDAMKEMGF

SEQ ID NO: 64 - protein, 763 aa, Saccharomyces cervisiae
MNDSQNCLRQREENSHLNPGNDFGHHQGAECTINHNNMPHRNAYTESTNDTEAKSIVMCDDPNAYQISYTNNEPAGDGAIETTSILLSQPLPLRSNVMSVLVGIFVAVGGFLFGYDTGLINSITDMPYVKTYIAPNHSYFTTSQIAILVSFLSLGTFFGALIAPYISDSYGRKPTIMFSTAVIFSIGNSLQVASGGLVLLIVGRVISGIGIGIISAVVPLYQAEAAQKNLRGAIISSYQWAITIGLLVSSAVSQGTHSKNGPSSYRIPIGLQYVWSSILAVGMIFLPESPRYYVLKDELNKAAKSLSFLRGLPIEDPRLLEELVEIKATYDYEASFGPSTLLDCFKTSENRPKQILRIFTGIAIQAFQQASGINFIFYYGVNFFNNTGVDNSYLVSFISYAVNVAFSIPGMYLVDRIGRRPVLLAGGVIMAIANLVIAIVGVSEGKTVVASKIMIAFICLFIAAFSATWGGVVWVVSAELYPLGVRSKCTAICAAANWLVNFTCALITPYIVDVGSHTSSMGPKIFFIWGGLNVVAVIVVYFAVYETRGLTLEEIDELFRKAPNSVISSKWNKKIRKRCLAFPISQQIEMKTNIKNAGKLDNNNSPIVQDDSHNIIDVDGFLENQIQSNDHMIAADKGSGSLVNIIDTAPLTSTEFKPVEHPPVNYVDLGNGLGLNTYNRGPPSIISDSTDEFYEENDSSYYNNNTERNGANSVNTYMAQLINSSSTTSNDTSFSPSHNSNARTSSNWTSDLASKHSQYTSPQ

SEQ ID NO: 65 - protein, 316 aa, Saccharomyces cervisiae
MAFSDFAAICSKTPLPLCSVIKSKTHLILSNSTIIHDFDPLNLNVGVLPRCYARSIDLANTVIFDVGNAFINIGALGVILIILYNIRQKYTAIGRSEYLYFFQLTLLLIIFTLVVDCGVSPPGSGSYPYFVAIQIGLAGACCWALLIIGFLGFNLWEDGTTKSMLLVRGTSMLGFIANFLASILTFKAWITDHKVATMNASGMIVVVYIINAIFLFVFVICQLLVSLLVVRNLWVTGAIFLGLFFFVAGQVLVYAFSTQICEGFKHYLDGLFFGSICNVFTLMMVYKTWDMTTDDDLEFGVSVSKDGDVVYDNGFM

SEQ ID NO: 66 - protein, 570 aa, Saccharomyces cervisiae
MVAALTYLPTELIQRIFEFTVVETDSQYWLYNLVALIDFSVSSRGGGSITEDFLTNYVRKNLMVLDLTCEATQDSILRAEYGFLKRLLPYIDMDAQYIRVVDLETNADKAQNLKAEKLIVIFDEFSDLKLIETFFPLANSNSNIIEFVFCVRNIKSSFYSPLEKLHIANIVADIDINTLYLDFVDSNIYSDQNFFGIFDPDIFQLINKNYRNFFSKTNEKGKKRPPICKKICFPFVETLNLDYMALDSFFNSILHKLTTKIKTFERNNEFDVDKNLNLNSTTTVAALIIKSILQQFFNNFHISFPNLVTLNFIKMSTYPNNNEITQCCNFIDLSSYVLNKCLSENISINFLFQLHSLKNWSMPKIKEFTGHKFKYDETTFSGSPERYIKSLRGNIKILQEMAINETNDGTCYFRVKLIPEGVEKTQIINWIPFTSSFSDDTFKQRHHLKRPMICLKNNSLRSLTVKIIRIEKCSSIRIQGFYLPNLQELFINNTLCDTTQHQKQASNDMSCIEFTSWNELPQCKKLGFAQLEDDSNYVLNISNLQDHLPNLDLRESFPTFFDIRQKFVVV

SEQ ID NO: 67 - protein, 123 aa, Saccharomyces cervisiae
MNINYYYCYKSICSWIFLNKLDLPVIYKTSSFDISPACDSMSCSPAIARVEKSLDQKFPIENLDLKSEIPCDSISGGVHFFNINELRTTLTELNAIAKPASIGGRVMPQGMSTPIAIGIMNIL

SEQ ID NO: 68 - protein, 55 aa, Saccharomyces cervisiae
MVKLTSIAAGVAAIAAGASAAATTTLSQSDERVNLVELGVYVSDIRAHLAEYYSF

SEQ ID NO: 69 - protein, 77 aa, Saccharomyces cervisiae
MAFERQGKIEKKISYSLFLNGPNVHFGSILFGAVDKSKYAEELCTHPMRQAYNTLDSNSRIIITVQSVAILDGKLVW

SEQ ID NO: 70 - protein, 382 aa, Saccharomyces cervisiae
MNNNSRNNENRSTINRNKRQLQQAKEKNENIHIPRYIRNQPWYYKDTPKEQEGKKPGNDDTSTAEGGEKSDYLVHHRQKAKGGALDIDNNSEPKIGMGIKDEFKLIRPQKMSVRDSHSLSFCRNCGEAGHKEKDCMEKPRKMQKLVPDLNSQKNNGTVLVRATDDDWDSRKDRWYGYSGKEYNELISKWERDKRNKIKGKDKSQTDETLWDTDEEIELMKLELYKDSVGSLKKDDADNSQLYRTSTRLREDKAAYLNDINSTESNYDPKSRLYKTETLGAVDEKSKMFRRHLTGEGLKLNELNQFARSHAKEMGIRDEIEDKEKVQHVLVANPTKYEYLKKKREQEETKQPKIVSIGDLEARKVDGTKQSEEQRNHLKDLYG

SEQ ID NO: 71 - protein, 438 aa, Saccharomyces cervisiae
METPPIVIDNGSYEIKFGPSTNKKPFRALNALAKDKFGTSYLSNHIKNIKDISSITFRRPHELGQLTLWELESCIWDYCLFNPSEFDGFDLKEGKGHHLVASESCMTLPELSKHADQVIFEEYEFDSLFKSPVAVFVPFTKSYKGEMRTISGKDEDIDIVRGNSDSTNSTSSESKNAQDSGSDYHDFQLVIDSGFNCTWIIPVLKGIPYYKAVKKLDIGGRFLTGLLKETLSFRHYNMMDETILVNNIKEQCLFVSPVSYFDSFKTKDKHALEYVLPDFQTSFLGYVRNPRKENVPLPEDAQIITLTDELFTIPETFFHPEISQITKPGIVEAILESLSMLPEIVRPLMVGNIVCTGGNFNLPNFAQRLAAELQRQLPTDWTCHVSVPEGDCALFGWEVMSQFAKTDSYRKARVTREEYYEHGPDWCTKHRFGYQNWI

SEQ ID NO: 72 - protein, 177 aa, Saccharomyces cervisiae
MLKSTLRLSRISLRRGFTTIDCLRQQNSDIDKIILNPIKLAQGSNSDRGQTSKSKTDNADILSMEIPVDMMQSAGRINKRELLSEAEIARSSVENAQMRFNSGKSIIVNKNNPAESFKRLNRIMFENNIPGDKRSQRFYMKPGKVAELKRSQRHRKEFMMGFKRLIEIVKDAKRKGY

SEQ ID NO: 73 - protein, 780 aa, Saccharomyces cervisiae
MAPKSSSSGSKKKSSASSNSADAKASKFKLPAEFITRPHPSKDHGKETCTAYIHPNVLSSLEINPGSFCTVGKIGENGILVIARAGDEEVHPVNVITLSTTIRSVGNLILGDRLELKKAQVQPPYATKVTVGSLQGYNILECMEEKVIQKLLDDSGVIMPGMIFQNLKTKAGDESIDVVITDASDDSLPDVSQLDLNMDDMYGGLDNLFYLSPPFIFRKGSTHITFSKETQANRKYNLPEPLSYAAVGGLDKEIESLKSAIEIPLHQPTLFSSFGVSPPRGILLHGPPGTGKTMLLRVVANTSNAHVLTINGPSIVSKYLGETEAALRDIFNEARKYQPSIIFIDEIDSIAPNRANDDSGEVESRVVATLLTLMDGMGAAGKVVVIAATNRPNSVDPALRRPGRFDQEVEIGIPDVDARFDILTKQFSRMSSDRHVLDSEAIKYIASKTHGYVGADLTALCRESVMKTIQRGLGTDANIDKFSLKVTLKDVESAMVDIRPSAMREIFLEMPKVYWSDIGGQEELKTKMKEMIQLPLEASETFARLGISAPKGVLLYGPPGCSKTLTAKALATESGINFLAVKGPEIFNKYVGESERAIREIFRKARSAAPSIIFFDEIDALSPDRDGSSTSAANHVLTSLLNEIDGVEELKGVVIVAATNRPDEIDAALLRPGRLDRHIYVGPPDVNARLEILKKCTKKFNTEESGVDLHELADRTEGYSGAEVVLLCQEAGLAAIMEDLDVAKVELRHFEKAFKGIARGITPEMLSYYEEFALRSGSSS

SEQ ID NO: 74 - protein, 119 aa, Saccharomyces cervisiae
MPHLAAEAHTWPPHISHSTLSIPHPTPEHRHVFHKKDVKNKRNEEKGNNLLYVLFRTTVIKSSFRSLSTAGRELLFVVHQGHIGTGLIVFIICWRLCLRFLCRVSFQVTVYGGRSRMSA

SEQ ID NO: 75 - protein, 173 aa, Saccharomyces cervisiae
MSFASRNIGRKIAGVGVDIVYLPRFAHILEKYSPFDPCGRSTLNKITRKFMHEKERFHFSNLLIEENCLTPRLHEYIAGVWALKECSLKALCCCVSKHDLPPAQVLYAGMLYKTQTDTGVPQLEFDKMFGKKYPKYQQLSKNYDSLFSTHEFLVSLSHDKDYLIAVTNLVERE

SEQ ID NO: 76 - protein, 521 aa, Saccharomyces cervisiae
MTTRLLQLTRPHYRLLSLPLQKPFNIKRQMSAANPSPFGNYLNTITKSLQQNLQTCFHFQAKEIDIIESPSQFYDLLKTKILNSQNRIFIASLYLGKSETELVDCISQALTKNPKLKVSFLLDGLRGTRELPSACSATLLSSLVAKYGSERVDCRLYKTPAYHGWKKVLVPKRFNEGLGLQHMKIYGFDNEVILSGANLSNDYFTNRQDRYYLFKSRNFSNYYFKLHQLISSFSYQIIKPMVDGSINIIWPDSNPTVEPTKNKRLFLREASQLLDGFLKSSKQSLPITAVGQFSTLVYPISQFTPLFPKYNDKSTEKRTILSLLSTITSNAISWTFTAGYFNILPDIKAKLLATPVAEANVITASPFANGFYQSKGVSSNLPGAYLYLSKKFLQDVCRYRQDHAITLREWQRGVVNKPNGWSYHAKGIWLSARDKNDANNWKPFITVIGSSNYTRRAYSLDLESNALIITRDEELRKKMKAELDNLLQYTKPVTLEDFQSDPERHVGTGVKIATSILGKKL

SEQ ID NO: 77 - protein, 231 aa, Saccharomyces cervisiae
MTRYYCEYCHSYLTHDTLSVRKSHLVGKNHLRITADYYRNKARDIINKHNHKRRHIGKRGRKERENSSQNETLKVTCLSNKEKRHIMHVKKMNQKELAQTSIDTLKLLYDGSPGYSKVFVDANRFDIGDLVKASKLPQRANEKSAHHSFKQTSRSRDETCESNPFPRLNNPKKLEPPKILSQWSNTIPKTSIFYSVDILQTTIKESKKRMHSDGIRKPSSANGYKRRRYGN

SEQ ID NO: 78 - protein, 634 aa, Saccharomyces cervisiae
MLSLKKGITKSYILQRTFTSSSVVRQIGEVKSESKPPAKYHIIDHEYDCVVVGAGGAGLRAAFGLAEAGYKTACLSKLFPTRSHTVAAQGGINAALGNMHPDDWKSHMYDTVKGSDWLGDQDAIHYMTREAPKSVIELEHYGMPFSRTEDGRIYQRAFGGQSKDFGKGGQAYRTCAVADRTGHAMLHTLYGQALKNNTHFFIEYFAMDLLTHNGEVVGVIAYNQEDGTIHRFRAHKTVIATGGYGRAYFSCTSAHTCTGDGNAMVSRAGFPLEDLEFVQFHPSGIYGSGCLITEGARGEGGFLLNSEGERFMERYAPTAKDLASRDVVSRAITMEIRAGRGVGKNKDHILLQLSHLPPEVLKERLPGISETAAVFAGVDVTQEPIPVLPTVHYNMGGIPTKWTGEALTIDEETGEDKVIPGLMACGEAACVSVHGANRLGANSLLDLVVFGRAVANTIADTLQPGLPHKPLASNIGHESIANLDKVRNARGSLKTSQIRLNMQRTMQKDVSVFRTQDTLDEGVRNITEVDKTFEDVHVSDKSMIWNSDLVETLELQNLLTCATQTAVSASKRKESRGAHAREDYAKRDDVNWRKHTLSWQKGTSTPVKIKYRNVIAHTLDENECAPVPPAVRSY

SEQ ID NO: 79 - protein, 554 aa, Saccharomyces cervisiae
MDRDISYQQNYTSTGATATSSRQPSTDNNADTNFLKVMSEFKYNFNSPLPTTTQFPTPYSSNQYQQTQDHFANTDAHNSSSNESSLVENSILPHHQQIQQQQQQQQQQQQQQQALGSLVPPAVTRTDTSETLDDINVQPSSVLQFGNSLPSEFLVASPEQFKEFLLDSPSTNFNFFHKTPAKTPLRFVTDSNGAQQSTTENPGQQQNVFSNVDLNNLLKSNGKTPSSSCTGAFSRTPLSKIDMNLMFNQPLPTSPSKRFSSLSLTPYGRKILNDVGTPYAKALISSNSALVDFQKARKDITTNATSIGLENANNILQRTPLRSNNKKLFIKTPQDTINSTSTLTKDNENKQDIYGSSPTTIQLNSSITKSISKLDNSRIPLLASRSDNILDSNVDDQLFDLGLTRLPLSPTPNCNSLHSTTTGTSALQIPELPKMGSFRSDTGINPISSSNTVSFKSKSGNNNSKGRIKKNGKKPSKFQIIVANIDQFNQDTSSSSLSSSLNASSSAGNSNSNVTKKRASKLKRSQSLLSDSGSKSQARKSCNSKSNGNLFNSQ

SEQ ID NO: 80 - protein, 814 aa, Saccharomyces cervisiae
MKVRKYITLCFWWAFSTSALVSSQQIPLKDHTSRQYFAVESNETLSRLEEMHPNWKYEHDVRGLPNHYVFSKELLKLGKRSSLEELQGDNNDHILSVHDLFPRNDLFKRLPVPAPPMDSSLLPVKEAEDKLSINDPLFERQWHLVNPSFPGSDINVLDLWYNNITGAGVVAAIVDDGLDYENEDLKDNFCAEGSWDFNDNTNLPKPRLSDDYHGTRCAGEIAAKKGNNFCGVGVGYNAKISGIRILSGDITTEDEAASLIYGLDVNDIYSCSWGPADDGRHLQGPSDLVKKALVKGVTEGRDSKGAIYVFASGNGGTRGDNCNYDGYTNSIYSITIGAIDHKDLHPPYSEGCSAVMAVTYSSGSGEYIHSSDINGRCSNSHGGTSAAAPLAAGVYTLLLEANPNLTWRDVQYLSILSAVGLEKNADGDWRDSAMGKKYSHRYGFGKIDAHKLIEMSKTWENVNAQTWFYLPTLYVSQSTNSTEETLESVITISEKSLQDANFKRIEHVTVTVDIDTEIRGTTTVDLISPAGIISNLGVVRPRDVSSEGFKDWTFMSVAHWGENGVGDWKIKVKTTENGHRIDFHSWRLKLFGESIDSSKTETFVFGNDKEEVEPAATESTVSQYSASSTSISISATSTSSISIGVETSAIPQTTTASTDPDSDPNTPKKLSSPRQAMHYFLTIFLIGATFLVLYFMFFMKSRRRIRRSRAETYEFDIIDTDSEYDSTLDNGTSGITEPEEVEDFDFDLSDEDHLASLSSSENGDAEHTIDSVLTNENPFSDPIKQKFPNDANAESASNKLQELQPDVPPSSGRS

SEQ ID NO: 81 - protein, 279 aa, Saccharomyces cervisiae
MVELTITGDDDDILSMFFDEEFVPHAFVDILLSNALNEDQIQTQSVSSLLLTRLDFYTKNLTKELESTIWNLDKLSQTLPRTWASSRYHKEAEQNDSSLYSTESLKSSKLEYYLDTLASAVRALETGMHNVTEKLSDLDNENNRNTNVRQQLQSLMLIKERIEKVVYYLEQVRTVTNISTVRENNTTSTGTDLSITDFRTSLKALEDTIDESLSSAIDNEAKDETNKDLIGRIDSLSELKCLFKGLDKFFAEYSNFSESIKSKAQSYLSTKNIDDGMIS

SEQ ID NO: 82 - protein, 379 aa, Saccharomyces cervisiae
MFSNRLPPPKHSQGRVSTALSSDRVEPAILTDQIAKNVKLDDFIPKRQSNFELSVPLPTKAEIQECTARTKSYIQRLVNAKLANSNNRASSRYVTETHQAPANLLLNNSHHIEVVSKQMDPLLPRFVGKKARKVVAPTENDEVVPVLHMDGSNDRGEADPNEWKIPAAVSNWKNPNGYTVALERRVGKALDNENNTINDGFMKLSEALENADKKARQEIRSKMELKRLAMEQEMLAKESKLKELSQRARYHNGTPQTGAIVKPKKQTSTVARLKELAYSQGRDVSEKIILGAAKRSEQPDLQYDSRFFTRGANASAKRHEDQVYDNPLFVQQDIESIYKTNYEKLDEAVNVKSEGASGSHGPIQFTKAESDDKSDNYGA

SEQ ID NO: 83 - protein, 261 aa, Saccharomyces cervisiae
MKTYHLNNDIIVTQEQLDHWNEQLIKLETPQEIIAWSIVTFPHLFQTTAFGLTGLVTIDMLSKLSEKYYMPELLFIDTLHHFPQTLTLKNEIEKKYYQPKNQTIHVYKPDGCESEADFASKYGDFLWEKDDDKYDYLAKVEPAHRAYKELHISAVFTGRRKSQGSARSQLSIIEIDELNGILKINPLINWTFEQVKQYIDANNVPYNELLDLGYRSIGDYHSTQPVKEGEDERAGRWKGKAKTECGIHEASRFAQFLKQDA

SEQ ID NO: 84 - protein, 129 aa, Saccharomyces cervisiae
MFSSFFGNTCSWVFIFIIIVDNEAFLHFSCLIFVFINIFVFLRGVKDIFSFFFLTRRFSFIVVIYYFFLVPRDQLRISRLFHKRQILCKDSRQLMTCSLGLFFKAQINIFLPPFALTVVQFLVNLVCHT

SEQ ID NO: 85 - protein, 164 aa, Saccharomyces cervisiae
MTKKDKKAKGPKMSTITTKSGESLKVFEDLHDFETYLKGETEDQEFDHVHCQLKYYPPFVLHDAHDDPEKIKETANSHSKKFVRHLHQHVEKHLLKDIKTAINKPELKFHDKKKQESFDRIVWNYGEETELNAKKFKVSVEVVCKHDGAMVDVDYKTEPLQPLI

SEQ ID NO: 86 - protein, 101 aa, Saccharomyces cervisiae
MCTPTTCLLADRDKSGEDRHAETNVLQGMDMLLELLLPVYARLNESGWLLWFVFHDVYEAVKMSTKESVHTRVINFPDILSTQQMRQGPSQIRTPLVMLLM

SEQ ID NO: 87 - protein, 316 aa, Saccharomyces cervisiae
MEEYDYSDVKPLPIETDLQDELCRIMYTEDYKRLMGLARALISLNELSPRALQLTAEIIDVAPAFYTIWNYRFNIVRHMMSESEDTVLYLNKELDWLDEVTLNNPKNYQIWSYRQSLLKLHPSPSFKRELPILKLMIDDDSKNYHVWSYRKWCCLFFSDFQHELAYASDLIETDIYNNSAWTHRMFYWVNAKDVISKVELADELQFIMDKIQLVPQNISPWTYLRGFQELFHDRLQWDSKVVDFATTFIGDVLSLPIGSPEDLPEIESSYALEFLAYHWGADPCTRDNAVKAYSLLAIKYDPIRKNLWHHKINNLN

SEQ ID NO: 88 - protein, 277 aa, Saccharomyces cervisiae
MVGIYRILASFVPLLGLLFAFHDDDMIDTVTIIKTVYETVTSTSTAPAPAATKSVSEKKLDDTKLTLQVIQTMVSCFSVGENPANMISCGLGVVILMFSLIIELINKLENDGINEPQRLYDLIKPKYVELPSNYVNEKIKTTFEPLDLYLGVNMNTSGSELNQNCLILKLGEKTALPFPGLAQQICYTKGASNEFTNYKLSDIQGNLNENSQGIANGVFQKISNIRKISGNFKSQLYQISEKITDENWDGSAVGFTAHGREKGPNKSQISVSFYRDN

SEQ ID NO: 89 - protein, 501 aa, Saccharomyces cervisiae
MLLQGMRLSQRLHKRHLFASKILTWTTNPAHIRHLHDIRPPASNFNTQESAPIPESPANSPTRPQMAPKPNLKKKNRSLMYSIIGVSIVGLYFWFKSNSRKQKLPLSAQKVWKEAIWQESDKMDFNYKEALRRYIEALDECDRSHVDLLSDDYTRIELKIAEMYEKLNMLEEAQNLYQELLSRFFEALNVPGKVDESERGEVLRKDLRILIKSLEINKDIESGKRKLLQHLLLAQEEILSKSPELKEFFENRKKKLSMVKDINRDPNDDFKTFVSEENIKFDEQGYMILDLEKNSSAWEPFKEEFFTARDLYTAYCLSSKDIAAALSCKITSVEWMVMADMPPGQILLSQANLGSLFYLQAEKLEADLNQLEQKKSKESNQELDMGTYIKAVRFVRKNRDLCLERAQKCYDSVIAFAKRNRKIRFHVKDQLDPSIAQSIALSTYGMGVLSLHEGVLAKAEKLFKDSITMAKETEFNELLAEAEKELEKTTVLKAAKKEGLN

SEQ ID NO: 90 - protein, 799 aa, Saccharomyces cervisiae
MSYKVNSSYPDSIPPTEQPYMASQYKQDLQSNIAMATNSEQQRQQQQQQQQQQQQWINQPTAENSDLKEKMNCKNTLNEYIFDFLTKSSLKNTAAAFAQDAHLDRDKGQNPVDGPKSKENNGNQNTFSKVVDTPQGFLYEWWQIFWDIFNTSSSRGGSEFAQQYYQLVLQEQRQEQIYRSLAVHAARLQHDAERRGEYSNEDIDPMHLAAMMLGNPMAPAVQMRNVNMNPIPIPMVGNPIVNNFSIPPYNNANPTTGATAVAPTAPPSGDFTNVGPTQNRSQNVTGWPVYNYPMQPTTENPVGNPCNNNTTNNTTNNKSPVNQPKSLKTMHSTDKPNNVPTSKSTRSRSATSKAKGKVKAGLVAKRRRKNNTATVSAGSTNACSPNITTPGSTTSEPAMVGSRVNKTPRSDIATNFRNQAIIFGEEDIYSNSKSSPSLDGASPSALASKQPTKVRKNTKKASTSAFPVESTNKLGGNSVVTGKKRSPPNTRVSRRKSTPSVILNADATKDENNMLRTFSNTIAPNIHSAPPTKTANSLPFPGINLGSFNKPAVSSPLSSVTESCFDPESGKIAGKNGPKRAVNSKVSASSPLSIATPRSGDAQKQRSSKVPGNVVIKPPHGFSTTNLNITLKNSKIITSQNNTVSQELPNGGNILEAQVGNDSRSSKGNRNTLSTPEEKKPSSNNQGYDFDALKNSSSLLFPNQAYASNNRTPNENSNVADETSASTNSGDNDNTLIQPSSNVGTTLGPQQTSTNENQNVHSQNLKFGNIGMVEDQGPDYDLNLLDTNENDFNFINWEG

SEQ ID NO: 91 - protein, 505 aa, Saccharomyces cervisiae
MKLGIIPYQEGTDIVYKNALQGQQEGKRPNLPQMEATHQIKSSVQGTSYEFVRTEDIPLNRRHFVYRPCSANPFFTILGYGCTEYPFDHSGMSVMDRSEGLSISRDGNDLVSVPDQYGWRTARSDVCIKEGMTYWEVEVIRGGNKKFADGVNNKENADDSVDEVQSGIYEKMHKQVNDTPHLRFGVCRREASLEAPVGFDVYGYGIRDISLESIHEGKLNCVLENGSPLKEGDKIGFLLSLPSIHTQIKQAKEFTKRRIFALNSHMDTMNEPWREDAENGPSRKKLKQETTNKEFQRALLEDIEYNDVVRDQIAIRYKNQLFFEATDYVKTTKPEYYSSDKRERQDYYQLEDSYLAIFQNGKYLGKAFENLKPLLPPFSELQYNEKFYLGYWQHGEARDESNDKNTTSAKKKKQQQKKKKGLILRNKYVNNNKLGYYPTISCFNGGTARIISEEDKLEYLDQIRSAYCVDGNSKVNTLDTLYKEQIAEDIVWDIIDELEQIALQQ

SEQ ID NO: 92 - protein, 264 aa, Saccharomyces cervisiae
MARDITFLTVFLESCGAVNNDEAGKLLSAWTSTVRIEGPESTDSNSLYIPLLPPGMLKIKLNFKMNDRLVTEEQELFTKLREIVGSSIRFWEEQLFYQVQDVSTIENHVILSLKCTILTDAQISTFISKPRELHTHAKGYPEIYYLSELSTTVNFFSKEGNYVEISQVIPHFNEYFSSLIVSQLEFEYPMVFSMISRLRLKWQQSSLAPISYALTSNSVLLPIMLNMIAQDKSSTTAYQILCRRRGPPIQNFQIFSLPAVTYNK

SEQ ID NO: 93 - protein, 420 aa, Saccharomyces cervisiae
MSKIKVVHPIVEMDGDEQTRVIWKLIKEKLILPYLDVDLKYYDLSIQERDRTNDQVTKDSSYATLKYGVAVKCATITPDEARMKEFNLKEMWKSPNGTIRNILGGTVFREPIIIPKIPRLVPHWEKPIIIGRHAFGDQYRATDIKIKKAGKLRLQFSSDDGKENIDLKVYEFPKSGGIAMAMFNTNDSIKGFAKASFELALKRKLPLFFTTKNTILKNYDNQFKQIFDNLFDKEYKEKFQALKITYEHRLIDDMVAQMLKSKGGFIIAMKNYDGDVQSDIVAQGFGSLGLMTSILITPDGKTFESEAAHGTVTRHFRKHQRGEETSTNSIASIFAWTRAIIQRGKLDNTDDVIKFGNLLEKATLDTVQVGGKMTKDLALMLGKTNRSSYVTTEEFIDEVAKRLQNMMLSSNEDKKGMCKL

SEQ ID NO: 94 - protein, 283 aa, Saccharomyces cervisiae
MNSNRCQTNEVNKFISSTEKGPFTGRDNTLSFNKIGSRLNSPPILKDKIELKFLQHSEDLNQSRSYVNIRPRTLEDQSYKFEAPNLNDNETSWAKDFRYNFPKNVEPPIENQIANLNINNGLRTSQTDFPLGFYSQKNFNIASFPVVDHQIFKTTGLEHPINSHIDSLINAEFSELEASSLEEDVHTEEENSGTSLEDEETAMKGLASDIIEFCDNNSANKDVKERLNSSKFMGLMGSISDGSIVLKKDNGTERNLQKHVGFCFQNSGNWAGLEFHDVEDRIA

SEQ ID NO: 95 - protein, 147 aa, Saccharomyces cervisiae
MAVQFILCFNKQGVVRLVRWFDVHSSDPQRSQDAIAQIYRLISSRDHKHQSNFVEFSDSTKLIYRRYAGLYFVMGVDLLDDEPIYLCHIHLFVEVLDAFFGNVCELDIVFNFYKVYMIMDEMFIGGEIQEISKDMLLERLSILDRLD

SEQ ID NO: 96 - protein, 68 aa, Saccharomyces cervisiae
MTMDQGLNPKQFFLDDVVLQDTLCSMSNRVNKSVKTGYLFPKDHVPSANIIAVERRGGLSDIGKNTSN

SEQ ID NO: 97 - protein, 437 aa, Saccharomyces cervisiae
MALNKLKNIPSLTNSSHSSINGIASNAANSKPSGADTDDIDENDESGQSILLNIISQLKPGCDLSRITLPTFILEKKSMLERITNQLQFPDVLLEAHSNKDGLQRFVKVVAWYLAGWHIGPRAVKKPLNPILGEHFTAYWDLPNKQQAFYIAEQTSHHPPESAYFYMIPESNIRVDGVVVPKSKFLGNSSAAMMEGLTVLQFLDIKDANGKPEKYTLSQPNVYARGILFGKMRIELGDHMVIMGPKYQVDIEFKTKGFISGTYDAIEGTIKDYDGKEYYQISGKWNDIMYIKDLREKSSKKTVLFDTHQHFPLAPKVRPLEEQGEYESRRLWKKVTDALAVRDHEVATEEKFQIENRQRELAKKRAEDGVEFHSKLFRRAEPGEDLDYYIYKHIPEGTDKHEEQIRSILETAPILPGQTFTEKFSIPAYKKHGIQKN

SEQ ID NO: 98 - protein, 368 aa, Saccharomyces cervisiae
MARTITFDIPSQYKLVDLIGEGAYGTVCSAIHKPSGIKVAIKKIQPFSKKLFVTRTIREIKLLRYFHEHENIISILDKVRPVSIDKLNAVYLVEELMETDLQKVINNQNSGFSTLSDDHVQYFTYQILRALKSIHSAQVIHRDIKPSNLLLNSNCDLKVCDFGLARCLASSSDSRETLVGFMTEYVATRWYRAPEIMLTFQEYTTAMDIWSCGCILAEMVSGKPLFPGRDYHHQLWLILEVLGTPSFEDFNQIKSKRAKEYIANLPMRPPLPWETVWSKTDLNPDMIDLLDKMLQFNPDKRISAAEALRHPYLAMYHDPSDEPEYPPLNLDDEFWKLDNKIMRPEEEEEVPIEMLKDMLYDELMKTME

SEQ ID NO: 99 - protein, 785 aa, Saccharomyces cervisiae [sequence omitted]

SEQ ID NO: 100 - protein, 114 aa, Saccharomyces cervisiae
MKASYLVLIFISIFSMAQASSLSSYIVTFPKTDNMATDQNSIIEDVKKYVVDIGGKITHEYSLIKGFTVDLPDSDQILDGLKERLSYIESEYGAKCNLEKDSEVHALNRDHLVA

SEQ ID NO: 101 - protein, 313 aa, Saccharomyces cervisiae
MGKGKNKMHEPKNGRPQRGANGHSSRQNHRRMEMKYDNSEKMKFPVKLAMWDFDHCDPKRCSGKKLERLGLIKSLRVGQKFQGIVVSPNGKGVVCPDDLEIVEQHGASVVECSWARLEEVPFNKIGGKHERLLPYLVAANQVNYGRPWRLNCVEALAACFAIVGRMDWASELLSHFSWGMGFLELNKELLEIYQQCTDCDSVKRAEEEWLQKLEKETQERKSRAKEEDIWMMGNINRRGNGSQSDTSESEENSEQSDLEGNNQCIEYDSLGNAIRIDNMKSREAQSEESEDEESGSKENGEPLSYDPLGNLIR

SEQ ID NO: 102 - protein, 280 aa, Saccharomyces cervisiae
MSQEIRQNEKISYRIEGPFFIIHLMNPDNLNALEGEDYIYLGELLELADRNRDVYFTIIQSSGRFFSSGADFKGIAKAQGDDTNKYPSETSKWVSNFVARNVYVTDAFIKHSKVLICCLNGPAIGLSAALVALCDIVYSINDKVYLLYPFANLGLITEGGTTVSLPLKFGTNTTYECLMFNKPFKYDIMCENGFISKNFNMPSSNAEAFNAKVLEELREKVKGLYLPSCLGMKKLLKSNHIDAFNKANSVEVNESLKYWVDGEPLKRFRQLGSKQRKHRL

SEQ ID NO: 103 - protein, 149 aa, Saccharomyces cervisiae
MFKHSTGILSRTVSARSPTLVLRTFTTKAPKIYTFDQVRNLVEHPNDKKLLVDVREPKEVKDYKMPTTINIPVNSAPGALGLPEKEFHKVFQFAKPPHDKELIFLCAKGVRAKTAEELARSYGYENTGIYPGSITEWLAKGGADVKPKK

SEQ ID NO: 104 - protein, 329 aa, Saccharomyces cervisiae
MTTVSINKPNLLKFKHVKSFQPQEKDCGPVTSLNFDDNGQFLLTSSSNDTMQLYSATNCKFLDTIASKKYGCHSAIFTHAQNECIYSSTMKNFDIKYLNLETNQYLRYFSGHGALVNDLKMNPVNDTFLSSSYDESVRLWDLKISKPQVIIPSLVPNCIAYDPSGLVFALGNPENFEIGLYNLKKIQEGPFLIIKINDATFSQWNKLEFSNNGKYLLVGSSIGKHLIFDAFTGQQLFELIGTRAFPMREFLDSGSACFTPDGEFVLGTDYDGRIAIWNHSDSISNKVLRPQGFIPCVSHETCPRSIAFNPKYSMFVTADETVDFYVYDE

SEQ ID NO: 105 - protein, 280 aa, Saccharomyces cervisiae
MKALVEEIDKKTYNPDIYFTSLDPQARRYTSKKINKQGTISTSRPVKRINYSLADLEARLYTSRSEGDGNSISRQDDRNSKNSHSFEERYTQQEILQSDRRFMELNTENFSDLPNVPTLLSDLTGVPRDRIESTTKPISQTSDGLSALMGGSSFVKEHSKYGHGWVLKPETLREIQLSYKSTKLPKPKRKNTNRIVALKKVLSSKRNLHSFLDSALLNLMDKNVIYHNVYNKRYFKVLPLITTCSICGGYDSISSCVNCGNKICSVSCFKLHNETRCRNR

SEQ ID NO: 106 - protein, 445 aa, Saccharomyces cervisiae
MMMLITMKSTVLLSVFTVLATWAGLLEAHPLGDTSDASKLSSDYSLPDLINARKVPNNWQTGEQASLEEGRIVLTSKQNSKGSLWLKQGFDLKDSFTMEWTFRSVGYSGQTDGGISFWFVQDSNVPRDKQLYNGPVNYDGLQLLVDNNGPLGPTLRGQLNDGQKPVDKTKIYDQSFASCLMGYQDSSVPSTIRVTYDLEDDNLLKVQVDNKVCFQTRKVRFPSGSYRIGVTAQNGAVNNNAESFEIFKMQFFNGVIEDSLIPNVNAMGQPKLITKYIDQQTGKEKLIEKTAFDADKDKITNYELYKKLDRVEGKILANDINALETKLNDVIKVQQELLSFMTTITKQLSSKPPANNEKGTSTDDAIAEDKENFKDFLSINQKLEKVLVEQEKYREATKRHGQDGPQVDEIARKLMIWLLPLIFIMLVMAYYTFRIRQEIIKTKLL

SEQ ID NO: 107 - protein, 482 aa, Saccharomyces cervisiae
MPDYDNYTTPLSSRYASKEMSATFSLRNRFSTWRKLWLNLAIAEKELGLTVVTDEAIEQMRKHVEITDDEIAKASAQEAIVRHDVMAHVHTFGETCPAAAGIIHLGATSCFVTDNADLIFIRDAYDIIIPKLVNVINRLAKFAMEYKDLPVLGWTHFQPAQLTTLGKRATLWIQELLWDLRNFERARNDIGLRGVKGTTGTQASFLALFHGNHDKVEALDERVTELLGFDKVYPVTGQTYSRKIDIDVLAPLSSFAATAHKMATDIRLLANLKEVEEPFEKSQIGSSAMAYKRNPMRCERVCSLARHLGSLFSDAVQTASVQWFERTLDDSAIRRISLPSAFLTADILLSTLLNISSGLVVYPKVIERRIKGELPFMATENIIMAMVEKNASRQEVHERIRVLSHQAAAVVKEEGGENDLIERVKRDEFFKPIWEELDSLLEPSTFVGRAPQQVEKFVQKDVNNALQPFQKYLNDEQVKLNV

SEQ ID NO: 108 - protein, 793 aa, Saccharomyces cervisiae
MQVLVTLWCLICTCLVLPVAAKKRTLTASSLVTCMENSQLSANSFDVSFSPDDRSLHYDLDMTTQIDSYIYAYVDVYAYGFKIITENFDVCSMGWKQFCPVHPGNIQIDSIEYIAQKYVKMIPGIAYQVPDIDAYVRLNIYNNVSENLACIQVFFSNGKTVSQIGVKWVTAVIAGIGLLTSAVLSTFGNSTAASHISANTMSLFLYFQSVAVVAMQHVDSVPPIAAAWSENLAWSMGLIRITFMQKIFRWYVEATGGSASLYLTATTMSVLTQRGLDYLKNTSVYKRAENVLYGNSNTLIFRGIKRMGYRMKIENTAIVCTGFTFFVLCGYFLAGFIMACKYSIELCIRCGWMRSDRFYQFRKNWRSVLKGSLLRYIYIGFTQLTILSFWEFTERDSAGVIVIACLFIVLSCGLMAWAAYRTIFFASKSVEMYNNPAALLYGDEYVLNKYGFFYTMFNAKHYWWNALLTTYILVKALFVGFAQASGKTQALAIFIIDLAYFVAIIRYKPYLDRPTNIVNIFICTVTLVNSFLFMFFSNLFNQKYAVSAIMGWVFFIMNAAFSLLLLLMILAFTTIILFSKNPDSRFKPAKDDRASFQKHAIPHEGALNKSVANELMALGNVAKDHTENWEYELKSQEGKSEDNLFGVEYDDEKTGTNSENAESSSKETTRPTFSEKVLRSLSIKRNKSKLGSFKRSAPDKITQQEVSPDRASSSPNSKSYPGVSHTRQESEANNGLINAYEDEQFSLMEPSILEDAASSTQMHAMPARDLSLSSVANAQDVTKKANILDPDYL

SEQ ID NO: 109 - protein, 347 aa, Saccharomyces cervisiae
MDMKVEKLSEDEIALYDRQIRLWGMTAQANMRSAKVLLINLGAIGSEITKSIVLSGIGHLTILDGHMVTEEDLGSQFFIGSEDVGQWKIDATKERIQDLNPRIELNFDKQDLQEKDEEFFQQFDLVVATEMQIDEAIKINTLTRKLNIPLYVAGSNGLFAYVFIDLIEFISEDEKLQSVRPTTVGPISSNRSIIEVTTRKDEEDEKKTYERIKTKNCYRPLNEVLSTATLKEKMTQRQLKRVTSILPLTLSLLQYGLNQKGKAISFEQMKRDAAVWCENLGVPATVVKDDYIQQFIKQKGIEFAPVAAIIGGAVAQDVINILGKRLSPLNNFIVFDGITLDMPLFEF

SEQ ID NO: 110 - protein, 307 aa, Saccharomyces cervisiae
MSEEFPSPQLIDDLEEHPQHDNARVVKDLLAGTAGGIAQVLVGQPFDTTKVRLQTSSTPTTAMEVVRKLLANEGPRGFYKGTLTPLIGVGACVSLQFGVNEAMKRFFHHRNADMSSTLSLPQYYACGVTGGIVNSFLASPIEHVRIRLQTQTGSGTNAEFKGPLECIKKLRHNKALLRGLTPTILREGHGCGTYFLVYEALIANQMNKRRGLERKDIPAWKLCIFGALSGTALWLMVYPLDVIKSVMQTDNLQKPKFGNSISSVAKTLYANGGIGAFFKGFGPTMLRAAPANGATFATFELAMRLLG

SEQ ID NO: 111 - protein, 195 aa, Saccharomyces cervisiae
MIGRGVCCRSFHTAGSAWKQFGFPKTQVTTIYNKTKSASNYKGYLKHRDAPGMYYQPSESIATGSVNSETIPRSFMAASDPRRGLDMPVQSTKAKQCPNVLVGKSTVNGKTYHLGPQEIDEIRKLRLDNPQKYTRKFLAAKYGISPLFVSMVSKPSEQHVQIMESRLQEIQSRWKEKRRIAREDRKRRKLLWYQA

SEQ ID NO: 112 - protein, 760 aa, Saccharomyces cervisiae
MKITCTDLVYVFILLFLNTSCVQAVFSDDAFITDWQLANLGPWEKVIPDSRDRNRVLILSNPTETSCLVSSFNVSSGQILFRNVLPFTIDEIQLDSNDHNAMVCVNSSSNHWQKYDLHDWFLLEEGVDNAPSTTILPQSSYLNDQVSIKNNELHILDEQSKLAEWKLELPQGFNKVEYFHREDPLALVLNVNDTQYMGFSANGTELIPVWQRDEWLTNVVDYAVLDVFDSRDVELNKDMKAELDSNSLWNAYWLRLTTNWNRLINLLKENQFSPGRVFTKLLALDAKDTTVSDLKFGFAKILIVLTHDGFIGGLDMVNKGQLIWKLDLEIDQGVKMFWTDKNHDELVVFSHDGHYLTIEVTKDQPIIKSRSPLSERKTVDSVIRLNEHDHQYLIKFEDKDHLLFKLNPGKNTDVPIVANNHSSSHIFVTEHDTNGIYGYIIENDTVKQTWKKAVNSKEKMVAYSKRETTNLNTLGITLGDKSVLYKYLYPNLAAYLIANEEHHTITFNLIDTITGEILITQEHKDSPDFRFPMDIVFGEYWVVYSYFSSEPVPEQKLVVVELYESLTPDERLSNSSDNFSYDPLTGHINKPQFQTKQFIFPEIIKTMSISKTTDDITTKAIVMELENGQITYIPKLLLNARGKPAEEMAKDKKKEFMATPYTPVIPINDNFIITHFRNLLPGSDSQLISIPTNLESTSIICDLGLDVFCTRITPSGQFDLMSPTFEKGKLLITIFVLLVITYFIRPSVSNKKLKSQWLIK

SEQ ID NO: 113 - protein, 547 aa, Saccharomyces cervisiae
MVKKHQNSKMGNTNHFGHLKSFVGGNVVALGAGTPYLFSFYAPQLLSKCHIPVSASSKLSFSLTIGSSLMGILAGIVVDRSPKLSCLIGSMCVFIAYLILNLCYKHEWSSTFLISLSLVLIGYGSVSGFYASVKCANTNFPQHRGTAGAFPVSLYGLSGMVFSYLCSKLFGENIEHVFIFLMVACGCMILVGYFSLDIFSNAEGDDASIKEWELQKSRETDDNIVPLYENSNDYIGSPVRSSSPATYETYALSDNFQETSEFFALEDRQLSNRPLLSPSSPHTKYDFEDENTSKNTVGENSAQKSMRLHVFQSLKSSTFIGYYIVLGILQGVGLMYIYSVGFMVQAQVSTPPLNQLPINAEKIQSLQVTLLSLLSFCGRLSSGPISDFLVKKFKAQRLWNIVIASLLVFLASNKISHDFSSIEDPSLRASKSFKNISVCSAIFGYSFGVLFGTFPSIVADRFGTNGYSTLWGVLTTGGVFSVSVFTDILGRDFKANTGDDDGNCKKGVLCYSYTFMVTKYCAAFNLLFVLGIIGYTYYRRRATANSL

SEQ ID NO: 114 - DNA, 1686 bp, Saccharomyces cervisiae
atgagtactcctgcaaactatacccgtgtgcccctgtgcgaaccagaggagctgccagacgacatacaaaaagaaaatgaatatggtacactagattctccggggcatttgtatcaagtcaagtcacgtcatgggaagccactacctgagcccgttgtcgacacccctccttattacatttctttgttaacatatctaaattatttgattctgattatattaggtcatgttcacgacttcttaggtatgaccttccaaaaaaacaaacatctggatcttttagagcatgatgggttagcaccttggttttcaaatttcgagagtttttatgtcaggagaattaaaatgagaattgatgattgcttttctagaccaactactggtgttcctggtagatttattcgttgtattgatagaatttctcataatataaatgagtattttacctactcaggcgcagtgtatccatgcatgaacttatcatcatataactatttaggcttcgcacaaagtaagggtcaatgtaccgatgccgccttggaatctgtcgataaatattctattcaatctggtggtccaagagctcaaatcggtaccacagatttgcacattaaagcagagaaattagttgctagatttatcggtaaggaggatgccctcgttttttcgatgggttatggtacaaatgcaaacttgttcaacgctttcctcgataaaaagtgtttagttatctctgacgaattgaaccacacctctattagaacaggtgttaggctttctggtgctgctgtgcgaactttcaagcatggtgatatggtgggtttagaaaagcttatcagagaacagatagtacttggtcaaccaaaaacaaatcgtccatggaagaaaattttaatttgcgcagaagggttgttttccatggaaggtactttgtgtaacttgccaaaattggttgaattgaagaagaaatataaatgttacttgtttatcgatgaagcccattctataggcgctatgggcccaactggtcgcggtgtttgtgaaatatttggcgttgatcccaaggacgtcgacattctaatgggtactttcactaagtcgtttggtgctgctggtggttacattgctgctgatcaatggattatcgatagactgaggttggatttaaccactgtgagttatagtgagtcaatgccggctcctgttttagctcaaactatttcctcattacaaaccattagtggtgaaatatgtcccggacaaggtactgaaagattgcaacgtatagcctttaattcccgttatctacgtttagctttgcaaaggttaggatttattgtctacggtgtggctgactcaccagttattcccttactactgtattgtccctcaaagatgcccgcattttcgagaatgatgttacaaagacggattgctgttgttgttgttgcttatcctgctactccgctgatcgaatcaagagtaagattctgtatgtctgcatctttaacaaaggaagatatcgattatttactgcgtcatgttagtgaagttggtgacaaattgaatttgaaatcaaattccggcaaatccagttacgacggtaaacgtcaaagatgggacatcgaggaagttatcaggagaacacctgaagattgtaaggacgacaagtattttgttaattga

SEQ ID NO: 115 - DNA, 1083 bp, Saccharomyces cervisiae
atgtcgatagtctacaataaaacaccattattacgtcaattcttccccggaaaggcttctgcacaatttttcttgaaatatgaatgccttcaaccaagtggctccttcaaaagtagaggaatcggtaatctcatcatgaaaagtgccattcgaattcaaaaggacggtaaaagatctcctcaggttttcgctagttctggcggtaatgccggttttgctgctgcaacagcatgtcaaagactgtctctaccatgtacagtcgtggttcctacagcgacaaagaagagaatggtagataaaatcaggaacaccggtgcccaggttatcgtgagtggtgcctactggaaagaagcagatacttttttaaaaacaaatgtcatgaataaaatagactctcaggtcattgagcccatttatgttcatcccttcgataatccggatatttgggaaggacattcatctatgatagatgaaatagtacaagatttgaaatcgcaacatatttccgtgaataaggttaaaggcatagtatgcagcgttggtggaggtggtttatacaatggtattattcaaggtttggaaaggtatggtttagctgataggatccctattgtgggggtggaaacgaatggatgtcatgttttcaatacttctttgaaaataggccaaccagttcaattcaagaagataacaagtattgctacttctctaggaacggccgtgatctctaatcaaactttcgaatacgctcgcaaatacaacaccagatccgttgtaatagaggacaaagatgttattgaaacctgtcttaaatatacacatcaattcaatatggtgattgaaccggcatgtggcgccgcattgcatttgggttacaacactaagatcctagaaaatgcactgggctcaaaattagctgcggatgacattgtgataattattgcttgtggcggctcctctaatactataaaggacttggaagaagcgttggatagcatgagaaaaaaagacactcctgtaatagaagtcgctgacaatttcatatttccagaaaaaaatattgtgaatttaaaaagtgcttga

SEQ ID NO: 116 - DNA, 1779 bp, Saccharomyces cervisiae
atgtcggaacttgaaaacgctcatcaaggccccttggaagggtctgctactgtgagcacaaattctaactcatacaacgagaagtcaggaaactcgactgctcctggtaccgccggttacaacgataatttggcacaagctaaacccgtctcaagttacatttcccatgaaggccctcccaaagacgaactggaagagcttcagaaggaggttgacaaacaactagagaagaaatcgaagtcggatttactatttgtatccgtctgctgtttgatggttgcttttggtgggttcgtgtttgggtgggatactggtactatatctggttttgtcaggcaaacagacttcattaggcgatttggcagcacccgtgcaaacgggactacctatctttccgatgtcagaaccggtttgatggtttctattttcaacatcggctgcgctatcggaggtatagttttgtcaaagctcggtgatatgtatggacgtaagattggtctgatgactgttgtcgtcatttactcaattgggatcatcatccaaatcgcctccattgacaaatggtatcaatatttcattggaagaatcatctcaggactgggcgttggtggtattacagttttggcgcctatgctaatttctgaagtgtcgcctaagcagttgcgtggtactctggtttcatgttaccaattaatgatcactttcggtatctttttgggatattgtactaattttggtaccaagaattactcaaactctgtccaatggagggtaccattaggcttatgctttgcatggtctatttttatgattgttggtatgacgttcgttcctgaatccccacgttatctggtagaagtgggaaaaattgaagaggccaagcggtccttagcaagagctaacaaaaccactgaagactctcctttagtaactttagaaatggagaactatcagtcttctattgaagctgagagattggcgggctctgcttcttggggggaattggttactggtaagccccagatgttcagacgtacactaatgggtatgatgattcaatctttacaacagctgacaggtgacaattacttcttttactatggtactacaattttccaggctgttggtttggaagattcatttgaaactgctattgttttgggtgttgttaattttgtttcgacttttttctcgctatataccgtcgatcgttttggtcgtcgtaattgtttgttatggggctgtgtaggtatgatttgttgctatgtcgtctatgcctctgttggtgttaccagattatggccaaacggtcaagatcaaccatcttcaaagggtgctggtaactgtatgattgttttcgcatgtttctacattttctgtttcgctaccacttgggcccccgttgcctatgtccttatctctgagtcgtatcccttaagagtacgtggtaaagcaatgtcgattgcaagtgcctgtaactggatttgggggttcttgatcagttttttcactccatttattacttcagcaatcaatttctattatggctatgtctttatgggttgtatggtgttcgcatacttttatgtgttcttctttgttccagagacaaagggcttaacattagaagaagtcaacgaaatgtatgaagaaaatgtgctaccttggaagtctaccaaatggatcccaccatctaggagaacaacagattatgacctagacgctactagaaatgatccgagaccattttataaaaggatgttcactaaagaaaaataa

SEQ ID NO: 117 - DNA, 963 bp, Saccharomyces cervisiae
atgtcgaagcctggtcgtactattttagcaagcaaggtcgccgaaactttcaataccgaaataattaacaacgtagaggaatacaagaagacacataatggtcaaggtccccttcttgtgggattcctagctaataatgatcctgctgcaaagatgtatgctacatggactcaaaagactagcgagtcaatggggttccgctatgacttaagggtcattgaagataaggattttttggaagaagcgataatacaagctaacggcgatgactctgtgaacggtatcatggtatactttcctgttttcggtaatgctcaagatcagtatttgcaacaggttgtgtgcaaggaaaaagatgtagaagggttaaatcatgtttactaccaaaacctgtaccataatgtcagatacctggacaaagaaaaccgtttgaaatccattctaccttgcacaccactagctatcgttaagatattggaattcttgaaaatttacaacaatttgttaccagaaggaaacagactgtatgggaagaaatgcatagtaattaacaggtcagaaatcgtcggtagaccactggcggcgctattagccaatgacggtgccacagtatactctgtggacgttaacaacattcaaaaattcacccgtggtgaaagtttgaaattaaacaagcatcatgtggaagaccttggggagtactctgaagatctgttgaaaaagtgttctcttgattcagatgtggtcatcactggtgtccctagtgaaaattacaaattccccaccgaatacatcaaagaaggtgccgtctgcatcaattttgcatgcaccaaaaattttagcgatgatgtcaaggaaaaagcttctctttacgttccaatgactggtaaagttaccattgcaatgttgttgagaaacatgttacgtttagtaaggaacgtagaactgtctaaagaaaaatag

SEQ ID NO: 118 - DNA, 2079 bp, Saccharomyces cervisiae
atgctttcccataatgctttaagggcctttgattgttcaaaggtgattatttcacgaagatgtctaacctcttcaacatcgatataccaacaaagcagcgttcacttacaagaaacagatgatggacattcaggaaatagagaaaagcacgtctcaccgtttgaaagggtacaaaatttggctgctgatttgaagaacgagttgaaagctccagattcagatatcaatgaagtttttaatgactttaaagataagattgaatcgttgaaacagaaattaaggaacccttcacctatggaaagatcacacttgttagcgaatttttcttcggatctcctacaggaattaagttacagaagcaaaaatatgacgctagatccttatcaagtattaaacacattgtgccaatacaaattggcacgctcacaacatttcacgattgttttaaagtaccttctatataatcaatcaccacaggacgttattgccttatgggtgaagtacttggaaaccatttccgaaaacccagtgatcttacttcaaaatagttcttctcgtgcacatatgcaaaatattgcaattaccaccattgcttacttatctttaccagagaatactgtggatatcaatattctgtataagattttacagatcgatcgtaaaatgggccaggttttaccttttaacatgattagaagaatgttaagtacagaatttagctctcttgaaagaagagacgtgattatcaaaaatctaaacactttgtactatcaatacacagtacaggatagtgatcatttcttaagtcaaattgaaaatgctcctagatggatagatttaagggatctttatggccaatacaataaacttgaaggtgagaaaaatgtagagatcataagcaagttcatggacaagtttattgatttggataaacccgaccaagttgttactatttataaccagtatagcaaggttttcccaaatagtacgtcgctgaaagattgtcttttaagagctgtgtcgcacttacgagctaaatcgagtaaagagaagttggacagaattctagcagtctggaacagtgttatcaaaccaggagataatattaaaaacacatcttatgcgacgctagttaacgcactaactgattctggaaatttcaaccatttaaaggaattttgggaagaagaacttcctaaaaagttcaagaaagatcccatcgtgaaggaagcatttctcctggccttatgtcaaacttcgcctctaaagtatgaccaagtcaaaggggagttagcagagactgttaaaaccaagaagttgttcaataaagttttattgctaatgttagatgatgaaaaagtgagcgaagaacaattcaacacattttactataaccattatccatcagatggtgtgttaccccctactttggatactctaagcattaaaatgtacgctaattataaatttcaggcagaagatacacgcccacaattcgatctattgcaaagtgtttccattaatcccaccgattatgaaaaggttgaaaagattacgaaagcctttatttcagtgtgccccactgtcgagccgattcgtcaactttacaaacaattgggaactcacttaaatgctaggaattatgcagactttatttccgcagagtttaataagcctgacggcacagtggccgaggcaaagaatttgttttctgattttctctcatatcaaaagactagaaagagaaacgtggataatacgcctctaaatgctttattattggggttctgtgataaactttacaagagtaaacatagcgagtacgttccctacatcgaaaagtactacaatctagctaaggattcaagtatcagggtgtcgaacttggccgtttcgaaaattctattcaacttggccacatttgcacgcaatactcagcagttatctgacaaagaggttgcttttattaaccagtttatgcgagatttaggcactaatgagggttttcgtcccaaccctaaggatattcaaattttaaaagaatgtgatggaattactgttccagaaaagttgacttaa

SEQ ID NO: 119 - DNA, 1170 bp, Saccharomyces cervisiae
atgcgtttttcaaatttcctaactgtatctgcattattaaccggagctctaggagctcctgctgttcgccataaacatgaaaagcgtgacgttgttactgccacagtccatgcgcaggttactgttgtcgtttccggtaacagcggcgaaactattgttccagtgaacgagaatgctgttgtagctactaccagcagtactgcagttgcttctcaagcaactacatccactttagaaccaacaacttccgctaatgtcgtcacttctcaacaacaaaccagcactcttcaatcttccgaggcagcatctacggttggttcttcgacttcatcctcaccctcatcctcatcctcaacttcatcttcagcttcatcctccgcttcatctagtatctcagcctccggtgctaagggtattacttacagtccttacaatgatgatgggtcctgtaaatctactgctcaagtcgcctcagatttagaacagttgactggttttgacaacatcagattatatggcgttgactgtagtcaggttgagaatgtcttgcaagctaaaacttcaagccagaaattattcttaggcatatattacgttgacaaaattcaagacgccgttgatactattaaatctgcagttgagtcttatggctcctgggatgatattaccactgtttctgtcggtaacgaactggtcaatggcggttctgccactacgacgcaagtcggtgaatacgtttccacggccaagtcagctttaacctctgctggttatacaggctcagtcgtttccgttgataccttcattgctgttataaataaccctgacctgtgtaattattctgactatatggctgtcaacgcccatgcatacttcgatgaaaatactgcggcccaagatgcaggaccatgggtactagaacaaatcgaaagggtttacactgcttgtggtgggaaaaaggacgtcgttattaccgaaactggttggccatctaagggtgatacttacggcgaagctgtcccatctaaagcaaaccaagaagccgccatttcttctatcaaaagctcctgcggctcttcagcttacttatttaccgccttcaatgatctatggaaagatgatgggcaatacggtgttgaaaaatactggggtattctatcaagtgattaa

SEQ ID NO: 120 - DNA, 387 bp, Saccharomyces cervisiae
atgaacagtgctaccagtgagacaacaaccaatactggagctgctgagacaactaccagtactggagctgctgagacgaaaacagtagtcacctcttcaatttcaagattcaatcatgctgaaacacagacggcttccgcgaccgatgtgattggtcacagcagtagtgttgtttctgtatccgaaactggcaacaccaagagtctaataacttccgggttaagtactatgtcgcaacagcctcgtagcacaccagcaagtagcataataggatctagtactgcctctttagaaatctcaacctacgttggtattgccaatggtctgttgaccaataatggcataagtgtttttatttccaccgtattgctggcaatcgtatggtaa

SEQ ID NO: 121 - DNA, 1164 bp, Saccharomyces cervisiae
atggacggtagaaatgaaaaaccaaccactcctgtgtcagattttcgggtgggaagctccgagcaaagtcaagcgggagtgaatcttgaagatagtagtgaccatcgcacttccaattcagccgagagcaaaaaaggcaatttaagtggtaaaagcatcagtgatctaggtatttctaataatgataacaaaaatgtaagattcactgctgatacggatgctctagaaaatgatttgtcttcaagatctacagaaaccagcgataattctaagggcacagatggacaagatgaagaagataggcctgctcgccacaagaggaagcctaaagtttctttcacacatttaaggaacaatggtaaggatggagacgatgagacgttcatcaagaagataataaataacctgactggaaatcaagggggtttggtccctggcttggcaccaataccttcagaaaatgaaaatgggaagaatgatatagaaaaaaataaccgtaatgaagaaattcccttatccgatctagctgatgcgtctaaaatcgtagacgttcatgagggcgacgataaagaaaaactggaggctctcaaattagaaggtgacgtaaattgtacgtcggatggcgaaacgttaggctcaagttcaaaaaattcatttctggctcctgcagtggatcattttgatgattatgcagaaaacaattcatccgacgataacgaagggtttattgaaacctccacatacgtaccccctccatctcaagtgaaaagtggagtactagggtcattattgaaactttaccaaaatgaagatcaaaattcaagctcaatcttttcagattcacaagctgtaacaacagatgatgaaggtatttcttctactgctggaaacaaagacgtaccagttgccaagcgtagcagattacaaaatttaaaaggcaaggctaaaaaaggcagaatgcctagactgaagaaaagactaaaaactgaagcgaaaattacggttcacattgcagacattttacaaagacaccggttcatcctacgcatgtgtagagctcttatgatgtatggtgctccgacgcataggcttgaagaatatatggttatgacttctagagtccttgaaatagatggtcagttttgtatcttccaggttgtatga

SEQ ID NO: 122 - DNA, 954 bp, Saccharomyces cervisiae
atggatcaagattttgacagtttattactaggtttcaatgactccgatagtgtccaaaaagacccaactgtaccaaatggcttggatggttcagtagttgatcctaccattgcggatccaaccgcaattacagctagaaagagaaggcctcaagtaaaattaacagccgaaaaactactcagtgataaaggtttaccatatgttttgaaaaatgcacataaaaggatacgaatttcctcaaaaaaaaactcatatgacaacttatcaaatattattcagttttaccagctttgggcacatgaattgtttcccaaggcaaaatttaaggattttatgaagatctgtcaaacagtaggtaaaacagatccagttcttagagaatatagagtcagcctttttagggacgagatgggcatgagtttcgatgttggcacacgggagactgggcaagacctggaaagacaatcacctatggttgaagaacatgtcacttccgcggaagagaggcctattgtcgcagatagttttgcgcaagacaaaaggaatgtaaacaatgtcgattacgataatgacgaagatgacgatatctatcacctttcttatcgcaacagaagaggacgagttttggacgaacgtgggaataatgaaacggtacttaacaacgttgtgccgcctaaggaagatttggatgcattattgaagacattcagggtacaagggcccgttggccttgaagaaaatgagaagaagctcttattaggatggctagatgcgcatagaaaaatggaaaaaggctctatgactgaagaagacgttcaactgattcaaagtttggaagagtgggaaatgaatgatatagagggacaacatactcattatgatttattgccagggggagatgagtttggcgtagatcaagatgagttggatgctatgaaggaaatgggcttttag

SEQ ID NO: 123 - DNA, 2292 bp, Saccharomyces cervisiae
atgaacgatagccaaaactgcctacgacagagggaagaaaatagtcatctgaatcctggaaatgacttcggccaccaccagggtgcagaatgtacgataaatcataacaacatgccacaccgcaatgcatacacagaatctacgaatgacacggaagcaaagtccatagtgatgtgcgacgatcctaacgcataccaaatttcctacacaaataatgagccggcgggagatggagctatagaaaccacgtccattctactatcgcaaccgctgccgctgcgatcgaatgtgatgtctgtcttggtaggcatatttgttgccgtggggggcttcttgtttgggtatgacactggacttataaacagtatcacggatatgccgtatgttaaaacctacattgctccgaaccattcatatttcaccactagccaaatagccatactcgtatcattcctctccctaggaacatttttcggtgcgttaatcgctccctatatttcagattcatatggtaggaagccaacaattatgtttagtaccgctgttatcttttccatcggaaactcattacaggtggcatccggtggcttggtgctattaatcgtcggaagagtgatctcaggtatcgggatcgggataatctctgctgtggttcctctttatcaagctgaagctgcgcagaagaaccttagaggtgccatcatttccagttatcagtgggctatcactattgggttactcgtgtccagtgcagtatcgcaaggaactcattccaaaaatggcccgtcttcatatagaataccaattggtttgcagtacgtttggtcaagtattttagctgtgggcatgatattccttccagagagtccaagatattacgtcttgaaggatgaactcaataaagctgcaaaatcgttatcctttttaagaggcctcccgatcgaagatccaagactcttagaggagcttgttgaaataaaagccacttacgattatgaagcatcgttcggcccgtcaacacttttagattgtttcaaaacaagtgaaaatagacccaaacagattttacgaatatttactggtatcgccatacaagcttttcaacaggcatctggtatcaattttatattctactatggagttaattttttcaacaacacaggggtggacaactcttacttggtttcttttatcagctatgccgtcaacgtcgccttcagtataccgggtatgtatttagtggatcgaattggtagaagaccagtccttcttgctggaggtgtcataatggcaatagcaaatttagtcattgccatcgttggtgtttccgagggaaaaactgttgttgctagtaaaattatgattgcttttatatgccttttcattgctgcattttcggcgacatggggtggtgtcgtgtgggtggtatctgctgaactgtacccacttggtgtcagatcgaaatgtaccgccatatgcgctgccgcaaattggctagttaatttcacctgtgccctgattacaccttacattgttgatgtcggatcacacacttcttcaatggggcccaaaatattcttcatttggggcggcttaaatgtcgtggccgttatcgttgtttatttcgctgtttatgaaacgaggggattgactttggaagagattgacgagttatttagaaaggccccaaatagcgtcatttctagcaaatggaacaaaaaaataaggaaaaggtgcttagcctttcccatttcacaacaaatagagatgaaaactaatatcaagaacgctggaaagttggacaacaacaacagtccaattgtacaggatgacagccacaacataatcgatgtggatggattcttggagaaccaaatacagtccaatgatcatatgattgcggcggataaaggaagtggctcgttagtaaacatcatcgatactgcccccctaacatctacagagtttaaacccgtggaacatccgccagtaaattacgtcgacttggggaatggtttgggtctgaatacatacaatagaggtcctccttctatcatttctgactctactgatgagttctatgaggaaaatgactcttcttattacaataacaacactgaacgaaatggagctaacagcgtcaatacatatatggctcaactaatcaatagctcatctactacaagcaacgacacatcgttctctccatcacacaatagcaatgcaagaacgtcctctaattggacgagtgacctcgctagtaagcacagccaatacacttccccccaataa

SEQ ID NO: 124 - DNA, 951 bp, Saccharomyces cervisiae
atggcatttagtgattttgctgccatatgctcaaagaccccgttgccattatgttcggtaataaagtctaaaacccatctaatactttcgaactcaacaattatacatgattttgatcctttaaatttgaatgtcggtgtactgccacgctgttatgctcggtcgattgatcttgccaatacagtcatctttgatgtcgggaacgcattcataaatattggtgctctaggtgtcattttaatcatactttataacataagacagaagtatactgctattggcaggtctgaatatctctactttttccaactaacattgctattgataatatttaccttggtggtagactgtggtgtatctccccccggctctgggtcatatccatacttcgtggctatacaaataggactggcgggtgcatgttgctgggccttattgataatcgggtttttaggtttcaatttatgggaagatgggactacaaagtccatgctgttggtccgtggaacgtccatgctaggattcatagccaattttttagcctctattttaaccttcaaagcatggatcaccgaccataaagtagcaacaatgaacgcttcagggatgattgtcgtcgtttacataataaacgccattttcttattcgttttcgttatttgtcaattactggtatccctattggtagttcgaaacttatgggtcacaggagctatctttttggggctatttttctttgtagcaggccaggtattggtttatgccttctctacacaaatttgtgaagggttcaagcactacttagatggcctcttttttggaagcatctgtaatgtgttcacattaatgatggtttacaagacttgggatatgactaccgacgacgacttggaatttggtgtaagtgttagcaaggacggtgacgtggtgtatgataatggatttatgtga

SEQ ID NO: 125 - DNA, 1713 bp, Saccharomyces cervisiae
atggttgccgctttaacgtatttgcctactgagcttatccaaaggatatttgagttcactgtggtggaaacagactctcaatattggttgtacaatttagtggctctaattgatttttctgtctcttcgagaggtggtggctctataacggaagacttcttgacaaattacgttaggaagaatttgatggttttagatctgacctgtgaggccacgcaagactcgattttacgagcggagtacgggtttctgaagagattgttgccatacattgacatggacgcacaatatatcagagttgttgatttggaaaccaatgctgacaaggcccagaatttaaaagcagaaaaacttattgttatatttgacgaattctcagatttgaaactcatagaaaccttcttccccttggcgaattccaattcaaacataatcgagttcgtattctgtgttcgcaatataaagagttcgttttattcacctttggaaaaattacatattgcgaacatagtcgcagatattgatattaacacattgtatctggacttcgtggattcaaatatctattcggatcaaaatttctttgggatttttgatcccgatatttttcagctgattaataaaaactatagaaacttcttttctaagactaacgaaaaggggaagaaaagaccccccatttgcaagaaaatctgttttccctttgttgaaacattgaatttggattatatggcccttgattcattctttaattcgatactgcataaactaacaacaaagataaaaacatttgaaaggaacaatgagtttgacgtggataaaaatttaaatttaaactcgacaacgacagtagcagctttaattatcaagtcgatcttgcaacaattcttcaacaattttcatatcagcttccctaatttggttaccttgaattttattaagatgtctacctacccaaacaataatgagattacccaatgttgtaacttcatagatttatcttcatatgttctaaacaaatgtttaagtgagaatatctcgataaatttcctctttcagttgcactctttgaaaaattggtcaatgcccaagattaaagaattcactgggcacaaattcaagtatgacgaaacaacattttcaggttcaccagaaaggtacatcaaatcattgaggggaaacattaaaattttgcaagaaatggcaattaacgaaaccaacgatggtacttgctatttcagagtcaagttgatacctgagggggtagaaaaaactcaaataatcaactggatcccctttacttcttcatttagcgatgataccttcaaacaaagacaccatttaaagaggccaatgatttgcttgaagaacaactctttaagatcgctcactgtcaaaatcatacgtattgaaaaatgttcatccatccgaatccagggattttacctaccaaatctgcaggaactgttcatcaacaataccctttgcgacaccacccaacaccaaaaacaagcgtcaaatgatatgagttgtatagagttcacttcatggaatgaactaccacaatgcaagaaattgggatttgctcaattagaggacgactctaactacgttcttaatatcagtaacctacaagaccatttaccaaatctggacctgcgggagagtttcccaactttcttcgatataagacagaagtttgtcgtggtttga

SEQ ID NO: 126 - DNA, 372 bp, Saccharomyces cervisiae
atgaatattaattattattattgttataaatctatatgctcgtggatttttttaaataaattagacttacctgttatttacaagacttcttcgtttgacattagtccggcctgtgattctatgtcttgctcacctgcaatagcccgcgtagaaaaaagccttgaccaaaaattcccaatcgaaaatttggacttgaaatctgaaatcccatgtgattcaatatccggtggagtccacttcttcaacatcaatgaacttagaacgacactgaccgagctgaatgccatcgctaaaccggcgagcattggaggaagagttatgccccaaggaatgagcacacccatagcaatcggaatcatgaatatattatag

SEQ ID NO: 127 - DNA, 168 bp, Saccharomyces cervisiae
atggtcaaattaacttcaatcgctgccggtgtcgccgccattgctgctggtgcctccgccgcagcaaccactacattatctcaatctgacgaaagagttaatttggttgaattaggtgtttatgtttccgatatcagagctcatttggctgaatactactctttctaa

SEQ ID NO: 128 - DNA, 234 bp, Saccharomyces cervisiae
atggcatttgaaagacaaggaaagatcgaaaagaagatatcgtattccttatttttgaatggacctaatgtacactttgggagcatcttattcggtgcagtcgataaaagtaagtacgcagaagagctctgcacacatcctatgcgtcaagcttataatacccttgattcaaactcaagaataattatcacagtacagagtgttgcaattttggacggcaaacttgtatggtaa

SEQ ID NO: 129 - DNA, 1149 bp, Saccharomyces cervisiae
atgaataataacagcagaaacaacgaaaatcgaagcactattaacagaaataaaaggcaactacaacaagcaaaagaaaaaaatgaaaatattcatatccccaggtatattagaaatcaaccatggtactataaggatacccccaaagaacaagaagggaagaagcccggcaatgatgatacgagcactgcagaaggaggagaaaaaagcgactacttggtgcatcataggcaaaaagcaaaagggggtgctttagatattgacaataattcagaaccaaaaattggtatgggtataaaggatgagttcaaactaatcagaccccagaagatgtccgtccgagattctcattcgctgtcattttgtaggaattgtggggaagcagggcataaggagaaagactgcatggaaaaacctcgtaagatgcagaagcttgttcccgatttaaattcacaaaaaaataatggcacagttttagtacgagctactgatgatgactgggactccagaaaagatagatggtacggttactcagggaaagaatacaatgaactgataagtaagtgggagcgtgataaaagaaataaaataaaaggaaaagacaaatcccaaactgatgaaacactatgggatacagatgaagagatagaactaatgaagttagaactttacaaggattccgtaggttcattgaagaaagatgatgctgataattctcagttgtataggacatcaacgagattgagagaagataaggctgcttacttgaacgacataaattcaacggagagtaattatgatcctaaatcaagattgtacaaaactgaaacactgggcgcagttgatgaaaaatcaaaaatgttccgcagacatttgacaggtgaaggcctaaaattaaacgaattgaaccagtttgctagatctcacgctaaggaaatgggtatacgtgatgaaattgaggataaggaaaaagtacaacatgttttagtcgccaatcctactaaatatgaatatctgaagaaaaaacgggaacaagaagaaaccaagcagcccaagattgtcagcattggagatctggaagctaggaaagtagatggtacaaagcaatctgaggaacaacggaaccacttaaaagatttatatggttaa

SEQ ID NO: 130 - DNA, 1317 bp, Saccharomyces cervisiae
atggaaacaccacccattgtgattgataatggctcatacgaaatcaagtttggtccttccacgaataagaaaccgttccgagctttaaatgcattggccaaagataaatttgggacatcgtatttatcaaatcatatcaaaaacatcaaagatatttcatctatcaccttcaggaggccacatgaactaggacagctcacattatgggaattagagagttgtatatgggattattgccttttcaatccttcagagtttgatgggtttgatctgaaagagggaaagggtcatcatttggttgctagcgagagctgtatgactttaccagaattaagtaagcatgccgaccaggtgatatttgaagaatatgaattcgacagtcttttcaagtctcctgtagcagtctttgtaccatttaccaagtcatataagggtgaaatgagaacaatttcaggtaaggacgaagatatcgatattgtccgtggcaactcagacagtacaaattccacatcaagcgagtccaagaatgcgcaggattcaggtagcgattatcatgatttccaattagttattgattccgggtttaattgtacttggataattcctgtcctgaagggaataccgtactataaagcggtaaaaaaattggacattggaggccgtttcctaactgggctactaaaggaaactctatcattcagacactacaatatgatggatgaaaccatacttgttaacaatatcaaggaacaatgcttgttcgttagcccggtgtcttattttgatagtttcaaaacgaaggataagcatgcactagaatatgtacttcctgacttccaaacaagctttcttggttacgtaagaaaccccagaaaagaaaatgtaccgttacctgaagatgcgcagatcataacactgacagatgagcttttcacaataccagaaacttttttccatccagaaatttcgcaaattactaaaccaggcattgtggaggccatcctagagagcctttccatgttgcccgaaatagtgcgacctcttatggtaggaaacattgtatgtacaggaggaaactttaatctgcccaatttcgcccaacggcttgcggcagaactacaaaggcaattacccacagattggacttgtcatgtttcggtgcccgaaggtgactgtgctctgtttgggtgggaagtgatgtcacagtttgcaaagacagattcctaccgaaaagcgagggtcacaagagaagaatactatgagcatggtcccgattggtgtacgaagcacaggtttggttaccagaattggatataa

SEQ ID NO: 131 - DNA, 534 bp, Saccharomyces cervisiae
atgttgaagagcacgctgaggctttcaagaatctctctcagaagaggtttcacaacgatcgactgtttacgccaacaaaattcggatatcgataaaatcatactaaatccaatcaaattagctcagggaagcaacagcgatcgtggccaaacctctaaaagcaaaactgataatgcagatattttatcaatggaaattccagtagatatgatgcaatctgctgggagaataaacaagagggagcttctatccgaggcggaaattgctagaagtagcgtggagaatgcacaaatgagattcaattctggaaaatctataatcgtgaataagaacaaccctgcagaatcatttaagagattaaacaggatcatgtttgagaacaatattcccggagataaaagaagtcaacggttttacatgaagccggggaaagtggctgaattgaagagatctcaaaggcataggaaggaattcatgatgggcttcaagaggttgattgaaattgttaaagatgccaagaggaaaggatactaa

SEQ ID NO: 132 - DNA, 954 bp, Saccharomyces cervisiae; identical to SEQ ID NO: 122

SEQ ID NO: 133 - DNA, 2343 bp, Saccharomyces cervisiae
atggctcctaaatctagttcttccggttccaaaaagaaatcatcggcaagttctaatagtgctgatgcaaaagcatccaaatttaaattgcctgctgaatttattaccagaccacatccttctaaagatcatggcaaggaaacatgcacagcatatattcatcctaacgtattatcctcgcttgagataaatccgggatcattttgtactgtcggtaagataggcgaaaatggtattttagtaatagctagagcgggtgatgaagaagtacatcctgttaatgttatcaccctttccacaactatacgatctgttgggaaccttatccttggtgatcgtctagaattaaagaaagcccaggtgcaaccaccttatgccactaaggttaccgtggggtccttacaaggatataatattttggaatgtatggaggaaaaagtaattcaaaagctactggatgatagtggcgttataatgcctggaatgatttttcaaaacttaaaaacaaaagcaggtgatgaaagcattgatgtcgtaattacagatgcgagcgatgattcgcttcccgacgtcagccaactagatcttaacatggacgatatgtacggtggattagataacctgttttatctatctccaccttttatattcagaaaaggctccacacatataactttttcgaaagaaacccaggcaaatcgtaaatacaatcttccggagcccttatcctatgcagcagtgggcggcttagacaaggagattgaatcactgaaaagtgctattgaaatacctcttcatcaaccgacgctatttagtagctttggtgtttctccccctcgaggtatacttcttcacggacccccaggtactggtaaaactatgcttttgagagttgtagcaaatacgtccaacgcacacgtcctaaccattaatggcccctcaatcgtctccaaatatcttggtgaaacggaagcggcattaagagatatttttaatgaagcaaggaagtaccagccttccattattttcattgacgaaattgattcaatagcaccaaatagagcaaacgatgactccggtgaagttgagagcagagtcgtggctacattgcttaccctaatggatggcatgggcgctgcaggtaaagtggtggtaattgctgctacaaacaggcctaattctgtcgaccctgctctcaggagacctggcaggtttgaccaagaagtagaaattggtataccagacgttgatgccagatttgacattttaactaagcaattctcaagaatgtcctcggatcgtcacgtattagattctgaagcgatcaagtacattgcttctaaaacgcatggctatgttggtgctgatttaactgctctctgcagagaatcagttatgaagacgatacaacgaggactaggaacagacgccaatattgacaagttttccctaaaagttacattgaaagatgtggagagcgccatggttgatatcagacccagcgcaatgagagaaatcttcttagaaatgccaaaagtttattggtctgacattggcggccaagaagagcttaaaacaaagatgaaagaaatgatacagttgcctttggaggcttcggagacttttgccaggctgggaatttctgcaccaaaaggtgtattactttacgggccgccaggttgctccaagacattaaccgcaaaagctctcgctacagaatcgggtatcaacttcttagctgtgaaagggcctgaaatttttaacaagtatgtaggggaatccgaaagagctataagagaaattttccgcaaagcacgctctgcagctccaagtattatcttctttgatgaaatcgatgcattatctcctgatagagacgggagttccacctctgcagctaatcacgtgctcacatctttactcaatgagattgatggtgttgaagagttaaagggtgtagttattgtagcggcgacgaatagacctgatgaaatagatgctgctcttctaaggcctggtaggttagatagacacatttacgttggccctccagacgtaaacgcccgcttggaaatcttaaagaagtgcacaaagaaatttaatacagaagagtctggagtcgatcttcatgaattggcagaccgtacagaaggttattccggagctgaagttgtgctgctttgtcaagaagcgggcttggctgccataatggaagatttagatgtcgcaaaagtggaattacgtcattttgagaaagcttttaaaggaattgctaggggcattactccagaaatgctctcttattatgaagagtttgctctaagaagcggttcatcttcgtaa

SEQ ID NO: 134 - DNA, 360 bp, Saccharomyces cervisiae
atgccgcatttagccgccgaagcgcatacttggcctccgcatatttcacattcaacactttcgattccgcatccaaccccggaacaccggcacgtatttcataaaaaggacgtgaagaacaaaaggaacgaagaaaaaggcaataatttactctatgtgttatttagaactacggtgataaagagctcgttccgatcactaagtacggccggaagagagctgttgtttgttgtccatcaagggcacatcggcaccggcctcatcgtcttcatcatatgctggaggctgtgcttgagattcctctgcagggtgagcttccaggtcacggtctacggcgggcgcagtcgcatgtctgcgtga

SEQ ID NO: 135 - DNA, 522 bp, Saccharomyces cervisiae
atgagttttgcatcgaggaatattggacgtaagatagcaggagtgggagttgacattgtatacttgccaagatttgcacatatactagagaaatattccccattcgacccatgtggccgttctaccttgaataaaataacacggaagttcatgcatgaaaaggaaagatttcatttcagtaatcttctcatcgaagaaaactgcttaactccacgattgcatgaatatatagcgggagtttgggctttgaaggaatgctcattgaaagcgttgtgttgctgtgtttcaaagcatgatctacctcctgcccaagtactgtacgctggaatgctatataaaacacaaaccgatacaggtgtacctcagttagagtttgataagatgtttggaaaaaagtatccaaagtatcaacagctctcgaaaaactacgattctctcttttccactcatgagtttttagtttcgctatcccatgataaagattatttaattgcagtaacaaacttggtagaaagagagtaa

SEQ ID NO: 136 - DNA, 1566 bp, Saccharomyces cervisiae
atgacgactcgtttgctccaactcactcgtcctcattacagattattatccctacctctccagaaacccttcaatataaaaaggcagatgtccgctgcgaacccttctccatttggcaattatttgaacacgatcactaagtccctacaacagaatttacaaacatgctttcatttccaagcaaaagaaatcgatataatcgaatctccatctcagttttacgatctcttgaagacaaaaatacttaattcacaaaatagaatattcattgcgtctctgtatttaggcaaaagcgagactgagttggtggactgcatatcccaggcattgaccaagaaccccaagttgaaagtttcttttctacttgatggccttcgaggaacaagagaattgccttccgcctgttccgccactttattatcgtctttagtagccaaatatgggtcagagagagtggattgccgattgtacaagacgcctgcttatcatggttggaaaaaagtcttggttcccaagagatttaatgaaggtttaggcttacaacatatgaaaatatatgggtttgataacgaggtcattctttcgggagccaacctttcgaacgactatttcaccaacagacaagatagatactatctctttaaatctcgaaacttctccaactattattttaaattacatcaactcataagttccttcagttatcagattataaagccaatggtggatggtagcatcaacatcatttggccagattcgaatcctactgttgaaccgacgaaaaataaaaggctgtttttaagggaagcatctcaattactagatggctttttaaagagttctaaacaaagcctcccgattactgccgtgggtcaattctccacattagtttacccaatttctcaattcactccactttttcccaaatataatgacaaatcgaccgaaaaaagaacaatattgtcattgctttccactataacaagcaatgccatttcttggacgttcactgcaggatacttcaatattttgccagacatcaaagcaaaactgctggcaacgccggttgctgaggcaaatgtaataacagcttccccctttgcaaacggcttttaccaatcaaagggcgtctcatcaaatttacctggtgcttacttgtacctgtcaaaaaaatttctacaagatgtatgtaggtacagacaagatcatgctattacattaagagaatggcaaagaggcgtagtaaataagccgaatggttggtcatatcacgcaaaaggtatttggctttccgctcgtgataaaaatgatgctaacaattggaaaccctttatcacggttataggatcttcaaactatacgagaagggcgtattcattagatttggaatcgaatgctctcattattacaagagatgaagagctaagaaaaaaaatgaaagcagagttagataatttattacaatatacaaaacctgtaactctagaagactttcaatcagacccagaaagacatgttggcactggtgtaaagatagctacctccattttgggtaaaaaactttag

SEQ ID NO: 137 - DNA, 696 bp, Saccharomyces cervisiae
atgacgagatactattgtgaatactgtcattcgtatttgacccatgacacgttgagcgttcgtaaatcgcacttggtcggtaagaatcaccttcgtataacagctgactattataggaacaaagcaagagacattattaataaacataatcataaaagacgccacattggaaaaagaggcaggaaagaaagagaaaacagtagtcaaaatgagacgctaaaagttacatgcctttcaaataaggagaaaagacacatcatgcatgtgaagaaaatgaaccaaaaagaactggcacaaacctcaatagataccttgaaattgttatacgatggctcaccaggatattccaaagtatttgtggatgctaacaggtttgatataggagatttggttaaagccagcaaattaccccaaagagccaatgaaaaatctgcacaccattccttcaagcaaacttcaagatccagagatgagacgtgcgagagcaatccatttcctaggttgaataacccaaagaagctagaacccccaaagatattatcacaatggagtaacaccattccaaaaacttctatattttacagtgtagatatactgcaaaccacgatcaaggagtccaagaagcggatgcattccgacggcatacggaaaccgtcgagtgccaacggatataaaaggaggcggtatggaaattaa

SEQ ID NO: 138 - DNA, 1905 bp, Saccharomyces cervisiae
atgttatctttgaaaaaaggaataacaaaatcatacatcttgcaaagaactttcacttcttcctctgttgttcgtcaaattggggaagtgaaatctgaatcgaaaccgccggccaaatatcatattatcgaccatgaatatgattgtgtggtggtaggcgctggcggtgcaggtttaagagcagctttcggtttggctgaagctggatacaagactgcttgtttatccaagttgtttccaacaaggtcacatactgtggctgctcagggtggaattaatgctgcgctgggaaatatgcatccagatgattggaaatcgcacatgtacgacactgtcaagggttctgactggctcggagaccaagatgcaatccattacatgacaagagaagcacctaagtctgtcattgaactagaacattacggtatgcccttttcgaggactgaagatggaaggatttaccagagagcatttgggggacaatccaaagattttggtaaaggtggacaggcctataggacttgtgcggtggcagatagaacaggtcacgcaatgcttcatacattgtatggacaagcgctgaaaaataatacacacttctttattgaatactttgcaatggatttgttgacccataatggcgaggttgtgggtgtcattgcctataatcaggaggacggtacaattcacagattcagagcacataagaccgtcatcgcgacaggcggatacggtagagcttacttctcttgcacttctgctcacacttgtacaggtgacggtaatgctatggtttctcgcgctggatttccactagaggatttagaatttgttcaatttcatccgtcaggaatttatgggtctggctgcctaatcactgaaggtgcccgtggtgagggtggatttttattgaattctgaaggagaaaggtttatggaacgctatgctcctactgccaaggacttggcaagcagggatgttgtttccagagcaatcaccatggaaatcagggctggcagaggtgtcgggaaaaacaaggatcatatccttttacaattaagccatctaccacctgaggtactaaaggaaaggctaccgggaatatctgaaacagctgctgtctttgcgggtgtcgatgtcacccaggagccaattcctgtcttgccaactgtccattataatatgggaggcattcccacaaaatggactggtgaagcattgaccattgacgaggaaactggagaggataaggtcatcccaggattgatggcgtgtggtgaagctgcttgcgtatcggttcatggagcgaacagattaggcgctaactcactactggatttagtcgttttcggtcgcgccgttgcaaataccattgctgacacattacagcctggcttgcctcataagccattggcttcaaacatcgggcacgagtcaattgctaatttggataaagtaagaaatgctcgcggctcactgaaaacctctcaaatcaggttgaacatgcaaaggacaatgcaaaaagatgtttctgttttcaggacgcaagacactctagatgaaggtgttagaaatattactgaagtggacaagacatttgaggatgtgcacgtttctgataagtcaatgatctggaattctgatctcgtagaaactctggaattgcaaaatttacttacttgtgccacacaaacggctgtttctgcttccaaaagaaaggagtctcgtggtgctcatgcgagagaggactatgcaaaaagagatgatgtgaattggagaaagcacacattatcatggcaaaaggggacatcaacacctgtaaaaatcaagtacaggaatgtaatcgcacatactttagatgagaatgaatgcgccccagtccctccagctgtcagatcctattaa

SEQ ID NO: 139 - DNA, 1665 bp, Saccharomyces cervisiae
atggacagagatataagctaccagcaaaattatacctcaactggggcaactgcaacttcctcaagacagccctctacggacaataatgcagatacaaattttttgaaggtaatgtcagaattcaaatataattttaacagtccgttacctacaacgactcaattccccacgccctattcttctaatcagtatcaacagactcaagatcattttgccaatacagacgctcacaacagttcgagcaacgaatcgtcgttggtagagaacagtatattaccgcatcatcagcagatacaacagcaacaacaacaacaacaacaacaacaacaacaacagcaagctctaggttcacttgtacctcctgctgtcacaaggacagatacaagtgagactttggacgatatcaacgttcaaccttcttctgttttgcagttcggcaactctttacccagcgaatttttggttgcatccccagagcaattcaaagaatttttgttggactctccgtccaccaatttcaatttctttcacaaaactccggcaaagacaccacttcgatttgtaacagattctaacggtgctcagcaaagcaccacagagaacccaggtcaacaacagaatgtttttagcaatgtcgatttgaacaatcttttgaagagtaatggaaaaacaccctcatcttcatgcaccggcgcattttcacgcactcctctgagtaagattgacatgaatctcatgttcaatcaaccgctgccgacatctccatcaaaaaggttctcctccctgtcgttgacaccatatggaagaaaaattctgaatgacgtcggtacaccttatgcaaaagcattgatatcgtctaacagcgcgttagtggattttcagaaggcaagaaaggatattaccactaatgcaacatccatagggctggaaaatgccaacaacatcttacagagaacgccgctaagatctaacaataaaaaattatttattaaaaccccccaggataccatcaatagcactagcacactaactaaggacaacgaaaataaacaggacatatacggctcttcaccgactaccatccaattaaattcatcaataactaaatctatctccaaattggataactctagaattcccttgttagcttcgagatcagataacattctggattccaatgtggatgaccaattgtttgatttggggttgacaagattacctttatcaccaacaccaaattgtaattctttgcatagtacaaccacaggtacatctgccttacaaattcctgagctacccaagatggggtcttttagaagtgatacgggaatcaatccaatttcaagttcaaacacagtttcttttaagagcaaatcaggcaataataattcaaagggtcgaatcaaaaaaaatgggaagaaaccttccaaatttcaaattattgtggcaaatattgatcaatttaaccaggatacatcatcgtcatctttatcatcatcattgaatgcaagttcgagtgcagggaattcaaattcaaacgtaacaaagaaaagagcaagtaaactcaaaagatcacagtctttactttctgattccggatcgaaatcacaagcaaggaaaagctgtaattctaaatctaatggaaatttattcaattcacagtaa

SEQ ID NO: 140 - DNA, 2445 bp, Saccharomyces cervisiae
atgaaagtgaggaaatatattactttatgcttttggtgggccttttcaacatccgctcttgtatcatcacaacaaattccattgaaggaccatacgtcacgacagtattttgctgtagaaagcaatgaaacattatcccgcttggaggaaatgcatccaaattggaaatatgaacatgatgttcgagggctaccaaaccattatgttttttcaaaagagttgctaaaattgggcaaaagatcatcattagaagagttacagggggataacaacgaccacatattatctgtccatgatttattcccgcgtaacgacctatttaagagactaccggtgcctgctccaccaatggactcaagcttgttaccggtaaaagaagctgaggataaactcagcataaatgatccgctttttgagaggcagtggcacttggtcaatccaagttttcctggcagtgatataaatgttcttgatctgtggtacaataatattacaggcgcaggggtcgtggctgccattgttgatgatggccttgactacgaaaatgaagacttgaaggataatttttgcgctgaaggttcttgggatttcaacgacaataccaatttacctaaaccaagattatctgatgactaccatggtacgagatgtgcaggtgaaatagctgccaaaaaaggtaacaatttttgcggtgtcggggtaggttacaacgctaaaatctcaggcataagaatcttatccggtgatatcactacggaagatgaagctgcgtccttgatttatggtctagacgtaaacgatatatattcatgctcatggggtcccgctgatgacggaagacatttacaaggccctagtgacctggtgaaaaaggctttagtaaaaggtgttactgagggaagagattccaaaggagcgatttacgtttttgccagtggaaatggtggaactcgtggtgataattgcaattacgacggctatactaattccatatattctattactattggggctattgatcacaaagatctacatcctccttattccgaaggttgttccgccgtcatggcagtcacgtattcttcaggttcaggcgaatatattcattcgagtgatatcaacggcagatgcagtaatagccacggtggaacgtctgcggctgctccattagctgccggtgtttacactttgttactagaagccaacccaaacctaacttggagagacgtacagtatttatcaatcttgtctgcggtagggttagaaaagaacgctgacggagattggagagatagcgccatggggaagaaatactctcatcgctatggctttggtaaaatcgatgcccataagttaattgaaatgtccaagacctgggagaatgttaacgcacaaacctggttttacctgccaacattgtatgtttcccagtccacaaactccacggaagagacattagaatccgtcataaccatatcagaaaaaagtcttcaagatgctaacttcaagagaattgagcacgtcacggtaactgtagatattgatacagaaattaggggaactacgactgtcgatttaatatcaccagcggggataatttcaaaccttggcgttgtaagaccaagagatgtttcatcagagggattcaaagactggacattcatgtctgtagcacattggggtgagaacggcgtaggtgattggaaaatcaaggttaagacaacagaaaatggacacaggattgacttccacagttggaggctgaagctctttggggaatccattgattcatctaaaacagaaactttcgtctttggaaacgataaagaggaggttgaaccagctgctacagaaagtaccgtatcacaatattctgccagttcaacttctatttccatcagcgctacttctacatcttctatctcaattggtgtggaaacgtcggccattccccaaacgactactgcgagtaccgatcctgattctgatccaaacactcctaaaaaactttcctctcctaggcaagccatgcattattttttaacaatatttttgattggcgccacatttttggtgttatacttcatgttttttatgaaatcaaggagaaggatcagaaggtcaagagcggaaacgtatgaattcgatatcattgatacagactctgagtacgattctactttggacaatggaacttccggaattactgagcccgaagaggttgaggacttcgattttgatttgtccgatgaagaccatcttgcaagtttgtcttcatcagaaaacggtgatgctgaacatacaattgatagtgtactaacaaacgaaaatccatttagtgaccctataaagcaaaagttcccaaatgacgccaacgcagaatctgcttccaataaattacaagaattacagcctgatgttcctccatcttccggacgatcgtga

SEQ ID NO: 141 - DNA, 840 bp, Saccharomyces cervisiae
atggtagagttgacaattacgggtgatgatgatgatatattgagtatgttttttgatgaggagttcgttccccatgcattcgttgatatactcttatcaaatgccttaaacgaagatcagattcaaacgcaatcagtatcctcattgctattaaccaggttggatttttacacaaagaaccttacaaaagagttggaaagcaccatatggaatttggataaattatctcaaacgttaccaagaacttgggcatcttctaggtatcacaaagaagcagaacagaacgattcctcattgtattctactgaatccttaaaatcatcgaagcttgaatattacttagatacgttggcaagtgctgtaagagcattagaaacaggaatgcataatgtaactgagaaactaagcgatctagataacgaaaataatcgcaataccaatgtgaggcaacaactgcaaagtttaatgttgattaaggagagaattgaaaaagtggtatattacctggaacaagttaggaccgttacgaatatttcgacagttagagaaaataatacaaccagcacggggacagatctttcgataacagattttagaacatcattgaaagcattagaggatacaatcgatgaatctttaagctctgcgattgataacgaggctaaagatgaaacaaacaaggatttgattgggagaattgattcactttctgaactgaaatgtctgtttaaaggcctagataagttctttgctgagtatagcaacttttcggagagcataaaatcaaaagcacaaagttatttatcaaccaagaatattgacgatggtatgatatcataa

SEQ ID NO: 142 - DNA, 1140 bp, Saccharomyces cervisiae
atgtttagtaacagactaccacctccaaaacattctcaaggacgagtttcgacggctttgagctcagatcgcgttgagccggcaatattgactgaccaaatcgctaaaaacgttaagctcgatgattttattccaaagagacagtctaatttcgaactatcggttcctttgccaacgaaagcagaaatccaagaatgtacagcaagaaccaagtcatacattcagcggcttgtgaatgcgaaactagccaactcaaataacagggcatcatcaaggtacgtcaccgaaacacatcaggcacccgcgaatctattattgaacaacagccaccatattgaggtagtgtccaagcaaatggatccattgttgccaaggttcgttgggaagaaggcgagaaaggttgtagcacccacagaaaacgacgaagtcgtgcctgttctccatatggatggcagcaatgataggggagaagctgatccaaatgagtggaagatacctgcagctgtgtcaaactggaaaaatccaaatggttataccgtggccttggaaagacgtgtaggtaaagctcttgacaacgaaaataataccatcaacgatgggtttatgaagctctccgaagcgttagaaaacgctgacaagaaggcaagacaagagatcaggtccaaaatggaattgaagcggcttgctatggaacaggaaatgcttgctaaagaatctaaattgaaagaattgagccaacgagccagataccacaacgggactccgcagacgggagcaatagttaagcccaaaaagcaaacgagcacagtggccagactaaaagagctggcgtactctcaaggaagagacgtatccgaaaagataattctgggcgcagcaaagcgttcagaacaaccggatctgcagtacgattcaagatttttcacaagaggggcaaatgcctccgccaaaaggcatgaagaccaggtttatgacaacccactgttcgtccaacaagatattgaaagcatatacaagaccaactacgaaaagctggacgaagcggtcaatgttaagagtgaaggtgccagtggttctcacggccccattcagtttactaaagctgaatccgatgataaatcggataactatggcgcctag

SEQ ID NO: 143 - DNA, 786 bp, Saccharomyces cervisiae
atgaagacctatcatttgaataatgatataattgtcacacaagaacagttggatcattggaatgaacaactaatcaagctggaaacgccacaggagattattgcatggtctatcgtaacgtttcctcaccttttccaaaccactgcatttggtttgactggcttggttactatcgatatgttgtcaaagctatctgaaaaatactacatgccagaactattatttatagacactttgcaccatttcccacaaactttaacactaaaaaacgagattgagaaaaaatactaccagcctaaaaatcaaaccattcacgtatataagccggatggatgtgaatcggaggcagattttgcctcgaaatacggggatttcttatgggagaaagatgatgacaagtacgattatctggccaaagtggaacctgcacatcgtgcctacaaagagctacatataagtgctgtgtttactggtagaagaaaatcacaaggttctgcccgctcccaactgtcgattattgaaatagacgaacttaatggaatcttaaaaataaatccattgatcaattggacgttcgagcaggttaaacagtatatagatgcaaacaatgtaccatacaacgaacttttggaccttggatatagatccattggtgattaccattccacacaacccgtcaaggaaggtgaagatgagagagcaggaagatggaagggcaaggccaagaccgagtgtggaattcatgaagccagccgattcgcgcaatttttaaagcaagatgcctag

SEQ ID NO: 144 - DNA, 390 bp, Saccharomyces cervisiae
atgttttcttctttttttggaaatacttgttcctgggtcttcattttcatcatcatcgttgacaatgaagccttcttgcacttttcttgcctcatcttcgtcttcatcaatatcttcgtcttcctcagaggagtcaaagacatcttctccttcttcttcctcactaggcgctttagtttcatcgttgtcatttactatttcttcctcgtccctagggaccagcttcgaatctcccgtctcttccataaaaggcaaattctatgtaaagattctaggcaattaatgacctgttcgcttgggttattcttcaaagcacaaatcaatatctttcttcctccttttgctttaaccgttgtccagtttcttgtcaatttggtttgccatacataa

SEQ ID NO: 145 - DNA, 495 bp, Saccharomyces cervisiae
atgacgaaaaaggataagaaagcaaagggtcctaagatgtccaccatcactacaaaaagtggtgagtccttaaaggtttttgaggatttgcatgattttgaaacatatttaaagggtgagacggaagatcaagagttcgaccatgtccattgccaactgaagtactatccaccctttgtcctgcatgatgcgcatgatgatccggaaaagatcaaagagactgccaattcgcactctaagaagtttgttcgccatttacaccagcatgttgagaagcacctgctaaaggacatcaaaaccgctatcaacaagccagaattgaaattccacgataagaaaaagcaggaatcctttgaccggattgtttggaattatggcgaagaaacggagttgaacgccaagaaattcaaggtgtctgtcgaagttgtatgtaaacacgatggcgcaatggtagatgttgattacaagacagaacccttgcagccactcatctaa

SEQ ID NO: 146 - DNA, 653 bp, Saccharomyces cervisiae
atgtgtatgaacgtcacaattttcataattttattgaatgggcaggcaaattattccaagtaaataaatggcgtagtccctgagtcctcgatattactgtctattagactatggttaatccaacgggaatatatttcatcacatgatagcgtactgctgcaaattgattagtaaattagttcccatctgccagacacaatgtgtcaacttcgcgtgctcgtaaaaaagtactaacaacggaacacctcaacaatcttgatacttagtactcttactacgtcatttctttgtcctagtaaatttgttgagaaaatttacataatctgaggaactacaatttctaatctccaggcacacccaccacgtgcctgttggctgaccgagacaaaagtggagaagacaggcacgcagaaacgaacgttttgcaaggaatggatatgcttctggagcttcttcttccagtctatgcgagattgaatgagagcggctggttgctatggtttgtcttccatgatgtgtacgaagctgtgaaaatgagtactaaggagtcagtgcacaccagggtaatcaatttccctgatattttgtcaactcaacaaatgagacagggtccatctcagatcaggacacctctggtaatgcttcttatgtga

SEQ ID NO: 147 - DNA, 951 bp, Saccharomyces cervisiae
atggaggagtacgattattcagacgttaaacctttgcccattgagacagacttgcaggatgaactgtgcaggattatgtataccgaggattataagcggttgatgggactcgcaagggctctgatcagccttaacgaactgtcacccagggcactacagctaacagccgaaattatcgacgtggcgccagccttctacaccatatggaactaccgattcaatatcgtcaggcacatgatgagtgaatccgaagacactgtcttgtacctgaacaaggaattagactggctagatgaagttacgctgaataatccaaagaactatcagatctggtcctatagacagtctcttttgaagctacatccgtctccttccttcaaaagagagctgcctatcttaaaactgatgattgatgatgattccaagaattatcacgtttggtcgtacagaaagtggtgctgtttgttcttcagtgactttcaacatgagctcgcctacgccagcgacctcatcgagacagacatttataacaacagcgcatggactcataggatgttttactgggtgaacgctaaagatgtcatttcaaaagtggaattggccgacgagctccagttcattatggacaagattcaattggttccgcagaacatcagtccgtggacctacctccgtggtttccaagagctattccatgataggctacagtgggatagcaaagtagtcgacttcgccacaaccttcatcggtgacgtattgtcacttccaattggctcaccagaggatttgcccgagatcgagtcctcatatgccctggaattcctggcatatcactggggggcagacccttgtacccgagacaacgctgttaaggcctatagcttgctagcaatcaaatacgatcctattagaaaaaacttgtggcaccacaaaataaataatctgaactga

SEQ ID NO: 148 - DNA, 834 bp, Saccharomyces cervisiae
atggttggtatttacagaatacttgcttcgttcgtcccactcctgggtcttctttttgcattccatgatgatgacatgatagatactgttacaatcatcaaaactgtatatgaaacggtgacatcaacttctactgcacctgcacctgccgctacaaaatctgttagtgaaaagaaactggatgacactaaactaacacttcaagtaattcaaactatggtatcatgtttttctgtaggtgaaaatccggccaatatgatatcctgtgggctaggagttgtaatcttaatgttttcattaatcatcgagcttatcaacaagctcgaaaatgatggtatcaatgaaccgcaaaggttatatgacctaattaaaccaaaatacgtcgagctaccttcaaattatgtgaatgaaaaaatcaaaacaacatttgaacctctcgacctatacttaggagtaaatatgaatacttcaggaagtgaactaaaccaaaactgtttgattctcaaacttggcgagaagacggctctgcctttcccaggcttggcccagcagatttgttatacaaaaggcgcttcaaatgagttcacaaattataaattatcggacatacagggcaatttaaacgaaaacagccaaggaattgctaatggcgttttccagaaaatatcaaacattagaaaaatatcaggtaattttaagtctcagctttatcaaatttcagaaaaaatcaccgacgaaaattgggacggttctgctgtaggcttcactgctcatgggagagaaaaaggcccaaacaaatctcaaatatcggtttcattttatagggataattaa

SEQ ID NO: 149 - DNA, 1506 bp, Saccharomyces cervisiae
atgcttttacaaggaatgcgtttatcgcaaaggttacataagagacatctatttgcttccaagattttaacgtggactacgaaccctgctcatatacgccacctacatgatataaggccgcctgcatcaaacttcaatacgcaagaatcggcccccataccggagtctccagcaaactcaccaactcgaccacagatggcacctaaacccaatttgaaaaaaaaaaatcgtagtttaatgtattctattattggggtttccatagtaggtttatatttttggtttaaaagtaactccaggaaacaaaaactacctctttcggcgcaaaaagtctggaaggaagccatatggcaagaaagtgataaaatggattttaattacaaagaagcgttaaggcggtatattgaggcgttggatgaatgcgatcgctctcatgtcgatttattgtcagatgattataccagaatagagctgaaaattgctgaaatgtatgaaaagctcaatatgcttgaagaagcccaaaatttgtaccaagaattattaagtcggtttttcgaagcgctgaatgttcctggcaaagttgatgagagtgaaagaggcgaggttttaagaaaagacttgagaatcttgattaaatcgttagaaatcaataaggacatagaaagtggcaagagaaaattgctacaacatttacttttagctcaagaggaaattttaagcaaatcgccagagttgaaggaattttttgaaaacagaaaaaagaagctctcgatggtaaaagacatcaatagagaccctaatgatgattttaaaacatttgttagtgaggaaaatattaagtttgatgagcaaggctatatgattttggatctggaaaagaatagcagcgcttgggaaccctttaaggaagaattttttactgcgagagatttatatacagcttattgtctgtcatcaaaagacatagctgcagctctaagttgcaagataactagtgtggaatggatggttatggcagacatgccaccaggacagatattgctatcacaggcaaatttggggtcattgttctatcttcaagcagaaaagctagaagctgacttaaatcaattagagcaaaagaaaagtaaagagtccaaccaagagttagatatgggaacatacataaaagccgttagattcgtacgcaaaaatcgtgacttatgtctggaaagagcacaaaaatgttacgacagcgttattgcgtttgccaaaagaaacagaaaaattaggtttcatgtgaaggatcaactggatccttcaattgcacagtcaattgctctatctacctatggaatgggggttttaagccttcatgaaggtgttttggctaaagctgaaaaactattcaaagattcgatcactatggccaaggagactgaatttaatgaactccttgcagaagctgaaaaggaactagaaaagacgacagtcttgaaagcggccaaaaaagagggtttaaactaa

SEQ ID NO: 150 - DNA, 2400 bp, Saccharomyces cervisiae
atgagttataaagtgaatagttcgtatccagattcaattcctcccacggaacaaccgtacatggcaagccagtataaacaagatttgcagagtaatattgcaatggcaacgaatagtgaacagcagcgacaacaacagcagcagcagcaacagcagcaacagcagtggataaatcaacctacggcggaaaattcggatttgaaggaaaaaatgaactgcaagaatacgctcaatgagtacatatttgactttcttacgaagtcgtctttgaaaaacactgcagcagcctttgctcaagatgcgcacctagatagagacaaaggccaaaacccagtcgacggacccaaatctaaagaaaacaatggtaaccagaatacgttctcgaaggtagtagatacacctcaaggctttttgtatgaatggtggcaaatattctgggacatctttaataccagttcttccagaggtggctcagagttcgctcagcaatattatcaactagttcttcaagaacaaaggcaggaacaaatatatagaagcttggctgttcatgcggcaaggctacaacacgatgcagaacgaagaggggaatatagtaacgaggacatagaccccatgcacttggctgctatgatgctaggaaatcctatggcacctgcggttcaaatgcgcaatgttaatatgaaccctataccaattcctatggttggtaaccctatcgttaataatttttccattccaccatacaataatgcaaaccccacgactggtgcaactgctgttgctcccacagcgccgccttccggcgattttacaaatgtagggccaacccagaatcggagtcaaaacgttactggctggccagtctataattatccaatgcaacccactacggaaaatccagtgggaaacccgtgtaacaataataccacaaataatacaactaataacaaatctccagtgaaccaacctaaaagtttaaaaactatgcattcaacagataaaccaaataatgtcccgacgtcaaaatctacaagaagtagatctgcaacctcaaaagcgaagggtaaagttaaagccggtctagtggctaagagacgaagaaaaaataataccgctacagtttccgcgggatcgacgaacgcttgttcgccaaatattaccacaccaggctcaacaacaagtgaacccgctatggtaggttcaagagtaaataagactccaagatcagatattgctactaacttccgcaatcaagcaataatatttggcgaggaagatatttattctaattccaaatctagcccatcgttggatggagcatcaccttccgctttagcttctaaacagcccacaaaggtaaggaaaaatacaaaaaaggcatccacctcagcttttccagtagagtctacgaataaactcggtggcaacagcgtggtgacaggtaaaaagcgcagtccccctaacactagagtgtcgaggaggaaatccactccttctgttattctgaatgctgatgccactaaggatgagaataatatgttaagaacattctcgaatactattgctccgaatattcattccgctccgcccactaaaactgcgaattctctcccttttccaggtataaatttgggaagtttcaacaagccggctgtatccagtccattatcttcagtgacagagagttgcttcgatccagaaagtggcaagattgccggaaagaatggacccaagcgagcagtaaactcaaaagtttcggcatcatccccattaagcatagcaacacctcggtctggtgacgctcagaagcaaagaagttctaaggtaccaggaaacgtggttataaagccgccacatgggttttcaaccaccaatttgaatattactttaaagaactctaaaataatcacttcacagaataatacagtatcccaagaattgccgaatgggggaaacatactggaggcgcaagtaggcaatgattcaagaagtagtaaaggcaatcgtaacacattatctactccagaggaaaaaaagccgagtagtaataatcaaggatatgattttgacgccctcaaaaattcaagttctttgttgtttcctaatcaagcttatgcttctaacaatagaacaccaaacgagaattcaaatgttgctgatgaaacctctgcatctacaaatagtggcgataatgataacacattaattcagccctcatccaatgtgggtacaactttgggtcctcagcaaaccagtactaatgaaaatcagaatgtacactctcagaacttgaagtttgggaatattggtatggttgaagaccaaggaccggattacgatctcaatttactggatacaaatgaaaatgatttcaattttattaattgggaaggctga

SEQ ID NO: 151 - DNA, 1518 bp, Saccharomyces cervisiae
atgaagttgggtattataccttaccaggaaggtactgatattgtttacaagaatgctctccagggtcagcaagaagggaagagacctaatttaccacagatggaagcaacgcaccaaatcaagtcatcggttcagggtacaagttatgagtttgtccgcacagaagatattccattgaatcgaagacattttgtgtacagaccgtgttccgcaaatccctttttcactattttggggtatggctgtacagaatacccatttgaccactctggaatgagcgtcatggacagatctgaagggttgtcaattagtcgagatggaaatgatctggtaagtgtcccggatcaatacggttggagaactgcaagaagcgatgtgtgtattaaagaaggaatgacgtattgggaagtggaggtaattcgtggaggaaacaagaaattcgcagacggtgttaataataaggaaaatgctgatgattcagtagacgaagtacaaagtggcatatacgaaaaaatgcacaaacaagtgaatgacaccccgcatctacgatttggagtttgcagaagagaggccagtttagaggctcccgtagggtttgatgtgtacgggtatggtattagagacatttcgttagaatctatccacgaaggaaaattgaattgcgtcctagaaaatggttcgccattgaaagagggtgataaaatcggatttctactgagtcttcctagcattcatacacaaatcaaacaagctaaggagtttaccaaaagaagaatttttgcactgaactcccatatggatacgatgaatgaaccatggagagaagatgctgagaatgggccttcaaggaaaaaattaaaacaagagacaacgaacaaagaatttcaaagggcgctattagaagatattgaatataacgacgtcgtccgcgatcaaatcgccatcaggtataagaaccagttgttctttgaggcaacggactatgtaaagacaacgaaaccggaatattattcttctgataagagggaaaggcaagactattaccagttagaggattcatatcttgctatctttcaaaatggtaagtacctaggcaaagcatttgaaaatttaaagccgttgttaccaccgttcagtgagttacaatacaatgaaaagttctatcttggatattggcaacatggtgaagctcgtgatgagtccaatgataaaaacacaaccagtgccaaaaagaaaaagcagcaacaaaagaaaaagaagggattgatactcagaaacaaatacgtaaataataacaaactgggttactatccaacaatcagctgttttaacggtggaacagcgaggataattagtgaagaagataaattggagtacctcgatcaaatccgatcagcttactgtgttgacgggaattcaaaagttaacacactggatacattgtacaaagaacagatagctgaagacatagtatgggatataatcgatgagttggagcaaattgccctacagcaataa

SEQ ID NO: 152 - DNA, 892 bp, Saccharomyces cervisiae
atggcaagagatatcacatttttgaccgtatttttagaaagttgtggcgctgtaaataatgatgaggcaggaaaattgttatctgcttggacttcaaccgtacgcattgagggaccggaatcaaccgactctaattcattatatattccactgctaccacctggaatgttgaaagtatgtttctcctagcaaaattaaaacccatccgtgaatgaagcgttactaactataataactggtagctttgtcactcgtaccaggaaaagtgaagattaaactgaattttaaaatgaacgatcgattagttacggaagagcaagagttgtttacaaaattgcgcgagattgtaggttcaagtattcgcttttgggaggaacaactgttttatcaagttcaagatgtaagcaccatagaaaaccacgtcattctcagtttaaaatgtacaattttaacggatgctcagataagtacgttcataagcaaacccagagagcttcatacgcatgccaaaggatatcctgaaatctattacctttccgagttatcaacaactgtcaattttttttctaaagagggaaactatgtcgaaataagccaggttattcctcattttaatgaatatttttcctctttaatagtgtctcaattggaatttgaatacccgatggtcttctccatgatttcaaggctccgattgaagtggcaacaaagttcgctcgctccgatatcctacgccctaacgagcaattcagtacttcttccaataatgcttaacatgattgcccaagacaaatcttcaacaaccgcgtatcaaattctgtgtcgaagaagaggtcctccaattcagaattttcaaattttttccttaccggctgtaacgtacaataagtag

SEQ ID NO: 153 - DNA, 1263 bp, Saccharomyces cervisiae
atgagtaaaattaaagttgttcatcccatcgtggaaatggacggtgatgagcagacaagagttatttggaaacttatcaaagaaaaattgatattgccatatttagatgtggatttaaaatactatgacctttcaatccaagagcgtgataggactaatgatcaagtaacaaaggattcttcttatgctaccctaaaatatggggttgctgtcaaatgtgccactataacacccgatgaggcaagaatgaaagaatttaaccttaaagaaatgtggaaatctccaaatggaacaatcagaaacatcctaggtggaactgtatttagagaacccatcattattccaaaaatacctcgtctagtccctcactgggagaaacctataattataggccgtcatgcttttggtgaccaatatagggctactgacatcaagattaaaaaagcaggcaaactaaggttacagtttagctcagatgacggtaaagaaaacatcgatttaaaggtttatgaatttcctaaaagtggtgggatcgcaatggcaatgtttaatacaaatgattccattaaagggttcgcaaaggcatccttcgaattagctctcaaaagaaaactaccgttattctttacaaccaaaaacactattctgaaaaattatgataatcagttcaaacaaattttcgataatttgttcgataaagaatataaggaaaagtttcaggctttaaaaataacgtacgagcatcgtttgattgatgatatggtagcacagatgctaaaatcaaagggcgggtttataatcgccatgaagaattatgatggcgatgtccagtctgacattgtggcacaaggatttgggtctcttggtttaatgacgtccatattgattacacctgatggtaaaacgtttgaaagcgaggctgcccatggtacggtgaccagacattttagaaaacatcaaagaggcgaagaaacatcaacaaattcaatagcctcaatatttgcctggacaagggcaattatacaaagaggaaaattagacaatacagatgatgttataaaatttggaaacttactagaaaaggctactttggacacagttcaagtgggcggaaaaatgaccaaggatttagcattgatgcttggaaagactaatagatcatcatatgtaaccacagaagagtttattgatgaagttgccaagaggcttcaaaacatgatgctcagctccaatgaagacaagaaaggtatgtgcaaactataa

SEQ ID NO: 154 - DNA, 852 bp, Saccharomyces cervisiae
atgaatagtaaccgatgccaaacgaatgaggtgaataaatttattagtagtacagaaaaggggccttttacgggcagggacaatacgctctcttttaacaaaatcgggagcagactgaattcaccaccgattctgaaggataaaattgagctgaaatttctacaacactcagaagatttgaatcaatcacggtcctacgtaaatattcgtcctagaaccttagaggatcaaagttacaaatttgaagcgccaaatctaaatgacaatgaaacttcttgggccaaggattttagatataacttccctaagaatgttgaaccgcccatcgaaaatcaaatcgcgaatcttaatataaacaacgggctacggacatctcagacagattttcccttaggcttttattcacagaaaaactttaacattgcttccttccctgtggttgaccatcagatattcaagacaacaggtttagaacatcctatcaacagccacattgattctttaattaatgctgaattttcggaactggaagccagtagtttggaagaagatgtccatacagaagaggaaaattcaggtacgagtctggaagatgaagaaactgccatgaaaggtttggcttccgatataattgagttttgcgataataatagtgccaataaagatgtaaaagaaagactaaacagttcaaagtttatggggctgatgggcagcattagtgatggttctatagttttaaagaaggataacggtacagaaagaaaccttcaaaaacacgtaggtttttgttttcagaattcaggaaactgggctggtcttgagttccatgatgttgaagacagaattgcttaa

SEQ ID NO: 155 - DNA, 444 bp, Saccharomyces cervisiae
atggcagtacagtttatactgtgctttaataagcagggtgtggtgcggttggtgagatggttcgatgtacacagttcggatcctcagcgtagccaggatgccattgcgcagatttatagactcatatcttccagagatcataagcatcagagtaacttcgtagagttttccgattcgacgaaactcatatacaggaggtatgcgggtctgtattttgtcatgggtgtggacttacttgacgatgaacccatatatttgtgccacatccatctgtttgtggaggtgctagatgcatttttcggcaatgtctgtgaactggatatcgtattcaacttttacaaagtctatatgataatggacgagatgtttattggaggggaaatacaagaaatttcaaaggatatgctgttagaaagactaagtattttagatagactagactag

SEQ ID NO: 156 - DNA, 207 bp, Saccharomyces cervisiae
atgaccatggaccaaggccttaacccaaagcaattcttccttgacgatgtcgtcctacaagacactttgtgctcaatgagcaaccgtgtcaacaagagtgtcaagaccggctacttattccccaaggatcacgttccttctgccaacatcattgccgtcgaacgtcgcggcggtctttctgacattggtaagaatacttccaactaa

SEQ ID NO: 157 - DNA, 1314 bp, Saccharomyces cervisiae
atggctctcaataaactaaagaatataccttctttaacaaacagttctcatagctcaattaacggcattgcatccaatgctgcaaattccaaaccaagcggagcagacacggatgatatcgatgagaatgatgaatctgggcaaagtattctattaaatattatttcccagctgaagccaggttgtgatttatctagaatcacacttccgacatttattctggaaaaaaaatcgatgttggagagaatcactaatcaattacaattcccagatgttcttttagaagcacactccaataaagacgggctgcaaaggttcgttaaagtggtagcatggtacctagcaggttggcacattgggcccagggctgtgaagaagcccctaaatcccattcttggagaacactttacagcttattgggatttgcctaacaagcaacaagccttttacattgcagaacaaacgagtcaccatcctcctgaatctgcgtatttttacatgattccagaatcgaatattagagttgatggagttgttgtgccaaaatcgaaatttttaggaaactcaagtgctgcaatgatggaggggttaactgtattgcaattccttgatatcaaggatgcaaatggtaaaccagagaaatatactctatcgcaaccaaatgtttacgcaaggggaattctgtttggcaagatgaggattgaattgggagatcacatggtcattatgggtcctaagtatcaagtggatattgagttcaaaacaaagggctttatttctggtacctatgatgcaattgaaggtacaattaaggattacgatggtaaggaatactaccaaattagtggtaagtggaatgatattatgtatatcaaagatttgagggaaaaaagctctaaaaagactgttctcttcgatactcatcagcattttcctctagctcctaaagtccgcccattggaggaacagggagaatacgaatcgagaaggctttggaagaaggttacggatgcgctggctgtacgtgaccatgaagtagctacagaagaaaagtttcagatagaaaaccgccaaagagagctggccaaaaagagggccgaagacggcgttgaatttcattcaaaactatttagaagggcagagccaggtgaggatttagattattatatttacaagcacatccctgaagggaccgacaagcatgaagaacagatcaggagcattttggaaactgccccgattttaccaggacagacattcactgaaaaattttctattccggcttataaaaagcatggaatccaaaagaattag

SEQ ID NO: 158 - DNA, 1107 bp, Saccharomyces cervisiae
atggctagaaccataacttttgatatcccttcccaatataaactcgtagatttaataggtgagggagcgtacggaacagtatgttcagcaattcataagccttccggcataaaggtagctatcaagaaaatacaaccgtttagcaaaaaattgtttgttacaagaactatacgtgagatcaagcttttacggtatttccatgaacacgaaaacataataagtatattggataaagtaaggccagtatccatagacaaactaaacgctgtttatttagtcgaagagttgatggaaaccgatttacaaaaagtaattaataatcagaatagcgggttttccactttaagtgatgaccatgttcaatactttacataccaaatcctcagagccttaaagtctattcacagtgcacaagttatccatagagacataaagccatcaaacctgttactaaattccaattgtgatctcaaagtctgcgattttggactagcgaggtgtttagctagcagtagcgattcaagagaaacattggtaggattcatgacggagtacgtcgcaacgcgatggtacagggcacccgagataatgctaacttttcaagagtacacaactgcgatggatatatggtcatgcggatgcattttggctgaaatggtctccgggaagcctttgttcccaggcagagactatcatcatcaattatggctaattctagaagtcttgggaactccatctttcgaagactttaatcagatcaaatccaagagggctaaagagtatatagcaaacttacctatgaggccacccttgccatgggagaccgtctggtcaaagaccgatctgaatccagatatgatagatttactagacaaaatgcttcaattcaatcctgacaaaagaataagcgcagcagaagctttaagacacccttacctggcaatgtaccatgacccaagtgatgagccggaatatcctccacttaatttggatgatgaattttggaaactggataacaagataatgcgtccggaagaggaggaagaagtgcccatagaaatgctcaaagacatgctttacgatgaactaatgaagaccatggaatag

SEQ ID NO: 159 - DNA, 2358 bp, Saccharomyces cervisiae
atgtcatctgcagagatggaacaattgttacaggccaagacactggccatgcacaacaatccaacggagatgctgcccaaggtgctcgaaactacggcatccatgtaccacaacggtaatctcagcaagctgaagttgcctttggccaagttttttacacagttagttctagacgtggtgtcgatggactctccaattgcgaatactgagagaccgtttattgctgctcaatatctgccactacttcttgctatggcgcaatccaccgcggacgtactagtgtacaagaatatcgtgcttattatgtgcgcttcatacccgctggtgttggatctggttgctaagacatcaaaccaggaaatgtttgatcagttgtgtatgctgaagaagttcgtgctctcgcactggagaactgcatatcctttgcgtgccaccgttgacgatgaaacggatgtcgaacaatggctggcgcagattgaccaaaatatcggcgtgaaattagcgaccatcaagttcatatctgaggtcgtgctgtcgcaaactaaatcacccagcggcaacgagattaattcatctaccatcccggataaccaccctgtgttgaacaaaccggctttggagagcgaggctaagaggcttcttgatatgttgctaaactacctaattgaggaacagtacatggtctcgtccgttttcattggtatcatcaattctttatccttcgtcatcaaaagaaggccgcagacaacaataagaattctttccgggctgttgcgtttcaacgtcgacgccaagtttcccctagagggcaagtctgacttgaactacaaactatccaagagatttgttgaaagggcgtacaagaactttgtgcaatttgggctaaaaaatcaaatcattacaaaatccctctcatccggatcagggtcatcgatctactccaagctgaccaagatttctcaaactttacacgttattggcgaagagaccaagagcaagggaattttgaacttcgacccttccaagggcaatagcaagaaaacgttgtccaggcaggacaaactaaaatacatctcactatggaaaaggcaattatccgcgttattgtctactctaggggtgtccacaaagacccccacgcctgtgtccgcacctgcaacgggctcttcaaccgaaaacatgcttgatcaactgaagatattgcaaaaatacaccctcaacaaggcttcacaccagggcaatacttttttcaacaactcacccaaaccaatcagcaacacctactcatctgtgtactcattgatgaacagttcgaactccaaccaggatgtgacccagctacccaatgacatacttatcaagctgtccacagaggccatcttgcaaatggacagcacgaaactgatcaccggattgtctatcgttgcttcgaggtacacggatttaatgaatacgtacatcaattctgtaccgtcctcgtcatcatcaaagaggaaatccgacgatgatgacgacggcaacgacaatgaagaagttggaaacgatggcccaacggctaatagcaagaaaatcaaaatggaaacagaaccactagcggaggaaccagaggagcccgaagacgatgaccgaatgcagaagatgcttcaagaagaggaaagcgcccaagaaatctcaggagatgccaacaaatcaacttctgccattaaggagatcgcacccccctttgaacctgactcattgacgcaggatgaaaaactaaagtacctctcaaagctgaccaagaaactgtttgaattatccggtcgccaggatactacccgggccaaatcttcgtcttcctcctccatattactggacgatgacgactcctcgtcatggttacacgtcttaatcagattggttacgagaggaatcgaagcacaagaggccagtgacctgattcgtgaagaactgcttggcttcttcatccaggatttcgagcaacgtgtcagtctgatcattgaatggctcaatgaagaatggttcttccaaacctcgctgcatcaagatccctctaactacaaaaaatggtccttaagagttctcgagtctctgggtccattccttgaaaacaaacacagacgattcttcatcagacttatgagcgaactgcccagtcttcaaagcgatcatcttgaggcactgaagcctatctgcctggatccggcaagaagttcccttggtttccaaacgctaaagtttctcattatgtttagacccccagtgcaggacactgttcgcgacctgctgcatcagctaaagcaagaagatgaaggcttacacaagcagtgcgattcactgcttgacaggctaaaatga

SEQ ID NO: 160 - DNA, 345 bp, Saccharomyces cervisiae
atgaaggccagttacttagttttgattttcattagcatattctccatggcacaggcatcttccttatcatcatacatcgtaactttccccaagacggataatatggctacggaccagaatagcattattgaagatgtcaaaaaatatgtggtggacatagggggtaaaataacacacgaatatagcttgataaagggctttacagtggacttacctgatagcgaccaaattttggacggtctgaaagaacgtttgagctatattgaaagcgagtacggtgctaaatgcaatttggaaaaggattcagaagttcatgctctaaaccgtgaccatttagttgcttag

SEQ ID NO: 161 - DNA, 942 bp, Saccharomyces cervisiae
atgggaaaaggtaaaaataagatgcacgaacccaaaaatggaagaccacagagaggcgctaatgggcacagttccaggcaaaaccataggcgcatggaaatgaagtacgataattcagaaaaaatgaagtttcctgttaaactggctatgtgggattttgatcattgcgatccaaagagatgcagtggtaaaaaacttgaaaggttgggcttaattaaatcattgagagttggacagaaatttcaaggtattgtcgtttcgccaaacggcaaaggtgttgtttgccctgatgatctagaaattgtcgaacaacacggcgcttcggtggtcgagtgttcttgggcacgtttagaagaggtacccttcaataaaataggcggtaagcacgaaaggctgctgccgtatttagtggccgctaatcaagtaaattacgggagaccatggaggctcaattgcgttgaggcattagcggcttgttttgctatcgtcgggagaatggattgggccagtgaattgttatcacacttctcgtggggaatgggatttttagaattaaacaaagaattgcttgaaatctatcagcagtgcactgactgcgactctgtaaagagggctgaagaggaatggttgcaaaaattagaaaaggaaactcaagaacgaaaatcccgagctaaagaagaagatatatggatgatgggtaacataaatagaaggggtaatggttcgcaatctgacacatcagagagtgaggaaaactcagaacaatctgatttggaaggcaataatcaatgtattgaatatgactctttaggtaatgctattcgtatagataacatgaaaagcagggaagcgcaatctgaggaatcagaagacgaggaaagtggttcaaaagaaaatggagagcctttaagttatgaccccttaggcaatttaattcgatag

SEQ ID NO: 162 - DNA, 843 bp, Saccharomyces cervisiae
atgtcgcaagaaattaggcaaaatgagaaaatcagttatcgtattgaaggaccattcttcattattcacttaatgaaccctgacaatttgaatgcactagaaggtgaagactatatttatttaggagagttactagaactagcggacagaaatcgtgatgtatattttacaattatacaaagcagtggtagatttttttccagtggtgctgatttcaagggtattgcaaaagcccaaggggatgataccaataaatatccttcggaaacaagcaagtgggtgtcaaattttgtcgctagaaatgtttatgtcactgatgccttcatcaagcattccaaagttttaatttgctgtttgaatggaccagcaatagggttgagcgcggcactggtagcgttatgtgacattgtgtacagtataaatgacaaggtttatttgctatacccctttgctaacttaggactaattaccgaaggtggtacaacggtctctttgccattgaagtttggcacaaatacgacgtatgaatgcctcatgttcaacaaaccattcaagtacgatataatgtgcgagaacggatttataagcaagaattttaacatgccatcttcaaacgctgaagcgttcaatgcaaaggtcttagaagaattgagggagaaagtgaaagggctatacctgcccagttgcttagggatgaaaaaattgctgaaatcgaaccacatcgatgcattcaataaggctaactcagtggaagtaaatgaatctctcaagtattgggtagatggagagcccttaaaaagatttaggcagctgggctcgaaacaaaggaagcatcgtttatga

SEQ ID NO: 163 - DNA, 450 bp, Saccharomyces cervisiae
atgttcaagcatagtacaggtattctctcgaggacagtttctgcaagatcgcctacattggtcctgagaacatttacaacgaaggctccaaagatctatacttttgaccaggtcaggaacctagtcgaacaccccaatgataaaaaactattggtagatgtaagggaacccaaggaagtaaaggattacaagatgccaactacaataaatattccggtgaatagtgcccctggcgctcttggattgcccgaaaaggagtttcacaaagttttccaatttgctaaaccacctcacgataaagaattgatttttctttgtgcgaaaggagtaagagccaaaactgccgaagagttggctcgatcttatgggtacgaaaacactggtatctatcctggttctattactgagtggttagctaaaggtggtgctgacgttaagcccaaaaaataa

SEQ ID NO: 164 - DNA, 990 bp, Saccharomyces cervisiae
atgaccaccgtgtccatcaataagcccaacctgctgaaattcaagcatgttaaaagctttcaacctcaagaaaaagactgcggacccgtaacctcattgaatttcgacgataatggccagtttctactgacctcttcttccaacgatacaatgcaattgtacagtgccacgaactgcaaattcttggacactatagcctctaagaaatatggctgtcactccgctatctttacgcacgcacaaaacgaatgtatctattcctctacaatgaaaaattttgacattaaataccttaatctggaaacaaaccaatatctaagatatttttccggtcatggcgccctagtgaatgatttgaagatgaaccccgtgaacgatacgtttctatcgtcgtcatacgatgaatccgttaggctttgggatttgaagatctctaaaccgcaagttattataccaagtctcgtaccaaattgtatcgcatatgatccaagtggccttgtattcgcattggggaacccagagaatttcgaaatagggctatataatctgaaaaaaattcaggagggtcctttcttgataattaaaattaatgatgcgactttcagtcaatggaataaattagaattttctaacaatggaaagtatttattagttggctcctcgataggaaagcatttaatttttgacgcattcacaggtcaacaattattcgaactaataggaacaagggccttcccgatgagagaatttctagattctggatctgcttgtttcacaccagatggtgaattcgtccttggaaccgattatgacggtaggattgccatttggaatcattctgattcaataagtaacaaagtattaaggccgcaagggttcattccctgtgtttctcatgagacctgccccaggtcaattgcattcaaccctaaatattcgatgtttgttaccgcagacgaaacagtagatttttacgtgtacgatgaatga

SEQ ID NO: 165 - DNA, 843 bp, Saccharomyces cervisiae
atgaaggcgctagttgaagagattgataagaaaacttacaatcctgacatatatttcacgtcattggatcctcaagcacgtcgatatacttcaaagaagattaataagcaaggcacaatatccacctctaggcccgtaaagcgcataaactactcactggcagatttagaggccaggttatatacttcgagatctgagggagatggcaatagtataagcagacaggatgaccgaaatagtaagaattcccattcatttgaagaaaggtacacacaacaagagattctccagtcggacaggaggtttatggaacttaacacagaaaatttctcggatttaccaaatgtaccgactttattaagtgatctcacaggcgtaccacgagatagaattgaatcaacaaccaaaccgatctcacagacctcggatggtctctctgcattaatgggtggttcttcttttgtaaaagagcattccaagtatggtcatggttgggtgcttaaaccagaaaccttacgggaaatacaattatcgtataaatctacaaaactacctaaaccaaagaggaagaataccaatcgtattgtggcgttaaagaaggttttaagttcaaaaagaaatttacattcgtttttagattctgcgctgctaaacttgatggataagaatgtcatttaccacaatgtttacaataaacgatacttcaaagtgttacccctaattacgacatgctctatttgcggtggctacgatagtatttcaagttgcgttaattgtggaaataagatttgttctgtaagttgttttaaattgcataatgaaactaggtgcagaaatagatag

SEQ ID NO: 166 - DNA, 1338 bp, Saccharomyces cervisiae
atgatgatgttaattactatgaaaagtacagtactgttgagtgtttttaccgtcttagcgacatgggctggattgctagaagctcatccattgggtgacacttcagatgcatccaaattaagctcagactactcgctccctgatctcattaatgcacgtaaagtgcccaataactggcaaactggagaacaagctagtctagaggaagggagaattgtattgacttctaagcaaaattccaagggttcactttggttgaagcaaggattcgatttgaaggattcttttactatggagtggacatttaggagtgttggttattctggccaaaccgacggtggcatatcattttggtttgttcaagattctaacgtaccacgcgataagcagttatacaatgggccagtgaactatgatggtttacaattattagtggataacaatggtccattgggcccaacacttcgtggtcaactaaatgatggtcaaaagcctgtagataagacgaaaatctatgatcagagttttgcatcttgtttgatgggttatcaggattcctccgttccttccacgatcagagtaacttatgatttggaagacgacaacttattaaaagttcaggtggacaataaagtctgtttccaaactaggaaggttcgctttccctctgggtcttaccgtattggtgtcaccgctcaaaatggagcagtgaataataatgcagagtcttttgaaatattcaaaatgcaattttttaatggcgtgattgaagattctttgatccctaatgtgaatgcaatgggtcagccaaaactgatcaccaaatacattgaccaacaaaccggcaaagaaaaattgattgaaaaaacagcatttgacgctgacaaagacaaaattacaaactatgaattgtataagaaactggatagagttgaaggtaaaattcttgcgaacgatatcaatgctttagaaacaaagctaaatgatgtcattaaggtccaacaagagctattatcattcatgactacgataactaaacagctctcttctaagccaccagctaataatgaaaagggaacgtccaccgatgatgcaatcgctgaggataaagaaaatttcaaagacttcttatcaatcaatcagaaattggagaaagtcctggttgaacaagaaaagtatagggaagctaccaaacgtcatggacaagatggtcctcaggtcgacgaaattgccagaaaactaatgatttggttacttccattaattttcattatgttggttatggcatattacacattcagaatcagacaagagatcataaagaccaaactgctatga

SEQ ID NO: 167 - DNA, 1449 bp, Saccharomyces cervisiae
atgcctgactatgacaattacactacgccattgtcttctagatatgcctccaaggaaatgtcagcaacgttttctttgagaaacagattttccacatggagaaaactatggttaaatttggctattgctgagaaggaattgggcttaactgttgttacagatgaagcaattgagcaaatgcgcaaacacgtcgaaatcactgatgatgaaatcgcaaaagcttctgctcaagaagccattgtaagacatgatgttatggcacatgttcatacatttggtgaaacttgtccggctgctgcgggtatcattcacttaggtgctacttcctgtttcgttacagacaatgctgatctaatctttattagggacgcctacgatattattattccaaaacttgttaacgtcatcaacagattggctaagtttgctatggaatacaaggatttgcctgtattgggttggactcactttcaaccagcacaattaacgaccttgggtaagagagctactttatggatacaagagctattgtgggatttgagaaactttgaaagagctagaaacgatatcggtctacgtggtgttaagggtactactggtactcaggcatcattcttggccttattccatggtaatcatgataaagttgaagcccttgacgaaagagtaactgaattattaggtttcgataaggtatatccagtcactggtcaaacctactcaagaaaaattgacattgacgtgttggctcctttgtcttcttttgctgctactgcacacaaaatggctactgacataagattattagccaacctgaaggaagttgaggaaccttttgagaaatcacaaatcggatcctctgctatggcttacaagagaaacccaatgcgttgtgagagagtgtgctccttggctagacacttaggttccttgtttagtgacgccgttcaaactgcatccgttcaatggttcgaaagaactctggatgattctgctattagaagaatttctttaccaagtgcatttttaaccgcagatattctattatctactttgttgaacatctcatccggtttagttgtgtatccaaaggttatcgaaaggagaattaagggtgaactaccttttatggctactgaaaatatcatcatggctatggtagaaaagaatgcctccagacaagaagtacatgagcgtattagagtgctctctcatcaagccgcagcagtagtcaaggaagaaggtggggaaaatgatttaattgaacgagtaaagagggatgaatttttcaagcctatctgggaagaattagattctttactggaaccatccacttttgttggtagagctccacaacaagttgagaaatttgttcaaaaagacgttaacaatgctttacaacctttccaaaagtacctaaacgatgaacaagtcaagttaaatgtttag

SEQ ID NO: 168 - DNA, 2382 bp, Saccharomyces cervisiae
atgcaagtactagtgacgctctggtgtctaatatgcacatgcctggtactaccagtggccgccaagaaaaggacactgacagcgagttcactggtcacgtgcatggagaactcacagctttcagccaatagtttcgatgtgtcgttttctccagacgatcgatcgctacattacgatctggatatgaccacgcagatcgactcttacatctacgcttatgtggacgtgtatgcctacgggttcaagattattacggagaacttcgacgtgtgttcaatgggttggaagcagttttgccctgtgcacccaggtaacatacaaatcgactccattgaatacattgcccagaagtacgtgaaaatgattccgggaattgcctaccaagtgcccgatattgatgcgtacgtaagattgaacatttataacaacgtaagtgaaaatttggcttgtatccaggttttcttttccaatgggaaaactgtatcacaaattggggttaaatgggtgacagctgttatcgccggtattggtttattaacttccgctgtcttgtccaccttcgggaactccacagcagcatctcacatttctgcaaacaccatgtcactgttcttatatttccaatctgtcgctgtggtcgcaatgcaacatgtagacagtgttccacccattgctgctgcctggtctgaaaaccttgcctggtcgatgggcttgatccgtattacatttatgcagaaaatcttccgttggtatgtagaggcgactggaggctccgcatctctatatttgaccgcgacaacaatgtcagtgctcactcaacgaggtctggattaccttaaaaatacttcggtttacaagagggcggaaaatgtcttgtacggtaactcaaacactttaatctttcgaggaattaaaagaatgggataccgtatgaagattgaaaatacggccatcgtttgtactgggttcacattctttgtgctgtgcggttattttttggccgggtttatcatggcctgcaaatacagtatcgagttatgtataagatgtggttggatgcggagtgataggttttaccaatttaggaaaaactggaggtcagttctgaaaggatcgttgttaagatacatctatattgggttcacgcaactgacaattttaagtttttgggagttcactgaacgggattccgccggtgttattgttattgcatgcctattcattgtattgtcatgcgggttgatggcgtgggctgcgtacagaaccatttttttcgcaagtaaatctgtggaaatgtacaataacccagctgctttattgtatggtgatgagtacgtcttaaacaagtacgggtttttctacaccatgttcaacgcaaaacattattggtggaatgctcttttaacgacgtatattcttgtaaaagctttatttgtcggattcgcacaggcatcaggtaaaacgcaagcattggctattttcattattgacttggcgtattttgttgccatcatccgttataaaccatatttggaccgtccaacgaatattgtcaacatttttatttgcactgtcaccttggtcaactctttccttttcatgtttttctcaaacttgtttaaccaaaagtatgctgtctctgccatcatgggctgggtgtttttcattatgaatgctgcgttttctttgcttctactgttgatgattctggcctttaccacaatcattctgttttctaagaatcctgactccaggttcaagccagcaaaggatgacagagcatctttccaaaagcatgctattcctcatgaaggtgccttgaataagtcagtggccaacgaattaatggccctaggtaatgtggcaaaggatcataccgaaaattgggaatacgaactgaagagtcaagaaggtaaaagtgaagataatcttttcggagttgaatacgatgacgagaaaacaggaactaattcagagaatgctgaaagtagcagtaaggaaaccacccgtccaaccttttctgaaaaggttttacgttcattatcaatcaaaaggaataagagtaaactgggcagtttcaagcgcagcgctccggataagataacacaacaagaggtttctcctgaccgcgccagctcttcgcctaacagcaagtcataccccggtgtctcgcacaccaggcaagaatctgaagcgaataatgggctaatcaatgcatatgaagatgagcaattcagtctgatggaaccaagcatactggaagacgctgctagttccacccaaatgcatgctatgccagcccgagatttgagcttgagcagtgttgcaaacgcccaagatgttactaaaaaagcaaacatcctggatcctgattatttgtaa

SEQ ID NO: 169 - DNA, 1044 bp, Saccharomyces cervisiae
atggatatgaaagtagaaaaattaagtgaagatgaaattgcactgtatgatagacagattcgtctatggggaatgacagcacaggccaatatgagatcagcaaaagtattgctgatcaatcttggagcaattggttctgaaattaccaaaagtatcgtccttagtggtatagggcatttaaccatattggatggacacatggtgactgaagaagatttaggatcccagttcttcataggctctgaagatgttggccaatggaagattgatgcaacaaaagagagaattcaagacttgaacccccgtatagagttgaactttgataagcaggatttgcaagaaaaggacgaagagttctttcagcagtttgatttagtcgtggctacagaaatgcagattgatgaagcaatcaaaattaacacattaactcgaaaactaaacattccattatatgttgctggttctaatggattgttcgcttatgtttttattgatttgattgaattcatttcagaggatgaaaaattgcaaagtgtaagacctaccaccgttggtcccatttcaagcaataggagcattatagaagttactactagaaaagatgaagaagatgaaaaaaaaacatatgaacgaatcaagaccaagaactgctataggccactgaacgaagttttaagcacagcaacattaaaggaaaaaatgacgcaaagacagttaaaaagagtcactagtatcttgccgttaaccctgtccttattgcaatatggtctcaaccaaaagggcaaggccataagctttgaacaaatgaaaagagatgcggccgtatggtgtgaaaatctgggtgtaccagcaacagtggtaaaggacgattacatacaacagtttatcaaacagaaaggtatcgagtttgctcctgtcgcggccattatagggggtgctgtagcgcaggatgtcattaacattctaggtaaaaggctatctccattaaataatttcattgtttttgatggtattacattagacatgccactttttgagttttag

SEQ ID NO: 170 - DNA, 924 bp, Saccharomyces cervisiae
atgtctgaagaatttccatctcctcaactaatcgatgatttggaagaacatccacagcatgataatgctcgagtcgtgaaagatttgcttgcaggtacagcgggtggtattgcgcaagtgctagtgggccagccctttgatacgacaaaagttaggttacaaacatcgagcaccccaacaacagccatggaagtcgtcagaaagctgcttgccaatgaagggcctcgcgggttttacaaaggaactctgacgccattaattggtgttggtgcatgtgtttcattacaatttggtgttaatgaagctatgaagagattttttcatcatcgcaatgctgatatgtcatcgactttgtcattgccacagtattacgcatgtggtgtcacaggcggtatagtaaactcattcttggcgtccccaattgagcatgtcaggattcgcttgcaaacacagactggctcaggcaccaacgcagaattcaagggtcctttggaatgcatcaaaaaattaagacataacaaggccttgctacgtggtttaacacctacaatattgagagaaggtcatggatgtggcacatatttcttagtgtatgaagcgttgattgctaaccaaatgaacaaaagacgtggactagagagaaaggacattcctgcatggaaactttgtatttttggagcattgtctggcactgccttatggttgatggtatatccattagatgtcatcaagtctgtcatgcaaacggataatttacaaaagcctaaatttggtaattctatttccagtgtagccaagactttatatgccaatggagggataggcgcttttttcaaagggtttggtcctaccatgctaagagctgctcccgccaatggtgccacttttgctacttttgaattagcgatgaggttattgggttga

SEQ ID NO: 171 - DNA, 588 bp, Saccharomyces cervisiae
atgattggcagaggtgtgtgctgcagatcgttccacactgctggatctgcctggaagcaatttggatttcccaaaacacaagtgacaacgatttacaacaagactaagagcgcatctaactataaagggtatttaaagcacagagatgctccaggaatgtactatcaaccatcagaatccatcgcaaccggatctgttaacagtgagaccattccacgtagctttatggcagccagtgaccctcgtagagggcttgacatgcctgttcaaagcactaaagcgaagcagtgtccaaatgttctcgtaggtaagagcacagtgaacggcaaaacctatcatctgggacctcaagaaattgatgagatccggaagttacgtcttgacaatcctcaaaagtatacacgcaaatttttggctgcaaaatatggcatttcgccattatttgtatccatggtctcgaaacctagtgaacaacatgtacaaattatggaaagtagattgcaagaaatccaatcacgctggaaggagaagaggcgtatagccagagaggaccgtaagcgtagaaaactcctgtggtaccaggcgtga

SEQ ID NO: 172 - DNA, 2283 bp, Saccharomyces cervisiae
atgaagataacgtgtacagacttggtgtacgtcttcattttactcttcctaaacacgagttgtgtccaagccgttttttcagatgatgcatttatcactgattggcaactggctaacttaggtccttgggagaaagtcatccctgattctcgagaccgcaacagggttctcatcttatcgaaccctaccgaaacttcctgcttagtttcttcgtttaacgtttcttccggacagattcttttcagaaacgttttaccctttaccattgatgagattcaactggatagtaatgaccataacgcaatggtttgtgtgaactcttcaagcaaccattggcagaaatatgatttacacgattggtttttactagaggaaggcgtagataatgccccttctacgaccattttacctcaatcctcatatttaaacgatcaagtatctattaagaacaatgaactacatattctcgatgagcagtcaaaactggcagaatggaaattggagttacctcaagggttcaataaagtggaatattttcatcgtgaagatcccctggcgttagtgttgaacgttaatgatacccaatatatgggattctctgccaatggcacagaattgatccccgtttggcaaagagatgaatggttgactaacgtggtagactatgctgtattggacgtcttcgattctagggatgtggagttgaacaaagatatgaaagcggaacttgattcaaattcgctttggaatgcttactggcttagattgacaactaattggaatcgccttatcaacttattgaaagaaaaccaattctcaccaggacgtgtcttcactaaactcctagctctagacgctaaggataccacggtatcagatttgaagttcggattcgccaaaatcttaattgttttgacgcatgatggctttatcggcggccttgatatggtcaataagggccaacttatctggaaactcgatttagaaattgatcagggcgtcaaaatgttctggacggataaaaaccatgacgaacttgttgttttttcgcatgatgggcattatttgacaattgaagttactaaagatcaaccgattatcaaatcaagatcccccctatctgaaaggaaaactgttgattccgttattaggctgaatgaacatgatcaccagtatctgattaagtttgaggataaggatcatttactgttcaaattgaatcccggcaagaatacggatgtaccaatagttgccaacaaccattctagttcccacatattcgtcacagagcatgacacgaatggcatttatggctacataatcgaaaacgatacggtaaaacaaacttggaaaaaagccgtaaattcgaaagagaaaatggtggcatatagcaagagggaaacaacaaacctaaacactcttggtattacactaggtgacaaatcggttctttataaatatttgtaccccaacctagcggcttatctgatcgctaatgaagaacatcatacaatcacttttaacttaattgataccattacaggagaaatcctcattacccaagagcacaaggattctccggattttaggtttccaatggatattgttttcggtgaatattgggtcgtttattcctatttcagttctgaacctgttccagaacaaaagttagtagtggtggaattatatgagtcactaaccccagatgagcgtttgtctaactcaagcgacaatttttcttatgatccattgactggacacattaacaaacctcaatttcaaactaaacaattcatttttcccgagattatcaaaacaatgtccatttccaagacaacggatgatattaccacaaaggcaatcgttatggaattagaaaatggacaaatcacctacataccaaagcttttattgaatgcaagaggtaaaccagcagaagaaatggccaaggataagaaaaaagagtttatggctaccccatacacgccagttatcccaattaatgataatttcattatcactcatttcagaaatctattgccaggatccgattcgcagttgatctccatcccaaccaatctggaatccacaagcattatatgtgatctaggccttgatgtattttgtacaaggatcacaccttcgggccaatttgatttaatgagtcctactttcgaaaagggtaaattgcttattactatattcgtcttgttggtgatcacgtattttatccgtccttctgtttcaaacaagaagttgaaatcccaatggctaattaaatag

SEQ ID NO: 173 - DNA, 1644 bp, Saccharomyces cervisiae
atggtaaagaaacaccaaaatagtaaaatgggtaatacaaatcactttggacatctcaaaagttttgtgggaggtaacgtggttgcccttggtgctggaacaccttatcttttctcattttatgctcctcagctactgagcaagtgccacatacctgtttctgcctcaagtaagctatccttctctttaacaataggaagctcactgatgggaattttagcaggaatagttgtcgatcgaagtcctaaactgtcctgtctaattggttcaatgtgtgttttcatcgcgtatttgattttgaatttatgctataagcacgaatggtctagtacttttctcatatcgttaagtttggtactcattggatatggttctgtctcaggtttttacgcttctgtgaaatgtgcaaatacaaattttcctcaacacaggggtacagctggggcatttcctgtgtccctatacggtttatcgggcatggtgttctcatatctttgctcaaagctttttggtgagaacatcgagcatgtcttcattttcttgatggttgcgtgtggttgcatgattttagtaggctatttctcattagatatattctctaatgcagaaggagatgatgctagcattaaggaatgggagcttcaaaaaagcagggaaacagacgataatatagtaccgttatatgagaacagtaatgactatataggttcacctgtgcgttcatcatcccctgctacctatgaaacttatgcattgtcagacaattttcaggaaacgtcagaattttttgcacttgaggatagacagttatcaaatcgaccattgttatcaccttcttccccacacacaaagtatgatttcgaggatgagaataccagcaaaaatacagtgggcgagaatagcgcacagaaaagtatgagattacatgtattccaaagcttaaaatcttcaacatttattggttactacatagtattgggtatactacaaggcgtgggtttaatgtacatatattctgtggggtttatggtacaagcccaggtttctactccacccttaaatcaattaccaattaatgcagaaaaaattcaatcattacaagtaactctcctgtctcttctttcattttgcggcagattatcatctgggcctatatcagattttttggtcaagaaattcaaagctcaaagactatggaatattgtcatagcatcgcttttggtatttcttgcatcgaataaaatatcccatgacttcagcagcattgaagatccttctttaagagcctccaaatcattcaagaatatttcggtatgctcagcgatcttcggttattcttttggcgttctatttggtactttcccctccatagtagcagatagatttggcacaaatgggtatagtacgctgtggggtgttttaacgactggtggtgtattttcagtgagtgtttttaccgatatattaggtagagatttcaaggcaaatacaggagatgatgatgggaactgtaaaaagggagtgctttgctacagctatacttttatggttacgaaatattgtgccgcttttaatcttttgttcgttttggggataattggatatacgtactatcgaagaagagcaactgcaaattccctgtag

(19) United States
(12) Patent Application Publication
(10) Pub. No.: US 2014/0322776 A1
(43) Pub. Date: Oct. 30, 2014

(54) OVEREXPRESSION OF GENES THAT IMPROVE FERMENTATION IN YEAST USING CELLULOSIC SUBSTRATES

(71) Applicants: Oscar Alvizo, Fremont, CA (US); Galit Meshulam-Simon, Los Altos, CA (US); Amy Lum, Redwood City, CA (US); Dayal Saran, Sunnyvale, CA (US)

(72) Inventors: Oscar Alvizo, Fremont, CA (US); Galit Meshulam-Simon, Los Altos, CA (US); Amy Lum, Redwood City, CA (US); Dayal Saran, Sunnyvale, CA (US)

(73) Assignee: CODEXIS, INC., Redwood City, CA (US)

(21) Appl. No.: 14/360,198

(22) PCT Filed: Aug. 31, 2012
(86) PCT No.: PCT/US2012/053515

§ 371 Date: May 22, 2014

Related U.S. Application Data
(60) Provisional application No. 61/564,772, filed on Nov. 29, 2011.

(51) Int. Cl. C12P 7/04

(52) U.S. Cl. CPC C12P 7/04
USPC 435/157; 435/254.2; 435/254.22; 435/254.21; 435/254.23

(57) ABSTRACT

The invention relates to recombinant yeast host cells that overexpress proteins to improve glucose utilization, pentose sugar utilization and/or production of a fermentation product in a fermentation reaction.

### CROSS-REFERENCE TO RELATED APPLICATIONS

This application claims priority benefit of U.S. provisional application No. 61/564,772, filed Nov. 29, 2011, which application is herein incorporated by reference.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 90834-850413_ST25.TXT, created on Aug. 31, 2012, 492,091 bytes, machine format IBM-PC, MS-Windows operating system, is hereby incorporated by reference.

### BACKGROUND OF THE INVENTION

The conversion of carbohydrates to ethanol by yeast is a well-known fermentation process used in the food and beverage industry and in the production of bioethanol. However, utilization of fermentable sugars in fermentation reactions using cellulosic substrates can be inefficient due to the presence of inhibitors in fermentation reactions, or because some sugars may have poor utilization rates. Accordingly, there is a need for improved fermentation reactions. This invention addresses that need.

### BRIEF SUMMARY OF THE INVENTION

The invention relates, in part, to overexpression of proteins in yeast to improve fermentation reactions. In some embodiments, overexpression of one or more of the proteins improves hexose sugar utilization, e.g., glucose utilization, in a fermentation reaction. In some embodiments, overexpression of one or more of the proteins improves pentose sugar utilization, e.g., improved xylose utilization, in a fermentation reaction. In some embodiments, overexpression of or more protein products provides increased yield of a fermentation product, such as an alcohol, e.g., ethanol, from fermentation reactions. Thus, in one aspect, the invention relates to a recombinant yeast cell that is genetically modified to overexpress at least one of the following proteins: an ERR3, FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, or PDR12 protein, or a homolog or variant of the protein. In some embodiments, the protein is ERR3, FOX2, LYS1, MET1, MIG2, RMD6, RME1, SIP1, SNP1, or TDH1; or a homolog or variant of the protein. In an additional aspect, the invention relates to a recombinant yeast cell that is genetically modified to overexpress at least one of the following proteins: LCB2, CHA1, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W protein, or a homolog or variant of the protein. In some embodiments, a recombinant yeast cell of the invention is genetically modified to overexpress a protein having at least 70% identity, at least 75%, at least 80%, at least 85%, at least 90%, at least 91%, at least 92%, at least 93%, at least 94%, at least 95%, at least 96%, at least 97%, at least 98%, or at least 99% identity, to an amino acid sequence selected from SEQ ID NOS:1-27 or SEQ ID NOS:55-113. In some embodiments, the recombinant yeast cell is genetically modified to overexpress a protein comprising an amino acid sequence selected from SEQ ID NOS:1-27 or SEQ ID NOS:55-113. In some embodiments, the protein has at least 70% identity, at least 75%, at least 80%, at least 85%, at least 90%, at least 91%, at least 92%, at least 93%, at least 94%, at least 95%, at least 96%, at least 97%, at least 98%, or at least 99% identity, to an amino acid sequence selected from SEQ ID NOS:1-10, or comprises an amino acid sequence selected from SEQ ID NOS:1-10. In some embodiments, the nucleic acid that encodes the protein has at least 70% identity, at least 75%, at least 80%, at least 85%, at least 90%, at least 91%, at least 92%, at least 93%, at least 94%, at least 95%, at least 96%, at least 97%, at least 98%, or at least 99% identity, to a nucleic acid sequence selected from SEQ ID NOS:28-54 or 114-173, or comprises a nucleic acid sequence selected from SEQ ID NOS:28-54 or 114-173.

In some embodiments, the recombinant yeast cell comprises a recombinant expression construct comprising a promoter operably linked to a nucleic acid sequence that encodes a protein having an amino acid sequence selected from SEQ ID NOS:1-27 or selected from SEQ ID NOS:55-113; or a homolog or variant of said protein that has at least 70% identity to an amino acid sequence selected from SEQ ID NOS:1-27 or SEQ ID NOS:55-113. In some embodiments, the protein has at least 75%, at least 80%, at least 85%, at least 90%, at least 91%, at least 92%, at least 93%, at least 94%, at least 95%, at least 96%, at least 97%, at least 98%, at least 99% to an amino acid sequence selected from SEQ ID NOS:1-27 or SEQ ID NOS:55-113. In some embodiments, the protein comprises an amino acid sequence selected from SEQ ID NOS:1-27 or SEQ ID NOS:55-113. The protein which the recombinant yeast cell is genetically modified to overexpress may be endogenous to the yeast cell, or may be exogenous to the yeast cell.

The promoter may be a constitutive promoter or an inducible promoter.

In some embodiments, the recombinant expression construct is integrated into a yeast chromosome. In other embodiments, the recombinant expression construct is episomal.

In some embodiments, the recombinant yeast cell comprises a heterologous promoter linked to the endogenous nucleic acid sequence that encodes the protein.

In some embodiments, the recombinant yeast cell that is genetically modified to overexpress a protein as described herein is a Candida sp., a Saccharomyces sp., e.g., a Saccharomyces cerevisiae, or a Pichia sp. In some embodiments the host cell is Saccharomyces cerevisiae CS-400, which was deposited with the American Type Culture Collection (ATCC), 10801 University Blvd., Manassas, Va. 20110, USA on Dec. 8, 2011 under the conditions of the Budapest Treaty and assigned patent deposit number PTA-12325. In some embodiments, the yeast cell has enhanced capability for using a fermentable sugar in a fermentation reaction. In some embodiments, the fermentable sugar comprises at least one hexose sugar, e.g., glucose, and/or at least one pentose sugar, e.g., xylose. In some embodiments, the fermentation reaction comprises a cellulosic hydrolysate or a fermentable sugar from a cellulosic hydrolysate. In some embodiments, the yeast cell is capable of utilizing xylose present in a cellulosic hydrolysate for fermentation. In some embodiments, the yeast cell expresses at least one xylose utilization enzyme selected from xylose isomerase, xylose reductase, xylitol dehydrogenase, xylulokinase, xylitol isomerase and xylose transporter.

In some embodiments, the yeast cell is genetically modified to overexpress two or more proteins, e.g., two, three, four, or five, or more proteins, selected from the group consisting of an ERR3, FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, PDR12, LCB2, CHAT, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, and YMR155W protein, or homologs or variants of said proteins, wherein the proteins have at least 70% identity, at least 75%, at least 80%, at least 85%, at least 90%, at least 91%, at least 92%, at least 93%, at least 94%, at least 95%, at least 96%, at least 97%, at least 98%, or at least 99% identity, to amino acid sequences selected from SEQ ID NOS:1-27 or SEQ ID NOS:55-113. In some embodiments, the proteins have amino acid sequences selected from SEQ ID NOS:1-27 or SEQ ID NOS:55-113. In some embodiments, the yeast cell is genetically modified to overexpress two or more proteins, e.g., two, three, four, or five or more proteins, selected from an ERR3, FOX2, LYS1, MET1, MIG2, RMD6, RME1, SIP1, SNP1, TDH1, GPD1, RSF2, GND2, TRK1, HSP31, HSP33, HSP30, HSP32, ADH6, UFD4, PRO1, SIA1, ARI1, LPP1, PMA2, or PDR12 protein; wherein the proteins have at least 75%, at least 80%, at least 85%, at least 90%, at least 91%, at least 92%, at least 93%, at least 94%, at least 95%, at least 96%, at least 97%, at least 98%, or at least 99% identity to amino acid sequences selected from SEQ ID NOS:1-27. In some embodiments, the proteins have amino acid sequence selected from SEQ ID NOS:1-27.

In a further aspect, the invention relates to a fermentation composition comprising a yeast cell that has been genetically modified to overexpress an ERR3, FOX2, LYS1, MET 1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, PDR12, LCB2, CHA1, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W protein, or a homolog or variant as described herein and at least one fermentable sugar, wherein said proteins has at least 75%, at least 80%, at least 85%, at least 90%, at least 91%, at least 92%, at least 93%, at least 94%, at least 95%, at least 96%, at least 97%, at least 98%, or at least 99 and the second protein have at least 75%, at least 80%, at least 85%, at least 90%, at least 91%, at least 92%, at least 93%, at least 94%, at least 95%, at least 96%, at least 97%, at least 98%, or at least 99% identity to an amino acid sequence selected from SEQ ID NOS:1-27 or SEQ ID NOS:55-113. In some embodiments, the protein has at least 75%, at least 80%, at least 85%, at least 90%, at least 91%, at least 92%, at least 93%, at least 94%, at least 95%, at least 96%, at least 97%, at least 98%, or at least 99 and the second protein have at least 75%, at least 80%, at least 85%, at least 90%, at least 91%, at least 92%, at least 93%, at least 94%, at least 95%, at least 96%, at least 97%, at least 98%, or at least 99% identity to an amino acid sequence selected from SEQ ID NOS:1-27. In some embodiments, the protein comprises an amino acid sequence of SEQ ID NOS:1-27 or SEQ ID NOS:55-113. In some embodiments, the fermentable sugar comprises at least one hexose sugar, e.g., glucose, and/or at least one pentose sugar, e.g., xylose. In some embodiments, the fermentation composition comprises a cellulosic hydrolysate. In some embodiments, the cellulosic hydrolysate comprises at least one hexose sugar, e.g., glucose, and/or at least one pentose sugar, e.g., xylose. In some embodiments, the cellulosic hydrolysate is a lignocellulose hydrolysate.

In another aspect, the invention relates to a method of producing at least one fermentation product, the method comprising maintaining a fermentation composition of the invention, e.g., as described hereinabove, under conditions in which the fermentation product is produced. In some embodiments, the fermentation product is an alcohol, such as ethanol. In some embodiments, the method further comprises a step of recovering the fermentation product from the fermentation composition, for example recovering an alcohol, e.g., ethanol, from the fermentation composition.

### DETAILED DESCRIPTION OF THE INVENTION

### Definitions

Unless defined otherwise, technical and scientific terms used herein generally have the same meaning as commonly understood by one of ordinary skill in the art to which this invention belongs.

The term “gene” is used to refer to a segment of DNA that is transcribed. A gene may be a cDNA sequence and may include regions preceding and following the protein coding region (5′ and 3′ untranslated sequence). A gene may also include introns. A “gene” in the context of this invention can encode a functional variant of full-length protein.

As used herein, the term “overexpress” with respect to a host cell that is genetically modified to overexpress a protein refers to increasing the amount of the protein in the cell to an amount that is greater than the amount that is produced in an unmodified host cell. A protein that is overexpressed may be endogenous to the host cell or exogenous to the host cell.

The terms “naturally occurring”, “native”, and “wild-type” are used interchangeably herein to refer to a protein or nucleic acid found in nature. For example, when used in reference to a yeast nucleotide or yeast polypeptide sequence, the term means the nucleotide or polypeptide sequence occurring in a naturally occurring yeast strain. When used in reference to a yeast cell or yeast strain, the term means a naturally occurring (not genetically modified) microorganism.

The terms “modifications” and “mutations” when used in the context of substitutions, deletions, insertions and the like with respect to polynucleotides and polypeptides are used interchangeably herein and refer to changes that are introduced by genetic manipulation to create variants, e.g., amino acid sequences comprising deletions, insertions, or substitutions relative to a wild-type sequence.

“Conservatively modified variants” applies to both amino acid and nucleic acid sequences. With respect to protein-encoding nucleic acid sequences, conservatively modified variants refers to those nucleic acids which encode identical amino acid sequences, or encode amino acid sequences having conservative substitutions that retain the function of the wildtype protein. Because of the degeneracy of the genetic code, a large number of functionally identical nucleic acids encode any given protein. For instance, the codons GCA, GCC, GCG and GCU all encode the amino acid alanine. Accordingly, each variation of a nucleic acid which encodes a polypeptide is implicit in the protein sequence.

Conservative substitution tables providing functionally similar amino acids are well known in the art. Such conservatively modified variants are in addition to and do not exclude polymorphic variants, interspecies homologs, and alleles of the invention. A functional way to define common properties between individual amino acids is to analyze the normalized frequencies of amino acid changes between corresponding proteins of homologous organisms. (See, e.g., Schulz, G. E. and R. H. Schirmer, Principles of Protein Structure, Springer-Verlag). According to such analyses, groups of amino acids may be defined where amino acids within a group exchange preferentially with each other and, therefore, resemble each other most in their impact on the overall protein structure. One example of a set of amino acid groups defined in this manner include: (i) a charged group, consisting of Glu and Asp, Lys, Arg and His; (ii) a positively-charged group, consisting of Lys, Arg and His; (iii) a negatively-charged group, consisting of Glu and Asp; (iv) an aromatic group, consisting of Phe, Tyr and Trp; (v) a nitrogen ring group, consisting of His and Trp; (vi) a large aliphatic nonpolar group, consisting of Val, Leu and Ile; (vii) a slightly-polar group, consisting of Met and Cys; (viii) a small-residue group, consisting of Ser, Thr, Asp, Asn, Gly, Ala, Glu, Gln and Pro; (ix) an aliphatic group consisting of Val, Leu, Ile, Met and Cys; and (x) a small hydroxyl group consisting of Ser and Thr. The following groups each contain amino acids that are examples of conservative substitutions for one another: 1) Alanine (A), Glycine (G); 2) Aspartic acid (D), Glutamic acid (E); 3) Asparagine (N), Glutamine (Q); 4) Arginine I, Lysine (K); 5) Isoleucine (I), Leucine (L), Methionine (M), Valine (V); 6) Phenylalanine (F), Tyrosine (Y), Tryptophan (W); and 7) Serine (S), Threonine (T); and (see, e.g., Creighton, Proteins (1984)).

The terms “polypeptide,” “peptide,” and “protein” are used interchangeably to refer to a polymer of amino acid residues.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine.

“Identity” or “percent identity” in the context of two or more polypeptide or nucleic acid sequences, refers to two or more sequences or subsequences that are the same or have a specified percentage of amino acid residues or nucleotides that are the same (e.g., share at least 60% identity, or at least 65% identity, or at least 70% identity, at least 75% identity, at least 80% identity, at least 85% identity, at least 88% identity, or at least 89%, at least 90%, at least 91%, at least 92%, at least 93%, at least 94%, at least 95%, at least 96%, at least 97%, at least 98%, or at least 99% identity over a specified region to a reference sequence, or over the full-length of the reference sequence, when compared and aligned for maximum correspondence over a comparison window, or designated region as measured using a sequence comparison algorithms or by manual alignment and visual inspection.

Optimal alignment of sequences for comparison and determination of sequence identity can be determined by a sequence comparison algorithm or by visual inspection (see, generally, Ausubel et al., infra). When using a sequence comparison algorithm, test and reference sequences are entered into a computer, subsequence coordinates and sequence algorithm program parameters are designated. The sequence comparison algorithm then calculates the percent sequence identities for the test sequences relative to the reference sequence, based on the program parameters.

The algorithm used to determine whether a protein has sequence identity to one of SEQ ID NOS:1-27 is the BLAST algorithm, which is described in Altschul et al., 1990, J. Mol. Biol. 215:403-410. Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. For amino acid sequences, the BLASTP program uses as defaults a word size (W) of 3, an expectation (E) of 10, and the BLOSUM62 scoring matrix (see Henikoff & Henikoff, 1989, Proc. Natl. Acad. Sci. USA 89:10915).

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art. See e.g., Dayhoff et al. (1978), “A model of evolutionary change in proteins”; “Atlas of Protein Sequence and Structure,” Vol. 5, Suppl. 3 (Ed. M. O. Dayhoff), pp. 345-352, Natl. Biomed. Res. Round, Washington, D.C.; and Henikoff et al. (1992) Proc. Natl. Acad. Sci. USA, 89:10915-10919, both of which are incorporated herein by reference. The BLOSUM62 matrix is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap. The alignment is defined by the amino acid position of each sequence at which the alignment begins and ends, and optionally by the insertion of a gap or multiple gaps in one or both sequences so as to arrive at the highest possible score.

A “reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a “comparison window” to identify and compare local regions of sequence similarity.

The term “transformed”, in the context of introducing a nucleic acid sequence into a cell, includes introducing a nucleic acid by transfection, transduction or transformation. The nucleic acid sequence may be maintained in the cell as an extrachromosomal element or may be integrated into the yeast DNA, e.g., integrated into a yeast chromosome or yeast episomal plasmid such as the 2 micron plasmid that is maintained through multiple generations.

The term “nucleic acid” “nucleotides” or “polynucleotide” refers to deoxyribonucleotides or ribonucleotides and polymers thereof in either single-stranded or double-stranded form. Except were specified or otherwise clear from context, reference to a nucleic acid sequence encompasses a double stranded molecule.

The term “endogenous” in the context of this invention refers to a gene or protein that is originally present in a naturally occurring yeast cell strain. Conversely, an “exogenous” gene or protein is one that originates outside the yeast cell strain, such as a gene from another species or a recombinant variant of a naturally occurring protein.

The term “operably linked” refers to a configuration in which a control sequence is appropriately placed at a position relative to the coding sequence of the DNA sequence such that the control sequence influences the expression of a polypeptide.

An amino acid or nucleotide sequence (e.g., a promoter sequence, a polypeptide encoding an enzyme, a signal peptide, terminator sequence, etc.) is “heterologous” to another sequence with which it is operably linked if the two sequences are not associated in nature. Thus, a “heterologous” gene may be endogenous to the host cell, but operably linked to a sequence with which it is not associated in nature, e.g., a promoter sequence.

The term “expression construct” refers to a polynucleotide comprising a promoter sequence operably linked to a protein encoding sequence. Expression cassettes and expression vectors are examples of “expression constructs”. The term “expression construct” includes constructs for targeting DNA to direct integration into the host cell DNA to a desired site such as a yeast episomal plasmid or a yeast chromosome. In some embodiments, an expression construct can encode an exogenous protein sequence operably linked to an endogenous promoter sequence. In some embodiments, an expression construct can comprise a heterologous promoter operably linked to an endogenous nucleic acid sequence encoding a protein.

An “expression cassette” refers to a nucleic acid containing a protein coding sequence and a promoter and other nucleic acid elements that permit transcription of the sequence in a host cell (e.g., termination/polyadenylation sequences).

The term “vector,” as used herein, refers to a recombinant nucleic acid designed to carry a nucleic acid sequence of interest to be introduced into a host cell. In some embodiments, a vector for use in the invention comprises an expression construct that comprises a promoter sequence and a heterologous polynucleotide encoding a protein of interest that is to be expressed. The term “vector” encompasses many different types of vectors, such as cloning vectors, expression vectors, shuttle vectors, plasmids, phage or virus particles, and the like. Vectors include PCR-based vehicles as well as plasmid vectors. Vectors typically include an origin of replication and usually includes a multicloning site and a selectable marker. A typical expression vector may also include, in addition to a coding sequence of interest, elements that direct the transcription and translation of the coding sequence, such as a promoter, enhancer, and termination/polyadenylation sequences. In some embodiments, a vector is an integration vector so that the sequence of interest is integrated into the host cell DNA, e.g., a yeast cell chromosome or yeast episomal plasmid.

As used herein, the term “promoter” refers to a polynucleotide sequence, particularly a DNA sequence, that initiates and facilitates the transcription of a target gene sequence in the presence of RNA polymerase and transcription regulators. Promoters may include DNA sequence elements that ensure proper binding and activation of RNA polymerase, influence where transcription will start, affect the level of transcription and, in the case of inducible promoters, regulate transcription in response to environmental conditions. In the present invention, the term “promoter” may also include other elements, such as an enhancer element.

The term “recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “host cell” is a cell into which a vector of the present invention may be introduced and expressed. The term encompasses both a cell transformed with the vector and progeny of such a cell. A “recombinant host cell” refers to a cell into which has been introduced a heterologous polynucleotide, gene, promoter, e.g., an expression vector, or to a cell having a heterologous polynucleotide or gene integrated into the host cell DNA, e.g., integrated into a yeast chromosome or yeast episomal plasmid. A “recombinant cell genetically modified to overexpress at least one protein” in accordance with the invention encompasses both a cell transformed with a nucleic acid to overexpress the protein and progeny of such a cell.

As used herein, a “parent” yeast cell refers to a yeast host cell that does not have the modification to overexpress the gene. The genetic modification to overexpress a protein of interest is introduced into the parent host cell. Thus, for example, overexpression of a gene, e.g., a gene encoding a protein set forth in one of SEQ ID NOS:1-27 or SEQ ID NOS:55-113, or a functional variant or homolog thereof, can be evaluated by comparing glucose utilization in a fermentation reaction using a yeast strain in which the gene is overexpressed compared to the parent yeast strain grown under identical conditions. A parent yeast strain may comprise other modifications, such as introduction of genes conferring drug resistance, encoding other proteins such as metabolic proteins, and the like.

A composition is “isolated” when it is in an environment different from naturally occurring environment. For example, an “isolated” polynucleotide, polypeptide, enzyme, compound, or cell can be one that is removed from the environment in which it naturally occurs. Also, an “isolated” recombinant cell can be a recombinant cell that has been isolated from the parent host cell and may be present in a clonal culture of cells or in a mixed population of cells, including other recombinant cells.

As used herein, the term “cellulosic hydrolysate” refers to a product of hydrolysis of a cellulosic biomass that comprises cellulose, including hemicellulose or lignocellulose. A cellulosic hydrolysate may be obtained by processing a cellulosic biomass to release sugars that can be fermented, e.g., to an alcohol such as ethanol. The hydrolytic process used to produce the cellulosic hydrolysate typically includes acid or enzymatically treating a cellulosic biomass to hydrolyze the cellulose to release monomeric sugars. The cellulosic biomass may comprise components other than cellulose such that both pentose sugars and hexose sugars may be present in the cellulosic hydrolysate. For example, a cellulosic biomass may comprise hemicellulose and/or lignocellulose.

An example of a cellulosic hydrolysate is a “lignocellulosic hydrolysate.” A lignocellulosic hydrolysate is a product of hydrolysis of lignocellulose, e.g., a lignocellulosic feedstock that has been processed to release sugars that can be fermented, e.g., to an alcohol such as ethanol. The hydrolytic process used to produce the lignocellulosic hydrolysate includes acid or enzymatically treating a lignocellulosic biomass to hydrolyze the cellulose, hemicellulose and other components to release monomeric sugars. Lignocellulosic hydrolysates contain fermentable sugars, e.g., hexose sugars such as glucose, and pentose sugars such as xylose or arabinose.

The term “lignocellulosic biomass” or “lignocellulosic feedstock” or “lignocellulosic substrate” refers to materials that contain cellulose, hemicellulose and lignocellulose. As used herein, a “cellulosic biomass” or “cellulosic feedstock” or “cellulosic substrate” refers to materials that contain cellulose (and, optionally, other components such as hemicellulose and lignocellulose).

“Saccharification” as used herein refers to the process in which cellulosic substrates e.g., hemicellulose or lignocellulose, are broken down via the action of cellulases to produce fermentable sugars. “Saccharification” also refers to the process in which cellulosic substrates are hydrolyzed by non-enzymatic methods to produce soluble sugars.

As used herein, the terms “ferment”, “fermenting” and “fermentation” refer to a biochemical process by which an organism uses substrates, e.g., sugars, as a carbon and energy source for production of a metabolic product. In a fermentation reaction a substrate (e.g., a sugar) is converted to at least one fermentation product, including but not limited to such products as alcohols (e.g., ethanol, butanol, isobutanol, etc.), fatty alcohols (e.g., C8-C20 fatty alcohols), acids (e.g., lactic acid, 3-hydroxypropionic acid, acrylic acid, acetic acid, succinic acid, citric acid, malic acid, fumaric acid, amino acids, etc.), fatty acids, butadiene, 1,3-propane diol, ethylene glycol, glycerol, terpenes, and antimicrobials (e.g., β-lactams such as cephalosporin), etc. In some embodiments in which ethanol is produced by fermentation, other products, including but not limited to lactate, acetic acid, hydrogen and carbon dioxide are also produced. Alcoholic fermentation is a process in which sugars such as xylulose, glucose, fructose, sucrose, xylose, and arabinose are converted into a fermentation end product, including but not limited to biofuel. For example, the fermentation product may comprise alcohol (such as ethanol or butanol) and/or a sugar alcohol, such as xylitol.

“Fermentable sugars” as used here means simple sugars (monosaccharides, disaccharides and short oligosaccharides) including, but not limited to, glucose, xylose, galactose, arabinose, mannose, and sucrose.

As used herein, “sugar utilization” in a fermentation reaction refers to the amount of a fermentable sugar, e.g., a hexose sugar such as glucose, or a pentose sugar such as xylose, that is converted into another chemical form in a metabolic process that yields a fermentation product. Increased sugar utilization in a yeast strain in comparison to the parent yeast strain means that sugar is used at a greater rate. Sugar utilization can be assessed by monitoring the level of sugar, e.g., glucose or xylose e.g., in a fermentation reaction (e.g., culture medium) using known techniques, e.g., HPLC. For example, after a fixed time period of a fermentation reaction, such as 24 hours, the amount of residual fermentable sugar remaining in the culture medium will be lower in a fermentation reaction using a yeast strain that has been genetically modified to overexpress a protein as described herein in comparison to a fermentation reaction using the unmodified parent strain.

As used herein “a”, “an”, and “the” include plural references unless the context clearly dictates otherwise.

The term “comprising” and its cognates are used in their inclusive sense; that is, equivalent to the term “including” and its corresponding cognates.

### General Methods

Unless indicated otherwise, the techniques and procedures described or referred to herein are generally performed according to conventional methods well known in the art. Texts disclosing general methods and techniques in the field of recombinant genetics include Sambrook and Russell, Molecular Cloning, A Laboratory Manual (3rd ed. 2001); Ausubel, ed., Current Protocols in Molecular Biology, John Wiley Interscience (1990-2011); each of which incorporated by reference herein, for all purposes. DNA sequences can be obtained by cloning, or by chemical synthesis.

Methods for recombinant expression of proteins in yeast and other organisms are well known in the art, and a number suitable expression vectors are available or can be constructed using routine methods. For example, methods, reagents and tools for transforming yeast are described in “Guide to Yeast Genetics and Molecular Biology,” C. Guthrie and G. Fink, Eds., Methods in Enzymology Vol. 350 (Academic Press, San Diego, 2002). Introduction of a DNA construct or vector o into a host cell can be effected using any known techniques, e.g., by calcium phosphate transfection, DEAE-Dextran mediated transfection, electroporation, lithium acetate and polyethylene glycol, or other common techniques.

### Overexpression of Genes

The invention relates, in part, to the identification, as described in the Examples, of genes and their corresponding protein products that when overexpressed in yeast, provide improved fermentation reactions, relative to yeast in which the genes or proteins are not overexpressed. The improvement can be increased hexose and/or pentose sugar utilization, e.g., increased glucose and/or xylose utilize, or improved yields in a fermentation reaction, e.g., an improved yield of an alcohol such as ethanol. In some embodiments, recombinant yeast that overexpress the proteins are used in fermentation reactions that comprise a cellulosic hydrolysate, such as a lignocellulosic hydrolysate

Proteins that are overexpressed include Saccharomyces cerevisiae proteins of SEQ ID NOS:1-27 and SEQ ID NOS:55-113 and homologs and functional variants of the Saccharomyces cerevisiae proteins of SEQ ID NOS:1-27 and SEQ ID NOS:55-113. A “homolog” as used herein refers to a gene or protein from another species or organism that corresponds to a Saccharomyces cerevisiae gene or protein. In the current invention, homologs that are useful in the invention encode a protein that has at least 50% identity, or at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to a Saccharomyces cerevisiae protein having an amino acid sequence selected from SEQ ID NOS:1-27 or SEQ ID NOS:55-113; and has the biological activity of the S. cerevisiae protein. As used herein, the term “homolog” includes orthologs and paralogs.

A “functional variant” refers to a variant of a Saccharomyces cerevisiae protein that has mutations (e.g., substitutions, deletions, and insertions) relative to the wildtype sequence and retains the biological activity of the wildtype protein. In the current invention, functional variants that are useful in the invention encode a protein that has at least 50% identity, or at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to a Saccharomyces cerevisiae protein having an amino acid sequence selected from SEQ ID NOS:1-27 or SEQ ID NOS:55-113; and has the protein activity of the S. cerevisiae protein. In the context of this invention, the term “variant”, when used with reference to a variant of a protein that is overexpressed in yeast in accordance with the invention, refers to a functional variant of the protein.

A functional variant or homolog useful in the invention typically has activity that is equivalent to the biological activity of the Saccharomyces cerevisiae wildtype sequence. In some embodiments, the functional variant or homolog has at least 90%, 80%, 70%, 60%, or 50% of the biological activity of the wildtype sequence.

As used herein, reference to “an ERR3 protein” may encompass homologs and functional variants of the illustrative ERR3 polypeptide SEQ ID NO:1. Similarly, reference to a FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, PDR12, LCB2, CHA1, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W protein may encompass homologs and functional variants of the corresponding illustrative polypeptides of SEQ ID NOS:2-27 and 55-113. For example, “an ERR3 protein comprising at least 70% identity to SEQ ID NO:1” encompasses homologs and variants of the ERR3 protein of SEQ ID NO:1.

In one aspect, the invention thus relates to yeast host cells, e.g., Saccharomyces sp. host cells, that are genetically modified to overexpress at least one of the following proteins ERR3, FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARIL LPP1, PMA2, PDR12 or a homolog or functional variant of the ERR3, FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, or PDR1 protein. A functional variant of a protein includes variants that have substitutions, deletions, and/or insertions relative to a reference sequence of SEQ ID NOS:1-27. A homolog or functional variant of the protein that is overexpressed has at least 50% identity, at least 60% identity, or at least 70%, at least 75%, at least 80%, at least 85%, at least 90%, at least 95%, at least 96%, at least 97%, at least 98%, or at least 99% identity to a Saccharomyces cerevisiae ERR3, FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, or PDR12 protein, e.g., a protein having an amino acid sequence selected from SEQ ID NOS:1-27.

In some embodiments, the ERR3, FOX2, LYS1, MET1, MIG2, RMD6, RME1, SIP1, SNP1, TDH1, ZWF1 GPD1, RSF2, GND2, TRK1, HSP31, HSP33, HSP30, HSP32, ADH6, UFD4, PRO1, SIA1, ARI1, LPP1, PMA2, or PDR12 gene that encodes the protein has at least 70%, at least 75%, at least 80%, at least 85%, at least 90%, at least 95%, at least 96%, at least 97%, at least 98%, or at least 99% identity to a nucleic acid sequence of SEQ ID NOS:28-54.

In one aspect, the invention thus relates to yeast host cells, e.g., Saccharomyces sp. host cells, that are genetically modified to overexpress at least one of the following proteins LCB2, CHAT, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W; or a homolog or functional variant of the LCB2, CHAT, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W protein. A functional variant of a protein includes variants that have substitutions, deletions, and/or insertions relative to a reference sequence of SEQ ID NOS:55-116. A homolog or functional variant of the protein that is overexpressed has at least 50% identity, at least 60% identity, or at least 70%, at least 75%, at least 80%, at least 85%, at least 90%, at least 95%, at least 96%, at least 97%, at least 98%, or at least 99% identity to a Saccharomyces cerevisiae LCB2, CHA1, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W protein, e.g., a protein having an amino acid sequence selected from SEQ ID NOS:55-113.

In some embodiments, the LCB2, CHAT, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W gene that encodes the protein has at least 70%, at least 75%, at least 80%, at least 85%, at least 90%, at least 95%, at least 96%, at least 97%, at least 98%, or at least 99% identity to a nucleic acid sequence set forth in SEQ ID NOS:114-173.

In some embodiments, a yeast host cell is genetically modified to overexpress at least one protein having at least 70%, at least 75%, at least 80%, at least 85%, at least 90%, at least 95%, at least 96%, at least 97%, at least 98%, or at least 99% identity to an amino acid sequence selected from SEQ ID NOS:1-10. In some embodiments, the protein has an amino acid sequence selected from SEQ ID NOS:1-10. In some embodiments, the yeast host cell is genetically modified to overexpress at least one protein having at least 70%, at least 75%, at least 80%, at least 85%, at least 90%, at least 95%, at least 96%, at least 97%, at least 98%, or at least 99% identity to an amino acid sequence selected from SEQ ID NO:13, SEQ ID NO:15, SEQ ID NO:16, SEQ ID NO:17, SEQ ID NO:19, SEQ ID NO:21, and SEQ ID NO:25. In some embodiments, the protein has an amino acid sequence of SEQ ID NO:13, SEQ ID NO:15, SEQ ID NO:16, SEQ ID NO:17, SEQ ID NO:19, SEQ ID NO:21, or SEQ ID NO:25. In some embodiments, the yeast host cell is genetically modified to overexpress at least one protein having at least 70%, at least 75%, at least 80%, at least 85%, at least 90%, at least 95%, at least 96%, at least 97%, at least 98%, or at least 99% identity to SEQ ID NO:55. In some embodiments, the protein has a sequence set forth in SEQ ID NO:55.

In the context of this invention, the product of a gene is considered to be overexpressed when the level of protein activity is increased by at least 5%, at least 10%, at least 20%, at least 30%, or at least 50% or greater in comparison to a yeast host cell of the same strain and genetic background that has not been genetically modified to overexpress the protein.

Overexpression may be assessed using any number of endpoints, including, e.g., measuring the level of mRNA encoded by the gene, the level of protein, protein activity, or a measure of a downstream endpoint that reflects protein activity, e.g., glucose utilization, pentose sugar utilization, and/or production of a fermentation product such as ethanol may be used to assess protein activity.

### Examples of Homologs

Illustrative Saccharomyces cerevisiae genes that can be overexpressed in yeast, e.g., a Saccharomyces cerevisiae strain, to be used in a fermentation reaction, with the yeast systematic name for the protein and examples of nucleic acid and protein sequence are provided in the Table of Illustrative Sequences, infra. Table 1, infra, provides accession numbers for the Saccharomyces cerevisiae protein and nucleic acid sequences; and accession numbers for illustrative homologs of Saccharomyces cerevisiae, that have at least 70% amino acid sequence identity to an amino acid sequence set forth in one of NOS:1-27, and which may be overexpressed according to the present invention.

TABLE 1

| | | | | | % Identity |
|---|---|---|---|---|---|
| | | | | | (Amino |
| Gene | (accession) | UniProtKB | SEQ | | acid |
| Name | Gi no. | AC | ID NO | Species | sequence) |
| MIG2 | 1709032 | P53035 | 5 | Saccharomyces cerevisiae S288c | 100 |
| | 49524590 | Q6FWV8 | | Candida glabrata CBS 138 | 75 |
| | 156113675 | A7TR92 | | Vanderwaltozyma polyspora DSM 70294 | 72 |
| LYS1 | 82654956 | P38998 | 3 | Saccharomyces cerevisiae S288c | 100 |
| | 238933633 | C5DDF5 | | Lachancea thermotolerans | 78 |
| | 49643445 | Q6CP29 | | Kluyveromyces lactis NRRL Y-1140 | 76 |
| | 126213197 | A3GF76 | | Scheffersomyces stipitis CBS 6054 | 74 |
| | 238029901 | C4QX51 | | Komagataella pastoris GS115 | 72 |
| TDH1 | 1169786 | P00360 | 10 | Saccharomyces cerevisiae S288c | 100 |
| | 120645 | P00358 | | Saccharomyces cerevisiae S288c | 89 |
| | 1169787 | P00359 | | Saccharomyces cerevisiae S288c | 88 |
| | 238933254 | C5DCC7 | | Lachancea thermotolerans | 84 |
| | 116668008 | P84998 | | K. Marxianus | 82 |
| | 68472462 | Q5ADM7 | | Candida albicans SC5314 | 78 |
| | 126094480 | A3LQ70 | | Scheffersomyces stipitis CBS 6054 | 77 |
| | 54035923 | Q6CCU7 | | Yarrowia lipolytica | 71 |
| FOX2 | 399508 | Q02207 | 2 | Saccharomyces cerevisiae S288c | 100 |
| | 49528177 | Q6FLN0 | | Candida glabrata CBS 138 | 75 |
| | 156113322 | A7TS49 | | Vanderwaltozyma polyspora DSM 70294 | 71 |
| ERR3 | 1706698 | P42222 | 1 | Saccharomyces cerevisiae S288c | 100 |
| | 74662255 | Q70CP7 | | Kluyveromyces lactis NRRL Y-1140 | 70 |
| | 74661257 | Q6FQY4 | | Candida glabrata CBS 138 | 70 |
| ZWF1 | 120734 | P11412 | 27 | Saccharomyces cerevisiae S288c | 100 |
| | 238940775 | C5DYT8 | | Zygosaccharomyces rouxii | 72 |
| | 1346071 | P48828 | | Kluyveromyces lactis NRRL Y-1140 | 71 |
| | 44980660 | Q75E77 | | Ashbya gossypii ATCC 10895 | 71 |
| | 238941862 | C5E1X3 | | Lachancea thermotolerans | 70 |
| GPD1 | 462197 | Q00055 | 11 | Saccharomyces cerevisiae S288c | 100 |
| | 156116589 | A7TI54 | | Vanderwaltozyma polyspora DSM 70294 | 81 |
| | 156115924 | A7TJU4 | | Vanderwaltozyma polyspora DSM 70294 | 78 |
| | 1708024 | P41911 | | Saccharomyces cerevisiae S288c | 75 |
| | 238935875 | C5DKQ4 | | Lachancea thermotolerans | 75 |
| | 31323264 | Q7ZA45 | | Lachancea thermotolerans | 75 |
| | | | | (Kluyveromyces thermotolerans) | |
| | 9857609 | Q9HGY2 | | Zygosaccharomyces rouxii (Candida mogli) | 74 |
| | 49641508 | Q6CUL4 | | Kluyveromyces lactis NRRL Y-1140 | 73 |
| RSF2 | 1177049 | P46974 | 12 | Saccharomyces cerevisiae S288c | 100 |
| | 149389118 | A3GI42 | | Scheffersomyces stipitis CBS 6054 | 75 |
| | 240133681 | C5MC70 | | Candida tropicalis MYA-3404 | 73 |
| | 146451047 | A5E1J5 | | Lodderomyces elongisporus NRRL YB-4239 | 72 |
| | 223641264 | B9WA06 | | Candida dubliniensis CD36 | 71 |
| | 68467592 | Q5AKV1 | | Candida albicans SC5314 | 71 |
| GND2 | 1703016 | P53319 | 13 | Saccharomyces cerevisiae S288c | 100 |
| | 728743 | P38720 | | Saccharomyces cerevisiae S288c | 88 |
| | 342302910 | G0VGR6 | | Naumovozyma castellii | 86 |
| | 28565046 | Q875M5 | | Kluyveromyces lactis NRRL Y-1140 | 82 |
| | 238850652 | C4Y7R6 | | Clavispora lusitaniae ATCC 42720 | 79 |
| | 218722634 | B8M376 | | Talaromyces stipitatus ATCC 10500 | 76 |
| | 29409963 | Q874Q3 | | Aspergillus niger CBS 513.88 | 75 |
| | 326461055 | F2SKQ0 | | Trichophyton rubrum CBS 118892 | 73 |
| | 238846145 | C5G104 | | Arthroderma otae CBS 113480 | 71 |
| HSP32 | 74627257 | Q08992 | 18 | Saccharomyces cerevisiae S288c | 100 |
| | 50400297 | Q04432 | | Saccharomyces cerevisiae S288c | 70 |
| TRK1 | 136231 | P12685 | 14 | Saccharomyces cerevisiae S288c | 100 |
| | 49528334 | Q6FL73 | | Candida glabrata CBS 138 | 82 |
| | 207343367 | B5VMJ7 | | Saccharomyces cerevisiae YJM789 | 74 |
| | 156113782 | A7TQY9 | | Vanderwaltozyma polyspora DSM 70294 | 72 |
| | 238031859 | C4R2Q8 | | Komagataella pastoris GS115 | 70 |
| HSP31 | 50400297 | Q04432 | 15 | Saccharomyces cerevisiae S288c | 100 |
| | 49524497 | Q6FX51 | | Candida glabrata CBS 138 | 77 |
| | 49642236 | Q6CSI7 | | Kluyveromyces lactis NRRL Y-1140 | 71 |
| | 74627257 | Q08992 | | Saccharomyces cerevisiae S288c | 70 |
| ADH6 | 2492777 | Q04894 | 19 | Saccharomyces cerevisiae S288c | 100 |
| | 49529269 | Q6FII9 | | Candida glabrata CBS 138 | 79 |
| | 156112876 | A7TTA3 | | Vanderwaltozyma polyspora DSM 70294 | 70 |
| | 465668 | P33202 | | Saccharomyces cerevisiae S288c | 100 |
| | 1709785 | P32264 | | Saccharomyces cerevisiae S288c | 100 |
| | 49641791 | Q6CTT1 | | Kluyveromyces lactis NRRL Y-1140 | 78 |
| | 49526170 | Q6FSD3 | | Candida glabrata CBS 138 | 74 |
| | 238936682 | C5DM50 | | Lachancea thermotolerans | 73 |
| | 156117414 | A7TFR8 | | Vanderwaltozyma polyspora DSM 70294 | 72 |
| | 238939130 | C5DU45 | | Zygosaccharomyces rouxii | 72 |
| | 44980109 | Q75EY9 | | Ashbya gossypii ATCC 10895 | 71 |
| ARI1 | 1723933 | P53111 | 23 | Saccharomyces cerevisiae S288c | 100 |
| | 1723822 | P53183 | | Saccharomyces cerevisiae S288c | 76 |
| PMA2 | 1709667 | P19657 | 25 | Saccharomyces cerevisiae S288c | 100 |
| | 1168544 | P05030 | | Saccharomyces cerevisiae S288c | 92 |
| | 238935207 | C5DHX7 | | Lachancea thermotolerans | 87 |
| | 223642354 | B9WD47 | | Candida dubliniensis CD36 | 83 |
| | 238029429 | C4QVS9 | | Komagataella pastoris GS115 | 83 |
| | 114347 | P07038 | | Neurospora crassa OR74A | 78 |
| | 150414445 | A6R9I6 | | Ajellomyces capsulatus NAm1 | 77 |
| | 239588203 | C5JTE5 | | Ajellomyces dermatitidis SLH14081 | 77 |
| PDR12 | 6093664 | Q02785 | 26 | Saccharomyces cerevisiae S288c | 100 |
| | 49528979 | Q6FJC9 | | Candida glabrata CBS 138 | 85 |
| | 156114992 | A7TMJ5 | | Vanderwaltozyma polyspora DSM 70294 | 83 |
| | 49641092 | Q6CVS9 | | Kluyveromyces lactis NRRL Y-1140 | 76 |
| | 238940476 | C5DXY9 | | Zygosaccharomyces rouxii | 76 |

### Activity of Functional Variants and Homologs

Functional variants and homologs have the biological activity of the wildtype protein. Assays that may be used to identify homologs and functional variants useful for the practice of the invention or homolog are known in the art. In some embodiments, activity of a functional variant or homolog of a protein, e.g., a functional variant of SEQ ID NOS:1-27 or SEQ ID NOS:55-113, is assessed by directly measuring enzymatic activity or other protein activity. For example, the activity of ZWF1, TDH1, MET1, LYS1, FOX2, GPD1, GND2, and PRO1 can be assessed by measuring enzymatic activity (see, Table 2).

TABLE 2

| | | Enzyme | |
|---|---|---|---|
| Common | Systematic | Commission | |
| Name | Name | (EC) No. | Protein Activity |
| ZWF1 | YNL241C | 1.1.1.49 | Glucose-6-phosphate dehydrogenase (G6PD), |
| | | | catalyzes the first step of the pentose phosphate |
| | | | pathway; involved in adapting to oxidative stress |
| TDH1 | YIL052W | 1.2.1.12 | Glyceraldehyde-3-phosphate dehydrogenase, |
| | | | isozyme 1, involved in glycolysis and |
| | | | gluconeogenesis; tetramer that catalyzes the reaction |
| | | | of glyceraldehyde-3-phosphate to 1,3 bis- |
| | | | phosphoglycerate |
| SNP1 | YIL061C | | Component of U1 snRNP required for mRNA |
| | | | splicing via spliceosome |
| SIP1 | YDR422C | | Alternate beta-subunit of the Snf1p kinase complex, |
| | | | may confer substrate specificity; vacuolar protein |
| | | | containing KIS (Kinase-Interacting Sequence) and |
| | | | ASC (Association with Snf1 kinase Complex) |
| | | | domains involved in protein interactions |
| RME1 | YGR044C | | Zinc finger protein involved in control of meiosis; |
| | | | prevents meiosis by repressing IME1 expression and |
| | | | promotes mitosis by activating CLN2 expression; |
| | | | directly repressed by a1-a2 regulator; mediates cell |
| | | | type control of sporulation |
| RMD6 | YEL072W | | Protein required for sporulation |
| MIG2 | YGL209W | | Protein containing zinc fingers, involved in |
| | | | repression, along with Mig1p, of SUC2 (invertase) |
| | | | expression by high levels of glucose; binds to |
| | | | Mig1p-binding sites in SUC2 promoter |
| MET1 | YKR069W | 2.1.1.107 | S-adenosyl-L-methionine uroporphyrinogen III |
| | | | transmethylase, involved in the biosynthesis of |
| | | | siroheme, a prosthetic group used by sulfite |
| | | | reductase; required for sulfate assimilation and |
| | | | methionine biosynthesis |
| LYS1 | YIR034C | 1.5.1.7 | Saccharopine dehydrogenase (NAD+, L-lysine- |
| | | | forming), catalyzes the conversion of saccharopine to |
| | | | L-lysine, which is the final step in the lysine |
| | | | biosynthesis pathway |
| FOX2 | YKR009C | 1.1.1.35 | Multifunctional enzyme of the peroxisomal fatty acid |
| | | | beta-oxidation pathway; has 3-hydroxyacyl-CoA |
| | | | dehydrogenase and enoyl-CoA hydratase activities |
| ERR3 | YMR323W | | Protein of unknown function, has similarity to |
| | | | enolases |
| GPD1 | YDL022W | 1.1.1.8 | NAD-dependent glycerol-3-phosphate |
| | | | dehydrogenase |
| RSF2 | YJR127C | | Zinc-finger protein involved in transcriptional |
| | | | control of both nuclear and mitochondrial genes |
| GND2 | YGR256W | 1.1.1.44 | 6-phosphogluconate dehydrogenase |
| | | | (decarboxylating), catalyzes an NADPH regenerating |
| | | | reaction in the pentose phosphate pathway; required |
| | | | for growth on D-glucono-delta-lactone |
| TRK1 | YJL129C | | Component of the Trk1p-Trk2p potassium transport |
| | | | system; 180 kDa high affinity potassium transporter; |
| | | | phosphorylated in vivo and interacts physically with |
| | | | the phosphatase Ppz1p |
| HSP31 | YDR533C | | Similar to E. coli Hsp31; member of the DJ- |
| | | | 1/ThiJ/PfpI superfamily |
| HSP33 | YOR391C | | Similar to E. coli Hsp31 and S. cerevisiae Hsp31p, |
| | | | Hsp32p, and Sno4p; member of the DJ-1/ThiJ/PfpI |
| | | | superfamily |
| HSP30 | YCR021C | | Hydrophobic plasma membrane localized, stress- |
| | | | responsive protein that negatively regulates the H(+)- |
| | | | ATPase Pma1p |
| HSP32 | YPL280W | | Similar to E. coli Hsp31 and S. cerevisiae Hsp31p, |
| | | | Hsp33p, and Sno4p; member of the DJ-1/ThiJ/PfpI |
| | | | superfamily |
| ADH6 | YMR318C | | NADPH-dependent medium chain alcohol |
| | | | dehydrogenase with broad substrate specificity; |
| | | | member of the cinnamyl family of alcohol |
| | | | dehydrogenases |
| UFD4 | YKL010C | | Ubiquitin-protein ligase (E3) that interacts with |
| | | | Rpt4p and Rpt6p, two subunits of the 19S particle of |
| | | | the 26S proteasome; cytoplasmic E3 involved in the |
| | | | degradation of ubiquitin fusion proteins |
| PRO1 | YDR300C | 2.7.2.11 | Gamma-glutamyl kinase, catalyzes the first step in |
| | | | proline biosynthesis |
| SIA1 | YOR137C | | Protein involved in activation of the Pma1p plasma |
| | | | membrane H+-ATPase by glucose |
| ARI1 | YGL157W | | Oxidoreductase, catalyzes NADPH-dependent |
| | | | reduction of the bicyclic diketone |
| | | | bicyclo[2.2.2]octane-2,6-dione (BCO2,6D) to the |
| | | | chiral ketoalcohol (1R,4S,6S)-6- |
| | | | hydroxybicyclo[2.2.2]octane-2-one (BCO2one6ol) |
| LPP1 | YDR503C | | Lipid phosphate phosphatase, catalyzes Mg(2+)- |
| | | | independent dephosphorylation of phosphatidic acid |
| | | | (PA), lysophosphatidic acid, and diacylglycerol |
| | | | pyrophosphate |
| PMA2 | YPL036W | | Plasma membrane H+-ATPase, isoform of Pma1p, |
| | | | involved in pumping protons out of the cell; |
| | | | regulator of cytoplasmic pH and plasma membrane |
| | | | potential |
| PDR12 | YPL058C | | Plasma membrane ATP-binding cassette (ABC) |
| | | | transporter |
| LCB1 | YDR062W | | Component of serine palmitoyltransferase, |
| | | | responsible along with Lcb1p for the first committed |
| | | | step in sphingolipid synthesis |
| CHA1 | YCL064C | | Catabolic L-serine (L-threonine) deaminase, |
| | | | catalyzes the degradation of both L-serine and L- |
| | | | threonine |
| HXT5 | YHR096C | | Hexose transporter with moderate affinity for |
| | | | glucose, induced in the presence of non-fermentable |
| | | | carbon sources, induced by a decrease in growth rate, |
| | | | contains an extended N-terminal domain relative to |
| | | | other HXTs |
| MTD1 | YKR080W | | NAD-dependent 5,10-methylenetetrahydrafolate |
| | | | dehydrogenase, plays a catalytic role in oxidation of |
| | | | cytoplasmic one-carbon units |
| MSC6 | YOR354C | | Mutant is defective in directing meiotic |
| | | | recombination events to homologous chromatids; the |
| | | | protein is detected in highly purified mitochondria in |
| | | | high-throughput studies |
| SCW10 | YMR305C | | Cell wall protein with similarity to glucanases |
| | YAL065C | | Has homology to FLO1 |
| | YJL107C | | Expression is induced by activation of the HOG1 |
| | | | mitogen-activated signaling pathway and this |
| | | | induction is Hog1p/Pbs2p dependent |
| CSM3 | YMR048W | | Protein required for accurate chromosome |
| | | | segregation during meiosis |
| RGT2 | YDL138W | | Plasma membrane glucose receptor, highly similar to |
| | | | Snf3p |
| CHS7 | YHR142W | | Involved in chitin biosynthesis by regulating Chs3p |
| | | | export from the ER |
| PAU7 | YAR020C | | Part of 23-member seripauperin multigene family, |
| | | | active during alcoholic fermentation |
| SLU7 | YDR088C | | RNA splicing factor, required for ATP-independent |
| | | | portion of 2nd catalytic step of spliceosomal RNA |
| | | | splicing; interacts with Prp18p; contains zinc |
| | | | knuckle domain |
| ARP6 | YLR085C | | Actin-related protein that binds nucleosomes; a |
| | | | component of the SWR1 complex |
| MRP21 | YBL090W | | Mitochondrial ribosomal protein of the small subunit |
| AFG2 | YLR397C | | ATPase of the CDC48/PAS1/SEC18 (AAA) family |
| PPT2 | YPL148C | | Phosphopantetheine:protein transferase (PPTase), |
| | | | activates mitochondrial acyl carrier protein (Acp1p) |
| | | | by phosphopantetheinylation |
| PGS1 | YCL004W | | Phosphatidylglycerolphosphate synthase, catalyzes |
| | | | the synthesis of phosphatidylglycerolphosphate from |
| | | | CDP-diacylglycerol and sn-glycerol 3- |
| YHC1 | YLR298C | | Component of the U1 snRNP complex required for |
| | | | pre-mRNA splicing; putative ortholog of human |
| | | | U1C protein, which is involved in formation of a |
| | | | complex between U1 snRNP and the pre-mRNA 5′ |
| | | | splice site |
| | YJL045W | | Minor succinate dehydrogenase isozyme; |
| | | | homologous to Sdh1p |
| NDD1 | YOR372C | | Transcriptional activator important for nuclear |
| | | | division; localized to the nucleus; component of the |
| | | | mechanism that activates the expression of a set of |
| | | | late-S-phase-specific genes |
| KEX2 | YNL238W | | Subtilisin-like protease (proprotein convertase), a |
| | | | calcium-dependent serine protease involved in the |
| | | | activation of proproteins of the secretory pathway |
| COG7 | YGL005C | | Component of the conserved oligomeric Golgi |
| | | | complex (Cog1p through Cog8p), a cytosolic |
| | | | tethering complex that functions in protein |
| | | | trafficking to mediate fusion of transport vesicles to |
| | | | Golgi compartments |
| PRP45 | YAL032C | | Protein required for pre-mRNA splicing; associates |
| | | | with the spliceosome and interacts with splicing |
| | | | factors Prp22p and Prp46p; orthologous to human |
| | | | transcriptional coactivator SKIP and can activate |
| | | | transcription of a reporter gene″ |
| MET16 | YPR167C | | ″3′-phosphoadenylsulfate reductase, reduces 3′- |
| | | | phosphoadenylyl sulfate to adenosine-3′,5′- |
| | | | bisphosphate and free sulfite using reduced |
| | | | thioredoxin as cosubstrate |
| RAM2 | YKL019W | | Alpha subunit of both the farnesyltransferase and |
| | | | type I geranylgeranyltransferase that catalyze |
| | | | prenylation of proteins containing a CAAX |
| | | | consensus motif |
| MGR3 | YMR115W | | Subunit of the mitochondrial (mt) i-AAA protease |
| | | | supercomplex, which degrades misfolded |
| | | | mitochondrial proteins |
| FLO8 | YER109C | | Transcription factor required for flocculation, diploid |
| | | | filamentous growth, and haploid invasive growth |
| BRE2 | YLR015W | | Subunit of the COMPASS (Set1C) complex, which |
| | | | methylates histone H3 on lysine 4 and is required in |
| | | | transcriptional silencing near telomeres ″ |
| REC102 | YLR329W | | Protein involved in early stages of meiotic |
| | | | recombination; required for chromosome synapsis; |
| | | | forms a complex with Rec104p and Spo11p |
| IDP3 | YNL009W | | Peroxisomal NADP-dependent isocitrate |
| | | | dehydrogenase, catalyzes oxidation of isocitrate to |
| | | | alpha-ketoglutarate with the formation of NADP(H+) |
| PEX18 | YHR160C | | Peroxin required for targeting of peroxisomal matrix |
| | | | proteins containing PTS2; interacts with Pex7p |
| APS2 | YJR058C | | Small subunit of the clathrin-associated adaptor |
| | | | complex AP-2, which is involved in protein sorting |
| | | | at the plasma membrane |
| HUG1 | YML058W-A | | Protein involved in the Mec1p-mediated checkpoint |
| | | | pathway that responds to DNA damage or replication |
| | | | arrest |
| OSH7 | YHR001W | | Member of an oxysterol-binding protein family with |
| | | | seven members in S. cerevisiae |
| KSS1 | YGR040W | | Mitogen-activated protein kinase (MAPK) involved |
| | | | in signal transduction pathways that control |
| | | | filamentous growth and pheromone response |
| PTA1 | YAL043C | | Subunit of holo-CPF, a multiprotein complex and |
| | | | functional homolog of mammalian CPSF, required |
| | | | for the cleavage and polyadenylation of mRNA and |
| | | | snoRNA 3′ ends ″ |
| ECI1 | YLR284C | | Peroxisomal delta3,delta2-enoyl-CoA isomerase, |
| | | | hexameric protein that converts 3-hexenoyl-CoA to |
| | | | trans-2-hexenoyl-CoA |
| SWD2 | YKL018W | | Subunit of the COMPASS (Set1C) complex, which |
| | | | methylates histone H3 on lys 4 and is involved in |
| | | | telomeric silencing; subunit of CPF (cleavage and |
| | | | polyadenylation factor), a complex involved in |
| | | | RNAP II transcription termination |
| VPS71 | YML041C | | Nucleosome-binding component of the SWR1 |
| | | | complex, which exchanges histone variant H2AZ |
| | | | (Htz1p) for chromatin-bound histone H2A; required |
| | | | for vacuolar protein sorting |
| EMP47 | YFL048C | | Integral membrane component of endoplasmic |
| | | | reticulum-derived COPII-coated vesicles |
| ADE13 | YLR259W | | Adenylosuccinate lyase, catalyzes two steps in the de |
| | | | novo purine nucleotide biosynthetic pathway |
| FLC1 | YPL221W | | Putative FAD transporter, required for uptake of |
| | | | FAD into endoplasmic reticulum |
| AOS1 | YPR180W | | Nuclear protein that acts as a heterodimer with |
| | | | Uba2p to activate Smt3p (SUMO |
| YMC1 | YPR058W | | Mitochondrial protein, putative inner membrane |
| | | | transporter with a role in oleate metabolism and |
| | | | glutamate biosynthesis; member of the mitochondrial |
| | | | carrier (MCF) family |
| MRPL20 | YKR085C | | Mitochondrial ribosomal protein of the large subunit |
| EMC1 | YCL045C | | Member of a transmembrane complex required for |
| | | | efficient folding of proteins in the ER; null mutant |
| | | | displays induction of the unfolded protein response; |
| | | | interacts with Gal80p |

In some embodiments, the activity of a functional variant or homolog of a protein to be overexpressed in accordance with the invention is determined by evaluating a yeast strain, e.g., a Saccharomyces cerevisiae yeast strain such as S. cerevisiae CS-400, that is genetically modified to overexpress the variant or homolog in a fermentation reaction. For example, the yeast strain modified to overexpress the variant may be evaluated to determine whether the variant has one or more of the following activities: increases hexose sugar utilization, e.g., glucose utilization; increases pentose sugar utilization, e.g., xylose utilization; or increases yield of a fermentation production, e.g., of an alcohol such as ethanol in a fermentation reaction, where the increase is in comparison to a control parent yeast strain that has not been genetically modified to overexpress the variant. For example, a yeast strain genetically modified to overexpress a variant having at least 70% identity, or at least 75%, at least 80%, at least 85%, at least 90%, at least 91%, at least 92%, at least 93%, at least 94%, at least 95%, at least 96%, at least 97%, at least 98%, or least 99% identity to one of SEQ ID NOS: 1-11 or SEQ ID NOS: 55-113 may be evaluated for the ability to increase glucose or xylose utilization in a fermentation reaction, optionally a fermentation reaction that comprises a cellulosic hydrolysate, e.g., as described in Example 1. In some embodiments, glucose and/or xyloseutilization (e.g., the amount of glucose and/or xylose consumed over a specific period of time or the rate at which a specified amount of glucose and/or xylose is consumed in a specified amount of time) in the modified host cell is increased by at least about 10%, at least 15%, at least 20%, at least 25%, at least 30%, at least 40%, or at least 50% greater than the amount of glucose and/or xylose consumed over the same specific period of time for a control cell that has not been genetically modified (e.g., an unmodified Saccharomyces cerevisiae cell of the same strain). Glucose and xylose consumption can be determined by methods described in the Examples section (e.g., Examples 1 and 2) and/or using any other methods known in the art. For example, a xylose-utilizing Saccharomyces cerevisiae strain transformed with a nucleic acid expression construct encoding a variant can be assayed for xylose utilization compared to a control of the same strain that was not transformed with a nucleic acid encoding the variant in a wheat straw biomass-derived sugar hydrolysate containing xylose at pH 5.5 or pH 5.8. The amount of residual sugars and, if desired, other products such as ethanol, in the supernatant is measured, e.g., using a spectrophotometric methods or using HPLC-based methods after a period of time, for example 48 hours and compared to the amount of residual sugars or other products produced by the control transformed with the antibiotic marker only.

In another example, a yeast strain genetically modified to overexpress a variant having at least 70% identity, or at least 75%, at least 80%, at least 85%, at least 90%, at least 91%, at least 92%, at least 93%, at least 94%, at least 95%, at least 96%, at least 97%, at least 98%, or least 99% identity to one of SEQ ID NOS:12-27 may be evaluated for the ability to increase glucose utilization in a fermentation reaction, optionally a fermentation reaction that comprises furfural, e.g., using an assay as described in Example 2.

In some embodiments, a fermentation reaction used to assess protein activity may also include ethanol as a component in the culture medium.

Hexose sugar utilization, e.g., glucose utilization; pentose sugar utilization, e.g., xylose utilization; yield of fermentation production, e.g., ethanol, from a fermentation reaction, or furfural reduction can be determined using known techniques. For example, to determine glucose or xylose utilization, the amount of glucose or xylose in a fermentation reaction after a specified time period, such as 24 hours, is determined, e.g., using HPLC. The reduction in the amount of residual glucose or xylose in the medium over time reflects the rate of sugar utilization. The amount of a fermentation product, e.g., ethanol, produced in a reaction after a specified period time can also be determined, e.g., using HPLC. Similarly, furfural levels in a fermentation reaction after a specified period of time can be assessed by HPLC. A variant ERR3, FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, or PDR12 protein; or a variant LCB2, CHA1, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W protein useful in the invention results in at least a 5% increase, relative to the parent yeast strain that is not modified to overexpress the protein, in at least one of the following in a fermentation reaction: hexose sugar, e.g., glucose, utilization; pentose sugar, e.g., xylose, utilization; or fermentation product, e.g., ethanol, yield. In some embodiments, the increase is at least 10% or at least 20%. In some embodiments, the increase obtained with the variant is equivalent to that obtained using the wildtype sequence, or at least 90%, 80%, 70%, 60%, or 50% of the activity achieved with the wildtype sequence.

### Genetic Modification of Yeast Host Cells

Yeast host cells can be modified to overexpress a gene using known techniques. In some embodiments, the host cell is engineered to overexpress a gene encoding a protein product that is endogenous to the cell. In one example of such an embodiment, the host cells may be transformed with an expression construct comprising a nucleic acid sequence that encodes the endogenous protein. In typical embodiments, the nucleic acid sequence encoding the endogenous protein is linked to a promoter, e.g., to its native promoter or to a heterologous promoter. In some embodiments, the expression construct may be targeted for integration into the host genome. In other embodiments, the expression construct introduced into the yeast host cell may be episomal, e.g., targeted for integration into a yeast 2 micron plasmid, or otherwise introduced as a plasmid construct that is episomal. In some embodiments, the host cell may be transformed with an expression construct to introduce a heterologous promoter into the yeast genome where the integrated promoter drives expression of the endogenous gene. In such embodiments, the promoter typically comprises enhancer sequences.

In some embodiments, a yeast host cell can be modified to overexpress a gene that encodes a protein product that is exogenous to the cell. In one example of such an embodiment, the host cell may be transformed with an expression construct comprising a nucleic acid sequence that encodes the exogenous protein. In typical embodiments, the nucleic acid sequence encoding the exogenous protein is operably linked to a heterologous promoter. In other embodiments, the expression construct may be targeted to a yeast host cell genome so that the exogenous gene is integrated into a yeast chromosome. In some embodiments, the expression construct may be targeted for integration into a yeast plasmid, e.g., yeast 2 micron plasmid, or other wise introduced in a plasmid vector that is episomally maintained.

In some embodiments, multiple copies of a polynucleotide encoding a protein to be overexpressed may be introduced into the yeast host cell where overexpression results from the presence of multiple copies.

In some embodiments, a single expression construct comprising two or more of the proteins to be overexpressed may be introduced into a cell. In such an embodiment, expression of the polynucleotides encoding the proteins may be driven by a single promoter or separate promoters.

Methods for recombinant expression of proteins in yeast are well known in the art, and a number of vectors are available or can be constructed using routine methods (See, e.g., Tkacz and Lange, Advances in Fungal Biotechnology for Industry, Agriculture, and Medicine, Kluwer Academic/Plenum Publishers, New York, 2004; Zhu et al., Plasmid 6:128-33, 2009; and Kavanagh, Fungi: Biology and Applications, John Wiley & Sons, Malden, Mass., 2005; all of which are incorporated herein by reference).

### Nucleic Acid Construct Components

In some embodiments, recombinant nucleic acid constructs for use in the invention contain a transcriptional regulatory element e.g., a promoter, a transcription termination sequence, etc., that is functional in a yeast cell. The choice of appropriate control sequences for use in the polynucleotide constructs of the present disclosure is within the skill in the art and in various embodiments is dependent on the recombinant host cell used and the desired method of recovering the fermentation products produced by the yeast host cells.

Promoters that are suitable for use include endogenous or heterologous promoters. A promoter may be either a constitutive or inducible promoter. In some embodiments, useful promoters are those that are insensitive to catabolite (glucose) repression and/or do not require xylose or glucose for induction. Promoters that are suitable for use invention include yeast promoters from glycolytic genes (e.g., yeast phosphofructokinase (PFK), triose phosphate isomerase (TPI), glyceraldehyde-3-phosphate dehydrogenase (GPD, TDH3 or GAPDH), pyruvate kinase (PYK), glucose transporters; ribosomal protein encoding gene promoters; alcohol dehydrogenase promoters (ADH1, ADH2, ADH4, etc.), enolase promoter (ENO), or phosphoglycerate kinase (PGK); See e.g., WO 93/03159, which is incorporated herein by reference). Other promoters include a galactokinase (GAL1) promoter, a fructose 1,6-bisphosphate aldolase (FBA1) promoter, a transcription elongation factor (TEF) promoter. In some embodiments, the promoter is from Saccharomyces cerevisiae. Other useful promoters for yeast host cells are well known in the art (see e.g., Romanos et al., Yeast 8:423-488, 1992, incorporated herein by reference).

A nucleic acid construct of the invention may also comprise additional sequences, such as transcription termination sequences, enhancers, origins of replication, or marker genes. Examples of transcription terminators that are functional in yeast host cells include those of the CYC1, ADH1 and ADH2 genes. For example, in some embodiments, the nucleic acid constructs optionally contain a ribosome binding site for translation initiation. The constructs may also optionally include additional sequences for increasing expression (e.g., an enhancer sequence). Suitable marker genes include, but are not limited to those coding for resistance to antibiotics or antimicrobials (e.g., ampicillin, kanamycin, chloramphenicol, tetracycline, streptomycin, spectinomycin, neomycin, geneticin, nourseothricin, hygromycin, and/or phleomycin).

In some embodiments, the nucleic acid constructs contain a yeast origin of replication. Examples include constructs containing autonomous replicating sequences, constructs containing 2 micron DNA including the autonomous replicating sequence and rep genes, constructs containing centromeres like the CEN6, CEN4, CEN11, CDN3 and autonomous replicating sequences, and other like sequences that are well known in the art. Suitable vectors include episomal vector constructs based on the yeast 2 microns or CEN origin based plasmids such as pYES2/CT, pYES3/CT, pESC/His, pESC/Ura, pESC/Trp, pESC/Leu, p427TEF, pRS405, pRS406, pRS413, and other yeast-based constructs known in the art.

### Random and Site-Specific Integration

A nucleic acid construct may also comprise elements to facilitate integration of a heterologous polynucleotide into the yeast DNA, e.g, a yeast chromosome or yeast episomal plasmid such as the 2 micron plasmid, by site-directed or random homologous or non-homologous recombination. In some embodiments, the nucleic acid constructs comprise elements that facilitate homologous integration. In some embodiments, the polynucleotide is integrated at one or more sites, to provide one or more copies of the sequence in the yeast host cell. In some embodiments, the nucleic acid constructs comprise a protein-coding polynucleotide and a promoter that is operatively linked to the polynucleotide and genetic elements to facilitate integration into the yeast chromosome at a location that is downstream of a native promoter in the host chromosome).

Genetic elements that facilitate integration by homologous recombination include those having sequence homology to targeted integration sites in the yeast DNA. Suitable sites that find use as targets for integration include, for example, the TY1 locus, the RDN locus, the ura3 locus, the GPD locus, aldose reductase (GRE3) locus, etc. Those of skill in the art appreciate that additional sites for integration can be readily identified by microarray analysis, metabolic flux analysis, comparative genome hybridization analysis, and other such methods that are well known in the art.

Genetic elements or techniques that facilitate integration by non-homologous recombination include restriction enzyme-mediated integration (REMI) (See e.g., Manivasakam et al., Mol. Cell Biol., 18:1736-1745 (1998), incorporated herein by reference), transposon-mediated integration, as well as additional elements and methods well known in the art.

In some embodiments, expression constructs may comprises sequences to target integration to a yeast episomal plasmid, e.g., the 2 micron plasmid. Examples of 2 micron plasmids are described in WO 2012/044868 and U.S. Patent Application Publication No. 2012/0088271, which are incorporated by reference. For example, a vector that contains regions of homology that target the R3 region on the native Saccharomyces 2 micron plasmid between the FLP and REP2 genes may be used.

### Additional Modifications for Expression of a Gene in a Host Cell

A DNA sequence can be optimized for expression in a yeast host cell. A variety of methods are known for determining the codon frequency and/or codon preference in specific organisms, including multivariate analysis, for example, using cluster analysis or correspondence analysis, and the effective number of codons used in a gene (see GCG CodonPreference, Genetics Computer Group Wisconsin Package; Codon W, John Peden, University of Nottingham; McInerney, J. O, 1998, Bioinformatics 14:372-73; Stenico et al., 1994, Nucleic Acids Res. 222437-46; Wright, F., 1990, Gene 87:23-29; Wada et al., 1992, Nucleic Acids Res. 20:2111-2118; Nakamura et al., 2000, Nucl. Acids Res. 28:292; Henaut and Danchin, all of which are incorporated herein be reference). The data source for obtaining codon usage may rely on any available nucleotide sequence capable of coding for a protein, e.g., complete protein coding sequences (CDSs), expressed sequence tags (ESTs), or predicted coding regions of genomic sequences.

### Host Cells

In certain embodiments, the yeast recombinant host cell comprising a nucleic acid encoding protein to be over-expressed in accordance with the invention is a species selected from the group consisting of Saccharomyces, Candida, Hansenula, Schizosaccharomyces, Pichia, Kluyveromyces, Rhodotorula, and Yarrowia. In some embodiments, the yeast host cell is a species of a genus selected from the group consisting of Saccharomyces, Candida, and Pichia. In some embodiments the yeast host cell is a Saccharomyces sp.

In various embodiments, the yeast host cell is selected from the group consisting of Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia ferniemtans, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, Candida krusei, Candida ethanolic and Hansenula polymorpha, and synonyms or taxonomic equivalents thereof. In some embodiments, the host cell is Saccharomyces cerevisiae.

In certain embodiments, the yeast host cell is a wild-type cell. In various embodiments, the wild-type yeast cell strain is selected from, but not limited to, Saccharomyces cerevisiae strain BY4741, strain FL100a, strain INVSC1, strain NRRL Y-390, strain NRRL Y-1438, strain NRRL YB-1952, strain NRRL Y-5997, strain NRRL Y-7567, strain NRRL Y-1532, strain NRRL YB-4149 and strain NRRL Y-567. Additional yeast strains that find use in the invention include, but are not limited, to SuperStart™, Thermosacc®, and EDV46 (all from Lallemand, Inc., Montreal, Canada).

In other embodiments, the yeast host cell into which the recombinant expression constructs are introduced in accordance with the invention has additional genetic modifications. Examples of genetically modified yeast useful as recombinant host cells include, but are not limited to, genetically modified yeast found in the Open Biosystems collection found at the www site openbiosystems.com/GeneExpression/Yeast/YKO/. See Winzeler et al. (1999) Science 285:901-906, available from Open Biosystems, part of Thermo Fisher Scientific.

In some embodiments, the yeast host cells is Y108-1 (ATCC Deposit No. PTA-10567; see, also U.S. Patent Application Publication No. 20110159560), or S. cerevisiae CS-400 (ATCC No. PTA-12325) strain, or a progeny strain thereof; or BY4741, SuperStart™, Thermosacc®, EDV4, BY4741, or a progeny strain thereof. In some embodiments, the yeast host cells have been engineered to ferment xylose, e.g., Y108-1 or CS-400. In some embodiments, the strain is an industrial yeast strain typically used in fuel ethanol fermentation, such as SuperStart™, Thermosacc®, or EDV4.

In some embodiments, the yeast host cells, e.g., Saccharomyces cerevisiae host cells, are optionally mutagenized and/or modified to exhibit further desired phenotypes (e.g., for further improvement in the utilization of glucose and/or pentose sugars, increased transport of sugar into the host cell, increased flux through the pentose phosphate pathway, decreased sensitivity to catabolite repression, increased tolerance to ethanol, increased tolerance to acetate, increased tolerance to increased osmolarity, increased tolerance to organic acids (low pH), reduced production of byproducts, etc.).

In some embodiments, suitable yeast host cells for use in the invention have been selected and/or engineered to enhance tolerance to inhibitors, e.g., acetic acid, furfural, and hydroxymethylfurfural that are present in lignocellulose hydrolysates. For example, strains of Pichia and Saccharomyces have been adapted to media containing furfural and/or hydroxymethylfurfural (Liu et al., J. Ind. Microbiol. Biotechnol. 31:345-52, 2004; Liu et al. Appl. Biochem. Biotechnol. 121-124:451-60, 2005; Huang et al., Bioresource Technol. 100:3914-20, 2009; Martin et al., Bioresource Technol. 98:1767-73, 2007).

In some embodiments, the recombinant yeast host cells that are modified to overexpress a gene in accordance with the invention also comprise recombinant polynucleotides that express proteins that confer the ability to ferment a pentose sugar (e.g., convert xylose into ethanol). Strategies for genetically modifying yeast host cells, e.g., Saccharomyces cerevisiae cells to ferment pentose sugars (particularly xylose) are known by those of skill in the art (see, e.g., Matsushika, Appl. Microbiol. Biotechnol., 84:37-53, 2009; van Maris, Adv. Biochem. Eng. Biotechnol. 108:179-204, 2007; Hahn-Hägerdal, Adv. Biochem. Eng. Biotechnol., 108:147-177, 2007; and Jeffries, Curr. Opin. Biotechnol. 17:320-3266, 2006). For example, in some embodiments the cells may be modified to express a recombinant polynucleotide that encodes a xylose isomerase, a xylose reductase, a xylitol dehydrogenase, a xylulokinase, a xylitol isomerase and/or a xylose transporter (see, e.g., Brat, Appl. Environ. Microbiol., 75:2304-11, 2009); Madhavan Appl. Microbiol. Biotechnol., 82:1067-7, 2009; and Kuyper FEMS Yeast Res. 4:69-78, 2003; Krahulec, Biotechnol. J., 4:684-694, 2009; Bettiga Biotechnol. Biofuels 1:16, 2008; and Matsushika, J. Biosci. Bioeng. 105:296-299, 20082008), alone or in combination with other components of the pentose catabolism or sugar uptake pathways, and/or other ethanologenic enzymes (e.g., pyruvate decarboxylase, aldehyde dehydrogenase, and/or an alcohol dehydrogease). See, also, e.g., WO2001088094 for examples of suitable yeast strains and xylose reductase, xylitol dehydrogenase and xylulokinase sequences. Examples of yeast transporters are GXF1, SUT1, At6g59250, HXT4, HXT5, HXT7, GAL2, AGT1, and GXF2. Examples of other modifications that may be made to yeast strains can be found, e.g., in U.S. Patent Application Publication No. 20110159560.

### Additional Modifications

Host cells engineered to overexpress a protein product of an ERR3, FOX2, LYS1, MET1, MIG2, RMD6, RME1, SIP1, SNP1, TDH1, or ZWF1 gene; or a protein product of a GPD1, RSF2, GND2, TRK1, HSP31, HSP33, HSP30, HSP32, ADH6, UFD4, PRO1, SIA1, ARI1, LPP1, PMA2, or PDR12 gene, or a protein product of a LCB2, CHA1, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W gene, may also be engineered to express at least one enzyme from the pentose phosphate pathway (e.g., a ribulose-5-phosphate 3-epimerase (RPE1), a ribose-5-phosphate keto-isomerase (RKI1), a transketolase (TKL1), a transaldolase (TAL1), and the like); at least one enzyme from the glycolysis metabolic pathway (e.g., a hexokinase (HXK1/HXK2), a glyceraldehyde-3-phosphate dehydrogenase (GAPDH), a pyruvate kinase (PVK2), and the like); and/or at least one ethanologenic enzyme (e.g., pyruvate decarboxylase and/or an alcohol dehydrogenase).

The recombinant host cells into which the expression constructs in accordance with the invention are introduced may also be engineered such that one or more endogenous genes are deleted or inactivated. For example, in some embodiments, yeast host cells for use in the invention may have at least one of their native genes deleted in order to improve the utilization of pentose sugars (e.g., xylose, arabinose, etc.), increase transport of xylose into the cell, increase xylulose kinase activity, increase flux through the pentose phosphate pathway, decrease sensitivity to catabolite repression, increase tolerance to ethanol, increase tolerant to acetate, increase tolerance to increased osmolarity, increase tolerance to organic acids (low pH), reduce production of by products, and other like properties related to increasing flux through the relevant pathways to produce ethanol and other desired metabolic products at higher levels, where comparison is made with respect to the corresponding cell without the deletion(s).

### Culture of Genetically Modified Yeast

A host cell, e.g., Saccharomyces cerevisiae, comprising a promoter operably linked to a nucleic acid encoding an ERR3, FOX2, LYS1, MET1, MIG2, RMD6, RME1, SIP1, SNP1, TDH1, ZWF1, GPD1, RSF2, GND2, TRK1, HSP31, HSP33, HSP30, HSP32, ADH6, UFD4, PRO1, SIA1, ARI1, LPP1, PMA2, PDR12, LCB2, CHA1, HXT5, MTD1, MSC6, SCW10, YAL065C, YJL107C, CSM3, RGT2, CHS7, BOP2, YDR271C, PAU7, YGL258W-A, SLU7, ARP6, MRP21, AFG2, YJL152W, PPT2, PGS1, YHC1, YJL045W, NDD1, KEX2, COG7, PRP45, MET16, YGR114C, RGI2, YOR318C, RAM2, YPR027C, MGR3, FLO8, BRE2, REC102, IDP3, PEX18, APS2, HUG1, OSH7, KSS1, PTA1, YHR138C, TSR3, ECI1, RDL2, SWD2, VPS71, EMP47, ADE13, FLC1, AOS1, YMC1, MRPL20, EMC1, or YMR155W polypeptide, e.g., an amino acid sequence selected from the group consisting of SEQ ID NOS:1-27 and SEQ ID NOS:55-116, or variant thereof, can be cultured under a variety of conditions. Conditions for culturing and maintaining yeast are well known in the art. Cell culture media in general are set forth in Atlas and Parks, eds., 1993, The Handbook of Microbiological Media. The individual components of media for cultivating yeast cells are available from commercial sources, e.g., under the Difco™ and BBL™ trademarks.

In some embodiments, the yeast cells are cultured under conditions (“fermentation conditions”) suitable for the production of the fermentation product. In these methods, the substrate present in the cell culture is converted by the cells to produce at least one fermentation product, such as an alcohol, e.g., ethanol. In some embodiments, the fermentation product(s) is collected from the culture. For examples, some methods comprise distilling the fermentation product from the culture using methods known in the art.

Fermentation conditions for obtaining fermentation products such as an alcohol are well known in the art. In some embodiments, the fermentation process is carried out under aerobic conditions, while in other embodiments microaerobic (i.e., where the concentration of oxygen is less than that in air) or anaerobic conditions are used. Typical anaerobic conditions are the absence of oxygen (i.e., no detectable oxygen), or less than about 5, about 2.5, or about 1 mmol/L/h oxygen. In the absence of oxygen, the NADH produced by glycolysis cannot be oxidized by oxidative phosphorylation. Under anaerobic conditions, pyruvate or a derivative thereof may be utilized by the host cell as an electron and hydrogen acceptor in order to generated NAD+. In some embodiments, when the fermentation process is carried out under anaerobic conditions, pyruvate is reduced to at least one fermentation product, including but not limited to ethanol, butanol, fatty alcohol (e.g., C8-C20 fatty alcohols), lactic acid, 3-hydroxypropionic acid, acrylic acid, acetic acid, succinic acid, citric acid, malic acid, fumaric acid, an amino acid, 1,3-propanediol, ethylene, glycerol, terpenes, and/or antimicrobials (e.g., β-lactams, such as cephalosporin).

In some embodiments, the fermentation involves batch processes, while in other embodiments, it is a continuous process. In some embodiments, after fermentation, the cells are separated from the fermented slurry and re-contacted with a fresh batch of saccharified lignocellulose. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out a temperature of about 10° C. to about 60° C., about 15° C. to about 50° C., about 20° C. to about 45° C., about 20° C. to about 40° C., about 20° C. to about 35° C., or about 25° C. to about 45° C. In one embodiment, the fermentation is carried out at a temperature of about 28° C. and/or about 30° C. It will be understood that, in certain embodiments where thermostable host cells are used, fermentations may be carried out at higher temperatures.

In some embodiments, the fermentation is carried out for a time period of about 8 hours to 240 hours, about 8 hours to about 168 hours, about 8 hours to 144 hours, about 16 hours to about 120 hours, or about 24 hours to about 72 hours.

In some embodiments, the fermentation will be carried out at a pH of about 3 to about 8, about 4.5 to about 7.5, about 5 to about 7, or about 5.5 to about 6.5.

In some embodiments, the fermentation product is separated from the culture using any suitable technique known in the art (e.g., stripping, membrane filtration, and/or distillation), in order to produce purified fermentation product that finds use as a fuel. In some embodiments, the purified fermentation product is present in a concentration in the range of about 5% to about 99.9% (e.g., in the range of about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% to 90%). In some embodiments, the purified fermentation product is present in a concentration of about 10 to about 15%. In some embodiments, the fermentation product is ethanol.

### Culture in the Presence of a Cellulosic Hydrolysate

In some embodiments, genetically modified yeast cells of the present invention are cultured in a reaction that comprises a cellulosic hydrolysate. A cellulosic hydrolysate may be obtained by chemical, e.g., acid or base, or enzymatic treatment of a cellulosic biomass before and/or during fermentation to produce monosaccharides, e.g., hexose sugars such as glucose and pentose sugars such as xylose. A yeast host cell thus may be contacted with the cellulosic hydrolysate that is produced during a fermentation reaction of prior to a fermentation reaction. In the present invention, “contacting” a yeast host cell with a cellulosic hydrolysate means that the yeast host cell is cultured in a media that has contains the cellulosic hydrolysate.

The cellulosic biomass from which a cellulosic hydrolysate is obtained may be from any number of sources. In some embodiments, the cellulosic biomass includes lignocellulosic substrates including but not limited to, wood, wood pulp, paper pulp, corn stover, corn fiber, rice, paper and pulp processing waste, woody or herbaceous plants, fruit or vegetable pulp, distillers grain, grasses, rice hulls, wheat straw, cotton, hemp, flax, sisal, corn cobs, sugar cane bagasse, switch grass and mixtures thereof. The biomass may optionally be pretreated to increase the susceptibility of cellulose to hydrolysis using methods known in the art such as chemical, physical and biological pretreatments (e.g., steam explosion, pulping, grinding, solvent exposure, and the like, as well as combinations thereof).

In certain embodiments a lignocellulosic biomass may contain at least about 50%, at least about 70% or at least about 90% (by dry weight) lignocellulose. It is understood that lignocellulosic feedstock may also contain other constituents in addition to lignocellulose, such as fermentable sugars, un-fermentable sugars, proteins, oil, carbohydrates, etc. Certain lignocellulosic feedstocks contain about 30% to about 50% cellulose, about 15% to about 35% hemicelluloses, and about 15% to about 30% lignin.

Processes for obtaining a cellulosic hydrolysate are chemical hydrolysis, which involves the hydrolysis of the cellulosic biomass using acid or base treatment, and enzymatic hydrolysis, which involves hydrolysis with cellulase or hemicellulase enzymes.

A cellulosic biomass may be treated with an acid to produce a hydrolysate. In such a method, the cellulosic biomass is subjected to steam and an acid (e.g., a mineral acid such as sulfuric acid, sulfurous acid, hydrochloric acid, or phosphoric acid). The temperature, acid concentration and duration of the acid hydrolysis are sufficient to hydrolyze the cellulose and hemicellulose to their monomeric constituents (i.e., glucose from cellulose and xylose and one or more of galactose, mannose, arabinose, acetic acid, galacturonic acid, and glucuronic acid from hemicelluloses). In some embodiments in which sulfuric acid is utilized, it can be utilized in concentrated (about 25-about 80% w/w) or dilute (about 3 to about 8% w/w) form. The resulting aqueous slurry contains unhydrolyzed fiber that is primarily lignin, and an aqueous solution of glucose, xylose, organic acids, including primarily acetic acid, as well as glucuronic acid, formic acid, lactic acid and galacturonic acid, and the mineral acid.

A cellulosic biomass may also be treated with one or more enzymes to obtain a hydrolysate. In such methods, steam and mild acid are also typically used. The steam temperature, acid (e.g., a mineral acid such as sulfuric acid) concentration and treatment time of the acid pretreatment step are chosen to be milder than that in the acid hydrolysis process. Similar to the acid hydrolysis process, the hemicellulose is hydrolyzed to one or more of xylose, galactose, mannose, arabinose, acetic acid, glucuronic acid, formic acid, and/or galacturonic acid. However, the milder pretreatment does not hydrolyze a large portion of the cellulose, but rather increases the cellulose surface area. The pretreated cellulose is then hydrolyzed to monosaccharides in a subsequent step that uses cellulase enzymes.

In some embodiments, prior to the addition of enzyme, the pH of the acidic feedstock is adjusted to a value that is suitable for the enzymatic hydrolysis reaction. In some embodiments, this involves the addition of alkali to a pH of between about 4 and about 6, which is the optimal pH range for cellulases, although the pH can be higher if alkalophilic cellulases are used and lower if acidic cellulases are used. Solutions that are most commonly used to adjust the pH of the acidified pretreated feedstock prior to hydrolysis by cellulase enzymes include ammonia, ammonium hydroxide and sodium hydroxide, although the use of carbonate salts such as potassium carbonate, potassium bicarbonate, sodium carbonate and sodium bicarbonate can also be used.

In some embodiments, “cellulases” are used to convert cellulose into monosaccharides. Cellulases are divided into three sub-categories of enzymes: 1,4-β-D-glucan glucanohydrolase (“endoglucanase” or “EG”); 1,4-β-D-glucan cellobiohydrolase (“exoglucanase,” “cellobiohydrolase,” or “CBH”); and 3-D-glucoside-glucohydrolase (“β-glucosidase,” “cellobiase,” or “BG”). See Methods in Enzymology, 1988, Vol. 160, p. 200-391 (Eds. Wood, W. A. and Kellogg, S. T.). These enzymes act in concert to catalyze the hydrolysis of cellulose containing substrates. Endoglucanases break internal bonds and disrupt the crystalline structure of cellulose, exposing individual cellulose polysaccharide chains (“glucans”). Cellobiohydrolases incrementally shorten the glucan molecules, releasing mainly cellobiose units (a water-soluble β-1,4-linked dimer of glucose) as well as glucose, cellotriose, and cellotetrose. β-glucosidases split the cellobiose into glucose monomers.

### Fermentation Systems

The present invention also provides fermentation systems comprising a genetically modified yeast cell. In some embodiments, the fermentation system comprises a fermentation tank containing the yeast cell culture. In some embodiments, the tank is closed (i.e., a sealed tank), while in other embodiments it is an open tank/system. In some additional embodiments, the system provides anaerobic growth conditions. In some embodiments, the system comprises a cellulosic biomass.

It is understood that the examples and embodiments described herein are for illustrative purposes only and that various modifications or changes in light thereof will be suggested to persons skilled in the art and are to be included within the spirit and purview of this application and scope of the appended claims.

### Examples

### Example 1

### Identification of Genes that Enhance Xylose Utilization

Transcriptomics profiles of six xylose-fermenting Saccharomyces strains were determined under fermentation conditions in lignocellulosic plant material using an Agilent microarray. The analysis of up- and down-regulated genes was used to generate a list of genes for overexpression. One hundred seventy two proteins were overexpressed in a xylose-fermenting strain, S. cerevisiae CS-400. For overexpression, the open reading frames (ORFs) were obtained from a yeast library (Open Biosystems (Cat#: YSC3868)) and the ORFs from the library were cloned into a vector compatible with the yeast strains employed in this example. The vector employed contains regions of homology that target the R3 region on the native Saccharomyces 2μ plasmid between the FLP and REP2 genes.

S. cerevisiae CS-400 competent cells were transformed with vectors containing the ORFs using the SIGMA YEAST-1 transformation kit. Transformants were selected on YPD+100 μg/mL Nourosthricin (ClonNAT) to obtain single colonies to prepare cultures for evaluation. Cultures were grown in YPD+100 μg/mL ClonNAT in 96-well plates Aliquots of the cultures were used to inoculate 96-well plates containing minimal media IMv3.0 IMv3.0-X (30 g/1 xylose, 60 g/L glucose; 3 g/L potassium phosphate, 5 g/L ammonium sulphate, 0.5 g/L magnesium sulphate, 19.8 g/L MES pH 6, vitamin solution (3 ml/L) and trace elements solution (3 ml/L)) or minimal media IMv3.0 of the same composition as IMV3.0X, but without xylose supplemented with 400 μg/mL ClonNAT. The plates were covered with airpore seals and incubated at 30° C., 85% relative humidity. For propagation, 20 μl to 150 μl of the saturated cultures were used to inoculate 96-deep well plates containing 380 μl to 850 μl of the IMv3.0 media supplemented with 400 μg/mL ClonNAT and the strains were grown for 24 hours 30° C., 85% relative humidity. At the end of this propagation process, the growth of the cultures was evaluated by optical density using a spectrophotometer at 600 nm.

For fermentation, cells were re-suspended in 400 μl of wheat straw biomass-derived sugar hydrolysates containing xylose at pH 5.5 or pH 5.8. The plates were sealed with silicone sealing mats. Plates were incubated at 30° C. Cells were harvested after 48 hours and the residual sugars in the supernatant and ethanol in the supernatant were measured by a standard HPLC-based method using an Aminex HPX 37H column (DuPont et al., Carb. Polym., 68:1-16, 2007) or an Ion Exclusion HPLC column from Waters Technologies. In some experiments, the residual xylose in the supernatant was measured using a spectrophotometric assay (e.g., Megazyme xylose assay; Cat no. K-XYLOSE, Megazyme International Ireland, Ltd., Wicklow, Ireland) performed according to the manufacture's protocol. The improvement in performance for xylose utilization of yeast that overexpressed the target genes was calculated based on comparison to performance of the control yeast strain, which was transformed with the antibiotic marker only.

TABLE 3

| | | Improvement in |
|---|---|---|
| Common | Systematic | Xylose |
| Name | Name | utilization |
| ZWF1 | YNL241C | + |
| TDH1 | YJL052W | ++ |
| SNP1 | YIL061C | + |
| SIP1 | YDR422C | ++ |
| RME1 | YGR044C | + |
| RMD6 | YEL072W | + |
| MIG2 | YGL209W | ++ |
| MET1 | YKR069W | + |
| LYS1 | YIR034C | + |
| FOX2 | YKR009C | ++ |
| ERR3 | YMR323W | + |

+ improvement up to 20% compared to controls
++ improvement of 20% or greater compared to controls

### Example 2

### Identification of Additional Genes to Improve Glucose Utilization and/or Ethanol Production

Genes were selected for overexpression to evaluate inhibitor tolerance and glucose consumption during fermentation processes. Glucose fermentation rates and furfural reduction in fermentation media were analyzed in this example.

Library construction processes were based on the Saccharomyces cerevisiae ORF collection from Open Biosystems. The yeast employed were Superstart™ yeast (Lallemand Ethanol Technology) and the experimental procedure for obtaining transformants was similar to that described in Example 1. In this example, a yeast vector containing a TEF 1 promoter to drive expression of the heterologous gene was employed.

In this example, 102 genes were overexpressed. Each gene was individually cloned from either the BG1805 plasmid in the Open Biosystems library or from a Saccharomyces cerevisiae genome. The primers were designed with overhangs to insert the ORFs between the TEF1 promoter and the CYC1 terminator in the vector using recombinational cloning. Transformants were selected on YPD+200 μg/mL G418. Single colonies were used to inoculate in YPD+200 μg/mL G418 in 96-well plates and were grown for 24 hours shaken at 30° C., 85% relative humidity. Aliquots of cultures were used to inoculate 96-deep well plates containing 360 μl of YPD plus 160 g/L glucose and 200 μg/mL G418. At the end of this propagation process the growth of the cultures was evaluated by optical density on a spectrophotometer at 600 nm. For fermentation, cells were re-suspended in 400 ul of synthetic fermentation media FM3.0 supplemented with 200 μg/mL G418 (20 g/L yeast extract, 140 g/L glucose, 60 g/L xylose, 9 g/L arabinose, 12 g/L acetic acid, 2 g/L furfural, 2 g/L HMF, pH 5). In some of the fermentation processes, additional fermentation cycles were performed with the addition of 7% ethanol to FM3.0 fermentation media. The plates were sealed with silicone sealing mats and were incubated at 30° C. Cells were harvested after 24 hours and the levels of glucose, furfural, and ethanol in the supernatant were measured by a standard HPLC-based method using an Aminex HPX 37H column (DuPont et al., Carb. Polym., 68:1-16, 2008); or using a Phenomenex Rezex ROA-Organic Acid H+ column. The improvement in performance for glucose consumption, ethanol yield and/or furfural reduction of yeast that overexpressed the target genes was calculated based on comparison to performance of the control yeast strain, which was transformed with the antibiotic marker only.

TABLE 4

| | | Improvement Over Parent Strain | | | | |
|---|---|---|---|---|---|---|
| | | | Glucose | | | |
| | | | consumption | | | |
| | | | rate with | | | |
| | | Glucose | Ethanol | | | |
| | | consumption | added to | Ethanol | Glycerol | Furfural |
| Gene | | rate | media | yield | production | reduction |
| Name | Category | (g/L * hr * OD) | (g/L * hr * OD) | Y E/G | (g/L * hr * OD) | (g/L) |
| GPD1 | Glycerol synthesis | (++++) | (+++) | (−) | (++++) | (−) |
| RSF2 | Transcription | (+++) | | (−) | (++) | (−) |
| | factor | | | | | |
| GND2 | PPP | (+++) | | (−) | (+) | (++) |
| TRK1 | Transporter | (++) | (++) | (−) | (++) | (++) |
| HSP31 | Chaperone | (++) | | (−) | (+++) | (−) |
| HSP33 | Chaperone | (++) | (++) | (−) | (++) | (−) |
| HSP30 | Chaperone | (−) | | (++) | (−) | |
| HSP32 | Chaperone | (++) | | (−) | (++) | (−) |
| ADH6 | Alcohol | (++) | (+++) | (+) | (+++) | (++++) |
| | dehydrogenase | | | | | |
| UFD4 | Protein | (++) | (+++) | (−) | (−) | (++) |
| | degradation | | | | | |
| PRO1 | Other | (++) | | (−) | (−) | |
| SIA1 | Proton pump | (−) | (++) | (++) | (−) | (+++) |
| | regulator | | | | | |
| ARl1 | Oxidoreductase | (+) | (++) | (−) | (−) | (+++) |
| LPP1 | Cell wall | (++) | | (−) | (−) | (−) |
| PMA2 | Pumps | (++) | | (−) | (−) | (−) |
| PDR12 | Pumps | (−) | | (++) | (−) | |

(−) less than or equal to control
(+) improvement up to 20% compared to control
(++) improvement of 20% or greater compared to control
(+++) improvement of 50% or greater compared to control
(++++) improvement over 100% compared to control

### Example 3

### Identification of Additional Genes to Improve Xylose Utilization in Yeast

An additional randomly selected set of 2866 Saccharomyces cerevisiae ORFs was overexpressed to identify genes that confer improvements in xylose fermentation rates in xylose-utilizing yeast. For overexpression, ORFs were obtained from a yeast library (Open Biosystems (Cat#: YSC3868)) and the ORFs from the library were cloned into a vector compatible with the yeast strains employed in this example. The vector employed contains regions of homology that target the R3 region on the native Saccharomyces 2μ plasmid between the FLP and REP2 genes. Multiple pools of approximately 212 randomly selected ORFs were separately transformed into S. cerevisiae CS-400 competent cells using the SIGMA YEAST-1 transformation kit. Screening for improvements in xylose fermentation rates was performed as described in Example 1. The improvement in performance for xylose utilization of yeast that overexpressed the target genes was calculated based on comparison to performance of the control yeast strain, which was transformed with the antibiotic marker only. Genes that improved xylose utilization are listed in Table 5.

TABLE 5

| | | Improvement |
|---|---|---|
| | | in Xylose |
| Common Name | Systematic Name | Utilization |
| LCB2 | YDR062W | ++ |
| CHA1 | YCL064C | + |
| HXT5 | YHR096C | + |
| MTD1 | YKR080W | + |
| MSC6 | YOR354C | + |
| SCW10 | YMR305C | + |
| YAL065C | YAL065C | + |
| YJL107C | YJL107C | + |
| CSM3 | YMR048W | + |
| RGT2 | YDL138W | + |
| CHS7 | YHR142W | + |
| BOP2 | YLR267W | + |
| YDR271C | YDR271C | + |
| PAU7 | YAR020C | + |
| YGL258W-A | YGL258W-A | + |
| SLU7 | YDR088C | + |
| ARP6 | YLR085C | + |
| MRP21 | YBL090W | + |
| CSM3 | YMR048W | + |
| AFG2 | YLR397C | + |
| YJL152W | YJL152W | + |
| PPT2 | YPL148C | + |
| PGS1 | YCL004W | + |
| YHC1 | YLR298C | + |
| YJL045W | YJL045W | + |
| NDD1 | YOR372C | + |
| KEX2 | YNL238W | + |
| COG7 | YGL005C | + |
| PRP45 | YAL032C | + |
| MET16 | YPR167C | + |
| YGR114C | YGR114C | + |
| RGI2 | YIL057C | + |
| YOR318C | YOR318C | + |
| YOR318C | YOR318C | + |
| RAM2 | YKL019W | + |
| MSC6 | YOR354C | + |
| COG7 | YGL005C | + |
| BOP2 | YLR267W | + |
| YPR027C | YPR027C | + |
| MGR3 | YMR115W | + |
| FLO8 | YER109C | + |
| BRE2 | YLR015W | + |
| REC102 | YLR329W | + |
| COG7 | YGL005C | + |
| IDP3 | YNL009W | + |
| PEX18 | YHR160C | + |
| MIG2 | YGL209W | + |
| COG7 | YGL005C | + |
| APS2 | YJR058C | + |
| HUG1 | YML058W-A | + |
| OSH7 | YHR001W | + |
| KSS1 | YGR040W | + |
| PTA1 | YAL043C | + |
| PPT2 | YPL148C | + |
| YHR138C | YHR138C | + |
| TSR3 | YOR006C | + |
| ECI1 | YLR284C | + |
| RDL2 | YOR286W | + |
| SWD2 | YKL018W | + |
| VPS71 | YML041C | + |
| PTA1 | YAL043C | + |
| EMP47 | YFL048C | + |
| ADE13 | YLR359W | + |
| FLC1 | YPL221W | + |
| PRP45 | YAL032C | + |
| AOS1 | YPR180W | + |
| YMC1 | YPR058W | + |
| MRPL20 | YKR085C | + |
| MRPL20 | YKR085C | + |
| EMC1 | YCL045C | + |
| YMR155W | YMR155W | + |

+ improvement up to 20% compared to control
++ improvement of 20% or greater compared to control

### Example 4

### Yeast Chromosomal Integration of Combinations of ORF's to Improve Xylose Fermentation Rates

ORF's that provided improvements in xylose fermentation rates in Example 1 were integrated into yeast host chromosomes and tested in combination to identify additive or synergistic effects on xylose fermentation rates. ORFs were integrated into various chromosomal locations in xylose utilizing yeasts of opposite mating types derived from Saccharomyces cerevisiae CS-400. Yeast mating was then used to generate libraries to test pairwise combinations of genes. Eight genes (MIG2, SIP1, SNP1, FOX2, TDH1, ZWF1, RGT2, AFG2) that were identified in Examples 1 and 3 were integrated into a specific chromosomal site previously shown to confer high levels of expression (site 1) in a haploid xylose utilizing industrial yeast (strain 1) derived from Saccharomyces cerevisiae CS-400 with mating type a. Seven genes (MIG2, SIP1, SNP1, FOX2, TDH1, ZWF1, AFG2) that were identified in Examples 1 and 3 were integrated into various TY elements in a haploid xylose utilizing industrial yeast (strain 2) derived from Saccharomyces cerevisiae CS-400 with mating type a. Cultures of the haploid integration strains were pooled, concentrated on a mixed cellulose ester filter, and then mated on YPD agar plates. After incubation on YPD, the mated population was sporulated on agar plates containing 0.2M potassium acetate. The sample was enriched for spores and then plated to single colonies for screening. The resulting haploid population contains either zero, one or pairwise combinations of integrated genes.

Cultures were grown in YPD in 96-well plates. Aliquots of the cultures were used to inoculate 96-well plates containing minimal media IMv3.0 IMv3.0-X (30 g/1 xylose, 60 g/L glucose; 3 g/L potassium phosphate, 5 g/L ammonium sulphate, 0.5 g/L magnesium sulphate, 19.8 g/L MES pH 6, vitamin solution (3 ml/L) and trace elements solution (3 ml/L)) or minimal media IMv3.0 of the same composition as IMV3.0X, but without xylose. The plates were covered with airpore seals and incubated at 30° C., 85% relative humidity. For propagation, 20 μA to 150 μl of the saturated cultures were used to inoculate 96-deep well plates containing 380 μl to 850 μl of the IMv3.0 media and the strains were grown for 24 hours 30° C., 85% relative humidity. At the end of this propagation process, the growth of the cultures was evaluated by optical density using a spectrophotometer at 600 nm.

For fermentation, cells were re-suspended in 400 μl of wheat straw biomass-derived sugar hydrolysates containing xylose at pH 5.5 or pH 5.8. The plates were sealed with silicone sealing mats. Plates were incubated at 30° C. Cells were harvested after 48 hours and used to inoculate a second fermentation in wheat straw biomass-derived sugar hydrolysates of 48 hours using the same process as above. Samples were taken at the end of the second fermentation cycle and the residual sugars in the supernatant and ethanol in the supernatant were measured by a standard HPLC-based method using an Aminex HPX 37H column (DuPont et al., Carb. Polym., 68:1-16, 2007) or an Ion Exclusion HPLC column from Waters Technologies. In some experiments, the residual xylose in the supernatant was measured using a spectrophotometric assay (Megazyme xylose assay; Cat no. K-XYLOSE, Megazyme International Ireland, Ltd., Wicklow, Ireland) performed according to the manufacture's protocol. The improvement performance for xylose utilization was calculated based on comparison to performance of a control xylose-utilizing yeast strain not containing any ORF integrations. The presence of integrated ORFs in top performing strains was detected by PCR using primers specific for each ORF. The following strains exhibited improved performance relative to the control strain:

TABLE 6

| | ORFs |
|---|---|
| Strain | Integrated |
| 3 | SIP1/FOX2 |
| 5 | TDH/AFG2 |
| 6 | ZWF1 |
| 7 | RGT2/AFG2 |
| 8 | SIP1 |
| 9 | SIP1 |

In order to identify additional ORF combinations that confer additive or synergistic benefits to xylose fermentation rates, the twenty best performing strains were pooled and subjected to an additional cycle of mating and sporulation as described above. Fermentation performance for these strains was evaluated as above. The following strains exhibited improved performance compared to the control strain.

TABLE 7

| Strain | ORFs Detected |
|---|---|
| 10 | FOX2/ZWF1 |
| 11 | SIP1 |
| 12 | SIP1/FOX2/ZWF1 |
| 13 | SIP1/SIP1/ZWF1 |
| 14 | ZWF1 |
| 15 | ZWF1 |

All publications, patents, patent applications, and accession numbers cited herein are hereby incorporated by reference in their entirety for all purposes.

### Illustrative Reference Sequences

SEQ ID NO: 1
ERR3 amino acid sequence; systematic name YMR323W

| | |
|---|---|
| 1 | MSITKVHART VYDSRGNPTV EVEITTENGL FRAIVPSGAS TGIHEAVELR |
| 51 | DGNKSEWMGK GVTKAVSNVN SIIGPALIKS DLCVTNQKGI DELMISLDGT |
| 101 | SNKSRLGANA ILGVSLCVAR AAAAQKGITL YKYIAELADA RQDPFVIPVP |
| 151 | FFNVLNGGAH AGGSLAMQEF KIAPVGAQSF AEAMRMGSEV YHHLKILAKE |
| 201 | QYGPSAGNVG DEGGVAPDID TAEDALDMIV KAINICGYEG RVKVGIDSAP |
| 251 | SVFYKDGKYD LNFKEPNSDP SHWLSPAQLA EYYHSLLKKY PIISLEDPYA |
| 301 | EDDWSSWSAF LKTVNVQIIA DDLTCTNKTR IARAIEEKCA NTLLLKLNQI |
| 351 | GTLTESIEAA NQAFDAGWGV MISHRSGETE DPFIADLVVG LRCGQIKSGA |
| 401 | LSRSERLAKY NELLRIEEEL GDDCIYAGHR FHDGNKL |

SEQ ID NO: 2
FOX2 amino acid sequence; systematic name YKR009C

| | |
|---|---|
| 1 | MPGNLSFKDR VVVITGAGGG LGKVYALAYA SRGAKVVVND LGGTLGGSGH |
| 51 | NSKAADLVVD EIKKAGGIAV ANYDSVNENG EKIIETAIKE FGRVDVLINN |
| 101 | AGILRDVSFA KMTEREFASV VDVHLTGGYK LSRAAWPYMR SQKFGRIINT |
| 151 | ASPAGLFGNF GQANYSAAKM GLVGLAETLA KEGAKYNINV NSIAPLARSR |
| 201 | MTENVLPPHI LKQLGPEKIV PLVLYLTHES TKVSNSIFEL AAGFFGQLRW |
| 251 | ERSSGQIFNP DPKTYTPEAI LNKWKEITDY RDKPFNKTQH PYQLSDYNDL |
| 301 | ITKAKKLPPN EQGSVKIKSL CNKVVVVTGA GGGLGKSHAI WFARYGAKVV |
| 351 | VNDIKDPFSV VEEINKLYGE GTAIPDSHDV VTEAPLIIQT AISKFQRVDI |
| 401 | LVNNAGILRD KSFLKMKDEE WFAVLKVHLF STFSLSKAVW PIFTKQKSGF |
| 451 | IINTTSTSGI YGNFGQANYA AAKAAILGFS KTIALEGAKR GIIVNVIAPH |
| 501 | AETAMTKTIF SEKELSNHFD ASQVSPLVVL LASEELQKYS GRRVIGQLFE |
| 551 | VGGGWCGQTR WQRSSGYVSI KETIEPEEIK ENWNHITDFS RNTINPSSTE |
| 601 | ESSMATLQAV QKAHSSKELD DGLFKYTTKD CILYNLGLGC TSKELKYTYE |
| 651 | NDPDFQVLPT FAVIPFMQAT ATLAMDNLVD NFNYAMLLHG EQYFKLCTPT |
| 701 | MPSNGTLKTL AKPLQVLDKN GKAALVVGGF ETYDIKTKKL IAYNEGSFFI |
| 751 | RGAHVPPEKE VRDGKRAKFA VQNFEVPHGK VPDFEAEIST NKDQAALYRL |
| 801 | SGDFNPLHID PTLAKAVKFP TPILHGLCTL GISAKALFEH YGPYEELKVR |
| 851 | FTNVVFPGDT LKVKAWKQGS VVVFQTIDTT RNVIVLDNAA VKLSQAKSKL |

SEQ ID NO: 3
LYS1amino acid sequence; systematic name YIR034C

| | |
|---|---|
| 1 | MAAVTLHLRA ETKPLEARAA LTPTTVKKLI AKGFKIYVED SPQSTFNINE |
| 51 | YRQAGAIIVP AGSWKTAPRD RIIIGLKEMP ETDTFPLVHE HIQFAHCYKD |
| 101 | QAGWQNVLMR FIKGHGTLYD LEFLENDQGR RVAAFGFYAG FAGAALGVRD |
| 151 | WAFKQTHSDD EDLPAVSPYP NEKALVKDVT KDYKEALATG ARKPTVLIIG |
| 201 | ALGRCGSGAI DLLHKVGIPD ANILKWDIKE TSRGGPFDEI PQADIFINCI |
| 251 | YLSKPIAPFT NMEKLNNPNR RLRTVVDVSA DTTNPHNPIP IYTVATVFNK |
| 301 | PTVLVPTTAG PKLSVISIDH LPSLLPREAS EFFSHDLLPS LELLPQRKTA |
| 351 | PVWVRAKKLF DRHCARVKRS SRL |

SEQ ID NO: 4
MET1 amino acid sequence; systematic name YKR069W

| | |
|---|---|
| 1 | MVRDLVTLPS SLPLITAGFA TDQVHLLIGT GSTDSVSVCK NRIHSILNAG |
| 51 | GNPIVVNPSS PSHTKQLQLE FGKFAKFEIV EREFRLSDLT TLGRVLVCKV |
| 101 | VDRVFVDLPI TQSRLCEEIF WQCQKLRIPI NTFHKPEFST FNMIPTWVDP |
| 151 | KGSGLQISVT TNGNGYILAN RIKRDIISHL PPNISEVVIN MGYLKDRIIN |
| 201 | EDHKALLEEK YYQTDMSLPG FGYGLDEDGW ESHKFNKLIR EFEMTSREQR |
| 251 | LKRTRWLSQI MEYYPMNKLS DIKLEDFETS SSPNKKTKQE TVTEGVVPPT |
| 301 | DENIENGTKQ LQLSEVKKEE GPKKLGKISL VGSGPGSVSM LTIGALQEIK |
| 351 | SADIILADKL VPQAILDLIP PKTETFIAKK FPGNAERAQQ ELLAKGLESL |
| 401 | DNGLKVVRLK QGDPYIFGRG GEEFNFFKDH GYIPVVLPGI SSSLACTVLA |
| 451 | QIPATQRDIA DQVLICTGTG RKGALPIIPE FVESRTTVFL MALHRANVLI |
| 501 | TGLLKHGWDG DVPAAIVERG SCPDQRVTRT LLKWVPEVVE EIGSRPPGVL |
| 551 | VVGKAVNALV EKDLINFDES RKFVIDEGFR EFEVDVDSLF KLY |

SEQ ID NO: 5
MIG2 amino acid sequence; systematic name YGL209W

| | |
|---|---|
| 1 | MPKKQTNFPV DNENRPFRCD TCHRGFHRLE HKKRHLRTHT GEKPHHCAFP |
| 51 | GCGKSFSRSD ELKRHMRTHT GQSQRRLKKA SVQKQEFLTV SGIPTIASGV |
| 101 | MIHQPIPQVL PANMAINVQA VNGGNIIHAP NAVHPMVIPI MAQPAPIHAS |
| 151 | AASFQPATSP MPISTYTPVP SQSFTSFQSS IGSIQSNSDV SSIFSNMNVR |
| 201 | VNTPRSVPNS PNDGYLHQQH IPQQYQHQTA SPSVAKQQKT FAHSLASALS |
| 251 | TLQKRTPVSA PSTTIESPSS PSDSSHTSAS SSAISLPFSN APSQLAVAKE |
| 301 | LESVYLDSNR YTTKTRRERA KFEIPEEQEE DTNNSSSGSN EEEHESLDHE |
| 351 | SSKSRKKLSG VKLPPVRNLL KQIDVFNGPK RV |

SEQ ID NO: 6
RMD6 amino acid sequence; systematic name YEL072W

| | |
|---|---|
| 1 | MSACPCNIVI LPVEILKNSS KDTKYSLYTT INRGYDVPRL KYGIIVSPRV |
| 51 | HSLETLFSDL GFDKNIEKSS LYLLLNDPTL AYPNFHEHFE QLKGETNKDL |
| 101 | SLPTYYIPKV QFLTEAFDSE HTLATIGYKP NNKESYEITG FTSMGNGYGI |
| 151 | KLFNYSVIHM MRSHKCKRVV ADIIMEHDLL GYYEKKLGFV EVQRFKVLKE |
| 201 | QHQVKVFDDK VDFTKDFHVI KMIKELGNHR L |

SEQ ID NO: 7
RME1 amino acid sequence; systematic name YGR044C

| | |
|---|---|
| 1 | MSPCYGQNSA IAKGSWNREV LQEVQPIYHW HDFGQNMKEY SASPLEGDSS |
| 51 | LPSSLPSSTE DCLLLSLENT ITVIAGNQRQ AYDSTSSTEE GTAPQLRPDE |
| 101 | IADSTHCITS LVDPEFRDLI NYGRQKGANP VFIESNTTEQ SHSQCILGYP |
| 151 | QKSHVAQLYH DPKVLSTISE GQTKRGSYHC SHCSEKFATL VEFAAHLDEF |
| 201 | NLERPCKCPI EQCPWKILGF QQATGLRRHC ASQHIGELDI EMEKSLNLKV |
| 251 | EKYPGLNCPF PICQKTFRRK DAYKRHVAMV HNNADSRFNK RLKKILNNTK |

SEQ ID NO: 8
SIP1 amino acid sequence; systematic name YDR422C

| | |
|---|---|
| 1 | MGNSPSTQDP SHSTKKEHGH HFHDAFNKDR QGSITSQLFN NRKSTHKRRA |
| 51 | SHTSEHNGAI PPRMQLLASH DPSTDCDGRM SSDTTIDKGP SHLFKKDYSL |
| 101 | SSAADVNDTT LANLTLSDDH DVGAPEEQVK SPSFLSPGPS MATVKRTKSD |
| 151 | LDDLSTLNYT MVDETTENER NDKPHHERHR SSIIALKKNL LESSATASPS |
| 201 | PTRSSSVHSA SLPALTKTDS IDIPVRQPYS KKPSIHAYQY QYLNNDETFS |
| 251 | ENSQMDKEGN SDSVDAEAGV LQSEDMVLNQ SLLQNALKKD MQRLSRVNSS |
| 301 | NSMYTAERIS HANNNGNIEN NTRNKGNAGG SNDDFTAPIS ATAKMMMKLY |
| 351 | GDKTLMERDL NKHHNKTKKA QNKKIRSVSN SRRSSFASLH SLQSRKSILT |
| 401 | NGLNLQPLHP LHPIINDNES QYSAPQHREI SHHSNSMSSM SSISSTNSTE |
| 451 | NTLVVLKWKD DGTVAATTEV FIVSTDIASA LKEQRELTLD ENASLDSEKQ |
| 501 | LNPRIRMVYD DVHKEWFVPD LFLPAGIYRL QFSINGILTH SNFLPTATDS |
| 551 | EGNFVNWFEV LPGYHTIEPF RNEADIDSQV EPTLDEELPK RPELKRFPSS |
| 601 | SRKSSYYSAK GVERPSTPFS DYRGLSRSSS INMRDSFVRL KASSLDLMAE |
| 651 | VKPERLVYSN EIPNLFNIGD GSTISVKGDS DDVHPQEPPS FTHRVVDCNQ |
| 701 | DDLFATLQQG GNIDAETAEA VFLSRYPVPD LPIYLNSSYL NRILNQSNQN |
| 751 | SESHERDEGA INHIIPHVNL NHLLTSSIRD EIISVACTTR YEGKFITQVV |
| 801 | YAPCYYKTQK SQISN* |

SEQ ID NO: 9
SNP1 amino acid sequence; systematic name YIL061C

| | |
|---|---|
| 1 | MNYNLSKYPD DVSRLFKPRP PLSYKRPTDY PYAKRQTNPN ITGVANLLST |
| 51 | SLKHYMEEFP EGSPNNHLQR YEDIKLSKIK NAQLLDRRLQ NWNPNVDPHI |
| 101 | KDTDPYRTIF IGRLPYDLDE IELQKYFVKF GEIEKIRIVK DKITQKSKGY |
| 151 | AFIVFKDPIS SKMAFKEIGV HRGIQIKDRI CIVDIERGRT VKYFKPRRLG |
| 201 | GGLGGRGYSN RDSRLPGRFA SASTSNPAER NYAPRLPRRE TSSSAYSADR |
| 251 | YGSSTLDARY RGNRPLLSAA TPTAAVTSVY KSRNSRTRES QPAPKEAPDY |

SEQ ID NO: 10
TDH1 amino acid sequence; systematic name YJL052W

| | |
|---|---|
| 1 | MIRIAINGFG RIGRLVLRLA LQRKDIEVVA VNDPFISNDY AAYMVKYDST |
| 51 | HGRYKGTVSH DDKHIIIDGV KIATYQERDP ANLPWGSLKI DVAVDSTGVF |
| 101 | KELDTAQKHI DAGAKKVVIT APSSSAPMFV VGVNHTKYTP DKKIVSNASC |
| 151 | TTNCLAPLAK VINDAFGIEE GLMTTVHSMT ATQKTVDGPS HKDWRGGRTA |
| 201 | SGNIIPSSTG AAKAVGKVLP ELQGKLTGMA FRVPTVDVSV VDLTVKLEKE |
| 251 | ATYDQIKKAV KAAAEGPMKG VLGYTEDAVV SSDFLGDTHA SIFDASAGIQ |
| 301 | LSPKFVKLIS WYDNEYGYSA RVVDLIEYVA KA |

SEQ ID NO: 11
GPD1 amino acid sequence; systematic name YDL022W

| | |
|---|---|
| 1 | MSAAADRLNL TSGHLNAGRK RSSSSVSLKA AEKPFKVTVI GSGNWGTTIA |
| 51 | KVVAENCKGY PEVFAPIVQM WVFEEEINGE KLTEIINTRH QNVKYLPGIT |
| 101 | LPDNLVANPD LIDSVKDVDI IVFNIPHQFL PRICSQLKGH VDSHVRAISC |
| 151 | LKGFEVGAKG VQLLSSYITE ELGIQCGALS GANIATEVAQ EHWSETTVAY |
| 201 | HIPKDFRGEG KDVDHKVLKA LFHRPYFHVS VIEDVAGISI CGALKNVVAL |
| 251 | GCGFVEGLGW GNNASAAIQR VGLGEIIRFG QMFFPESREE TYYQESAGVA |
| 301 | DLITTCAGGR NVKVARLMAT SGKDAWECEK ELLNGQSAQG LITCKEVHEW |
| 351 | LETCGSVEDF PLFEAVYQIV YNNYPMKNLP DMIEELDLHE D |

SEQ ID NO: 12
RSF2 amino acid sequence; systematic name YJR127C

| | |
|---|---|
| 1 | MEPFAFGRGA PALCILTAAA RINLDNFVPC CWALFRLSFF FPLDPAYIRN |
| 51 | ENKETRTSWI SIEFFFFVKH CLSQHTFFSK TLAPKRNFRA KKLKDIGDTR |
| 101 | IDRADKDFLL VPEPSMFVNG NQSNFAKPAG QGILPIPKKS RIIKTDKPRP |
| 151 | FLCPTCTRGF VRQEHLKRHQ HSHTREKPYL CIFCGRCFAR RDLVLRHQQK |
| 201 | LHAALVGTGD PRRMTPAPNS TSSFASKRRH SVAADDPTDL HIIKIAGNKE |
| 251 | TILPTPKNLA GKTSEELKEA VVALAKSNNV ELPVSAPVMN DKREKTPPSK |
| 301 | AGSLGFREFK FSTKGVPVHS ASSDAVIDRA NTPSSMHKTK RHASFSASSA |
| 351 | MTYMSSSNSP HHSITNFELV EDAPHQVGFS TPQMTAKQLM ESVSELDLPP |
| 401 | LTLDEPPQAI KFNLNLFNND PSGQQQQQQQ QQQNSTSSTI VNSNNGSTVA |
| 451 | TPGVYLLSSG PSLTDLLTMN SAHAGAGGYM SSHHSPFDLG CFSHDKPTVS |
| 501 | EFNLPSSFPN TIPSNSTTAS NSYSNLANQT YRQMSNEQPL MSLSPKNPPT |
| 551 | TVSDSSSTIN FNPGTNNLLE PSMEPNDKDS NIDPAAIDDK WLSEFINNSD |
| 601 | PKSTFKINFN HFNDIGFIYS PPSSRSSIPN KSPPNHSATS LNHEKASLSP |
| 651 | RLNLSLNGST DLPSTPQNQL KEPSYSDPIS HSSHKRRRDS VMMDYDLSNF |
| 701 | FSSRQLDISK VLNGTEQNNS HVNDDVLTLS FPGETDSNAT QKQLPVLTPS |
| 751 | DLLSPFSVPS VSQVLFTNEL RSMMLADNNI DSGAFPTTSQ LNDYVTYYKE |
| 801 | EFHPFFSFIH LPSIIPNMDS YPLLLSISMV GALYGFHSTH AKVLANAAST |
| 851 | QIRKSLKVSE KNPETTELWV IQTLVLLTFY CIFNKNTAVI KGMHGQLTTI |
| 901 | IRLLKASRLN LPLESLCQPP IESDHIMEYE NSPHMFSKIR EQYNAPNQMN |
| 951 | KNYQYFVLAQ SRIRTCHAVL LISNLFSSLV GADCCFHSVD LKCGVPCYKE |
| 1001 | ELYQCRNSDE WSDLLCQYKI TLDSKFSLIE LSNGNEAYEN CLRFLSTGDS |
| 1051 | FFYGNARVSL STCLSLLISI HEKILIERNN ARISNNNTNS NNIELDDIEW |
| 1101 | KMTSRQRIDT MLKYWENLYL KNGGILTPTE NSMSTINANP AMRLIIPVYL |
| 1151 | FAKMRRCLDL AHVIEKIWLK DWSNMNKALE EVCYDMGSLR EATEYALNMV |
| 1201 | DAWTSFFTYI KQGKRRIFNT PVFATTCMFT AVLVISEYMK CVEDWARGYN |
| 1251 | ANNPNSALLD FSDRVLWLKA ERILRRLQMN LIPKECDVLK SYTDFLRWQD |
| 1301 | KDALDLSALN EEQAQRAMDP NTDINETIQL IVAASLSSKC LYLGVQILGD |
| 1351 | APIWPIILSF AHGLQSRAIY SVTKKRNTRI |

SEQ ID NO: 13
GND2 amino acid sequence; systematic name YGR256W

| | |
|---|---|
| 1 | MSKAVGDLGL VGLAVMGQNL ILNAADHGFT VVAYNRTQSK VDRFLANEAK |
| 51 | GKSIIGATSI EDLVAKLKKP RKIMLLIKAG APVDTLIKEL VPHLDKGDII |
| 101 | IDGGNSHFPD TNRRYEELTK QGILFVGSGV SGGEDGARFG PSLMPGGSAE |
| 151 | AWPHIKNIFQ SIAAKSNGEP CCEWVGPAGS GHYVKMVHNG IEYGDMQLIC |
| 201 | EAYDIMKRIG RFTDKEISEV FDKWNTGVLD SFLIEITRDI LKFDDVDGKP |
| 251 | LVEKIMDTAG QKGTGKWTAI NALDLGMPVT LIGEAVFARC LSAIKDERKR |
| 301 | ASKLLAGPTV PKDAIHDREQ FVYDLEQALY ASKIISYAQG FMLIREAARS |
| 351 | YGWKLNNPAI ALMWRGGCII RSVFLAEITK AYRDDPDLEN LLFNEFFASA |
| 401 | VTKAQSGWRR TIALAATYGI PTPAFSTALA FYDGYRSERL PANLLQAQRD |
| 451 | YFGAHTFRIL PECASAHLPV DKDIHINWTG HGGNISSSTY QA |

SEQ ID NO: 14
TRK1 amino acid sequence; systematic name YJL129C

| | |
|---|---|
| 1 | MHFRRTMSRV PTLASLEIRY KKSFGHKFRD FIALCGHYFA PVKKYIFPSF |
| 51 | IAVHYFYTIS LTLITSILLY PIKNTRYIDT LFLAAGAVTQ GGLNTVDINN |
| 101 | LSLYQQIVLY IVCCISTPIA VHSCLAFVRL YWFERYFDGI RDSSRRNFKM |
| 151 | RRTKTILERE LTARTMTKNR TGTQRTSYPR KQAKTDDFQE KLFSGEMVNR |
| 201 | DEQDSVHSDQ NSHDISRDSS NNNTNHNGSS GSLDDFVKED ETDDNGEYQE |
| 251 | NNSYSTVGSS SNTVADESLN QKPKPSSLRF DEPHSKQRPA RVPSEKFAKR |
| 301 | RGSRDISPAD MYRSIMMLQG KHEATAEDEG PPLVIGSPAD GTRYKSNVNK |
| 351 | LKKATGINGN KIKIRDKGNE SNTDQNSVSS EANSTASVSD ESSLHTNFGN |
| 401 | KVPSLRTNTH RSNSGPIAIT DNAETDKKHG PSIQFDITKP PRKISKRVST |
| 451 | FDDLNPKSSV LYRKKASKKY LMKHFPKARR IRQQIKRRLS TGSIEKNSSN |
| 501 | NVSDRKPITD MDDDDDDDDN DGDNNEEYFA DNESGDEDER VQQSEPHSDS |
| 551 | ELKSHQQQQE KHQLQQNLHR MYKTKSFDDN RSRAVPMERS RTIDMAEAKD |
| 601 | LNELARTPDF QKMVYQNWKA HHRKKPNFRK RGWNNKIFEH GPYASDSDRN |
| 651 | YPDNSNTGNS ILHYAESILH HDGSHKNGSE EASSDSNENI YSTNGGSDHN |
| 701 | GLNNYPTYND DEEGYYGLHF DTDYDLDPRH DLSKGSGKTY LSWQPTIGRN |
| 751 | SNFLGLTRAQ KDELGGVEYR AIKLLCTILV VYYVGWHIVA FVMLVPWIIL |
| 801 | KKHYSEVVRD DGVSPTWWGF WTAMSAFNDL GLTLTPNSMM SFNKAVYPLI |
| 851 | VMIWFIIIGN TGFPILLRCI IWIMFKISPD LSQMRESLGF LLDHPRRCFT |
| 901 | LLFPKAATWW LLLTLAGLNI TDWILFIILD FGSTVVKSLS KGYRVLVGLF |
| 951 | QSVSTRTAGF SVVDLSQLHP SIQVSYMLMM YVSVLPLAIS IRRTNVYEEQ |
| 1001 | SLGLYGDMGG EPEDTDTEDD GNDEDDDEEN ESHEGQSSQR SSSNNNNNNN |
| 1051 | RKKKKKKKTE NPNEISTKSF IGAHLRKQLS FDLWFLFLGL FIICICEGDK |
| 1101 | IKDVQEPNFN IFAILFEIVS AYGTVGLSLG YPDTNQSFSR QFTTLSKLVI |
| 1151 | IAMLIRGKNR GLPYSLDRAI ILPSDRLEHI DHLEGMKLKR QARTNTEDPM |
| 1201 | TEHFKRSFTD VKHRWGALKR KTTHSRNPKR SSTTL |

SEQ ID NO: 15
HSP31 amino acid sequence; systematic name YDR533C

| | |
|---|---|
| 1 | MAPKKVLLAL TSYNDVFYSD GAKTGVFVVE ALHPFNTFRK EGFEVDFVSE |
| 51 | TGKFGWDEHS LAKDFLNGQD ETDFKNKDSD FNKTLAKIKT PKEVNADDYQ |
| 101 | IFFASAGHGT LFDYPKAKDL QDIASEIYAN GGVVAAVCHG PAIFDGLTDK |
| 151 | KTGRPLIEGK SITGFTDVGE TILGVDSILK AKNLATVEDV AKKYGAKYLA |
| 201 | PVGPWDDYSI TDGRLVTGVN PASAHSTAVR SIDALKN |

SEQ ID NO: 16
HSP33 amino acid sequence; systematic name YOR391C

| | |
|---|---|
| 1 | MTPKRALISL TSYHGPFYKD GAKTGVFVVE ILRSFDTFEK HGFEVDFVSE |
| 51 | TGGFGWDEHY LPKSFIGGED KMNFETKNSA FNKALARIKT ANEVNASDYK |
| 101 | VFFASAGHGA LFDYPKAKNL QDIASKIYAN GGVIAAICHG PLLFDGLIDI |
| 151 | KTTRPLIEGK AITGFPLEGE IALGVDDILR SRKLTTVERV ANKNGAKYLA |
| 201 | PIHPWDDYSI TDGKLVTGVN ANSSYSTTIR AINALYS |

SEQ ID NO: 17
HSP30 amino acid sequence; systematic name YCR021C

| | |
|---|---|
| 1 | MNDTLSSFLN RNEALGLNPP HGLDMHITKR GSDWLWAVFA VFGFILLCYV |
| 51 | VMFFIAENKG SRLTRYALAP AFLITFFEFF AFFTYASDLG WTGVQAEFNH |
| 101 | VKVSKSITGE VPGIRQIFYS KYIAWFLSWP CLLFLIELAA STTGENDDIS |
| 151 | ALDMVHSLLI QIVGTLFWVV SLLVGSLIKS TYKWGYYTIG AVAMLVTQGV |
| 201 | ICQRQFFNLK TRGFNALMLC TCMVIVWLYF ICWGLSDGGN RIQPDGEAIF |
| 251 | YGVLDLCVFA IYPCYLLIAV SRDGKLPRLS LTGGFSHHHA TDDVEDAAPE |
| 301 | TKEAVPESPR ASGETAIHEP EPEAEQAVED TA |

SEQ ID NO: 18
HSP32 amino acid sequence; systematic name YPL280W

| | |
|---|---|
| 1 | MTPKRALISL TSYHGPFYKD GAKTGVFVVE ILRSFDTFEK HGFEVDFVSE |
| 51 | TGGFGWDEHY LPKSFIGGED KMNFETKNSA FNKALARIKT ANEVNASDYK |
| 101 | IFFASAGHGA LFDYPKAKNL QDIASKIYAN GGVIAAICHG PLLFDGLIDI |
| 151 | KTTRPLIEGK AITGFPLEGE IALGVDDILR SRKLTTVERV ANKNGAKYLA |
| 201 | PIHPWDDYSI TDGKLVTGVN ANSSYSTTIR AINALYS |

SEQ ID NO: 19
ADH6 amino acid sequence; systematic name YMR318C

| | |
|---|---|
| 1 | MSYPEKFEGI AIQSHEDWKN PKKTKYDPKP FYDHDIDIKI EACGVCGSDI |
| 51 | HCAAGHWGNM KMPLVVGHEI VGKVVKLGPK SNSGLKVGQR VGVGAQVFSC |
| 101 | LECDRCKNDN EPYCTKFVTT YSQPYEDGYV SQGGYANYVR VHEHFVVPIP |
| 151 | ENIPSHLAAP LLCGGLTVYS PLVRNGCGPG KKVGIVGLGG IGSMGTLISK |
| 201 | AMGAETYVIS RSSRKREDAM KMGADHYIAT LEEGDWGEKY FDTFDLIVVC |
| 251 | ASSLTDIDFN IMPKAMKVGG RIVSISIPEQ HEMLSLKPYG LKAVSISYSA |
| 301 | LGSIKELNQL LKLVSEKDIK IWVETLPVGE AGVHEAFERM EKGDVRYRFT |

SEQ ID NO: 20
UFD4 amino acid sequence; systematic name YKL010C

| | |
|---|---|
| 1 | MSENNSHNLD EHESHSENSD YMMDTQVEDD YDEDGHVQGE YSYYPDEDED |
| 51 | EHMLSSVGSF EADDGEDDDN DYHHEDDSGL LYGYHRTQNG SDEDRNEEED |
| 101 | GLERSHDNNE FGSNPLHLPD ILETFAQRLE QRRQTSEGLG QHPVGRTLPE |
| 151 | ILSMIGGRME RSAESSARNE RISKLIENTG NASEDPYIAM ESLKELSENI |
| 201 | LMMNQMVVDR IIPMETLIGN IAAILSDKIL REELELQMQA CRCMYNLFEV |
| 251 | CPESISIAVD EHVIPILQGK LVEISYIDLA EQVLETVEYI SRVHGRDILK |
| 301 | TGQLSIYVQF FDFLTIHAQR KAIAIVSNAC SSIRTDDFKT IVEVLPTLKP |
| 351 | IFSNATDQPI LTRLVNAMYG ICGALHGVDK FETLFSLDLI ERIVQLVSIQ |
| 401 | DTPLENKLKC LDILTVLAMS SDVLSRELRE KTDIVDMATR SFQHYSKSPN |
| 451 | AGLHETLIYV PNSLLISISR FIVVLFPPED ERILSADKYT GNSDRGVISN |
| 501 | QEKFDSLVQC LIPILVEIYT NAADFDVRRY VLIALLRVVS CINNSTAKAI |
| 551 | NDQLIKLIGS ILAQKETASN ANGTYSSEAG TLLVGGLSLL DLICKKFSEL |
| 601 | FFPSIKREGI FDLVKDLSVD FNNIDLKEDG NENISLSDEE GDLHSSIEEC |
| 651 | DEGDEEYDYE FTDMEIPDSV KPKKISIHIF RTLSLAYIKN KGVNLVNRVL |
| 701 | SQMNVEQEAI TEELHQIEGV VSILENPSTP DKTEEDWKGI WSVLKKCIFH |
| 751 | EDFDVSGFEF TSTGLASSIT KRITSSTVSH FILAKSFLEV FEDCIDRFLE |
| 801 | ILQSALTRLE NFSIVDCGLH DGGGVSSLAK EIKIKLVYDG DASKDNIGTD |
| 851 | LSSTIVSVHC IASFTSLNEF LRHRMVRMRF LNSLIPNLTS SSTEADREEE |
| 901 | ENCLDHMRKK NFDFFYDNEK VDMESTVFGV IFNTFVRRNR DLKTLWDDTH |
| 951 | TIKFCKSLEG NNRESEAAEE ANEGKKLRDF YKKREFAQVD TGSSADILTL |
| 1001 | LDFLHSCGVK SDSFINSKLS AKLARQLDEP LVVASGALPD WSLFLTRRFP |
| 1051 | FLFPFDTRML FLQCTSFGYG RLIQLWKNKS KGSKDLRNDE ALQQLGRITR |
| 1101 | RKLRISRKTI FATGLKILSK YGSSPDVLEI EYQEEAGTGL GPTLEFYSVV |
| 1151 | SKYFARKSLN MWRCNSYSYR SEMDVDTTDD YITTLLFPEP LNPFSNNEKV |
| 1201 | IELFGYLGTF VARSLLDNRI LDFRFSKVFF ELLHRMSTPN VTTVPSDVET |
| 1251 | CLLMIELVDP LLAKSLKYIV ANKDDNMTLE SLSLTFTVPG NDDIELIPGG |
| 1301 | CNKSLNSSNV EEYIHGVIDQ ILGKGIEKQL KAFIEGFSKV FSYERMLILF |
| 1351 | PDELVDIFGR VEEDWSMATL YTNLNAEHGY TMDSSIIHDF ISIISAFGKH |
| 1401 | ERRLFLQFLT GSPKLPIGGF KSLNPKFTVV LKHAEDGLTA DEYLPSVMTC |
| 1451 | ANYLKLPKYT SKDIMRSRLC QAIEEGAGAF LLS |

SEQ ID NO: 21
PRO1 amino acid sequence; systematic name YDR300C

| | |
|---|---|
| 1 | MKDANESKSY TIVIKLGSSS LVDEKTKEPK LAIMSLIVET VVKLRRMGHK |
| 51 | VIIVSSGGIA VGLRTMRMNK RPKHLAEVQA IAAIGQGRLI GRWDLLFSQF |
| 101 | DQRIAQILLT RNDILDWTQY KNAQNTINEL LNMGVIPIVN ENDTLSVREI |
| 151 | KFGDNDTLSA ITSALIHADY LFLLTDVDCL YTDNPRTNPD AMPILVVPDL |
| 201 | SKGLPGVNTA GGSGSDVGTG GMETKLVAAD LATNAGVHTL IMKSDTPANI |
| 251 | GRIVEYMQTL ELDDENKVKQ AYNGDLTDLQ KREFEKLKAL NVPLHTKFIA |
| 301 | NDNKHHLKNR EFWILHGLVS KGAVVIDQGA YAALTRKNKA GLLPAGVIDV |
| 351 | QGTFHELECV DIKVGKKLPD GTLDPDFPLQ TVGKARCNYT SSELTKIKGL |
| 401 | HSDQIEEELG YNDSEYVAHR ENLAFPPR |

SEQ ID NO: 22
SIA1 amino acid sequence; systematic name YOR137C

| | |
|---|---|
| 1 | MRLHYRRRFN FLRRILFILC ITSLYLSRDS LKLHAKNVLM DHNVAEYHGG |
| 51 | MIDDIQILRC YHWYRQCSSL YAPKLHPSNT AKKIKDKNSI LWTRVSKNIT |
| 101 | VETLYSLQSG PFYNSYLYVH LKDFQSNPKN TIKELAIARD SALIPLQVLR |
| 151 | DINKLVKSSD SSVFHNHVYL REKPTSSWWK LLFGISVDTD NIAVFGEEWV |
| 201 | YKGSGIWCKY ILNDDDNDAP ITNLEIYLGS SFIESRPSWK EVIHEFHRNN |
| 251 | IPSLPISITR KLETKNHHHK FSNGLLGSLR TPSKDINIQV DADYKITSPH |
| 301 | IQFSRGQRSF KILQITDFHF KCTDNSMTVI NEIKTVNFID RVLASENPDL |
| 351 | VVITGDLLDS HNTIDYQTCI MKVVQPMISN KIPYAISLGV SDESNLATSA |
| 401 | QIRDFIRNLP YTFNNVASEE GHMAIEVSFK KKLTKNTLLE RDIDTEDETN |
| 451 | PSEALFFVFD SFAPVNNFLQ DYNDLIGKID FGLAFQYFPL SEYRPHGLFP |
| 501 | IIGQYNERST LTVDTPRSRG QVSMTINGKH YKSFLDILSL WNIKGVSCGH |
| 551 | EHNNDCCLQS KNEMWLCYGG SAGIGLPRIQ GIYPTVRLFN LDDILDEITS |
| 601 | WKRNSNLVDE VYDYQYIYKG KQ* |

SEQ ID NO: 23
ARI1amino acid sequence; systematic name YGL157W

| | |
|---|---|
| 1 | MTTDTTVFVS GATGFIALHI MNDLLKAGYT VIGSGRSQEK NDGLLKKFNN |
| 51 | NPKLSMEIVE DIAAPNAFDE VFKKHGKEIK IVLHTASPFH FETTNFEKDL |
| 101 | LTPAVNGTKS ILEAIKKYAA DTVEKVIVTS STAALVTPTD MNKGDLVITE |
| 151 | ESWNKDTWDS CQANAVAAYC GSKKFAEKTA WEFLKENKSS VKFTLSTINP |
| 201 | GFVFGPQMFA DSLKHGINTS SGIVSELIHS KVGGEFYNYC GPFIDVRDVS |
| 251 | KAHLVAIEKP ECTGQRLVLS EGLFCCQEIV DILNEEFPQL KGKIATGEPA |
| 301 | TGPSFLEKNS CKFDNSKTKK LLGFQFYNLK DCIVDTAAQM LEVQNEA* |

SEQ ID NO: 24
LPP1 amino acid sequence; systematic name YDR503C

| | |
|---|---|
| 1 | MISVMADEKH KEYFKLYYFQ YMIIGLCTIL FLYSEISLVP RGQNIEFSLD |
| 51 | DPSISKRYVP NELVGPLECL ILSVGLSNMV VFWTCMFDKD LLKKNRVKRL |
| 101 | RERPDGISND FHFMHTSILC LMLIISINAA LTGALKLIIG NLRPDFVDRC |
| 151 | IPDLQKMSDS DSLVFGLDIC KQTNKWILYE GLKSTPSGHS SFIVSTMGFT |
| 201 | YLWQRVFTTR NTRSCIWCPL LALVVMVSRV IDHRHHWYDV VSGAVLAFLV |
| 251 | IYCCWKWTFT NLAKRDILPS PVSV |

SEQ ID NO: 25
PMA2 amino acid sequence; systematic name YPL036W

| | |
|---|---|
| 1 | MSSTEAKQYK EKPSKEYLHA SDGDDPANNS AASSSSSSST STSASSSAAA |
| 51 | VPRKAAAASA ADDSDSDEDI DQLIDELQSN YGEGDESGEE EVRTDGVHAG |
| 101 | QRVVPEKDLS TDPAYGLTSD EVARRRKKYG LNQMAEENES LIVKFLMFFV |
| 151 | GPIQFVMEAA AILAAGLSDW VDVGVICALL LLNASVGFIQ EFQAGSIVDE |
| 201 | LKKTLANTAT VIRDGQLIEI PANEVVPGEI LQLESGTIAP ADGRIVTEDC |
| 251 | FLQIDQSAIT GESLAAEKHY GDEVFSSSTV KTGEAFMVVT ATGDNTFVGR |
| 301 | AAALVGQASG VEGHFTEVLN GIGIILLVLV IATLLLVWTA CFYRTVGIVS |
| 351 | ILRYTLGITI IGVPVGLPAV VTTTMAVGAA YLAKKQAIVQ KLSAIESLAG |
| 401 | VEILCSDKTG TLTKNKLSLH EPYTVEGVSP DDLMLTACLA ASRKKKGLDA |
| 451 | IDKAFLKSLI EYPKAKDALT KYKVLEFHPF DPVSKKVTAV VESPEGERIV |
| 501 | CVKGAPLFVL KTVEEDHPIP EDVHENYENK VAELASRGFR ALGVARKRGE |
| 551 | GHWEILGVMP CMDPPRDDTA QTINEARNLG LRIKMLTGDA VGIAKETCRQ |
| 601 | LGLGTNIYNA ERLGLGGGGD MPGSELADFV ENADGFAEVF PQHKYRVVEI |
| 651 | LQNRGYLVAM TGDGVNDAPS LKKADTGIAV EGATDAARSA ADIVFLAPGL |
| 701 | SAIIDALKTS RQIFHRMYSY VVYRIALSLH LEIFLGLWIA ILNNSLDINL |
| 751 | IVFIAIFADV ATLTIAYDNA PYAPEPVKWN LPRLWGMSII LGIVLAIGSW |
| 801 | ITLTTMFLPN GGIIQNFGAM NGVMFLQISL TENWLIFVTR AAGPFWSSIP |
| 851 | SWQLAGAVFA VDIIATMFTL FGWWSENWTD IVSVVRVWIW SIGIFCVLGG |
| 901 | FYYIMSTSQA FDRLMNGKSL KEKKSTRSVE DFMAAMQRVS TQHEKSS |

SEQ ID NO: 26
PDR12 amino acid sequence; systematic name YPL058C

| | |
|---|---|
| 1 | MSSTDEHIEK DISSRSNHDD DYANSVQSYA ASEGQVDNED LAATSQLSRH |
| 51 | LSNILSNEEG IERLESMARV ISHKTKKEMD SFEINDLDFD LRSLLHYLRS |
| 101 | RQLEQGIEPG DSGIAFKNLT AVGVDASAAY GPSVEEMFRN IASIPAHLIS |
| 151 | KFTKKSDVPL RNIIQNCTGV VESGEMLFVV GRPGAGCSTF LKCLSGETSE |
| 201 | LVDVQGEFSY DGLDQSEMMS KYKGYVIYCP ELDFHFPKIT VKETIDFALK |
| 251 | CKTPRVRIDK MTRKQYVDNI RDMWCTVFGL RHTYATKVGN DFVRGVSGGE |
| 301 | RKRVSLVEAQ AMNASIYSWD NATRGLDAST ALEFAQAIRT ATNMVNNSAI |
| 351 | VAIYQAGENI YELFDKTTVL YNGRQIYFGP ADKAVGYFQR MGWVKPNRMT |
| 401 | SAEFLTSVTV DFENRTLDIK PGYEDKVPKS SSEFEEYWLN SEDYQELLRT |
| 451 | YDDYQSRHPV NETRDRLDVA KKQRLQQGQR ENSQYVVNYW TQVYYCMIRG |
| 501 | FQRVKGDSTY TKVYLSSFLI KALIIGSMFH KIDDKSQSTT AGAYSRGGML |
| 551 | FYVLLFASVT SLAEIGNSFS SRPVIVKHKS YSMYHLSAES LQEIITEFPT |
| 601 | KFVAIVILCL ITYWIPFMKY EAGAFFQYIL YLLTVQQCTS FIFKFVATMS |
| 651 | KSGVDAHAVG GLWVLMLCVY AGFVLPIGEM HHWIRWLHFI NPLTYAFESL |
| 701 | VSTEFHHREM LCSALVPSGP GYEGISIANQ VCDAAGAVKG NLYVSGDSYI |
| 751 | LHQYHFAYKH AWRNWGVNIV WTFGYIVFNV ILSEYLKPVE GGGDLLLYKR |
| 801 | GHMPELGTEN ADARTASREE MMEALNGPNV DLEKVIAEKD VFTWNHLDYT |
| 851 | IPYDGATRKL LSDVFGYVKP GKMTALMGES GAGKTTLLNV LAQRINMGVI |
| 901 | TGDMLVNAKP LPASFNRSCG YVAQADNHMA ELSVRESLRF AAELRQQSSV |
| 951 | PLEEKYEYVE KIITLLGMQN YAEALVGKTG RGLNVEQRKK LSIGVELVAK |
| 1001 | PSLLLFLDEP TSGLDSQSAW SIVQFMRALA DSGQSILCTI HQPSATLFEQ |
| 1051 | FDRLLLLKKG GKMVYFGDIG PNSETLLKYF ERQSGMKCGV SENPAEYILN |
| 1101 | CIGAGATASV NSDWHDLWLA SPECAAARAE VEELHRTLPG RAVNDDPELA |
| 1151 | TRFAASYMTQ IKCVLRRTAL QFWRSPVYIR AKFFECVACA LFVGLSYVGV |
| 1201 | NHSVGGAIEA FSSIFMLLLI ALAMINQLHV FAYDSRELYE VREAASNTFH |
| 1251 | WSVLLLCHAA VENFWSTLCQ FMCFICYYWP AQFSGRASHA GFFFFFYVLI |
| 1301 | FPLYFVTYGL WILYMSPDVP SASMINSNLF AAMLLFCGIL QPREKMPAFW |
| 1351 | RRLMYNVSPF TYVVQALVTP LVHNKKVVCN PHEYNIMDPP SGKTCGEFLS |
| 1401 | TYMDNNTGYL VNPTATENCQ YCPYTVQDQV VAKYNVKWDH RWRNFGFMWA |
| 1451 | YICFNIAAML ICYYVVRVKV WSLKSVLNFK KWFNGPRKER HEKDTNIFQT |
| 1501 | VPGDENKITK K |

SEQ ID NO: 27
ZWF1 amino acid sequence; systematic name YNL241C

| | |
|---|---|
| 1 | MSEGPVKFEK NTVISVFGAS GDLAKKKTFP ALFGLFREGY LDPSTKIFGY |
| 51 | ARSKLSMEED LKSRVLPHLK KPHGEADDSK VEQFFKMVSY ISGNYDTDEG |
| 101 | FDELRTQIEK FEKSANVDVP HRLFYLALPP SVFLTVAKQI KSRVYAENGI |
| 151 | TRVIVEKPFG HDLASARELQ KNLGPLFKEE ELYRIDHYLG KELVKNLLVL |
| 201 | RFGNQFLNAS WNRDNIQSVQ ISFKERFGTE GRGGYFDSIG IIRDVMQNHL |
| 251 | LQIMTLLTME RPVSFDPESI RDEKVKVLKA VAPIDTDDVL LGQYGKSEDG |
| 301 | SKPAYVDDDT VDKDSKCVTF AAMTFNIENE RWEGVPIMMR AGKALNESKV |
| 351 | EIRLQYKAVA SGVFKDIPNN ELVIRVQPDA AVYLKFNAKT PGLSNATQVT |
| 401 | DLNLTYASRY QDFWIPEAYE VLIRDALLGD HSNFVRDDEL DISWGIFTPL |
| 451 | LKHIERPDGP TPEIYPYGSR GPKGLKEYMQ KHKYVMPEKH PYAWPVTKPE |
| 501 | DTKDN |

SEQ ID NO: 28
nucleic acid sequence ERR3
ATGTCCATCACGAAGGTACATGCTAGAACGGTGTATGATTCTCGCGGTAATCCGACTGTT
GAGGTTGAAATTACAACAGAGAATGGTCTCTTCAGAGCGATCGTCCCATCTGGTGCCTCC
ACCGGCATTCACGAAGCTGTTGAACTTAGAGACGGGAACAAGTCCGAATGGATGGGAAAA
GGGGTGACCAAGGCAGTCAGTAACGTCAATAGTATCATAGGGCCTGCTTTAATCAAGTCC
GACTTATGTGTAACCAATCAGAAGGGCATAGACGAGCTCATGATATCGTTAGACGGAACT
TCTAACAAGTCAAGGTTGGGCGCCAATGCTATCCTTGGTGTTTCCTTGTGCGTTGCTCGA
GCTGCTGCCGCACAAAAGGGAATTACTCTCTACAAGTATATAGCCGAGTTAGCGGATGCT
AGACAGGACCCCTTTGTTATTCCTGTTCCTTTTTTCAATGTTTTGAATGGTGGAGCCCAC
GCCGGTGGCTCTTTAGCTATGCAAGAATTCAAGATCGCGCCAGTCGGGGCTCAGAGCTTT
GCAGAAGCCATGAGGATGGGTTCGGAGGTTTACCATCATTTGAAGATATTGGCGAAGGAG
CAATATGGACCTTCCGCTGGAAATGTTGGTGACGAGGGTGGAGTCGCCCCCGATATCGAC
ACTGCCGAGGACGCCTTGGACATGATTGTGAAAGCCATTAACATATGCGGTTACGAGGGT
AGAGTGAAAGTAGGAATCGATAGTGCTCCTTCTGTTTTTTATAAGGACGGGAAATACGAC
CTAAATTTCAAGGAACCGAACTCTGACCCATCTCACTGGCTCAGTCCAGCTCAGTTAGCA
GAATATTATCATTCATTGCTAAAGAAATACCCAATCATTTCCCTGGAAGACCCCTACGCC
GAAGATGATTGGTCCTCGTGGTCTGCCTTCCTAAAGACTGTCAATGTTCAGATAATTGCA
GATGACCTGACATGCACCAACAAGACCAGGATCGCCCGTGCTATAGAGGAGAAATGTGCG
AATACTCTGTTGCTGAAACTCAACCAGATCGGTACTCTGACTGAGTCTATTGAAGCCGCC
AATCAGGCTTTCGATGCTGGATGGGGTGTAATGATATCACATAGATCAGGTGAAACCGAA
GATCCGTTTATCGCTGATTTGGTCGTTGGTTTAAGATGTGGTCAAATTAAATCGGGCGCT
TTGTCGAGATCAGAAAGACTGGCCAAGTATAATGAACTTTTGCGTATCGAAGAGGAACTG
GGGGACGATTGTATATATGCTGGTCATAGGTTTCATGATGGAAACAAACTATAA
SEQ ID NO: 29
nucleic acid sequence FOX2
ATGCCTGGAAATTTATCCTTCAAAGATAGAGTTGTTGTAATCACGGGCGCTGGAGGGGGC
TTAGGTAAGGTGTATGCACTAGCTTACGCAAGCAGAGGTGCAAAAGTGGTCGTCAATGAT
CTAGGTGGCACTTTGGGTGGTTCAGGACATAACTCCAAAGCTGCAGACTTAGTGGTGGAT
GAGATAAAAAAAGCCGGAGGTATAGCTGTGGCAAATTACGACTCTGTTAATGAAAATGGA
GAGAAAATAATTGAAACGGCTATAAAAGAATTCGGCAGGGTTGATGTACTAATTAACAAC
GCTGGAATATTAAGGGATGTTTCATTTGCAAAGATGACAGAACGTGAGTTTGCATCTGTG
GTAGATGTTCATTTGACAGGTGGCTATAAGCTATCGCGTGCTGCTTGGCCTTATATGCGC
TCTCAGAAATTTGGTAGAATCATTAACACCGCTTCCCCTGCCGGTCTATTTGGAAATTTT
GGTCAAGCTAATTATTCAGCAGCTAAAATGGGCTTAGTTGGTTTGGCGGAAACCCTCGCG
AAGGAGGGTGCCAAATACAACATTAATGTTAATTCAATTGCGCCATTGGCTAGATCACGT
ATGACAGAAAACGTGTTACCACCACATATCTTGAAACAGTTAGGACCGGAAAAAATTGTT
CCCTTAGTACTCTATTTGACACACGAAAGTACGAAAGTGTCAAACTCCATTTTTGAACTC
GCTGCTGGATTCTTTGGACAGCTCAGATGGGAGAGGTCTTCTGGACAAATTTTCAATCCA
GACCCCAAGACATATACTCCTGAAGCAATTTTAAATAAGTGGAAGGAAATCACAGACTAT
AGGGACAAGCCATTTAACAAAACTCAGCATCCATATCAACTCTCGGATTATAATGATTTA
ATCACCAAAGCAAAAAAATTACCTCCCAATGAACAAGGCTCAGTGAAAATCAAGTCGCTT
TGCAACAAAGTCGTAGTAGTTACGGGTGCAGGAGGTGGTCTTGGGAAGTCTCATGCAATC
TGGTTTGCACGGTACGGTGCGAAGGTAGTTGTAAATGACATCAAGGATCCTTTTTCAGTT
GTTGAAGAAATAAATAAACTATATGGTGAAGGCACAGCCATTCCAGATTCCCATGATGTG
GTCACCGAAGCTCCTCTCATTATCCAAACTGCAATAAGTAAGTTTCAGAGAGTAGACATC
TTGGTCAATAACGCTGGTATTTTGCGTGACAAATCTTTTTTAAAAATGAAAGATGAGGAA
TGGTTTGCTGTCCTGAAAGTCCACCTTTTTTCCACATTTTCATTGTCAAAAGCAGTATGG
CCAATATTTACCAAACAAAAGTCTGGATTTATTATCAATACTACTTCTACCTCAGGAATT
TATGGTAATTTTGGACAGGCCAATTATGCCGCTGCAAAAGCCGCCATTTTAGGATTCAGT
AAAACTATTGCACTGGAAGGTGCCAAGAGAGGAATTATTGTTAATGTTATCGCTCCTCAT
GCAGAAACGGCTATGACAAAGACTATATTCTCGGAGAAGGAATTATCAAACCACTTTGAT
GCATCTCAAGTCTCCCCACTTGTTGTTTTGTTGGCATCTGAAGAACTACAAAAGTATTCT
GGAAGAAGGGTTATTGGCCAATTATTCGAAGTTGGCGGTGGTTGGTGTGGGCAAACCAGA
TGGCAAAGAAGTTCCGGTTATGTTTCTATTAAAGAGACTATTGAACCGGAAGAAATTAAA
GAAAATTGGAACCACATCACTGATTTCAGTCGCAACACTATCAACCCGAGCTCCACAGAG
GAGTCTTCTATGGCAACCTTGCAAGCCGTGCAAAAAGCGCACTCTTCAAAGGAGTTGGAT
GATGGATTATTCAAGTACACTACCAAGGATTGTATCTTGTACAATTTAGGACTTGGATGC
ACAAGCAAAGAGCTTAAGTACACCTACGAGAATGATCCAGACTTCCAAGTTTTGCCCACG
TTCGCCGTCATTCCATTTATGCAAGCTACTGCCACACTAGCTATGGACAATTTAGTCGAT
AACTTCAATTATGCAATGTTACTGCATGGAGAACAATATTTTAAGCTCTGCACGCCGACA
ATGCCAAGTAATGGAACTCTAAAGACACTTGCTAAACCTTTACAAGTACTTGACAAGAAT
GGTAAAGCCGCTTTAGTTGTTGGTGGCTTCGAAACTTATGACATTAAAACTAAGAAACTC
ATAGCTTATAACGAAGGATCGTTCTTCATCAGGGGCGCACATGTACCTCCAGAAAAGGAA
GTGAGGGATGGGAAAAGAGCCAAGTTTGCTGTCCAAAATTTTGAAGTGCCACATGGAAAG
GTACCAGATTTTGAGGCGGAGATTTCTACGAATAAAGATCAAGCCGCATTGTACAGGTTA
TCTGGCGATTTCAATCCTTTACATATCGATCCCACGCTAGCCAAAGCAGTTAAATTTCCT
ACGCCAATTCTGCATGGGCTTTGTACATTAGGTATTAGTGCGAAAGCATTGTTTGAACAT
TATGGTCCATATGAGGAGTTGAAAGTGAGATTTACCAATGTTGTTTTCCCAGGTGATACT
CTAAAGGTTAAAGCTTGGAAGCAAGGCTCGGTTGTCGTTTTTCAAACAATTGATACGACC
AGAAACGTCATTGTATTGGATAACGCCGCTGTAAAACTATCGCAGGCAAAATCTAAACTA
TAA
SEQ ID NO: 30
nucleic acid sequence LYS1
ATGGCTGCCGTCACATTACATCTAAGAGCTGAAACTAAACCCCTAGAGGCACGTGCTGCC
TTAACACCTACCACGGTTAAAAAACTGATAGCTAAGGGCTTCAAAATATATGTAGAGGAC
AGTCCACAATCTACTTTCAATATTAACGAATATCGTCAAGCAGGTGCCATTATAGTGCCT
GCAGGTTCATGGAAAACCGCTCCACGCGACAGAATCATTATAGGTTTGAAGGAAATGCCT
GAAACCGATACTTTCCCTCTAGTCCACGAACACATCCAGTTTGCTCACTGCTACAAAGAC
CAAGCTGGGTGGCAAAATGTCCTTATGAGATTTATTAAGGGACACGGTACTCTATATGAT
TTGGAATTTTTGGAAAATGACCAAGGTAGAAGAGTTGCTGCCTTTGGATTTTACGCTGGG
TTCGCAGGTGCAGCCCTTGGTGTAAGAGACTGGGCATTCAAGCAAACGCATTCTGACGAT
GAAGACTTGCCTGCAGTGTCGCCTTACCCCAATGAAAAGGCATTGGTTAAAGATGTTACC
AAAGATTATAAAGAAGCCTTAGCCACCGGAGCCAGAAAGCCAACCGTGTTAATCATTGGT
GCGCTAGGAAGATGTGGTTCCGGTGCCATCGATCTGTTGCACAAAGTTGGTATTCCAGAT
GCTAACATATTAAAATGGGATATCAAAGAAACTTCCCGTGGTGGTCCCTTTGACGAAATT
CCACAAGCTGATATTTTTATCAATTGTATATATCTATCGAAGCCAATTGCTCCTTTCACT
AACATGGAGAAACTGAATAATCCTAACAGAAGACTAAGGACCGTGGTGGACGTATCAGCA
GACACTACCAACCCTCACAACCCCATCCCAATATACACTGTGGCTACTGTGTTTAACAAA
CCTACCGTTCTGGTACCTACCACTGCCGGGCCTAAATTATCTGTCATCTCTATTGATCAC
TTGCCTTCTTTGCTGCCAAGAGAAGCTTCAGAATTTTTCTCTCATGATCTCTTACCATCT
TTAGAGCTCCTACCTCAAAGAAAAACTGCTCCTGTCTGGGTTAGAGCCAAGAAATTGTTC
GATAGACATTGCGCTCGTGTTAAAAGATCTTCAAGATTGTAG
SEQ ID NO: 31
nucleic acid sequence MET1
ATGGTACGAGACTTAGTGACATTGCCTTCATCACTGCCCTTGATTACTGCTGGTTTTGCT
ACTGATCAGGTTCATTTGCTTATTGGTACAGGGTCCACGGACTCAGTAAGCGTTTGTAAG
AATAGAATCCACTCCATTTTGAATGCTGGTGGTAATCCCATAGTAGTGAATCCCTCGTCA
CCAAGCCATACTAAACAATTACAATTGGAATTTGGTAAGTTTGCAAAGTTCGAAATAGTA
GAAAGGGAGTTTAGGTTATCTGATTTAACTACTTTGGGGAGAGTTCTGGTATGCAAGGTA
GTGGATAGAGTATTCGTAGATCTACCCATAACACAAAGTCGCCTATGCGAGGAGATCTTT
TGGCAATGCCAAAAACTGAGAATTCCCATAAATACATTCCACAAACCAGAGTTTTCTACC
TTCAATATGATTCCTACGTGGGTCGACCCAAAAGGAAGTGGTTTACAAATCTCAGTTACT
ACGAATGGGAATGGATACATCTTGGCAAACAGGATAAAAAGAGATATAATATCACACTTA
CCTCCAAACATATCTGAGGTGGTGATAAACATGGGGTATTTGAAAGACCGTATTATAAAC
GAAGACCATAAGGCCTTGTTAGAGGAAAAGTACTACCAGACTGACATGTCATTACCTGGA
TTTGGCTACGGCTTAGATGAGGACGGTTGGGAGAGCCATAAGTTTAATAAGCTAATTCGT
GAATTTGAAATGACCAGTAGAGAACAGAGACTTAAGAGAACCAGATGGTTATCTCAGATA
ATGGAGTATTACCCGATGAACAAGCTGAGTGACATCAAGTTGGAAGATTTCGAGACTTCA
TCTTCTCCAAATAAAAAGACAAAGCAGGAAACTGTCACAGAGGGTGTAGTACCTCCTACC
GATGAAAATATTGAAAACGGTACAAAACAACTACAATTATCGGAAGTGAAAAAAGAGGAG
GGACCTAAAAAACTAGGGAAGATTTCTTTAGTCGGAAGTGGTCCAGGCTCGGTATCTATG
CTAACGATAGGTGCATTACAAGAAATAAAGTCTGCAGATATAATACTGGCAGATAAACTG
GTACCGCAAGCCATTTTAGATTTAATACCTCCAAAAACTGAAACCTTCATAGCCAAAAAA
TTTCCCGGTAATGCAGAACGAGCACAACAGGAATTACTAGCTAAAGGTTTAGAATCGTTG
GATAATGGATTGAAAGTAGTCCGTTTGAAGCAAGGTGATCCGTATATTTTTGGCCGTGGT
GGCGAGGAATTTAATTTCTTCAAAGATCACGGATATATTCCTGTGGTTTTACCGGGCATA
AGCTCATCCCTAGCTTGTACTGTATTGGCTCAGATACCCGCTACTCAACGTGATATAGCA
GACCAAGTGCTCATATGTACTGGGACTGGGAGAAAGGGCGCTCTGCCTATAATTCCTGAA
TTTGTTGAAAGCAGAACCACCGTCTTTCTAATGGCACTGCATCGCGCCAACGTTCTGATC
ACGGGATTATTGAAGCATGGCTGGGATGGTGATGTCCCCGCTGCAATTGTCGAGAGAGGA
TCGTGCCCTGACCAGCGTGTTACTAGAACTCTTCTTAAATGGGTACCAGAAGTCGTGGAG
GAGATTGGTTCAAGGCCCCCCGGTGTCTTGGTTGTAGGCAAGGCTGTGAATGCATTGGTT
GAAAAAGATCTGATAAATTTTGACGAATCAAGAAAATTTGTCATTGATGAAGGTTTTAGA
GAATTTGAGGTTGATGTAGATAGTCTATTTAAGTTATACTAA
SEQ ID NO: 32
nucleic acid sequence MIG2
ATGCCTAAAAAGCAAACGAATTTCCCAGTAGATAACGAAAACAGACCTTTTAGATGTGAT
ACCTGTCACCGTGGTTTCCATCGGTTAGAACATAAAAAGAGACACTTGAGAACACACACT
GGGGAAAAACCTCATCATTGCGCATTTCCTGGTTGTGGGAAAAGTTTCAGTAGAAGCGAT
GAACTGAAAAGGCACATGAGAACGCATACAGGGCAATCTCAAAGGAGATTGAAGAAAGCT
AGCGTACAGAAACAGGAGTTTTTGACAGTAAGCGGAATTCCTACCATTGCATCGGGCGTG
ATGATACACCAACCAATACCGCAAGTCCTACCAGCAAATATGGCCATAAATGTTCAGGCA
GTAAATGGAGGTAACATTATACACGCTCCCAATGCGGTGCACCCAATGGTGATACCAATC
ATGGCCCAACCAGCCCCCATTCATGCCTCCGCTGCATCTTTCCAGCCTGCAACTTCTCCT
ATGCCAATTTCTACATACACTCCAGTTCCATCGCAATCATTCACCTCTTTCCAGAGCTCT
ATTGGCTCCATACAGTCAAATAGTGATGTTTCATCTATCTTCTCGAACATGAATGTTCGC
GTAAACACTCCACGCTCTGTGCCAAACTCTCCGAATGATGGATATTTACACCAGCAACAT
ATCCCACAGCAGTATCAGCATCAAACTGCCAGCCCTTCTGTTGCCAAGCAGCAGAAAACT
TTTGCACATTCTCTTGCATCTGCATTATCTACCTTACAAAAAAGAACGCCTGTAAGTGCC
CCTTCCACCACTATAGAATCACCATCCTCACCAAGTGATTCCAGTCATACCTCTGCATCC
AGCAGCGCTATCTCTTTGCCTTTCAGCAATGCTCCTTCTCAGCTCGCCGTGGCCAAAGAA
CTTGAGTCCGTCTATTTAGATTCCAATAGATACACCACCAAGACTAGGAGGGAAAGAGCA
AAATTCGAAATTCCTGAAGAACAAGAAGAAGATACCAATAACAGCAGCAGTGGTAGTAAT
GAGGAGGAGCACGAGTCGCTAGATCATGAATCTAGCAAAAGCCGAAAGAAATTGTCAGGC
GTAAAATTGCCGCCTGTACGTAACCTACTGAAACAAATTGATGTTTTCAACGGTCCCAAA
AGAGTTTAA
SEQ ID NO: 33
nucleic acid sequence RMD6
ATGTCAGCTTGCCCTTGCAACATCGTTATACTCCCAGTCGAGATTTTGAAGAATTCATCT
AAAGATACTAAGTATAGCTTGTATACAACAATTAATCGAGGATATGATGTCCCAAGACTC
AAATATGGCATCATAGTTAGCCCTCGAGTGCACAGCCTTGAGACTTTATTCAGTGATCTG
GGCTTTGACAAGAATATAGAGAAATCCTCGCTTTACTTATTATTAAATGATCCTACCTTA
GCATACCCTAATTTCCATGAACATTTTGAACAGCTTAAAGGTGAAACAAACAAAGATTTA
TCTCTACCGACATATTATATTCCGAAGGTCCAGTTTTTGACAGAGGCATTCGATTCAGAA
CATACCCTAGCAACCATCGGCTACAAACCAAATAATAAGGAGAGTTATGAGATCACAGGT
TTTACATCCATGGGTAATGGTTATGGTATAAAACTATTCAATTACAGTGTAATTCATATG
ATGCGGTCTCATAAGTGTAAAAGAGTGGTTGCAGATATTATCATGGAGCATGACCTATTG
GGTTACTATGAAAAGAAGCTTGGCTTTGTAGAGGTGCAAAGGTTCAAAGTTCTCAAAGAA
CAGCACCAAGTAAAGGTATTTGACGATAAAGTTGACTTTACCAAAGACTTTCATGTGATC
AAAATGATTAAAGAGTTGGGAAATCATAGATTGTAG
SEQ ID NO: 34
nucleic acid sequence RME1
ATGTCACCGTGTTATGGACAAAACAGTGCCATCGCCAAGGGGTCTTGGAACAGAGAGGTT
TTACAAGAGGTGCAACCGATTTATCATTGGCACGATTTCGGGCAAAACATGAAAGAATAT
TCGGCATCACCCTTAGAGGGGGATTCCAGCCTGCCTTCCAGCCTGCCTTCCAGCACTGAG
GACTGTTTACTACTATCATTAGAAAACACAATCACAGTTATAGCCGGAAATCAGAGACAG
GCTTATGACTCTACGTCGTCTACTGAGGAAGGTACAGCACCTCAATTACGGCCGGATGAA
ATAGCGGACAGTACACACTGTATCACGTCATTAGTTGATCCGGAGTTCAGAGATCTTATT
AATTATGGACGTCAAAAAGGAGCAAATCCTGTATTTATTGAGAGCAATACAACAGAACAA
TCCCATTCACAGTGTATTCTAGGCTATCCCCAAAAATCGCACGTGGCACAGCTATATCAC
GACCCCAAAGTACTCAGCACAATTTCCGAAGGGCAAACAAAAAGAGGAAGTTACCACTGT
TCTCATTGTTCTGAAAAGTTCGCAACGTTAGTTGAGTTTGCCGCGCACTTAGACGAATTC
AACCTTGAAAGACCGTGTAAGTGTCCCATAGAGCAATGTCCCTGGAAAATATTGGGTTTC
CAACAAGCAACTGGTCTGAGAAGACATTGTGCTTCCCAACATATAGGAGAGCTTGATATA
GAGATGGAGAAATCATTAAATCTAAAAGTAGAAAAATATCCAGGACTGAATTGCCCATTT
CCTATCTGTCAGAAAACGTTTAGGCGCAAAGACGCCTATAAGAGACATGTGGCCATGGTG
CATAACAACGCTGATTCAAGATTTAACAAGCGTTTGAAGAAAATTTTGAACAATACCAAA
TAG
SEQ ID NO: 35
nucleic acid sequence SIP1
ATGGGAAACAGTCCTTCTACTCAGGATCCATCGCATTCAACCAAAAAGGAGCATGGACAT
CATTTTCATGATGCATTCAATAAAGATCGTCAGGGAAGCATAACCTCTCAACTGTTTAAC
AATAGGAAGAGTACTCATAAGAGACGCGCCAGTCATACTAGCGAACATAATGGTGCCATT
CCCCCTAGAATGCAATTACTTGCATCTCACGATCCATCGACAGATTGTGATGGGCGCATG
AGCAGTGATACTACTATCGATAAGGGCCCGTCCCATCTATTCAAAAAGGATTATTCTTTG
TCCTCTGCCGCAGACGTAAATGACACTACGTTGGCCAATTTGACTTTAAGTGATGATCAC
GATGTGGGTGCACCCGAAGAGCAGGTGAAATCTCCGTCGTTTTTGAGCCCAGGTCCATCA
ATGGCCACTGTCAAACGAACTAAAAGTGATCTGGATGATCTGTCTACTTTGAATTACACC
ATGGTTGACGAAACAACAGAAAATGAAAGAAACGACAAACCACACCATGAAAGGCATCGC
TCAAGCATCATAGCTTTGAAAAAAAATCTTTTAGAAAGTTCAGCTACTGCTTCCCCTTCT
CCAACGAGGTCTTCATCGGTGCATTCAGCATCACTTCCCGCCTTGACCAAGACGGATTCC
ATTGATATTCCTGTAAGACAACCCTATTCAAAGAAACCATCTATCCATGCATACCAATAT
CAATATCTCAATAACGACGAAACATTTTCTGAGAACTCTCAAATGGATAAAGAGGGAAAC
AGTGATAGTGTAGATGCAGAAGCAGGCGTACTTCAAAGTGAAGATATGGTTTTGAACCAG
TCTCTTTTACAAAATGCTTTAAAAAAGGATATGCAACGTCTTTCAAGGGTGAATTCCTCT
AATTCAATGTATACTGCAGAAAGGATAAGCCACGCTAATAACAATGGAAATATTGAAAAT
AACACCCGTAACAAGGGAAACGCGGGAGGCTCCAACGACGATTTTACCGCACCTATATCT
GCTACTGCAAAAATGATGATGAAACTGTACGGTGATAAGACCTTGATGGAAAGAGATTTA
AATAAGCACCATAATAAGACAAAGAAAGCCCAGAATAAAAAAATAAGGTCGGTTTCAAAT
TCGAGAAGATCCTCATTTGCTTCTTTGCATTCTTTACAATCAAGAAAGAGTATCTTAACA
AACGGTTTGAATTTGCAGCCTTTGCATCCACTACATCCAATTATTAACGACAATGAAAGT
CAATACTCTGCACCGCAGCATAGAGAAATATCGCATCACTCTAATTCCATGTCGAGTATG
TCTTCAATATCATCAACAAACTCCACCGAAAATACTTTAGTTGTTCTAAAATGGAAAGAC
GATGGTACCGTGGCTGCAACCACGGAAGTCTTTATAGTAAGCACGGATATTGCTTCTGCT
CTTAAAGAGCAAAGAGAGCTTACCTTGGATGAGAATGCAAGTTTAGACTCAGAAAAACAG
TTAAATCCTAGGATCCGCATGGTTTATGATGATGTGCATAAGGAATGGTTTGTTCCAGAT
CTTTTTTTGCCTGCAGGAATTTATAGACTGCAATTCTCCATCAATGGTATATTGACTCAC
TCAAATTTTCTTCCTACAGCTACGGATTCAGAAGGCAATTTTGTCAACTGGTTTGAAGTA
TTGCCCGGTTATCATACGATCGAGCCATTTAGAAACGAAGCAGACATCGACTCGCAAGTA
GAGCCAACACTGGATGAAGAATTGCCCAAGAGACCGGAACTTAAAAGATTTCCTTCTTCC
TCTCGAAAATCTTCCTACTATTCTGCTAAGGGTGTTGAAAGGCCAAGTACACCGTTTTCA
GATTATAGAGGCTTAAGTAGGTCCAGTTCAATAAATATGCGTGACTCATTTGTACGTCTG
AAGGCTAGCAGTTTGGATTTGATGGCCGAGGTCAAGCCTGAGAGGTTGGTGTATTCGAAC
GAAATACCAAATTTATTTAATATAGGTGACGGCTCTACGATTTCTGTAAAAGGAGATTCT
GATGACGTGCATCCCCAAGAACCTCCCAGCTTTACACATAGAGTTGTTGACTGTAATCAA
GATGATTTATTTGCTACTTTACAGCAAGGCGGTAATATTGATGCAGAAACAGCAGAGGCG
GTTTTTCTAAGTAGATACCCAGTTCCTGACTTGCCCATATACCTGAATTCATCCTATCTG
AACAGAATACTAAACCAAAGCAATCAGAATTCAGAATCACATGAGAGGGATGAAGGTGCG
ATAAATCATATTATACCCCACGTGAATTTGAACCATTTACTGACAAGCAGTATTAGAGAT
GAAATAATCAGCGTAGCTTGCACTACTAGATATGAGGGGAAATTTATCACTCAAGTAGTT
TATGCACCTTGTTATTATAAAACACAAAAGTCTCAGATCAGTAATTAG
SEQ ID NO: 36
nucleic acid sequence SNP1
ATGAATTATAATCTATCCAAGTATCCAGACGACGTGTCGAGACTTTTCAAGCCAAGGCCA
CCTTTATCTTACAAAAGACCAACCGATTACCCATATGCGAAGAGACAAACAAATCCAAAT
ATCACTGGCGTTGCAAACTTACTATCAACCTCTTTGAAGCACTATATGGAGGAGTTTCCT
GAAGGATCTCCAAACAACCATCTCCAAAGATACGAAGACATCAAACTTTCCAAGATCAAA
AATGCTCAATTGTTAGACCGGAGACTACAAAATTGGAATCCTAACGTTGACCCTCATATC
AAGGACACAGATCCCTACAGAACGATATTTATTGGGAGGCTACCATACGATCTTGACGAA
ATTGAACTGCAAAAGTATTTTGTTAAGTTTGGCGAGATCGAAAAAATTAGGATAGTCAAG
GACAAGATAACCCAGAAGAGTAAAGGCTACGCCTTCATAGTTTTCAAAGACCCAATAAGT
AGTAAAATGGCATTCAAGGAGATTGGAGTACACAGAGGTATCCAAATCAAAGACAGAATC
TGCATAGTCGACATAGAAAGAGGCAGAACCGTTAAATATTTCAAGCCAAGAAGATTGGGC
GGCGGCCTAGGAGGCAGAGGCTATTCCAACAGAGACAGCAGGCTTCCAGGAAGGTTTGCA
AGCGCAAGTACATCAAATCCCGCCGAAAGAAATTATGCTCCCAGGCTGCCACGCAGGGAA
ACTTCTTCCTCCGCATATAGCGCTGATAGATACGGCAGTTCCACATTGGACGCGAGGTAC
CGTGGAAACAGGCCATTGCTCTCCGCCGCCACTCCTACTGCTGCTGTTACTTCTGTATAT
AAATCTAGAAACTCACGGACTCGAGAGTCTCAACCAGCTCCCAAAGAAGCGCCCGACTAT
TGA
SEQ ID NO: 37
nucleic acid sequence TDH1
ATGATCAGAATTGCTATTAACGGTTTCGGTAGAATCGGTAGATTGGTCTTGAGATTGGCT
TTGCAAAGAAAAGACATTGAGGTTGTTGCTGTCAACGATCCATTTATCTCTAACGATTAT
GCTGCTTACATGGTCAAGTACGATTCTACTCATGGTAGATACAAGGGTACTGTTTCCCAT
GACGACAAGCACATCATCATTGATGGTGTCAAGATCGCTACCTACCAAGAAAGAGACCCA
GCTAACTTGCCATGGGGTTCTCTAAAGATCGATGTCGCTGTTGACTCCACTGGTGTTTTC
AAGGAATTGGACACCGCTCAAAAGCACATTGACGCTGGTGCCAAGAAGGTTGTCATCACT
GCTCCATCTTCTTCTGCTCCAATGTTTGTTGTTGGTGTTAACCACACTAAATACACTCCA
GACAAGAAGATTGTCTCCAACGCTTCTTGTACCACCAACTGTTTGGCTCCATTGGCCAAG
GTTATCAACGATGCTTTCGGTATTGAAGAAGGTTTGATGACCACTGTTCACTCCATGACC
GCCACTCAAAAGACTGTTGATGGTCCATCCCACAAGGACTGGAGAGGTGGTAGAACCGCT
TCCGGTAACATTATCCCATCCTCTACCGGTGCTGCTAAGGCTGTCGGTAAGGTCTTGCCA
GAATTGCAAGGTAAGTTGACCGGTATGGCTTTCAGAGTCCCAACCGTCGATGTTTCCGTT
GTTGACTTGACTGTCAAGTTGGAAAAGGAAGCTACTTACGACCAAATCAAGAAGGCTGTT
AAGGCTGCCGCTGAAGGTCCAATGAAGGGTGTTTTGGGTTACACCGAAGATGCCGTTGTC
TCCTCTGATTTCTTGGGTGACACTCACGCTTCCATCTTCGATGCCTCCGCTGGTATCCAA
TTGTCTCCAAAGTTCGTCAAGTTGATTTCCTGGTACGATAACGAATACGGTTACTCCGCC
AGAGTTGTTGACTTGATCGAATATGTTGCCAAGGCTTAA
SEQ ID NO: 38
nucleic acid sequence GPD1
ATGTCTGCTGCTGCTGATAGATTAAACTTAACTTCCGGCCACTTGAATGCTGGTAGAAAG
AGAAGTTCCTCTTCTGTTTCTTTGAAGGCTGCCGAAAAGCCTTTCAAGGTTACTGTGATT
GGATCTGGTAACTGGGGTACTACTATTGCCAAGGTGGTTGCCGAAAATTGTAAGGGATAC
CCAGAAGTTTTCGCTCCAATAGTACAAATGTGGGTGTTCGAAGAAGAGATCAATGGTGAA
AAATTGACTGAAATCATAAATACTAGACATCAAAACGTGAAATACTTGCCTGGCATCACT
CTACCCGACAATTTGGTTGCTAATCCAGACTTGATTGATTCAGTCAAGGATGTCGACATC
ATCGTTTTCAACATTCCACATCAATTTTTGCCCCGTATCTGTAGCCAATTGAAAGGTCAT
GTTGATTCACACGTCAGAGCTATCTCCTGTCTAAAGGGTTTTGAAGTTGGTGCTAAAGGT
GTCCAATTGCTATCCTCTTACATCACTGAGGAACTAGGTATTCAATGTGGTGCTCTATCT
GGTGCTAACATTGCCACCGAAGTCGCTCAAGAACACTGGTCTGAAACAACAGTTGCTTAC
CACATTCCAAAGGATTTCAGAGGCGAGGGCAAGGACGTCGACCATAAGGTTCTAAAGGCC
TTGTTCCACAGACCTTACTTCCACGTTAGTGTCATCGAAGATGTTGCTGGTATCTCCATC
TGTGGTGCTTTGAAGAACGTTGTTGCCTTAGGTTGTGGTTTCGTCGAAGGTCTAGGCTGG
GGTAACAACGCTTCTGCTGCCATCCAAAGAGTCGGTTTGGGTGAGATCATCAGATTCGGT
CAAATGTTTTTCCCAGAATCTAGAGAAGAAACATACTACCAAGAGTCTGCTGGTGTTGCT
GATTTGATCACCACCTGCGCTGGTGGTAGAAACGTCAAGGTTGCTAGGCTAATGGCTACT
TCTGGTAAGGACGCCTGGGAATGTGAAAAGGAGTTGTTGAATGGCCAATCCGCTCAAGGT
TTAATTACCTGCAAAGAAGTTCACGAATGGTTGGAAACATGTGGCTCTGTCGAAGACTTC
CCATTATTTGAAGCCGTATACCAAATCGTTTACAACAACTACCCAATGAAGAACCTGCCG
GACATGATTGAAGAATTAGATCTACATGAAGATTAG
SEQ ID NO: 39
nucleic acid sequence RSF2
ATGGAACCGTTCGCATTTGGACGAGGGGCGCCTGCATTATGCATACTAACCGCGGCCGCT
CGAATAAATCTGGACAATTTTGTTCCGTGTTGCTGGGCACTTTTCCGTCTGTCTTTCTTT
TTCCCGCTTGACCCTGCATATATTAGAAACGAAAACAAAGAAACAAGGACTTCTTGGATT
TCCATAGAGTTTTTTTTCTTCGTTAAACATTGCCTCTCTCAACACACGTTTTTCTCGAAG
ACTCTTGCACCAAAAAGAAACTTTAGGGCGAAGAAGCTAAAAGACATTGGCGATACTAGA
ATAGATAGGGCAGATAAAGATTTTTTATTAGTGCCGGAGCCAAGTATGTTTGTGAACGGT
AATCAATCTAATTTCGCTAAGCCCGCTGGTCAAGGTATTCTGCCCATTCCTAAAAAATCT
CGAATTATTAAGACTGATAAGCCAAGACCGTTCTTGTGTCCCACATGCACTAGGGGTTTT
GTCAGGCAGGAGCATTTGAAGAGACATCAGCATTCGCATACCCGTGAGAAACCGTATCTT
TGTATCTTTTGCGGTAGGTGTTTTGCTCGTAGAGATTTAGTGCTCAGGCATCAGCAAAAA
CTTCATGCTGCTCTTGTAGGTACGGGGGATCCACGGCGAATGACGCCAGCACCAAATTCG
ACTTCTTCTTTTGCCTCCAAGCGGCGCCATTCCGTGGCGGCGGATGATCCAACCGACCTT
CATATCATTAAAATAGCCGGAAATAAAGAGACTATTCTACCCACCCCGAAGAACCTTGCT
GGTAAGACATCTGAAGAATTGAAAGAGGCCGTGGTTGCCTTGGCCAAATCAAATAATGTA
GAACTTCCCGTCTCGGCCCCAGTAATGAACGATAAGCGAGAGAAAACTCCTCCTAGTAAG
GCAGGCTCCCTAGGATTTCGAGAGTTCAAGTTCAGCACGAAAGGCGTGCCAGTTCACTCT
GCATCAAGCGATGCTGTTATCGACAGGGCGAACACTCCCTCTTCCATGCATAAGACGAAA
AGACATGCGTCTTTCTCTGCATCCAGTGCAATGACTTACATGTCTAGTAGCAATAGCCCC
CACCATTCAATTACCAATTTCGAGCTCGTTGAAGACGCTCCGCATCAAGTCGGCTTTTCT
ACTCCACAAATGACCGCGAAGCAGCTCATGGAAAGCGTGTCAGAATTGGATTTACCTCCG
TTAACCCTGGACGAACCACCGCAAGCTATCAAGTTTAACTTAAATCTATTTAACAATGAC
CCCTCCGGACAGCAACAACAACAACAACAACAACAGCAAAATTCCACCTCTAGTACCATA
GTGAACAGCAACAATGGAAGTACAGTTGCTACACCTGGAGTGTATCTCTTAAGTAGCGGT
CCATCTTTAACCGATCTTTTGACAATGAACTCTGCACATGCAGGTGCGGGAGGATACATG
TCTAGCCACCATTCGCCATTTGATTTGGGCTGCTTCAGTCATGATAAACCGACAGTTTCT
GAATTTAACCTTCCGTCAAGCTTCCCGAATACTATACCGTCTAATTCTACTACGGCTTCT
AATAGTTACAGTAATTTGGCAAATCAAACTTATAGGCAAATGAGCAATGAGCAGCCGCTT
ATGTCACTATCTCCTAAAAACCCACCAACAACTGTTTCAGATTCCTCTTCCACGATCAAT
TTCAATCCAGGCACAAATAATTTACTGGAACCATCAATGGAGCCCAATGATAAGGATAGT
AATATCGATCCTGCTGCCATAGATGACAAGTGGTTATCAGAGTTTATTAACAACTCTGAT
CCAAAATCTACCTTCAAGATCAACTTCAATCATTTCAATGACATTGGGTTTATTTATTCT
CCACCTTCATCAAGGTCATCTATACCAAACAAGTCACCTCCAAACCATTCTGCTACCTCA
TTAAATCATGAAAAAGCTTCTTTATCACCTCGCTTAAACTTGAGTTTGAATGGAAGCACA
GATTTACCAAGTACACCACAAAACCAACTAAAGGAGCCTTCCTATTCTGACCCTATTTCC
CATAGTTCTCATAAGAGGCGTCGTGATAGCGTCATGATGGACTACGATCTATCCAATTTT
TTCAGCTCAAGGCAATTGGATATTTCCAAGGTATTAAACGGGACAGAGCAAAATAATTCT
CATGTGAACGACGATGTTCTCACTTTGTCTTTCCCCGGCGAAACTGATTCTAATGCAACA
CAGAAACAGCTGCCTGTTCTTACTCCTTCGGATTTGTTATCTCCGTTTTCTGTCCCTTCA
GTATCTCAAGTGCTTTTTACCAATGAGCTAAGGAGTATGATGCTAGCCGACAATAATATC
GATTCAGGAGCCTTCCCCACAACTAGTCAATTGAACGATTATGTGACTTACTATAAGGAA
GAATTCCATCCATTTTTTTCATTTATTCATCTTCCTTCTATCATACCTAATATGGACAGT
TATCCCTTGTTATTATCTATCTCCATGGTCGGAGCATTGTATGGGTTTCATTCGACGCAT
GCAAAAGTGTTAGCTAATGCAGCTAGCACCCAAATTAGGAAAAGCTTGAAAGTTAGTGAG
AAAAACCCGGAGACGACAGAGTTATGGGTTATACAGACATTAGTATTGCTAACGTTCTAC
TGTATTTTCAATAAAAATACAGCCGTGATCAAGGGGATGCATGGTCAGTTGACGACTATT
ATTCGTCTCTTGAAGGCCTCTCGTTTAAATTTGCCCCTAGAGTCCCTATGCCAGCCGCCT
ATTGAGAGTGATCATATTATGGAATATGAAAACAGTCCTCATATGTTTTCAAAAATAAGA
GAGCAATACAACGCGCCGAATCAAATGAACAAAAACTACCAATATTTTGTATTGGCGCAG
TCACGTATCAGGACTTGCCATGCGGTATTACTTATATCTAACTTATTTTCTTCACTGGTA
GGTGCTGATTGCTGTTTTCATTCAGTCGATTTAAAATGTGGTGTTCCATGCTATAAAGAA
GAATTATATCAGTGCCGAAATTCCGATGAATGGTCGGACCTATTATGTCAATACAAAATA
ACGTTAGATTCGAAATTTTCGTTGATTGAATTGTCTAATGGTAACGAGGCATATGAAAAT
TGTTTGAGGTTTCTTTCTACAGGCGATAGTTTTTTTTACGGAAATGCTAGGGTTTCGTTA
AGTACATGTCTATCATTGTTGATATCTATCCATGAGAAAATACTTATTGAAAGAAATAAC
GCAAGGATCAGTAATAACAACACCAATAGCAATAACATTGAGTTGGACGATATTGAGTGG
AAGATGACTTCCAGACAACGGATCGATACAATGTTAAAATACTGGGAAAACCTTTATTTG
AAAAATGGTGGCATCTTGACACCTACCGAGAATAGCATGTCAACAATAAACGCCAATCCA
GCAATGAGGTTAATAATTCCGGTATATTTGTTTGCCAAAATGAGACGGTGTTTGGACCTG
GCACATGTTATTGAGAAAATCTGGTTGAAAGATTGGTCCAATATGAATAAAGCTTTGGAG
GAAGTTTGCTATGACATGGGTTCATTGAGGGAAGCTACCGAGTATGCACTGAATATGGTG
GATGCGTGGACTTCATTTTTTACGTACATTAAACAGGGCAAGCGCAGAATTTTCAATACT
CCTGTATTTGCGACCACATGTATGTTCACTGCAGTATTAGTGATTTCGGAATACATGAAA
TGTGTAGAGGATTGGGCACGCGGGTACAATGCCAACAACCCTAACTCAGCATTATTGGAT
TTTTCGGACCGTGTCTTATGGCTAAAAGCAGAAAGGATTTTGAGAAGATTACAAATGAAC
TTGATACCGAAGGAGTGTGATGTGTTGAAATCGTACACTGATTTCTTAAGATGGCAGGAC
AAGGATGCCCTAGATTTGTCAGCACTAAATGAAGAACAAGCACAAAGGGCCATGGACCCG
AATACCGATATAAATGAGACAATTCAACTAATTGTAGCGGCAAGTCTATCCTCCAAATGT
TTATATTTGGGTGTTCAAATATTGGGTGATGCGCCAATTTGGCCTATAATATTATCGTTC
GCTCATGGTTTGCAATCAAGAGCTATCTATAGTGTTACGAAAAAAAGAAACACTAGAATA
TAA
SEQ ID NO: 40
nucleic acid sequence GND2
ATGTCAAAGGCAGTAGGTGATTTAGGCTTAGTTGGTTTAGCCGTGATGGGTCAAAATTTG
ATCTTAAACGCAGCGGATCACGGATTTACCGTGGTTGCTTATAATAGGACGCAATCAAAG
GTAGATAGGTTTCTAGCTAATGAGGCAAAAGGAAAATCAATAATTGGTGCAACTTCAATT
GAGGACTTGGTTGCGAAACTAAAGAAACCTAGAAAGATTATGCTTTTAATCAAAGCCGGT
GCTCCGGTCGACACTTTAATAAAGGAACTTGTACCACATCTTGATAAAGGCGACATTATT
ATCGACGGTGGTAACTCACATTTCCCGGACACTAACAGACGCTACGAAGAGCTAACAAAG
CAAGGAATTCTTTTTGTGGGCTCTGGTGTCTCAGGCGGTGAAGATGGTGCACGTTTTGGT
CCATCTTTAATGCCTGGTGGGTCAGCAGAAGCATGGCCGCACATCAAGAACATCTTTCAA
TCTATTGCCGCCAAATCAAACGGTGAGCCATGCTGCGAATGGGTGGGGCCTGCCGGTTCT
GGTCACTATGTGAAGATGGTACACAACGGTATCGAGTACGGTGATATGCAGTTGATTTGC
GAGGCTTACGATATCATGAAACGAATTGGCCGGTTTACGGATAAAGAGATCAGTGAAGTA
TTTGACAAGTGGAACACTGGAGTTTTGGATTCTTTCTTGATTGAAATCACGAGGGACATT
TTAAAATTCGATGACGTCGACGGTAAGCCATTGGTGGAAAAAATTATGGATACTGCCGGT
CAAAAGGGTACTGGTAAATGGACTGCAATCAACGCCTTGGATTTAGGAATGCCAGTCACT
TTAATTGGGGAGGCTGTTTTCGCTCGTTGTTTGTCAGCCATAAAGGACGAACGTAAAAGA
GCTTCGAAACTTCTGGCAGGACCAACAGTACCAAAGGATGCAATACATGATAGAGAACAA
TTTGTGTATGATTTGGAACAAGCATTATACGCTTCAAAGATTATTTCATATGCTCAAGGT
TTCATGCTGATCCGCGAAGCTGCCAGATCATACGGCTGGAAATTAAACAACCCAGCTATT
GCTCTAATGTGGAGAGGTGGCTGTATAATCAGATCTGTGTTCTTAGCTGAGATTACGAAG
GCTTATAGGGACGATCCAGATTTGGAAAATTTATTATTCAACGAGTTCTTCGCTTCTGCA
GTTACTAAGGCCCAATCCGGTTGGAGAAGAACTATTGCCCTTGCTGCTACTTACGGTATT
CCAACTCCAGCTTTCTCTACTGCTTTAGCGTTTTACGACGGCTATAGATCTGAGAGGCTA
CCAGCAAACTTGTTACAAGCGCAACGTGATTATTTTGGCGCTCATACATTTAGAATTTTA
CCTGAATGTGCTTCTGCCCATTTGCCAGTAGACAAGGATATTCATATCAATTGGACTGGG
CACGGAGGTAATATATCTTCCTCAACCTACCAAGCTTAA
SEQ ID NO: 41
nucleic acid sequence TRK1
ATGCATTTTAGAAGAACGATGAGTAGAGTGCCCACATTGGCATCTCTTGAAATACGATAT
AAAAAATCTTTCGGCCATAAATTTCGTGATTTTATTGCTCTATGTGGTCACTATTTTGCT
CCAGTTAAAAAATATATCTTCCCCAGTTTTATCGCGGTTCACTACTTCTACACGATATCC
CTGACATTAATAACTTCAATCCTGCTATATCCCATTAAGAATACCAGATACATTGATACA
TTGTTTTTAGCAGCGGGCGCAGTTACACAAGGTGGCTTAAATACTGTGGATATCAACAAT
CTAAGCTTATACCAACAAATTGTTCTGTATATCGTATGCTGCATATCAACACCAATTGCA
GTTCATAGTTGCTTGGCATTTGTACGGCTTTACTGGTTTGAGCGCTACTTCGATGGTATT
AGAGACTCTTCTAGACGAAATTTTAAGATGAGAAGAACGAAAACAATCTTAGAAAGGGAA
CTAACAGCAAGAACCATGACCAAGAATAGAACAGGTACCCAAAGAACGTCTTATCCTAGG
AAACAAGCTAAAACAGATGATTTCCAAGAAAAATTGTTCAGCGGAGAAATGGTTAATAGA
GATGAGCAGGACTCAGTTCACAGCGACCAGAATTCTCATGACATTAGTAGGGACAGCAGC
AATAATAATACGAATCACAATGGTAGCAGTGGCAGTTTAGATGATTTCGTTAAGGAAGAC
GAAACGGATGACAATGGAGAATATCAGGAGAACAACTCCTACTCGACGGTAGGTAGTTCG
TCTAACACAGTTGCAGACGAAAGTTTAAATCAGAAGCCCAAGCCAAGCAGTCTTCGGTTT
GATGAGCCACACAGCAAACAAAGACCCGCAAGAGTTCCCTCAGAGAAATTTGCAAAAAGA
AGGGGTTCAAGAGATATTAGCCCAGCCGATATGTATCGATCCATTATGATGCTACAAGGT
AAGCATGAAGCAACTGCTGAAGATGAAGGTCCCCCTTTAGTCATCGGGTCCCCTGCGGAT
GGCACAAGATATAAAAGTAATGTCAATAAGCTAAAGAAGGCCACCGGCATAAATGGTAAC
AAAATCAAGATTCGAGATAAGGGAAATGAAAGTAACACTGATCAAAATTCCGTGTCAAGT
GAAGCAAACAGTACGGCGAGCGTTTCGGACGAAAGCTCGTTACACACAAATTTTGGTAAC
AAAGTACCTTCATTAAGAACAAATACTCATAGATCAAATTCGGGCCCGATAGCCATTACT
GATAACGCAGAAACAGACAAAAAGCATGGGCCATCAATTCAATTCGATATAACTAAACCT
CCTAGAAAAATTTCAAAAAGAGTTTCAACCTTCGATGATTTGAACCCAAAATCTTCCGTT
CTTTATCGAAAAAAAGCATCGAAGAAGTACCTCATGAAACATTTTCCTAAAGCGCGGCGA
ATACGGCAACAAATTAAGAGAAGGCTTTCTACTGGTTCAATTGAGAAAAACAGCAGTAAC
AATGTTTCAGATAGAAAACCTATTACTGATATGGATGATGATGATGATGACGATGACAAC
GACGGCGATAACAACGAAGAATACTTTGCTGACAACGAAAGCGGCGATGAAGATGAACGA
GTACAGCAGTCTGAACCACATTCTGATTCAGAACTCAAATCGCACCAACAACAGCAAGAA
AAACACCAACTGCAGCAGAACCTGCACCGCATGTATAAAACCAAATCATTTGATGATAAT
CGTTCAAGAGCAGTTCCTATGGAACGTTCCAGGACCATCGATATGGCAGAGGCTAAGGAT
CTAAATGAGCTCGCAAGGACGCCTGATTTTCAAAAAATGGTCTATCAAAATTGGAAAGCC
CATCATAGAAAAAAACCGAACTTTAGGAAGAGGGGATGGAATAACAAGATATTTGAACAT
GGTCCCTATGCATCTGACAGCGATCGCAATTATCCTGATAATAGTAATACTGGAAACAGT
ATTCTTCATTACGCAGAGTCTATTTTACATCATGATGGCTCTCATAAAAATGGAAGCGAA
GAAGCCTCTTCCGACTCTAATGAGAATATCTATTCCACGAATGGAGGAAGCGACCACAAT
GGTCTTAACAACTATCCTACTTACAACGACGATGAAGAAGGCTATTATGGTTTACATTTC
GATACCGATTATGACCTAGATCCTCGTCATGATTTATCTAAAGGCAGTGGTAAAACGTAT
CTATCATGGCAACCAACTATTGGACGTAACTCAAACTTCCTTGGATTAACAAGAGCCCAG
AAAGATGAATTAGGTGGTGTCGAGTACAGAGCAATCAAACTTTTATGCACCATATTGGTT
GTCTACTACGTTGGATGGCATATTGTTGCTTTTGTTATGTTAGTACCTTGGATTATTTTG
AAAAAGCATTATAGTGAAGTTGTTAGAGATGATGGTGTTTCACCTACATGGTGGGGATTT
TGGACAGCAATGAGTGCATTTAATGATTTAGGTTTGACATTAACTCCAAATTCAATGATG
TCGTTTAACAAAGCTGTATACCCATTGATCGTTATGATTTGGTTTATCATTATCGGAAAT
ACAGGGTTTCCCATCCTTCTTAGATGCATCATTTGGATAATGTTTAAAATTTCTCCTGAT
TTATCACAGATGAGAGAAAGTTTAGGTTTTCTCTTAGACCATCCACGTCGTTGTTTCACC
TTGCTATTTCCTAAGGCAGCTACATGGTGGCTACTTTTAACGCTTGCAGGATTGAATATA
ACTGATTGGATTTTATTTATTATTCTAGATTTTGGCTCAACAGTTGTGAAATCATTATCG
AAAGGCTATAGAGTCCTTGTCGGCCTGTTTCAATCTGTTAGCACAAGAACTGCTGGATTC
AGCGTTGTCGATTTAAGTCAACTGCATCCTTCTATCCAAGTCTCCTATATGCTAATGATG
TATGTCTCCGTATTACCATTGGCCATCTCTATTCGACGGACAAATGTTTACGAGGAGCAA
TCTTTAGGACTATATGGAGATATGGGGGGAGAACCAGAAGATACGGATACTGAAGACGAT
GGTAACGATGAAGATGACGACGAGGAAAACGAGAGTCACGAAGGTCAAAGTAGTCAAAGA
AGTAGTTCGAACAACAACAACAATAACAACAGGAAAAAGAAAAAGAAAAAGAAAACTGAA
AATCCAAATGAAATATCTACAAAATCCTTTATCGGTGCCCATTTAAGGAAACAGCTTTCA
TTTGACTTGTGGTTTCTATTTTTAGGGTTATTTATCATTTGCATTTGTGAAGGGGACAAG
ATAAAGGACGTACAAGAACCAAACTTTAATATATTTGCAATTCTTTTTGAAATTGTTAGC
GCTTACGGTACAGTTGGGCTATCGCTAGGTTATCCGGACACCAACCAATCGTTTTCAAGA
CAGTTTACTACATTATCTAAGTTGGTGATCATAGCTATGCTGATCAGAGGCAAGAATAGA
GGTCTACCATACTCACTGGATCGTGCAATTATCTTGCCTAGTGATAGACTTGAACATATT
GACCACCTTGAGGGCATGAAATTGAAGAGACAGGCTAGAACCAATACAGAAGACCCAATG
ACGGAACATTTCAAGAGAAGTTTCACTGATGTGAAACATCGTTGGGGAGCTCTTAAGCGT
AAGACCACACATTCCCGAAATCCTAAAAGGAGCAGCACAACGCTCTAA
SEQ ID NO: 42
nucleic acid sequence HSP31
ATGGCCCCAAAAAAAGTTTTACTCGCTCTTACCTCATATAACGATGTATTCTACAGTGAC
GGCGCCAAGACCGGTGTTTTTGTTGTAGAAGCCTTACATCCCTTCAACACATTCCGAAAA
GAAGGTTTTGAAGTCGATTTTGTATCTGAAACCGGAAAATTTGGTTGGGATGAGCATTCC
TTAGCCAAAGATTTTCTAAATGGTCAAGACGAAACGGATTTTAAAAATAAAGACTCAGAT
TTCAACAAGACATTGGCTAAAATTAAGACACCAAAAGAGGTGAATGCCGATGATTACCAA
ATTTTTTTTGCATCTGCAGGCCACGGTACCTTATTTGACTATCCTAAGGCTAAAGACTTG
CAGGACATTGCTTCCGAAATTTATGCTAACGGTGGTGTTGTCGCAGCTGTTTGTCACGGT
CCTGCTATTTTTGATGGGTTAACAGACAAAAAAACAGGAAGACCATTGATCGAAGGTAAA
TCTATCACCGGTTTTACTGATGTTGGTGAAACCATTTTGGGTGTTGATAGTATTTTGAAA
GCCAAGAATTTGGCAACCGTTGAAGATGTTGCTAAAAAATATGGCGCTAAGTATTTAGCT
CCGGTTGGGCCCTGGGATGATTACTCTATTACTGACGGAAGACTGGTAACAGGTGTGAAT
CCTGCTTCTGCGCACTCCACTGCCGTAAGATCCATCGACGCTTTAAAAAACTGA
SEQ ID NO: 43
nucleic acid sequence HSP33
ATGACTCCAAAAAGAGCGCTAATATCTCTTACTTCATACCACGGTCCCTTCTACAAAGAT
GGTGCGAAAACAGGCGTTTTTGTAGTTGAGATTTTGCGATCGTTCGATACATTCGAAAAG
CATGGTTTCGAAGTGGACTTCGTTTCTGAGACTGGTGGATTTGGCTGGGATGAACATTAC
TTGCCAAAGAGCTTTATTGGTGGCGAAGATAAGATGAACTTTGAAACGAAAAATTCCGCC
TTCAATAAGGCGTTAGCGAGGATCAAGACCGCAAATGAAGTCAACGCCAGCGACTATAAA
GTATTCTTTGCATCTGCTGGACATGGTGCTCTATTTGACTATCCCAAAGCTAAAAATCTG
CAAGATATTGCATCCAAGATATATGCCAATGGGGGTGTGATCGCTGCCATCTGTCATGGA
CCGCTCCTTTTCGATGGATTAATAGATATCAAAACAACAAGACCATTAATCGAAGGCAAA
GCTATAACAGGTTTCCCACTCGAGGGTGAAATCGCCCTGGGAGTTGACGACATCTTGAGG
AGCAGAAAATTGACAACGGTTGAACGCGTTGCAAACAAGAATGGAGCCAAGTACTTGGCG
CCAATCCATCCCTGGGATGACTACTCTATTACAGATGGAAAGCTAGTTACGGGTGTTAAC
GCAAATTCTTCCTATTCGACCACAATTAGAGCTATAAACGCATTATATAGCTGA
SEQ ID NO: 44
nucleic acid sequence HSP30
ATGAACGATACGCTATCAAGCTTTTTAAATCGTAACGAGGCTTTAGGGCTTAATCCACCA
CATGGCCTGGATATGCACATTACCAAGAGAGGTTCGGATTGGTTATGGGCAGTGTTTGCA
GTCTTTGGCTTTATATTGCTATGCTATGTTGTGATGTTCTTCATTGCGGAGAACAAGGGC
TCCAGATTGACTAGATATGCCTTAGCTCCTGCATTTTTGATCACTTTCTTTGAATTTTTT
GCTTTCTTCACTTATGCTTCTGATTTAGGTTGGACTGGTGTTCAAGCTGAATTTAACCAC
GTCAAGGTTAGCAAGTCTATCACAGGTGAAGTTCCCGGTATTAGACAAATCTTTTACTCG
AAATATATTGCCTGGTTCTTGTCCTGGCCATGCCTTTTATTTTTAATCGAGTTAGCCGCT
AGTACTACTGGTGAGAATGACGACATTTCCGCCTTGGATATGGTACATTCGCTGTTAATT
CAAATCGTGGGTACCTTATTCTGGGTTGTTTCGCTATTAGTTGGTTCATTGATCAAGTCC
ACCTACAAGTGGGGTTATTACACCATTGGTGCTGTCGCTATGTTGGTTACCCAAGGTGTG
ATATGCCAACGTCAATTCTTCAATTTGAAAACTAGAGGGTTCAATGCACTTATGCTGTGT
ACCTGCATGGTAATCGTTTGGTTGTACTTTATCTGTTGGGGTCTAAGTGATGGTGGTAAC
CGTATTCAACCAGACGGTGAGGCTATCTTTTATGGTGTTTTGGATTTATGTGTATTTGCC
ATTTATCCATGTTACTTGCTAATTGCAGTCAGCCGTGATGGCAAATTGCCAAGGCTATCT
TTGACAGGAGGATTCTCTCATCACCATGCTACGGACGATGTGGAAGATGCGGCTCCTGAA
ACAAAAGAAGCTGTTCCAGAGAGCCCAAGAGCATCTGGAGAGACTGCAATCCACGAACCC
GAACCTGAAGCAGAGCAAGCTGTCGAAGATACTGCTTAG
SEQ ID NO: 45
nucliec acid sequence HSP32
ATGACTCCAAAAAGAGCGCTAATATCTCTTACTTCATACCACGGTCCCTTCTATAAAGAT
GGTGCGAAAACAGGCGTTTTTGTAGTTGAGATTTTGCGGTCGTTCGATACTTTCGAAAAG
CATGGTTTCGAAGTGGACTTCGTTTCTGAGACTGGTGGATTTGGCTGGGATGAACATTAC
TTGCCAAAGAGCTTTATTGGTGGCGAAGATAAGATGAACTTTGAAACGAAAAATTCCGCC
TTCAATAAGGCGTTAGCGAGGATCAAGACCGCAAATGAAGTCAACGCCAGCGACTATAAA
ATATTCTTTGCATCTGCTGGACATGGTGCTCTATTTGACTATCCCAAAGCTAAAAATCTG
CAAGATATTGCATCCAAGATATATGCCAATGGGGGTGTGATCGCTGCCATCTGTCATGGA
CCGCTCCTTTTCGATGGATTAATAGATATCAAAACAACAAGACCATTAATCGAAGGCAAA
GCTATAACAGGTTTCCCACTCGAGGGTGAAATCGCCCTGGGAGTTGACGACATCTTGAGG
AGCAGAAAATTGACAACGGTTGAACGCGTTGCAAACAAGAATGGAGCCAAGTACTTGGCG
CCAATCCATCCCTGGGATGACTACTCTATTACAGATGGAAAGCTAGTTACGGGTGTTAAC
GCAAATTCTTCCTATTCGACCACAATTAGAGCTATAAACGCATTATATAGCTGA
SEQ ID NO: 46
nucleic acid sequence ADH6
ATGTCTTATCCTGAGAAATTTGAAGGTATCGCTATTCAATCACACGAAGATTGGAAAAAC
CCAAAGAAGACAAAGTATGACCCAAAACCATTTTACGATCATGACATTGACATTAAGATC
GAAGCATGTGGTGTCTGCGGTAGTGATATTCATTGTGCAGCTGGTCATTGGGGCAATATG
AAGATGCCGCTAGTCGTTGGTCATGAAATCGTTGGTAAAGTTGTCAAGCTAGGGCCCAAG
TCAAACAGTGGGTTGAAAGTCGGTCAACGTGTTGGTGTAGGTGCTCAAGTCTTTTCATGC
TTGGAATGTGACCGTTGTAAGAATGATAATGAACCATACTGCACCAAGTTTGTTACCACA
TACAGTCAGCCTTATGAAGACGGCTATGTGTCGCAGGGTGGCTATGCAAACTACGTCAGA
GTTCATGAACATTTTGTGGTGCCTATCCCAGAGAATATTCCATCACATTTGGCTGCTCCA
CTATTATGTGGTGGTTTGACTGTGTACTCTCCATTGGTTCGTAACGGTTGCGGTCCAGGT
AAAAAAGTTGGTATAGTTGGTCTTGGTGGTATCGGCAGTATGGGTACATTGATTTCCAAA
GCCATGGGGGCAGAGACGTATGTTATTTCTCGTTCTTCGAGAAAAAGAGAAGATGCAATG
AAGATGGGCGCCGATCACTACATTGCTACATTAGAAGAAGGTGATTGGGGTGAAAAGTAC
TTTGACACCTTCGACCTGATTGTAGTCTGTGCTTCCTCCCTTACCGACATTGACTTCAAC
ATTATGCCAAAGGCTATGAAGGTTGGTGGTAGAATTGTCTCAATCTCTATACCAGAACAA
CACGAAATGTTATCGCTAAAGCCATATGGCTTAAAGGCTGTCTCCATTTCTTACAGTGCT
TTAGGTTCCATCAAAGAATTGAACCAACTCTTGAAATTAGTCTCTGAAAAAGATATCAAA
ATTTGGGTGGAAACATTACCTGTTGGTGAAGCCGGCGTCCATGAAGCCTTCGAAAGGATG
GAAAAGGGTGACGTTAGATATAGATTTACCTTAGTCGGCTACGACAAAGAATTTTCAGAC
TAG
SEQ ID NO: 47
nucleic acid sequence UFD4
ATGTCTGAAAATAATTCGCACAACCTTGATGAACATGAGTCCCATAGCGAAAACAGTGAT
TATATGATGGATACGCAGGTAGAAGATGACTATGATGAGGATGGCCATGTACAGGGTGAG
TACTCTTATTATCCTGATGAAGATGAAGATGAACATATGCTTTCTAGCGTCGGAAGTTTT
GAGGCAGATGATGGTGAAGATGACGATAACGATTACCATCATGAAGATGATTCTGGACTT
TTATATGGATATCATAGAACTCAGAATGGCAGTGACGAAGACAGAAATGAAGAAGAAGAT
GGACTTGAACGTTCTCACGATAATAATGAATTTGGCAGCAACCCCCTACATTTACCTGAC
ATTTTGGAAACATTCGCACAAAGACTAGAACAAAGAAGACAAACAAGTGAAGGACTTGGG
CAACACCCGGTTGGAAGAACACTACCCGAGATTTTATCGATGATTGGAGGAAGGATGGAG
AGGAGCGCAGAGAGTTCGGCAAGGAATGAGCGGATTTCTAAATTGATAGAGAATACTGGG
AATGCCTCCGAGGATCCTTATATTGCAATGGAGAGTTTAAAAGAACTTTCTGAAAACATA
TTAATGATGAATCAAATGGTTGTCGATAGAATTATACCGATGGAAACCTTAATAGGTAAT
ATAGCTGCCATACTCTCTGATAAAATTTTACGGGAAGAATTAGAATTACAAATGCAAGCT
TGTAGATGCATGTATAATCTTTTTGAGGTCTGCCCTGAATCTATTTCAATAGCTGTTGAT
GAACACGTTATACCAATTTTACAAGGAAAATTGGTAGAGATCAGTTACATTGACCTCGCA
GAACAAGTTTTAGAAACGGTGGAATATATTTCTAGAGTACATGGGAGAGACATTTTAAAA
ACGGGCCAATTATCAATCTACGTCCAATTCTTCGATTTTTTAACTATACATGCGCAGAGG
AAGGCTATCGCAATTGTTTCGAACGCCTGTAGCAGTATCCGAACGGATGACTTTAAGACC
ATTGTTGAAGTACTTCCAACGCTGAAGCCAATTTTCTCGAATGCGACAGACCAACCAATA
TTAACCAGGCTTGTAAATGCCATGTACGGTATTTGCGGGGCGTTGCATGGGGTTGACAAA
TTTGAGACTTTGTTTTCGTTGGATTTAATCGAAAGAATAGTTCAGCTAGTTTCTATTCAG
GATACCCCCTTGGAGAATAAACTGAAATGTTTGGATATTTTAACCGTATTAGCGATGAGT
AGTGATGTACTTTCAAGAGAACTGAGAGAGAAAACTGACATTGTCGACATGGCAACACGA
TCATTCCAGCATTATAGTAAAAGTCCTAACGCAGGGTTACATGAAACACTGATTTATGTC
CCAAACAGTTTATTGATTAGCATTTCTAGATTTATAGTTGTATTGTTTCCTCCCGAGGAT
GAAAGAATACTGTCAGCGGATAAATATACCGGAAATAGCGACCGTGGCGTAATTTCTAAC
CAGGAAAAGTTTGATTCCCTAGTTCAATGTCTAATTCCAATTCTCGTTGAAATTTATACA
AATGCTGCTGACTTTGACGTAAGAAGATACGTACTTATTGCTTTACTGAGGGTTGTATCA
TGCATAAATAATTCCACAGCAAAGGCAATCAATGATCAACTTATTAAGTTAATCGGATCT
ATCCTGGCCCAAAAAGAAACAGCGTCTAACGCTAATGGTACTTACTCATCAGAAGCTGGT
ACACTGTTGGTTGGTGGTCTCTCGTTGCTTGACTTAATTTGTAAAAAGTTTTCCGAACTG
TTCTTTCCTTCCATCAAAAGAGAGGGCATTTTTGATTTGGTTAAGGATTTGTCTGTGGAT
TTCAATAACATTGATTTAAAGGAAGACGGGAATGAAAATATTTCACTTTCTGACGAAGAA
GGGGATTTGCATAGCAGTATTGAGGAATGTGATGAGGGTGATGAAGAATATGATTACGAA
TTTACTGATATGGAAATTCCTGATTCAGTAAAACCAAAGAAAATTTCAATACACATTTTC
AGAACTCTATCTCTAGCTTATATTAAAAACAAGGGTGTGAACCTAGTTAATAGAGTACTT
TCTCAGATGAACGTTGAGCAAGAAGCAATAACAGAGGAGCTCCATCAAATCGAAGGCGTT
GTTTCTATTTTAGAAAATCCTTCCACTCCGGACAAAACTGAAGAGGATTGGAAGGGAATT
TGGTCTGTTTTAAAAAAATGTATTTTCCATGAAGATTTCGACGTGTCAGGTTTCGAATTT
ACTTCTACAGGGCTAGCTTCCTCCATAACTAAAAGAATTACATCCTCAACGGTATCCCAT
TTCATTCTTGCTAAATCATTTTTAGAGGTATTTGAGGATTGTATTGACAGATTTTTAGAA
ATCCTACAATCTGCTCTCACAAGGCTGGAGAATTTCTCTATAGTTGATTGCGGTTTACAC
GATGGTGGTGGTGTATCTTCACTGGCTAAAGAGATAAAAATTAAGTTGGTTTATGATGGC
GATGCAAGCAAAGATAATATTGGTACTGATTTATCATCTACTATCGTTTCGGTCCATTGC
ATAGCTTCTTTTACCTCACTTAATGAGTTTTTGAGACACAGAATGGTAAGAATGCGTTTT
TTGAATTCATTAATCCCAAACCTTACATCTTCCAGTACCGAAGCTGATAGGGAAGAAGAA
GAAAATTGCTTGGATCATATGAGAAAAAAGAACTTTGACTTTTTTTATGATAATGAAAAA
GTTGACATGGAGTCTACAGTATTTGGTGTGATATTTAATACATTCGTCAGGCGAAATCGT
GACTTAAAAACTTTATGGGATGATACACATACAATCAAATTTTGCAAAAGTTTAGAAGGT
AACAATAGAGAGAGTGAGGCAGCCGAGGAAGCTAATGAGGGGAAAAAGTTAAGAGATTTT
TATAAAAAAAGAGAATTCGCACAGGTTGATACTGGATCTTCAGCGGATATTCTGACATTG
CTGGATTTTCTACATAGCTGCGGTGTTAAAAGCGACAGTTTTATCAACTCAAAACTAAGC
GCTAAGCTCGCTAGACAACTAGATGAACCATTGGTAGTAGCAAGTGGAGCTTTGCCGGAT
TGGTCACTATTTTTGACCAGGAGATTCCCATTTTTGTTTCCGTTTGATACCAGGATGCTT
TTCCTACAATGTACTTCATTTGGTTACGGAAGATTGATTCAACTTTGGAAGAATAAGAGT
AAAGGCTCGAAAGATTTAAGGAATGACGAAGCTTTACAACAACTTGGGAGAATTACTAGG
CGTAAGCTGCGGATTTCAAGAAAAACAATATTCGCTACCGGTCTCAAGATTTTATCCAAG
TACGGAAGTAGCCCTGACGTACTGGAAATTGAATATCAAGAAGAAGCAGGAACAGGTTTA
GGACCGACTTTGGAATTTTACTCCGTAGTTTCCAAGTATTTTGCAAGAAAGTCGTTAAAT
ATGTGGCGTTGTAACTCTTATAGTTACAGAAGCGAAATGGATGTTGATACTACTGACGAT
TATATTACCACTTTATTGTTCCCAGAGCCCCTCAACCCGTTTTCCAATAATGAAAAAGTT
ATTGAACTTTTTGGATATTTGGGGACATTTGTTGCCAGATCGTTGCTTGATAATAGAATT
CTTGACTTTAGATTTAGCAAAGTCTTTTTTGAGTTATTGCACAGAATGTCTACGCCCAAT
GTGACGACAGTGCCGAGCGACGTTGAAACCTGTCTGTTAATGATCGAATTGGTAGATCCG
TTACTCGCAAAATCCCTTAAATACATAGTAGCGAATAAGGATGACAATATGACCCTAGAA
TCGTTGTCCTTGACATTTACCGTTCCTGGAAATGATGACATTGAGTTGATTCCGGGGGGT
TGTAATAAATCCTTGAACTCTTCTAATGTTGAAGAATATATCCATGGCGTTATCGACCAA
ATATTAGGTAAGGGCATTGAAAAACAGTTAAAAGCATTTATTGAAGGTTTTTCAAAGGTG
TTTTCCTATGAGAGGATGCTAATACTTTTTCCGGATGAATTAGTGGATATTTTCGGACGA
GTTGAGGAAGACTGGTCTATGGCAACTTTATACACAAACTTGAACGCTGAACATGGCTAT
ACAATGGATTCTTCAATCATTCATGATTTTATATCAATAATATCCGCGTTTGGTAAGCAT
GAAAGAAGATTATTTTTGCAATTTTTAACGGGATCTCCCAAGCTTCCAATTGGGGGATTT
AAAAGTTTGAACCCAAAGTTTACAGTTGTGTTAAAGCATGCTGAAGATGGCCTAACAGCA
GACGAATATCTACCAAGTGTAATGACATGTGCTAATTATTTGAAATTGCCGAAGTATACT
AGCAAAGATATTATGCGGTCTCGTCTTTGTCAAGCCATTGAAGAGGGTGCAGGAGCTTTT
CTACTTTCCTAA
SEQ ID NO: 48
nucleic acid sequence PRO1
ATGAAGGATGCTAATGAGAGTAAATCGTATACTATAGTGATCAAATTAGGCTCTTCATCG
CTAGTAGATGAAAAAACCAAAGAACCTAAGTTAGCTATCATGTCGCTTATTGTCGAAACT
GTAGTCAAATTGAGAAGAATGGGACACAAAGTTATCATCGTGTCCAGTGGTGGTATTGCT
GTTGGTTTGAGGACTATGCGTATGAATAAAAGACCAAAACATTTAGCAGAAGTTCAGGCC
ATCGCAGCTATTGGGCAGGGTAGATTGATCGGGAGATGGGATCTTCTGTTTTCGCAATTT
GATCAACGTATCGCTCAAATTCTATTGACCAGAAATGATATTCTGGACTGGACCCAATAT
AAGAACGCTCAAAACACAATTAATGAATTGTTGAACATGGGCGTTATTCCCATTGTGAAT
GAAAACGACACACTATCTGTTAGAGAAATCAAATTTGGTGACAATGACACTTTATCAGCA
ATTACTTCTGCTTTAATCCATGCAGATTATCTTTTCTTACTGACAGATGTTGACTGTTTG
TATACTGATAATCCAAGGACAAACCCAGATGCCATGCCGATCTTAGTTGTCCCAGATCTC
TCAAAGGGTTTGCCCGGTGTGAATACTGCTGGTGGTTCAGGTTCTGACGTTGGGACCGGT
GGTATGGAAACTAAATTGGTTGCTGCAGATTTGGCAACGAATGCCGGTGTTCATACGTTG
ATCATGAAAAGCGATACACCTGCGAATATAGGTAGAATTGTCGAGTATATGCAAACTCTA
GAACTTGACGATGAAAATAAAGTTAAACAAGCATATAATGGCGATTTAACGGATTTGCAA
AAAAGAGAATTTGAGAAATTAAAGGCTCTTAACGTTCCACTACATACGAAGTTCATTGCT
AATGATAATAAACACCATCTAAAGAATAGAGAGTTTTGGATTTTACACGGTCTTGTCTCT
AAAGGCGCTGTTGTTATAGACCAAGGTGCGTACGCAGCCTTAACAAGGAAAAATAAGGCG
GGATTATTGCCAGCAGGTGTTATTGATGTTCAGGGCACTTTCCATGAGTTAGAATGTGTT
GACATAAAAGTTGGTAAAAAGTTACCAGATGGCACGTTAGATCCAGATTTTCCCTTGCAA
ACAGTAGGCAAGGCAAGATGCAATTACACGAGTTCTGAATTAACTAAAATTAAAGGTTTG
CACAGTGACCAAATCGAAGAGGAATTGGGCTATAATGACAGCGAATATGTCGCTCATAGA
GAAAATTTGGCATTCCCACCTCGTTGA
SEQ ID NO: 49
nucleic acid sequence SIA1
ATGAGATTACATTATAGAAGAAGATTTAATTTTTTAAGGAGAATACTTTTTATATTATGC
ATTACTTCATTGTATTTATCGAGAGATTCACTAAAGCTACATGCAAAAAATGTATTAATG
GATCATAATGTAGCAGAATATCATGGCGGAATGATAGACGATATTCAAATCCTGCGGTGC
TACCATTGGTACAGGCAATGTAGTTCTTTGTATGCCCCGAAATTACACCCCTCCAATACT
GCTAAAAAGATCAAAGACAAAAACAGCATTCTGTGGACCAGAGTTTCTAAGAATATTACT
GTAGAGACATTGTATTCACTTCAGTCTGGACCATTCTACAACAGTTACTTATATGTTCAT
CTGAAAGATTTCCAAAGTAATCCAAAAAACACAATAAAAGAACTAGCCATAGCAAGGGAC
TCAGCACTAATACCCCTACAAGTGCTGAGAGACATTAATAAGTTGGTGAAATCGAGCGAC
AGTTCTGTCTTTCACAATCATGTGTATCTACGAGAAAAGCCTACTTCGTCATGGTGGAAG
CTGCTTTTCGGCATATCCGTTGATACAGATAACATAGCTGTGTTCGGTGAGGAGTGGGTA
TACAAGGGGAGCGGCATATGGTGTAAGTATATCCTTAATGATGATGATAATGACGCTCCT
ATAACTAATTTGGAAATATATCTAGGATCATCGTTTATTGAATCGAGGCCTTCTTGGAAA
GAAGTTATCCATGAATTTCATAGAAATAACATACCTTCTCTGCCCATATCAATTACAAGA
AAGCTTGAAACCAAAAACCATCATCACAAATTTTCTAATGGATTGCTAGGTTCTTTGAGA
ACACCCAGCAAAGACATTAATATCCAAGTCGATGCAGATTACAAAATAACATCTCCCCAT
ATACAATTTTCGAGGGGACAAAGATCATTCAAAATTCTCCAAATAACTGATTTTCATTTC
AAATGTACGGATAATAGCATGACCGTAATCAATGAAATAAAAACAGTAAATTTTATTGAT
AGGGTACTCGCATCAGAAAACCCTGATTTAGTTGTGATCACAGGTGATTTGTTAGACTCA
CATAATACTATCGACTATCAAACGTGCATTATGAAAGTTGTCCAACCAATGATTTCTAAT
AAAATACCCTACGCAATTTCATTGGGTGTTTCTGACGAATCCAATTTGGCCACATCGGCA
CAAATTAGAGACTTTATCAGGAATTTACCTTACACATTTAACAACGTTGCATCAGAAGAG
GGTCATATGGCCATAGAAGTCTCATTTAAAAAGAAGCTCACGAAGAATACTCTTTTGGAA
AGAGACATTGATACCGAAGACGAAACAAACCCATCAGAGGCTTTATTTTTCGTCTTTGAT
TCATTTGCGCCCGTCAATAATTTCCTACAAGATTATAACGACCTGATTGGGAAAATAGAC
TTTGGCTTGGCATTTCAATATTTTCCATTATCGGAATATAGGCCTCATGGTTTATTTCCT
ATTATTGGGCAGTATAATGAGAGGTCTACCTTAACAGTAGATACGCCAAGGTCTAGAGGA
CAAGTTTCAATGACGATCAATGGCAAACATTACAAAAGCTTCCTTGATATCCTGAGTCTT
TGGAATATAAAGGGTGTCAGTTGCGGACATGAACATAATAATGACTGTTGCTTACAGTCA
AAAAATGAGATGTGGTTATGTTACGGTGGGTCCGCTGGTATAGGCTTGCCGAGAATCCAA
GGTATATATCCAACCGTTAGATTATTTAACTTGGATGATATTTTGGACGAAATAACTTCG
TGGAAGAGGAATAGCAATCTTGTTGACGAGGTTTACGATTATCAGTACATCTATAAGGGG
AAGCAATAA
SEQ ID NO: 50
nucleic acid sequence ARI1
ATGACTACTGATACCACTGTTTTCGTTTCTGGCGCAACCGGTTTCATTGCTCTACACATT
ATGAACGATCTGTTGAAAGCTGGCTATACAGTCATCGGCTCAGGTAGATCTCAAGAAAAA
AATGATGGCTTGCTCAAAAAATTTAATAACAATCCCAAACTATCGATGGAAATTGTGGAA
GATATTGCTGCTCCAAACGCCTTTGATGAAGTTTTCAAAAAACATGGTAAGGAAATTAAG
ATTGTGCTACACACTGCCTCCCCATTCCATTTTGAAACTACCAATTTTGAAAAGGATTTA
CTAACCCCTGCAGTGAACGGTACAAAATCTATCTTGGAAGCGATTAAAAAATATGCTGCA
GACACTGTTGAAAAAGTTATTGTTACTTCGTCTACTGCTGCTCTGGTGACACCTACAGAC
ATGAACAAAGGAGATTTGGTGATCACGGAGGAGAGTTGGAATAAGGATACATGGGACAGT
TGTCAAGCCAACGCCGTTGCCGCATATTGTGGCTCGAAAAAGTTTGCTGAAAAAACTGCT
TGGGAATTTCTTAAAGAAAACAAGTCTAGTGTCAAATTCACACTATCCACTATCAATCCG
GGATTCGTTTTTGGTCCTCAAATGTTTGCAGATTCGCTAAAACATGGCATAAATACCTCC
TCAGGGATCGTATCTGAGTTAATTCATTCCAAGGTAGGTGGAGAATTTTATAATTACTGT
GGCCCATTTATTGACGTGCGTGACGTTTCTAAAGCCCACCTAGTTGCAATTGAAAAACCA
GAATGTACCGGCCAAAGATTAGTATTGAGTGAAGGTTTATTCTGCTGTCAAGAAATCGTT
GACATCTTGAACGAGGAATTCCCTCAATTAAAGGGCAAGATAGCTACAGGTGAACCTGCG
ACCGGTCCAAGCTTTTTAGAAAAAAACTCTTGCAAGTTTGACAATTCTAAGACAAAAAAA
CTACTGGGATTCCAGTTTTACAATTTAAAGGATTGCATAGTTGACACCGCGGCGCAAATG
TTAGAAGTTCAAAATGAAGCCTAA
SEQ ID NO: 51
nucleic acid sequence LPP1
ATGATCTCTGTCATGGCGGATGAGAAACATAAGGAGTATTTTAAGCTATACTACTTTCAG
TACATGATAATTGGTCTATGTACGATATTATTCCTCTATTCGGAGATATCCCTGGTACCT
AGGGGCCAAAACATCGAATTTAGTCTTGATGACCCCAGTATATCAAAACGTTATGTACCT
AACGAACTCGTGGGCCCACTAGAATGTTTGATTTTGAGTGTTGGACTGAGTAACATGGTC
GTCTTCTGGACCTGCATGTTTGACAAGGACTTACTGAAGAAGAATAGAGTAAAGAGACTA
AGAGAGAGGCCGGACGGAATCTCGAACGATTTTCACTTCATGCATACTAGCATTCTATGT
CTGATGCTGATTATAAGCATAAATGCTGCCCTAACAGGCGCCTTAAAGTTGATTATAGGA
AACTTGAGGCCTGACTTTGTTGATAGATGTATACCTGACCTCCAAAAGATGAGTGATTCA
GATTCTTTGGTTTTTGGCTTGGACATTTGCAAGCAGACTAACAAATGGATTCTATACGAA
GGCTTAAAAAGCACTCCAAGCGGACATTCAAGTTTCATAGTCAGTACCATGGGCTTTACA
TATCTTTGGCAAAGGGTTTTCACCACACGCAATACAAGAAGTTGCATTTGGTGCCCTTTA
TTAGCTCTAGTAGTAATGGTTTCAAGGGTTATCGATCACAGACATCATTGGTACGATGTT
GTCTCTGGAGCTGTTCTAGCATTTTTAGTCATTTATTGTTGCTGGAAATGGACATTTACA
AACTTGGCGAAAAGAGACATACTTCCTTCACCGGTTAGTGTTTAG
SEQ ID NO: 52
nucleic acid sequence PMA2
ATGTCTTCCACTGAAGCAAAGCAATACAAGGAGAAACCCTCGAAAGAGTACCTCCATGCC
AGTGATGGCGATGACCCTGCAAATAATTCTGCCGCTTCTTCGTCATCTTCGTCTTCTACA
TCAACTTCCGCCTCGTCATCGGCTGCAGCCGTTCCACGGAAGGCCGCAGCCGCTTCTGCC
GCTGATGATTCTGACTCAGATGAAGATATAGACCAATTGATTGATGAACTACAATCTAAC
TACGGTGAGGGTGATGAATCTGGTGAAGAAGAAGTACGTACTGATGGGGTGCACGCTGGC
CAAAGGGTTGTTCCTGAAAAGGACCTTTCTACGGACCCTGCGTATGGTTTGACTTCGGAT
GAAGTCGCCAGGAGAAGAAAGAAATATGGGTTAAATCAAATGGCTGAGGAGAATGAATCG
TTGATTGTGAAGTTTTTGATGTTCTTCGTAGGGCCTATTCAATTCGTTATGGAGGCTGCT
GCTATTTTGGCTGCCGGTTTGTCTGATTGGGTTGATGTCGGTGTCATCTGTGCTTTACTG
CTATTAAACGCATCTGTCGGATTTATTCAAGAATTCCAGGCAGGTTCCATCGTAGACGAG
CTGAAAAAGACGTTGGCCAATACTGCAACAGTTATTAGAGATGGCCAATTGATCGAAATT
CCGGCTAATGAGGTAGTTCCTGGTGAGATTTTGCAATTGGAAAGTGGCACAATTGCTCCC
GCAGATGGTCGTATTGTCACTGAAGACTGTTTTTTGCAGATCGATCAATCGGCCATCACT
GGTGAATCCTTAGCCGCTGAAAAGCATTACGGTGATGAGGTGTTCTCCTCATCCACTGTG
AAAACCGGCGAGGCTTTTATGGTTGTTACTGCCACTGGTGACAATACCTTCGTCGGTAGG
GCTGCCGCCTTAGTGGGGCAGGCTTCCGGTGTAGAGGGCCATTTCACTGAAGTATTGAAT
GGAATTGGTATTATCTTACTTGTTCTAGTTATCGCTACTTTGTTGTTGGTCTGGACCGCA
TGTTTCTATAGAACGGTCGGTATTGTAAGCATTTTGAGATATACTTTGGGTATAACCATC
ATTGGTGTCCCAGTCGGTTTGCCAGCAGTTGTTACCACGACCATGGCTGTCGGTGCAGCT
TACTTGGCTAAGAAGCAAGCCATTGTTCAAAAGTTATCTGCTATTGAATCCCTTGCTGGT
GTTGAGATTTTATGTTCTGACAAGACTGGTACTTTAACCAAAAACAAGTTATCTTTACAC
GAACCCTACACTGTCGAAGGCGTTTCTCCGGACGACTTGATGTTGACCGCTTGTTTAGCT
GCCTCTAGAAAGAAGAAAGGTTTGGATGCTATTGATAAGGCTTTTTTGAAGTCATTGATT
GAGTATCCAAAAGCTAAAGACGCCCTGACCAAGTACAAAGTTTTGGAATTCCATCCGTTC
GACCCTGTCTCAAAAAAGGTTACCGCTGTTGTAGAATCCCCAGAAGGTGAAAGAATTGTT
TGTGTCAAGGGAGCCCCATTGTTTGTCTTGAAGACTGTTGAAGAAGATCACCCAATTCCG
GAAGATGTGCATGAAAACTACGAAAATAAGGTTGCTGAACTAGCTTCTAGAGGTTTCCGT
GCTTTAGGTGTTGCTAGAAAGAGAGGGGAAGGTCACTGGGAAATCTTGGGTGTTATGCCA
TGTATGGACCCCCCTAGAGATGACACCGCTCAAACAATCAATGAGGCCAGAAACCTTGGT
TTGAGAATCAAGATGTTAACCGGTGACGCTGTTGGTATCGCGAAAGAAACGTGTAGGCAA
TTAGGACTTGGTACAAACATTTATAACGCAGAAAGGTTAGGTCTGGGAGGTGGAGGTGAT
ATGCCTGGTTCAGAGTTGGCTGATTTTGTTGAAAATGCCGATGGTTTCGCAGAAGTTTTC
CCACAGCATAAATACAGAGTCGTTGAAATCTTGCAAAACAGAGGTTACTTGGTTGCTATG
ACTGGTGATGGTGTTAACGATGCCCCATCTTTGAAGAAGGCTGATACTGGTATTGCTGTC
GAAGGTGCTACCGATGCTGCCAGATCAGCCGCTGATATTGTTTTCTTGGCCCCTGGTCTC
TCTGCTATTATTGATGCCTTAAAGACTTCTAGACAGATTTTCCACAGAATGTACTCCTAT
GTTGTTTATCGTATTGCCCTATCCTTACATTTGGAGATTTTCCTGGGTTTATGGATTGCT
ATTTTAAACAACTCTTTGGATATCAATTTGATCGTTTTTATTGCTATTTTCGCAGACGTT
GCCACTTTAACTATTGCTTATGACAATGCTCCTTATGCTCCTGAACCTGTGAAATGGAAC
CTACCAAGATTATGGGGTATGTCTATTATTTTGGGCATAGTTTTAGCTATAGGTTCTTGG
ATTACTTTAACCACCATGTTCTTGCCTAATGGTGGTATTATCCAAAATTTTGGTGCCATG
AATGGTGTCATGTTCCTGCAGATTTCACTAACTGAAAATTGGTTAATTTTTGTCACTAGA
GCTGCTGGCCCATTCTGGTCTTCCATTCCATCGTGGCAGTTAGCCGGTGCCGTTTTCGCC
GTTGATATTATTGCTACCATGTTTACCTTATTTGGCTGGTGGTCTGAAAACTGGACTGAT
ATTGTGTCAGTCGTTCGTGTCTGGATTTGGTCCATTGGTATTTTTTGTGTATTGGGAGGA
TTTTACTATATTATGTCCACGTCTCAAGCCTTTGATAGGTTGATGAATGGTAAGTCATTA
AAGGAAAAGAAGTCTACAAGAAGTGTCGAAGATTTCATGGCTGCTATGCAAAGAGTTTCT
ACTCAACACGAAAAAAGCAGTTAG
SEQ ID NO: 53
nucleic acid sequence PDR12
ATGTCTTCGACTGACGAACATATTGAGAAAGACATTTCGTCGAGATCGAACCATGACGAT
GATTATGCTAATTCGGTACAATCCTACGCTGCCTCCGAAGGCCAAGTTGATAATGAGGAT
TTGGCAGCCACTTCTCAGCTATCCCGTCACCTTTCAAACATTCTTTCCAATGAAGAAGGT
ATTGAAAGGTTGGAGTCTATGGCGAGAGTCATTTCACATAAGACAAAGAAGGAAATGGAC
TCTTTTGAAATTAATGACTTAGATTTTGATTTGCGCTCACTATTACATTATTTGAGGTCT
CGTCAATTGGAACAGGGAATTGAACCTGGTGATTCTGGTATTGCCTTTAAAAACCTAACA
GCAGTCGGTGTTGATGCCTCTGCTGCATATGGGCCTAGTGTTGAAGAGATGTTTAGAAAT
ATTGCTAGTATACCGGCACATCTCATAAGTAAATTTACCAAGAAATCTGATGTCCCATTA
AGGAATATTATTCAAAATTGTACGGGTGTCGTTGAATCTGGTGAAATGTTATTTGTCGTC
GGTAGGCCAGGTGCAGGTTGCTCCACTTTCCTAAAGTGTCTATCTGGTGAAACTTCAGAA
TTAGTTGATGTACAAGGTGAATTCTCCTATGATGGTCTGGACCAAAGCGAAATGATGTCT
AAGTATAAAGGTTACGTTATTTACTGTCCCGAGCTTGATTTCCATTTCCCAAAAATTACT
GTGAAGGAAACAATCGATTTTGCCCTAAAATGTAAGACTCCTCGTGTTAGAATTGACAAA
ATGACGAGAAAGCAATACGTTGATAACATCAGAGATATGTGGTGTACCGTTTTTGGTTTA
AGACACACATATGCCACCAAAGTCGGTAACGATTTCGTAAGAGGTGTTTCTGGTGGTGAA
CGTAAGCGTGTTTCCTTGGTTGAAGCTCAGGCAATGAATGCCTCCATCTACTCTTGGGAT
AACGCCACAAGAGGTTTGGATGCCTCTACTGCTTTAGAGTTTGCCCAAGCCATTAGAACG
GCTACAAATATGGTAAACAACTCTGCTATTGTTGCTATTTACCAAGCTGGTGAAAATATT
TATGAATTATTTGATAAAACTACTGTTCTATATAACGGTAGACAGATTTACTTCGGTCCT
GCTGATAAAGCTGTTGGATATTTCCAAAGAATGGGTTGGGTTAAACCAAACAGAATGACC
TCTGCGGAATTTTTAACATCCGTCACGGTCGATTTTGAAAATAGGACATTGGATATTAAA
CCTGGCTATGAAGATAAAGTTCCAAAATCTAGTTCAGAGTTTGAGGAATACTGGTTGAAC
TCTGAGGATTATCAGGAACTTTTAAGAACTTATGATGATTATCAAAGTAGACACCCTGTT
AATGAAACGAGAGATAGACTGGATGTGGCCAAGAAGCAAAGACTGCAACAAGGCCAAAGA
GAAAATTCTCAATATGTTGTCAATTATTGGACACAAGTTTATTATTGTATGATTCGTGGT
TTTCAAAGGGTTAAGGGTGATTCAACGTATACTAAGGTCTACTTAAGTTCTTTTTTGATC
AAAGCTTTGATTATCGGTTCTATGTTCCACAAAATTGATGACAAAAGTCAATCCACCACG
GCAGGTGCTTATTCTCGTGGTGGTATGTTATTCTATGTTTTATTGTTCGCTTCTGTTACT
TCCTTGGCCGAAATTGGTAACTCTTTTTCTAGTAGACCTGTTATTGTCAAACACAAATCA
TATTCCATGTACCATTTGTCTGCGGAATCGTTACAAGAGATTATCACTGAGTTCCCTACT
AAATTTGTCGCTATTGTGATACTATGTTTGATTACTTACTGGATTCCATTTATGAAATAT
GAAGCTGGTGCTTTCTTCCAGTATATTTTATATCTACTGACTGTGCAACAATGTACTTCT
TTCATTTTCAAGTTTGTTGCTACTATGAGTAAATCTGGTGTGGATGCCCATGCCGTCGGT
GGTTTATGGGTCCTGATGCTTTGTGTTTATGCTGGTTTTGTCTTGCCAATTGGTGAAATG
CATCATTGGATTAGATGGCTTCATTTCATTAATCCTTTAACTTATGCTTTTGAAAGTTTA
GTTTCCACTGAATTTCACCACAGGGAAATGTTGTGTAGCGCCTTAGTCCCATCTGGTCCT
GGTTATGAAGGTATTTCTATTGCTAACCAAGTCTGTGATGCTGCTGGTGCGGTTAAGGGT
AACTTGTATGTTAGCGGTGACTCTTACATCTTACACCAATATCATTTCGCATATAAGCAT
GCTTGGAGAAATTGGGGTGTGAACATTGTGTGGACTTTTGGTTATATTGTGTTCAATGTC
ATCTTATCAGAATATTTGAAACCTGTTGAGGGAGGAGGTGACTTGCTGTTATATAAGAGA
GGTCATATGCCGGAGTTAGGTACCGAAAATGCAGATGCAAGAACCGCTTCCAGAGAGGAA
ATGATGGAGGCTCTGAATGGTCCAAATGTCGATTTAGAAAAGGTCATTGCAGAAAAGGAC
GTTTTCACCTGGAACCATCTGGACTACACCATTCCATACGACGGAGCTACAAGAAAATTA
TTATCGGATGTCTTTGGTTACGTTAAGCCTGGTAAGATGACCGCCTTGATGGGTGAATCC
GGTGCTGGTAAAACTACCTTGTTAAATGTTTTAGCACAAAGAATCAATATGGGTGTCATC
ACTGGTGATATGTTAGTCAATGCCAAGCCCTTGCCTGCTTCTTTCAACAGATCATGTGGT
TATGTTGCGCAAGCCGATAATCATATGGCCGAATTATCTGTTAGGGAATCCCTGAGATTT
GCAGCCGAGTTAAGACAGCAAAGTTCCGTTCCGTTAGAGGAGAAATATGAATATGTTGAA
AAAATTATCACATTGCTAGGTATGCAAAATTACGCTGAAGCCTTAGTTGGTAAGACTGGT
AGAGGTTTGAACGTTGAACAGAGAAAGAAGTTATCTATTGGTGTTGAACTGGTTGCTAAA
CCATCATTATTATTGTTTTTGGATGAGCCTACCTCTGGTCTGGACTCTCAGTCTGCTTGG
TCAATTGTTCAATTCATGAGAGCCTTAGCTGATTCTGGTCAATCCATTTTGTGTACGATT
CATCAACCCTCTGCTACCTTGTTTGAACAGTTTGACAGATTGTTGTTGTTAAAGAAAGGT
GGTAAGATGGTTTACTTTGGTGACATTGGTCCAAATTCTGAAACTTTGTTGAAGTATTTT
GAACGTCAATCTGGTATGAAGTGTGGTGTTTCTGAAAATCCAGCTGAATATATTTTGAAT
TGTATTGGTGCCGGTGCCACTGCTAGTGTTAACTCTGATTGGCACGACTTATGGCTTGCT
TCCCCAGAATGTGCCGCTGCAAGGGCTGAAGTTGAAGAATTACATCGTACTTTACCTGGT
AGAGCAGTTAATGATGATCCTGAGTTAGCTACAAGATTTGCTGCCAGTTACATGACTCAA
ATCAAATGTGTTTTACGTAGAACAGCTCTTCAATTTTGGAGATCGCCTGTCTATATCAGG
GCCAAATTCTTTGAATGTGTCGCATGTGCTTTGTTCGTCGGTTTATCATATGTTGGTGTA
AATCACTCTGTTGGTGGTGCCATTGAGGCCTTTTCGTCTATTTTCATGCTATTATTGATT
GCTCTGGCTATGATCAATCAACTGCACGTCTTCGCTTATGATAGTAGGGAATTATATGAG
GTTAGAGAAGCCGCTTCTAACACTTTCCATTGGAGTGTCTTGTTATTATGTCATGCTGCT
GTTGAAAACTTTTGGTCCACACTTTGTCAGTTTATGTGTTTCATTTGCTACTACTGGCCA
GCTCAATTCAGTGGACGTGCATCTCATGCAGGTTTCTTCTTCTTCTTCTATGTTTTAATT
TTCCCATTATATTTTGTCACATATGGTCTATGGATCCTGTACATGTCTCCTGATGTTCCC
TCAGCTTCTATGATTAATTCCAATTTGTTTGCTGCTATGTTACTGTTCTGTGGTATTTTA
CAACCAAGAGAGAAAATGCCTGCCTTCTGGAGAAGATTGATGTATAATGTATCACCATTT
ACCTACGTGGTTCAAGCTTTGGTTACACCATTAGTTCACAATAAAAAGGTCGTTTGTAAT
CCTCATGAATACAACATCATGGACCCACCAAGCGGAAAAACTTGTGGTGAGTTTTTATCT
ACCTATATGGACAATAATACCGGTTATTTGGTAAATCCAACTGCCACCGAAAACTGTCAA
TATTGCCCATACACTGTTCAAGATCAAGTTGTGGCTAAATACAATGTCAAATGGGATCAC
AGATGGAGAAACTTTGGTTTCATGTGGGCTTATATTTGCTTCAATATTGCCGCTATGTTG
ATTTGTTACTATGTTGTAAGAGTTAAGGTGTGGTCTTTGAAGTCTGTTTTGAATTTCAAG
AAATGGTTTAATGGGCCAAGAAAGGAAAGACATGAAAAAGATACCAACATTTTCCAAACA
GTTCCAGGTGACGAAAATAAAATCACGAAGAAATAA
SEQ ID NO: 54
nucleic acid sequence ZWF1
ATGAGTGAAGGCCCCGTCAAATTCGAAAAAAATACCGTCATATCTGTCTTTGGTGCGTCA
GGTGATCTGGCAAAGAAGAAGACTTTTCCCGCCTTATTTGGGCTTTTCAGAGAAGGTTAC
CTTGATCCATCTACCAAGATCTTCGGTTATGCCCGGTCCAAATTGTCCATGGAGGAGGAC
CTGAAGTCCCGTGTCCTACCCCACTTGAAAAAACCTCACGGTGAAGCCGATGACTCTAAG
GTCGAACAGTTCTTCAAGATGGTCAGCTACATTTCGGGAAATTACGACACAGATGAAGGC
TTCGACGAATTAAGAACGCAGATCGAGAAATTCGAGAAAAGTGCCAACGTCGATGTCCCA
CACCGTCTCTTCTATCTGGCCTTGCCGCCAAGCGTTTTTTTGACGGTGGCCAAGCAGATC
AAGAGTCGTGTGTACGCAGAGAATGGCATCACCCGTGTAATCGTAGAGAAACCTTTCGGC
CACGACCTGGCCTCTGCCAGGGAGCTGCAAAAAAACCTGGGGCCCCTCTTTAAAGAAGAA
GAGTTGTACAGAATTGACCATTACTTGGGTAAAGAGTTGGTCAAGAATCTTTTAGTCTTG
AGGTTCGGTAACCAGTTTTTGAATGCCTCGTGGAATAGAGACAACATTCAAAGCGTTCAG
ATTTCGTTTAAAGAGAGGTTCGGCACCGAAGGCCGTGGCGGCTATTTCGACTCTATAGGC
ATAATCAGAGACGTGATGCAGAACCATCTGTTACAAATCATGACTCTCTTGACTATGGAA
AGACCGGTGTCTTTTGACCCGGAATCTATTCGTGACGAAAAGGTTAAGGTTCTAAAGGCC
GTGGCCCCCATCGACACGGACGACGTCCTCTTGGGCCAGTACGGTAAATCTGAGGACGGG
TCTAAGCCCGCCTACGTGGATGATGACACTGTAGACAAGGACTCTAAATGTGTCACTTTT
GCAGCAATGACTTTCAACATCGAAAACGAGCGTTGGGAGGGCGTCCCCATCATGATGCGT
GCCGGTAAGGCTTTGAATGAGTCCAAGGTGGAGATCAGACTGCAGTACAAAGCGGTCGCA
TCGGGTGTCTTCAAAGACATTCCAAATAACGAACTGGTCATCAGAGTGCAGCCCGATGCC
GCTGTGTACCTAAAGTTTAATGCTAAGACCCCTGGTCTGTCAAATGCTACCCAAGTCACA
GATCTGAATCTAACTTACGCAAGCAGGTACCAAGACTTTTGGATTCCAGAGGCTTACGAG
GTGTTGATAAGAGACGCCCTACTGGGTGACCATTCCAACTTTGTCAGAGATGACGAATTG
GATATCAGTTGGGGCATATTCACCCCATTACTGAAGCACATAGAGCGTCCGGACGGTCCA
ACACCGGAAATTTACCCCTACGGATCAAGAGGTCCAAAGGGATTGAAGGAATATATGCAA
AAACACAAGTATGTTATGCCCGAAAAGCACCCTTACGCTTGGCCCGTGACTAAGCCAGAA
GATACGAAGGATAATTAG
SEQ ID NO: 55
LCB2 amino acid sequence; systematic name YDR062W
MSTPANYTRVPLCEPEELPDDIQKENEYGTLDSPGHLYQVKSRHGKPLPEPVVDTPPYYI
SLLTYLNYLILIILGHVHDFLGMTFQKNKHLDLLEHDGLAPWFSNFESFYVRRIKMRIDD
CFSRPTTGVPGRFIRCIDRISHNINEYFTYSGAVYPCMNLSSYNYLGFAQSKGQCTDAAL
ESVDKYSIQSGGPRAQIGTTDLHIKAEKLVARFIGKEDALVFSMGYGTNANLFNAFLDKK
CLVISDELNHTSIRTGVRLSGAAVRTFKHGDMVGLEKLIREQIVLGQPKTNRPWKKILIC
AEGLFSMEGTLCNLPKLVELKKKYKCYLFIDEAHSIGAMGPTGRGVCEIFGVDPKDVDIL
MGTFTKSFGAAGGYIAADQWIIDRLRLDLTTVSYSESMPAPVLAQTISSLQTISGEICPG
QGTERLQRIAFNSRYLRLALQRLGFIVYGVADSPVIPLLLYCPSKMPAFSRMMLQRRIAV
VVVAYPATPLIESRVRFCMSASLTKEDIDYLLRHVSEVGDKLNLKSNSGKSSYDGKRQRW
DIEEVIRRTPEDCKDDKYFVN*
SEQ ID NO: 56
CHAT amino acid sequence; systematic name YCL064C
MSIVYNKTPLLRQFFPGKASAQFFLKYECLQPSGSFKSRGIGNLIMKSAIRIQKDGKRSP
QVFASSGGNAGFAAATACQRLSLPCTVVVPTATKKRMVDKIRNTGAQVIVSGAYWKEADT
FLKTNVMNKIDSQVIEPIYVHPFDNPDIWEGHSSMIDEIVQDLKSQHISVNKVKGIVCSV
GGGGLYNGIIQGLERYGLADRIPIVGVETNGCHVFNTSLKIGQPVQFKKITSIATSLGTA
VISNQTFEYARKYNTRSVVIEDKDVIETCLKYTHQFNMVIEPACGAALHLGYNTKILENA
LGSKLAADDIVIIIACGGSSNTIKDLEEALDSMRKKDTPVIEVADNFIFPEKNIVNLKSA
*
SEQ ID NO: 57
HXT5 amino acid sequence; systematic name YHR096C
MSELENAHQGPLEGSATVSTNSNSYNEKSGNSTAPGTAGYNDNLAQAKPVSSYISHEGPP
KDELEELQKEVDKQLEKKSKSDLLFVSVCCLMVAFGGFVFGWDTGTISGFVRQTDFIRRF
GSTRANGTTYLSDVRTGLMVSIFNIGCAIGGIVLSKLGDMYGRKIGLMTVVVIYSIGIII
QIASIDKWYQYFIGRIISGLGVGGITVLAPMLISEVSPKQLRGTLVSCYQLMITFGIFLG
YCTNFGTKNYSNSVQWRVPLGLCFAWSIFMIVGMTFVPESPRYLVEVGKIEEAKRSLARA
NKTTEDSPLVTLEMENYQSSIEAERLAGSASWGELVTGKPQMFRRTLMGMMIQSLQQLTG
DNYFFYYGTTIFQAVGLEDSFETAIVLGVVNFVSTFFSLYTVDRFGRRNCLLWGCVGMIC
CYVVYASVGVTRLWPNGQDQPSSKGAGNCMIVFACFYIFCFATTWAPVAYVLISESYPLR
VRGKAMSIASACNWIWGFLISFFTPFITSAINFYYGYVFMGCMVFAYFYVFFFVPETKGL
TLEEVNEMYEENVLPWKSTKWIPPSRRTTDYDLDATRNDPRPFYKRMFTKEK*
SEQ ID NO: 58
MTD1 amino acid sequence; systematic name YKR080W
MSKPGRTILASKVAETFNTEIINNVEEYKKTHNGQGPLLVGFLANNDPAAKMYATWTQKT
SESMGFRYDLRVIEDKDFLEEAIIQANGDDSVNGIMVYFPVFGNAQDQYLQQVVCKEKDV
EGLNHVYYQNLYHNVRYLDKENRLKSILPCTPLAIVKILEFLKIYNNLLPEGNRLYGKKC
IVINRSEIVGRPLAALLANDGATVYSVDVNNIQKFTRGESLKLNKHHVEDLGEYSEDLLK
KCSLDSDVVITGVPSENYKFPTEYIKEGAVCINFACTKNFSDDVKEKASLYVPMTGKVTI
AMLLRNMLRLVRNVELSKEK*
SEQ ID NO: 59
MSC6 amino acid sequence; systematic name YOR354C
MLSHNALRAFDCSKVIISRRCLTSSTSIYQQSSVHLQETDDGHSGNREKHVSPFERVQNL
AADLKNELKAPDSDINEVFNDFKDKIESLKQKLRNPSPMERSHLLANFSSDLLQELSYRS
KNMTLDPYQVLNTLCQYKLARSQHFTIVLKYLLYNQSPQDVIALWVKYLETISENPVILL
QNSSSRAHMQNIAITTIAYLSLPENTVDINTLYKILQIDRKMGQVLPFNMIRRMLSTEFS
SLERRDVIIKNLNTLYYQYTVQDSDHFLSQIENAPRWIDLRDLYGQYNKLEGEKNVEIIS
KFMDKFIDLDKPDQVVTIYNQYSKVFPNSTSLKDCLLRAVSHLRAKSSKEKLDRILAVWN
SVIKPGDNIKNTSYATLVNALTDSGNFNHLKEFWEEELPKKFKKDPIVKEAFLLALCQTS
PLKYDQVKGELAETVKTKKLFNKVLLLMLDDEKVSEEQFNTFYYNHYPSDGVLPPTLDTL
SIKMYANYKFQAEDTRPQFDLLQSVSINPTDYEKVEKITKAFISVCPTVEPIRQLYKQLG
THLNARNYADFISAEFNKPDGTVAEAKNLFSDFLSYQKTRKRNVDNTPLNALLLGFCDKL
YKSKHSEYVPYIEKYYNLAKDSSIRVSNLAVSKILFNLATFARNTQQLSDKEVAFINQFM
RDLGTNEGFRPNPKDIQILKECDGITVPEKLT*
SEQ ID NO: 60
SCW10 amino acid sequence; systematic name YMR305C
MRFSNFLTVSALLTGALGAPAVRHKHEKRDVVTATVHAQVTVVVSGNSGETIVPVNENAV
VATTSSTAVASQATTSTLEPTTSANVVTSQQQTSTLQSSEAASTVGSSTSSSPSSSSSTS
SSASSSASSSISASGAKGITYSPYNDDGSCKSTAQVASDLEQLTGFDNIRLYGVDCSQVE
NVLQAKTSSQKLFLGIYYVDKIQDAVDTIKSAVESYGSWDDITTVSVGNELVNGGSATTT
QVGEYVSTAKSALTSAGYTGSVVSVDTFIAVINNPDLCNYSDYMAVNAHAYFDENTAAQD
AGPWVLEQIERVYTACGGKKDVVITETGWPSKGDTYGEAVPSKANQEAAISSIKSSCGSS
AYLFTAFNDLWKDDGQYGVEKYWGILSSD*
SEQ ID NO: 61
YAL065C amino acid sequence; systematic name YAL065C
MNSATSETTTNTGAAETTTSTGAAETKTVVTSSISRFNHAETQTASATDVIGHSSSVVSV
SETGNTKSLITSGLSTMSQQPRSTPASSIIGSSTASLEISTYVGIANGLLTNNGISVFIS
TVLLAIVW*
SEQ ID NO: 62
YJL107C amino acid sequence; systematic name YJL107C
MDGRNEKPTTPVSDFRVGSSEQSQAGVNLEDSSDHRTSNSAESKKGNLSGKSISDLGISN
NDNKNVRFTADTDALENDLSSRSTETSDNSKGTDGQDEEDRPARHKRKPKVSFTHLRNNG
KDGDDETFIKKIINNLTGNQGGLVPGLAPIPSENENGKNDIEKNNRNEEIPLSDLADASK
IVDVHEGDDKEKLEALKLEGDVNCTSDGETLGSSSKNSFLAPAVDHFDDYAENNSSDDNE
GFIETSTYVPPPSQVKSGVLGSLLKLYQNEDQNSSSIFSDSQAVTTDDEGISSTAGNKDV
PVAKRSRLQNLKGKAKKGRMPRLKKRLKTEAKITVHIADILQRHRFILRMCRALMMYGAP
THRLEEYMVMTSRVLEIDGQFCIFQVV*
SEQ ID NO: 63
CSM3 amino acid sequence; systematic name YMR048W
MDQDFDSLLLGFNDSDSVQKDPTVPNGLDGSVVDPTIADPTAITARKRRPQVKLTAEKLL
SDKGLPYVLKNAHKRIRISSKKNSYDNLSNIIQFYQLWAHELFPKAKFKDFMKICQTVGK
TDPVLREYRVSLFRDEMGMSFDVGTRETGQDLERQSPMVEEHVTSAEERPIVADSFAQDK
RNVNNVDYDNDEDDDIYHLSYRNRRGRVLDERGNNETVLNNVVPPKEDLDALLKTFRVQG
PVGLEENEKKLLLGWLDAHRKMEKGSMTEEDVQLIQSLEEWEMNDIEGQHTHYDLLPGGD
EFGVDQDELDAMKEMGF*
SEQ ID NO: 64
RGT2 amino acid sequence; systematic name YDL138W
MNDSQNCLRQREENSHLNPGNDFGHHQGAECTINHNNMPHRNAYTESTNDTEAKSIVMCD
DPNAYQISYTNNEPAGDGAIETTSILLSQPLPLRSNVMSVLVGIFVAVGGFLFGYDTGLI
NSITDMPYVKTYIAPNHSYFTTSQIAILVSFLSLGTFFGALIAPYISDSYGRKPTIMFST
AVIFSIGNSLQVASGGLVLLIVGRVISGIGIGIISAVVPLYQAEAAQKNLRGAIISSYQW
AITIGLLVSSAVSQGTHSKNGPSSYRIPIGLQYVWSSILAVGMIFLPESPRYYVLKDELN
KAAKSLSFLRGLPIEDPRLLEELVEIKATYDYEASFGPSTLLDCFKTSENRPKQILRIFT
GIAIQAFQQASGINFIFYYGVNFFNNTGVDNSYLVSFISYAVNVAFSIPGMYLVDRIGRR
PVLLAGGVIMAIANLVIAIVGVSEGKTVVASKIMIAFICLFIAAFSATWGGVVWVVSAEL
YPLGVRSKCTAICAAANWLVNFTCALITPYIVDVGSHTSSMGPKIFFIWGGLNVVAVIVV
YFAVYETRGLTLEEIDELFRKAPNSVISSKWNKKIRKRCLAFPISQQIEMKTNIKNAGKL
DNNNSPIVQDDSHNIIDVDGFLENQIQSNDHMIAADKGSGSLVNIIDTAPLTSTEFKPVE
HPPVNYVDLGNGLGLNTYNRGPPSIISDSTDEFYEENDSSYYNNNTERNGANSVNTYMAQ
LINSSSTTSNDTSFSPSHNSNARTSSNWTSDLASKHSQYTSPQ*
SEQ ID NO: 65
CHS7 amino acid sequence; systematic name YHR142W
MAFSDFAAICSKTPLPLCSVIKSKTHLILSNSTIIHDFDPLNLNVGVLPRCYARSIDLAN
TVIFDVGNAFINIGALGVILIILYNIRQKYTAIGRSEYLYFFQLTLLLIIFTLVVDCGVS
PPGSGSYPYFVAIQIGLAGACCWALLIIGFLGFNLWEDGTTKSMLLVRGTSMLGFIANFL
ASILTFKAWITDHKVATMNASGMIVVVYIINAIFLFVFVICQLLVSLLVVRNLWVTGAIF
LGLFFFVAGQVLVYAFSTQICEGFKHYLDGLFFGSICNVFTLMMVYKTWDMTTDDDLEFG
VSVSKDGDVVYDNGFM*
SEQ ID NO: 66
BOP2 amino acid sequence; systematic name YLR267W
MVAALTYLPTELIQRIFEFTVVETDSQYWLYNLVALIDFSVSSRGGGSITEDFLTNYVRK
NLMVLDLTCEATQDSILRAEYGFLKRLLPYIDMDAQYIRVVDLETNADKAQNLKAEKLIV
IFDEFSDLKLIETFFPLANSNSNIIEFVFCVRNIKSSFYSPLEKLHIANIVADIDINTLY
LDFVDSNIYSDQNFFGIFDPDIFQLINKNYRNFFSKTNEKGKKRPPICKKICFPFVETLN
LDYMALDSFFNSILHKLTTKIKTFERNNEFDVDKNLNLNSTTTVAALIIKSILQQFFNNF
HISFPNLVTLNFIKMSTYPNNNEITQCCNFIDLSSYVLNKCLSENISINFLFQLHSLKNW
SMPKIKEFTGHKFKYDETTFSGSPERYIKSLRGNIKILQEMAINETNDGTCYFRVKLIPE
GVEKTQIINWIPFTSSFSDDTFKQRHHLKRPMICLKNNSLRSLTVKIIRIEKCSSIRIQG
FYLPNLQELFINNTLCDTTQHQKQASNDMSCIEFTSWNELPQCKKLGFAQLEDDSNYVLN
ISNLQDHLPNLDLRESFPTFFDIRQKFVVV*
SEQ ID NO: 67
YDR271C amino acid sequence; systematic name YDR271C
MNINYYYCYKSICSWIFLNKLDLPVIYKTSSFDISPACDSMSCSPAIARVEKSLDQKFPI
ENLDLKSEIPCDSISGGVHFFNINELRTTLTELNAIAKPASIGGRVMPQGMSTPIAIGIM
NIL*
SEQ ID NO: 68
PAU7 amino acid sequence; systematic name YAR020C
MVKLTSIAAGVAAIAAGASAAATTTLSQSDERVNLVELGVYVSDIRAHLAEYYSF*
SEQ ID NO: 69
YGL258W-A amino acid sequence; systematic name YGL258W-A
MAFERQGKIEKKISYSLFLNGPNVHFGSILFGAVDKSKYAEELCTHPMRQAYNTLDSNSR
IIITVQSVAILDGKLVW*
SEQ ID NO: 70
SLU7 amino acid sequence; systematic name YDR088C
MNNNSRNNENRSTINRNKRQLQQAKEKNENIHIPRYIRNQPWYYKDTPKEQEGKKPGNDD
TSTAEGGEKSDYLVHHRQKAKGGALDIDNNSEPKIGMGIKDEFKLIRPQKMSVRDSHSLS
FCRNCGEAGHKEKDCMEKPRKMQKLVPDLNSQKNNGTVLVRATDDDWDSRKDRWYGYSGK
EYNELISKWERDKRNKIKGKDKSQTDETLWDTDEEIELMKLELYKDSVGSLKKDDADNSQ
LYRTSTRLREDKAAYLNDINSTESNYDPKSRLYKTETLGAVDEKSKMFRRHLTGEGLKLN
ELNQFARSHAKEMGIRDEIEDKEKVQHVLVANPTKYEYLKKKREQEETKQPKIVSIGDLE
ARKVDGTKQSEEQRNHLKDLYG*
SEQ ID NO: 71
ARP6 amino acid sequence; systematic name YLR085C
METPPIVIDNGSYEIKFGPSTNKKPFRALNALAKDKFGTSYLSNHIKNIKDISSITFRRP
HELGQLTLWELESCIWDYCLFNPSEFDGFDLKEGKGHHLVASESCMTLPELSKHADQVIF
EEYEFDSLFKSPVAVFVPFTKSYKGEMRTISGKDEDIDIVRGNSDSTNSTSSESKNAQDS
GSDYHDFQLVIDSGFNCTWIIPVLKGIPYYKAVKKLDIGGRFLTGLLKETLSFRHYNMMD
ETILVNNIKEQCLFVSPVSYFDSFKTKDKHALEYVLPDFQTSFLGYVRNPRKENVPLPED
AQIITLTDELFTIPETFFHPEISQITKPGIVEAILESLSMLPEIVRPLMVGNIVCTGGNF
NLPNFAQRLAAELQRQLPTDWTCHVSVPEGDCALFGWEVMSQFAKTDSYRKARVTREEYY
EHGPDWCTKHRFGYQNWI*
SEQ ID NO: 72
MRP21 amino acid sequence; systematic name YBL090W
MLKSTLRLSRISLRRGFTTIDCLRQQNSDIDKIILNPIKLAQGSNSDRGQTSKSKTDNAD
ILSMEIPVDMMQSAGRINKRELLSEAEIARSSVENAQMRFNSGKSIIVNKNNPAESFKRL
NRIMFENNIPGDKRSQRFYMKPGKVAELKRSQRHRKEFMMGFKRLIEIVKDAKRKGY*
SEQ ID NO: 73
AFG2 amino acid sequence; systematic name YLR397C
MAPKSSSSGSKKKSSASSNSADAKASKFKLPAEFITRPHPSKDHGKETCTAYIHPNVLSS
LEINPGSFCTVGKIGENGILVIARAGDEEVHPVNVITLSTTIRSVGNLILGDRLELKKAQ
VQPPYATKVTVGSLQGYNILECMEEKVIQKLLDDSGVIMPGMIFQNLKTKAGDESIDVVI
TDASDDSLPDVSQLDLNMDDMYGGLDNLFYLSPPFIFRKGSTHITFSKETQANRKYNLPE
PLSYAAVGGLDKEIESLKSAIEIPLHQPTLFSSFGVSPPRGILLHGPPGTGKTMLLRVVA
NTSNAHVLTINGPSIVSKYLGETEAALRDIFNEARKYQPSIIFIDEIDSIAPNRANDDSG
EVESRVVATLLTLMDGMGAAGKVVVIAATNRPNSVDPALRRPGRFDQEVEIGIPDVDARF
DILTKQFSRMSSDRHVLDSEAIKYIASKTHGYVGADLTALCRESVMKTIQRGLGTDANID
KFSLKVTLKDVESAMVDIRPSAMREIFLEMPKVYWSDIGGQEELKTKMKEMIQLPLEASE
TFARLGISAPKGVLLYGPPGCSKTLTAKALATESGINFLAVKGPEIFNKYVGESERAIRE
IFRKARSAAPSIIFFDEIDALSPDRDGSSTSAANHVLTSLLNEIDGVEELKGVVIVAATN
RPDEIDAALLRPGRLDRHIYVGPPDVNARLEILKKCTKKFNTEESGVDLHELADRTEGYS
GAEVVLLCQEAGLAAIMEDLDVAKVELRHFEKAFKGIARGITPEMLSYYEEFALRSGSSS
*
SEQ ID NO: 74
YJL152W amino acid sequence; systematic name YJL152W
MPHLAAEAHTWPPHISHSTLSIPHPTPEHRHVFHKKDVKNKRNEEKGNNLLYVLFRTTVI
KSSFRSLSTAGRELLFVVHQGHIGTGLIVFIICWRLCLRFLCRVSFQVTVYGGRSRMSA*
SEQ ID NO: 75
PPT2 amino acid sequence; systematic name YPL148C
MSFASRNIGRKIAGVGVDIVYLPRFAHILEKYSPFDPCGRSTLNKITRKFMHEKERFHFS
NLLIEENCLTPRLHEYIAGVWALKECSLKALCCCVSKHDLPPAQVLYAGMLYKTQTDTGV
PQLEFDKMFGKKYPKYQQLSKNYDSLFSTHEFLVSLSHDKDYLIAVTNLVERE*
SEQ ID NO: 76
PGS1 amino acid sequence; systematic name YCL004W
MTTRLLQLTRPHYRLLSLPLQKPFNIKRQMSAANPSPFGNYLNTITKSLQQNLQTCFHFQ
AKEIDIIESPSQFYDLLKTKILNSQNRIFIASLYLGKSETELVDCISQALTKNPKLKVSF
LLDGLRGTRELPSACSATLLSSLVAKYGSERVDCRLYKTPAYHGWKKVLVPKRFNEGLGL
QHMKIYGFDNEVILSGANLSNDYFTNRQDRYYLFKSRNFSNYYFKLHQLISSFSYQIIKP
MVDGSINTIWPDSNPTVEPTKNKRLFLREASQLLDGFLKSSKQSLPITAVGQFSTLVYPI
SQFTPLFPKYNDKSTEKRTILSLLSTITSNAISWTFTAGYFNILPDIKAKLLATPVAEAN
VITASPFANGFYQSKGVSSNLPGAYLYLSKKFLQDVCRYRQDHAITLREWQRGVVNKPNG
WSYHAKGIWLSARDKNDANNWKPFITVIGSSNYTRRAYSLDLESNALIITRDEELRKKMK
AELDNLLQYTKPVTLEDFQSDPERHVGTGVKIATSILGKKL*
SEQ ID NO: 77
YHC1 amino acid sequence; systematic name YLR2989C
MTRYYCEYCHSYLTHDTLSVRKSHLVGKNHLRITADYYRNKARDIINKHNHKRRHIGKRG
RKERENSSQNETLKVTCLSNKEKRHIMHVKKMNQKELAQTSIDTLKLLYDGSPGYSKVFV
DANRFDIGDLVKASKLPQRANEKSAHHSFKQTSRSRDETCESNPFPRLNNPKKLEPPKIL
SQWSNTIPKTSIFYSVDILQTTIKESKKRMHSDGIRKPSSANGYKRRRYGN*
SEQ ID NO: 78
YJL045W amino acid sequence; systematic name YJL045W
MLSLKKGITKSYILQRTFTSSSVVRQIGEVKSESKPPAKYHIIDHEYDCVVVGAGGAGLR
AAFGLAEAGYKTACLSKLFPTRSHTVAAQGGINAALGNMHPDDWKSHMYDTVKGSDWLGD
QDAIHYMTREAPKSVIELEHYGMPFSRTEDGRIYQRAFGGQSKDFGKGGQAYRTCAVADR
TGHAMLHTLYGQALKNNTHFFIEYFAMDLLTHNGEVVGVIAYNQEDGTIHRFRAHKTVIA
TGGYGRAYFSCTSAHTCTGDGNAMVSRAGFPLEDLEFVQFHPSGIYGSGCLITEGARGEG
GFLLNSEGERFMERYAPTAKDLASRDVVSRAITMEIRAGRGVGKNKDHILLQLSHLPPEV
LKERLPGISETAAVFAGVDVTQEPIPVLPTVHYNMGGIPTKWTGEALTIDEETGEDKVIP
GLMACGEAACVSVHGANRLGANSLLDLVVFGRAVANTIADTLQPGLPHKPLASNIGHESI
ANLDKVRNARGSLKTSQIRLNMQRTMQKDVSVFRTQDTLDEGVRNITEVDKTFEDVHVSD
KSMIWNSDLVETLELQNLLTCATQTAVSASKRKESRGAHAREDYAKRDDVNWRKHTLSWQ
KGTSTPVKIKYRNVIAHTLDENECAPVPPAVRSY*
SEQ ID NO: 79
NDD1 amino acid sequence; systematic name YOR372C
MDRDISYQQNYTSTGATATSSRQPSTDNNADTNFLKVMSEFKYNFNSPLPTTTQFPTPYS
SNQYQQTQDHFANTDAHNSSSNESSLVENSILPHHQQIQQQQQQQQQQQQQQQALGSLVP
PAVTRTDTSETLDDINVQPSSVLQFGNSLPSEFLVASPEQFKEFLLDSPSTNFNFFHKTP
AKTPLRFVTDSNGAQQSTTENPGQQQNVFSNVDLNNLLKSNGKTPSSSCTGAFSRTPLSK
IDMNLMFNQPLPTSPSKRFSSLSLTPYGRKILNDVGTPYAKALISSNSALVDFQKARKDI
TTNATSIGLENANNILQRTPLRSNNKKLFIKTPQDTINSTSTLTKDNENKQDIYGSSPTT
IQLNSSITKSISKLDNSRIPLLASRSDNILDSNVDDQLFDLGLTRLPLSPTPNCNSLHST
TTGTSALQIPELPKMGSFRSDTGINPISSSNTVSFKSKSGNNNSKGRIKKNGKKPSKFQI
IVANIDQFNQDTSSSSLSSSLNASSSAGNSNSNVTKKRASKLKRSQSLLSDSGSKSQARK
SCNSKSNGNLFNSQ*
SEQ ID NO: 80
KEX2 amino acid sequence; systematic name YNL238W
MKVRKYITLCFWWAFSTSALVSSQQIPLKDHTSRQYFAVESNETLSRLEEMHPNWKYEHD
VRGLPNHYVFSKELLKLGKRSSLEELQGDNNDHILSVHDLFPRNDLFKRLPVPAPPMDSS
LLPVKEAEDKLSINDPLFERQWHLVNPSFPGSDINVLDLWYNNITGAGVVAAIVDDGLDY
ENEDLKDNFCAEGSWDFNDNTNLPKPRLSDDYHGTRCAGEIAAKKGNNFCGVGVGYNAKI
SGIRILSGDITTEDEAASLIYGLDVNDIYSCSWGPADDGRHLQGPSDLVKKALVKGVTEG
RDSKGAIYVFASGNGGTRGDNCNYDGYTNSIYSITIGAIDHKDLHPPYSEGCSAVMAVTY
SSGSGEYIHSSDINGRCSNSHGGTSAAAPLAAGVYTLLLEANPNLTWRDVQYLSILSAVG
LEKNADGDWRDSAMGKKYSHRYGFGKIDAHKLIEMSKTWENVNAQTWFYLPTLYVSQSTN
STEETLESVITISEKSLQDANFKRIEHVTVTVDIDTEIRGTTTVDLISPAGIISNLGVVR
PRDVSSEGFKDWTFMSVAHWGENGVGDWKIKVKTTENGHRIDFHSWRLKLFGESIDSSKT
ETFVFGNDKEEVEPAATESTVSQYSASSTSISISATSTSSISIGVETSAIPQTTTASTDP
DSDPNTPKKLSSPRQAMHYFLTIFLIGATFLVLYFMFFMKSRRRIRRSRAETYEFDIIDT
DSEYDSTLDNGTSGITEPEEVEDFDFDLSDEDHLASLSSSENGDAEHTIDSVLTNENPFS
DPIKQKFPNDANAESASNKLQELQPDVPPSSGRS*
SEQ ID NO: 81
COG7 amino acid sequence; systematic name YGL005C
MVELTITGDDDDILSMFFDEEFVPHAFVDILLSNALNEDQIQTQSVSSLLLTRLDFYTKN
LTKELESTIWNLDKLSQTLPRTWASSRYHKEAEQNDSSLYSTESLKSSKLEYYLDTLASA
VRALETGMHNVTEKLSDLDNENNRNTNVRQQLQSLMLIKERIEKVVYYLEQVRTVTNIST
VRENNTTSTGTDLSITDFRTSLKALEDTIDESLSSAIDNEAKDETNKDLIGRIDSLSELK
CLFKGLDKFFAEYSNFSESIKSKAQSYLSTKNIDDGMIS*
SEQ ID NO: 82
PRP45 amino acid sequence; systematic name YAL032C
MFSNRLPPPKHSQGRVSTALSSDRVEPAILTDQIAKNVKLDDFIPKRQSNFELSVPLPTK
AEIQECTARTKSYIQRLVNAKLANSNNRASSRYVTETHQAPANLLLNNSHHIEVVSKQMD
PLLPRFVGKKARKVVAPTENDEVVPVLHMDGSNDRGEADPNEWKIPAAVSNWKNPNGYTV
ALERRVGKALDNENNTINDGFMKLSEALENADKKARQEIRSKMELKRLAMEQEMLAKESK
LKELSQRARYHNGTPQTGAIVKPKKQTSTVARLKELAYSQGRDVSEKIILGAAKRSEQPD
LQYDSRFFTRGANASAKRHEDQVYDNPLFVQQDIESIYKTNYEKLDEAVNVKSEGASGSH
GPIQFTKAESDDKSDNYGA*
SEQ ID NO: 83
MET16 amino acid sequence; systematic name YPR167C
MKTYHLNNDIIVTQEQLDHWNEQLIKLETPQEITAWSIVTFPHLFQTTAFGLTGLVTIDM
LSKLSEKYYMPELLFIDTLHHFPQTLTLKNEIEKKYYQPKNQTIHVYKPDGCESEADFAS
KYGDFLWEKDDDKYDYLAKVEPAHRAYKELHISAVFTGRRKSQGSARSQLSIIEIDELNG
ILKINPLINWTFEQVKQYIDANNVPYNELLDLGYRSIGDYHSTQPVKEGEDERAGRWKGK
AKTECGIHEASRFAQFLKQDA*
SEQ ID NO: 84
YGR114C amino acid sequence; systematic name YGR114C
MFSSFFGNTCSWVFIFIIIVDNEAFLHFSCLIFVFINIFVFLRGVKDIFSFFFLTRRFSF
IVVIYYFFLVPRDQLRISRLFHKRQILCKDSRQLMTCSLGLFFKAQINIFLPPFALTVVQ
FLVNLVCHT*
SEQ ID NO: 85
RGI2 amino acid sequence; systematic name YIL057C
MTKKDKKAKGPKMSTITTKSGESLKVFEDLHDFETYLKGETEDQEFDHVHCQLKYYPPFV
LHDAHDDPEKIKETANSHSKKFVRHLHQHVEKHLLKDIKTAINKPELKFHDKKKQESFDR
IVWNYGEETELNAKKFKVSVEVVCKHDGAMVDVDYKTEPLQPLI*
SEQ ID NO: 86
YOR318C amino acid sequence; systematic name YOR318C
MCTPTTCLLADRDKSGEDRHAETNVLQGMDMLLELLLPVYARLNESGWLLWFVFHDVYEA
VKMSTKESVHTRVINFPDILSTQQMRQGPSQIRTPLVMLLM*
SEQ ID NO: 87
RAM2 amino acid sequence; systematic name YKL019W
MEEYDYSDVKPLPIETDLQDELCRIMYTEDYKRLMGLARALISLNELSPRALQLTAEIID
VAPAFYTINNYRFNIVRHMMSESEDTVLYLNKELDWLDEVTLNNPKNYQINSYRQSLLKL
HPSPSFKRELPILKLMIDDDSKNYHVWSYRKWCCLFFSDFQHELAYASDLIETDIYNNSA
WTHRMFYWVNAKDVISKVELADELQFIMDKIQLVPQNISPWTYLRGFQELFHDRLQWDSK
VVDFATTFIGDVLSLPIGSPEDLPEIESSYALEFLAYHWGADPCTRDNAVKAYSLLAIKY
DPIRKNLWHHKINNLN*
SEQ ID NO: 88
YPR027C amino acid sequence; systematic name YPR027C
MVGIYRILASFVPLLGLLFAFHDDDMIDTVTIIKTVYETVTSTSTAPAPAATKSVSEKKL
DDTKLTLQVIQTMVSCFSVGENPANMISCGLGVVILMFSLIIELINKLENDGINEPQRLY
DLIKPKYVELPSNYVNEKIKTTFEPLDLYLGVNMNTSGSELNQNCLILKLGEKTALPFPG
LAQQICYTKGASNEFTNYKLSDIQGNLNENSQGIANGVFQKISNIRKISGNFKSQLYQIS
EKITDENWDGSAVGFTAHGREKGPNKSQISVSFYRDN*
SEQ ID NO: 89
MGR3 amino acid sequence; systematic name YMR115W
MLLQGMRLSQRLHKRHLFASKILTWTTNPAHIRHLHDIRPPASNFNTQESAPIPESPANS
PTRPQMAPKPNLKKKNRSLMYSIIGVSIVGLYFWFKSNSRKQKLPLSAQKVWKEAIWQES
DKMDFNYKEALRRYIEALDECDRSHVDLLSDDYTRIELKIAEMYEKLNMLEEAQNLYQEL
LSRFFEALNVPGKVDESERGEVLRKDLRILIKSLEINKDIESGKRKLLQHLLLAQEEILS
KSPELKEFFENRKKKLSMVKDINRDPNDDFKTFVSEENIKFDEQGYMILDLEKNSSANEP
FKEEFFTARDLYTAYCLSSKDIAAALSCKITSVEWMVMADMPPGQILLSQANLGSLFYLQ
AEKLEADLNQLEQKKSKESNQELDMGTYIKAVRFVRKNRDLCLERAQKCYDSVIAFAKRN
RKIRFHVKDQLDPSIAQSIALSTYGMGVLSLHEGVLAKAEKLFKDSITMAKETEFNELLA
EAEKELEKTTVLKAAKKEGLN*
SEQ ID NO: 90
FLO8 amino acid sequence; systematic name YER109C
MSYKVNSSYPDSIPPTEQPYMASQYKQDLQSNIAMATNSEQQRQQQQQQQQQQQQWINQP
TAENSDLKEKMNCKNTLNEYIFDFLTKSSLKNTAAAFAQDAHLDRDKGQNPVDGPKSKEN
NGNQNTFSKVVDTPQGFLYEWWQIFWDIFNTSSSRGGSEFAQQYYQLVLQEQRQEQIYRS
LAVHAARLQHDAERRGEYSNEDIDPMHLAAMMLGNPMAPAVQMRNVNMNPIPIPMVGNPI
VNNFSIPPYNNANPTTGATAVAPTAPPSGDFTNVGPTQNRSQNVTGWPVYNYPMQPTTEN
PVGNPCNNNTTNNTTNNKSPVNQPKSLKTMHSTDKPNNVPTSKSTRSRSATSKAKGKVKA
GLVAKRRRKNNTATVSAGSTNACSPNITTPGSTTSEPAMVGSRVNKTPRSDIATNFRNQA
IIFGEEDIYSNSKSSPSLDGASPSALASKQPTKVRKNTKKASTSAFPVESTNKLGGNSVV
TGKKRSPPNTRVSRRKSTPSVILNADATKDENNMLRTFSNTIAPNIHSAPPTKTANSLPF
PGINLGSFNKPAVSSPLSSVTESCFDPESGKIAGKNGPKRAVNSKVSASSPLSIATPRSG
DAQKQRSSKVPGNVVIKPPHGFSTTNLNITLKNSKIITSQNNTVSQELPNGGNILEAQVG
NDSRSSKGNRNTLSTPEEKKPSSNNQGYDFDALKNSSSLLFPNQAYASNNRTPNENSNVA
DETSASTNSGDNDNTLIQPSSNVGTTLGPQQTSTNENQNVHSQNLKFGNIGMVEDQGPDY
DLNLLDTNENDFNFINWEG*
SEQ ID NO: 91
BRE2 amino acid sequence; systematic name YLR015W
MKLGIIPYQEGTDIVYKNALQGQQEGKRPNLPQMEATHQIKSSVQGTSYEFVRTEDIPLN
RRHFVYRPCSANPFFTILGYGCTEYPFDHSGMSVMDRSEGLSISRDGNDLVSVPDQYGWR
TARSDVCIKEGMTYWEVEVIRGGNKKFADGVNNKENADDSVDEVQSGIYEKMHKQVNDTP
HLRFGVCRREASLEAPVGFDVYGYGIRDISLESIHEGKLNCVLENGSPLKEGDKIGFLLS
LPSIHTQIKQAKEFTKRRIFALNSHMDTMNEPWREDAENGPSRKKLKQETTNKEFQRALL
EDIEYNDVVRDQTATRYKNQLFFEATDYVKTTKPEYYSSDKRERQDYYQLEDSYLAIFQN
GKYLGKAFENLKPLLPPFSELQYNEKFYLGYWQHGEARDESNDKNTTSAKKKKQQQKKKK
GLILRNKYVNNNKLGYYPTISCFNGGTARIISEEDKLEYLDQIRSAYCVDGNSKVNTLDT
LYKEQIAEDIVWDIIDELEQIALQQ*
SEQ ID NO: 92
REC102 amino acid sequence; systematic name YLR329W
MARDITFLTVFLESCGAVNNDEAGKLLSAWTSTVRIEGPESTDSNSLYIPLLPPGMLKIK
LNFKMNDRLVTEEQELFTKLREIVGSSIRFWEEQLFYQVQDVSTIENHVILSLKCTILTD
AQISTFISKPRELHTHAKGYPEIYYLSELSTTVNFFSKEGNYVEISQVIPHFNEYFSSLI
VSQLEFEYPMVFSMISRLRLKWQQSSLAPISYALTSNSVLLPIMLNMIAQDKSSTTAYQI
LCRRRGPPIQNFQIFSLPAVTYNK*
SEQ ID NO: 93
IDP3 amino acid sequence; systematic name YNL009W
MSKIKVVHPIVEMDGDEQTRVIWKLIKEKLILPYLDVDLKYYDLSIQERDRTNDQVTKDS
SYATLKYGVAVKCATITPDEARMKEFNLKEMWKSPNGTIRNILGGTVFREPIIIPKIPRL
VPHWEKPIIIGRHAFGDQYRATDIKIKKAGKLRLQFSSDDGKENIDLKVYEFPKSGGIAM
AMFNTNDSIKGFAKASFELALKRKLPLFFTTKNTILKNYDNQFKQIFDNLFDKEYKEKFQ
ALKITYEHRLIDDMVAQMLKSKGGFIIAMKNYDGDVQSDIVAQGFGSLGLMTSILITPDG
KTFESEAAHGTVTRHFRKHQRGEETSTNSIASIFAWTRAIIQRGKLDNTDDVIKFGNLLE
KATLDTVQVGGKMTKDLALMLGKTNRSSYVTTEEFIDEVAKRLQNMMLSSNEDKKGMCKL
*
SEQ ID NO: 94
PEX18 amino acid sequence; systematic name YHR160C
MNSNRCQTNEVNKFISSTEKGPFTGRDNTLSFNKIGSRLNSPPILKDKIELKFLQHSEDL
NQSRSYVNIRPRTLEDQSYKFEAPNLNDNETSWAKDFRYNFPKNVEPPIENQIANLNINN
GLRTSQTDFPLGFYSQKNFNIASFPVVDHQIFKTTGLEHPINSHIDSLINAEFSELEASS
LEEDVHTEEENSGTSLEDEETAMKGLASDIIEFCDNNSANKDVKERLNSSKFMGLMGSIS
DGSIVLKKDNGTERNLQKHVGFCFQNSGNWAGLEFHDVEDRIA*
SEQ ID NO: 95
APS2 amino acid sequence; systematic name YJR058C
MAVQFILCFNKQGVVRLVRWFDVHSSDPQRSQDAIAQIYRLISSRDHKHQSNFVEFSDST
KLIYRRYAGLYFVMGVDLLDDEPIYLCHIHLFVEVLDAFFGNVCELDIVFNFYKVYMIMD
EMFIGGEIQEISKDMLLERLSILDRLD*
SEQ ID NO: 96
HUG1 amino acid sequence; systematic name YML058W-A
MTMDQGLNPKQFFLDDVVLQDTLCSMSNRVNKSVKTGYLFPKDHVPSANIIAVERRGGLS
DIGKNTSN*
SEQ ID NO: 97
OSH7 amino acid sequence; systematic name YHR001W
MALNKLKNIPSLTNSSHSSINGIASNAANSKPSGADTDDIDENDESGQSILLNIISQLKP
GCDLSRITLPTFILEKKSMLERITNQLQFPDVLLEAHSNKDGLQRFVKVVAWYLAGWHIG
PRAVKKPLNPILGEHFTAYWDLPNKQQAFYIAEQTSHHPPESAYFYMIPESNIRVDGVVV
PKSKFLGNSSAAMMEGLTVLQFLDIKDANGKPEKYTLSQPNVYARGILFGKMRIELGDHM
VIMGPKYQVDIEFKTKGFISGTYDAIEGTIKDYDGKEYYQISGKWNDIMYIKDLREKSSK
KTVLFDTHQHFPLAPKVRPLEEQGEYESRRLWKKVTDALAVRDHEVATEEKFQIENRQRE
LAKKRAEDGVEFHSKLFRRAEPGEDLDYYIYKHIPEGTDKHEEQIRSILETAPILPGQTF
TEKFSIPAYKKHGIQKN*
SEQ ID NO: 98
KSS1 amino acid sequence; systematic name YGR040W
MARTITFDIPSQYKLVDLIGEGAYGTVCSAIHKPSGIKVAIKKIQPFSKKLFVTRTIREI
KLLRYFHEHENIISILDKVRPVSIDKLNAVYLVEELMETDLQKVINNQNSGFSTLSDDHV
QYFTYQILRALKSIHSAQVIHRDIKPSNLLLNSNCDLKVCDFGLARCLASSSDSRETLVG
FMTEYVATRWYRAPEIMLTFQEYTTAMDINSCGCILAEMVSGKPLFPGRDYHHQLWLILE
VLGTPSFEDFNQIKSKRAKEYIANLPMRPPLPWETVWSKTDLNPDMIDLLDKMLQFNPDK
RISAAEALRHPYLAMYHDPSDEPEYPPLNLDDEFWKLDNKIMRPEEEEEVPIEMLKDMLY
DELMKTME*
SEQ ID NO: 99
PTA1 amino acid sequence; systematic name YAL043C
MSSAEMEQLLQAKTLAMHNNPTEMLPKVLETTASMYHNGNLSKLKLPLAKFFTQLVLDVV
SMDSPIANTERPFIAAQYLPLLLAMAQSTADVLVYKNIVLIMCASYPLVLDLVAKTSNQE
MFDQLCMLKKFVLSHWRTAYPLRATVDDETDVEQWLAQIDQNIGVKLATIKFISEVVLSQ
TKSPSGNEINSSTIPDNHPVLNKPALESEAKRLLDMLLNYLIEEQYMVSSVFIGIINSLS
FVIKRRPQTTIRILSGLLRFNVDAKFPLEGKSDLNYKLSKRFVERAYKNFVQFGLKNQII
TKSLSSGSGSSIYSKLTKISQTLHVIGEETKSKGILNFDPSKGNSKKTLSRQDKLKYISL
WKRQLSALLSTLGVSTKTPTPVSAPATGSSTENMLDQLKILQKYTLNKASHQGNTFFNNS
PKPISNTYSSVYSLMNSSNSNQDVTQLPNDILIKLSTEAILQMDSTKLITGLSIVASRYT
DLMNTYINSVPSSSSSKRKSDDDDDGNDNEEVGNDGPTANSKKIKMETEPLAEEPEEPED
DDRMQKMLQEEESAQEISGDANKSTSAIKEIAPPFEPDSLTQDEKLKYLSKLTKKLFELS
GRQDTTRAKSSSSSSILLDDDDSSSWLHVLIRLVTRGIEAQEASDLIREELLGFFIQDFE
QRVSLIIEWLNEEWFFQTSLHQDPSNYKKWSLRVLESLGPFLENKHRRFFIRLMSELPSL
QSDHLEALKPICLDPARSSLGFQTLKFLIMFRPPVQDTVRDLLHQLKQEDEGLHKQCDSL
LDRLK*
SEQ ID NO: 100
YHR138C amino acid sequence; systematic name YHR138C
MKASYLVLIFISIFSMAQASSLSSYIVTFPKTDNMATDQNSIIEDVKKYVVDIGGKITHE
YSLIKGFTVDLPDSDQILDGLKERLSYIESEYGAKCNLEKDSEVHALNRDHLVA*
SEQ ID NO: 101
TSR3 amino acid sequence; systematic name YOR006C
MGKGKNKMHEPKNGRPQRGANGHSSRQNHRRMEMKYDNSEKMKFPVKLAMWDFDHCDPKR
CSGKKLERLGLIKSLRVGQKFQGIVVSPNGKGVVCPDDLEIVEQHGASVVECSWARLEEV
PFNKIGGKHERLLPYLVAANQVNYGRPWRLNCVEALAACFAIVGRMDWASELLSHFSWGM
GFLELNKELLEIYQQCTDCDSVKRAEEEWLQKLEKETQERKSRAKEEDIWMMGNINRRGN
GSQSDTSESEENSEQSDLEGNNQCIEYDSLGNAIRIDNMKSREAQSEESEDEESGSKENG
EPLSYDPLGNLIR*
SEQ ID NO: 102
ECI1 amino acid sequence; systematic name YLR284C
MSQEIRQNEKISYRIEGPFFIIHLMNPDNLNALEGEDYIYLGELLELADRNRDVYFTIIQ
SSGRFFSSGADFKGIAKAQGDDTNKYPSETSKWVSNFVARNVYVTDAFIKHSKVLICCLN
GPAIGLSAALVALCDIVYSINDKVYLLYPFANLGLITEGGTTVSLPLKFGTNTTYECLMF
NKPFKYDIMCENGFISKNFNMPSSNAEAFNAKVLEELREKVKGLYLPSCLGMKKLLKSNH
IDAFNKANSVEVNESLKYWVDGEPLKRFRQLGSKQRKHRL*
SEQ ID NO: 103
RDL2 (AIM42) amino acid sequence; systematic name YOR286W
MFKHSTGILSRTVSARSPTLVLRTFTTKAPKIYTFDQVRNLVEHPNDKKLLVDVREPKEV
KDYKMPTTINIPVNSAPGALGLPEKEFHKVFQFAKPPHDKELIFLCAKGVRAKTAEELAR
SYGYENTGIYPGSITEWLAKGGADVKPKK*
SEQ ID NO: 104
SWD2 amino acid sequence; systematic name YKL018W
MTTVSINKPNLLKFKHVKSFQPQEKDCGPVTSLNFDDNGQFLLTSSSNDTMQLYSATNCK
FLDTIASKKYGCHSAIFTHAQNECIYSSTMKNFDIKYLNLETNQYLRYFSGHGALVNDLK
MNPVNDTFLSSSYDESVRLWDLKISKPQVIIPSLVPNCIAYDPSGLVFALGNPENFEIGL
YNLKKIQEGPFLIIKINDATFSQWNKLEFSNNGKYLLVGSSIGKHLIFDAFTGQQLFELI
GTRAFPMREFLDSGSACFTPDGEFVLGTDYDGRIAIWNHSDSISNKVLRPQGFIPCVSHE
TCPRSIAFNPKYSMFVTADETVDFYVYDE*
SEQ ID NO: 105
VPS71 amino acid sequence; systematic name YML041C
MKALVEEIDKKTYNPDIYFTSLDPQARRYTSKKINKQGTISTSRPVKRINYSLADLEARL
YTSRSEGDGNSISRQDDRNSKNSHSFEERYTQQEILQSDRRFMELNTENFSDLPNVPTLL
SDLTGVPRDRIESTTKPISQTSDGLSALMGGSSFVKEHSKYGHGWVLKPETLREIQLSYK
STKLPKPKRKNTNRIVALKKVLSSKRNLHSFLDSALLNLMDKNVIYHNVYNKRYFKVLPL
ITTCSICGGYDSISSCVNCGNKICSVSCFKLHNETRCRNR*
SEQ ID NO: 106
EMP47 amino acid sequence; systematic name YFL048C
MMMLITMKSTVLLSVFTVLATWAGLLEAHPLGDTSDASKLSSDYSLPDLINARKVPNNWQ
TGEQASLEEGRIVLTSKQNSKGSLWLKQGFDLKDSFTMEWTFRSVGYSGQTDGGISFWFV
QDSNVPRDKQLYNGPVNYDGLQLLVDNNGPLGPTLRGQLNDGQKPVDKTKIYDQSFASCL
MGYQDSSVPSTIRVTYDLEDDNLLKVQVDNKVCFQTRKVRFPSGSYRIGVTAQNGAVNNN
AESFEIFKMQFFNGVIEDSLIPNVNAMGQPKLITKYIDQQTGKEKLIEKTAFDADKDKIT
NYELYKKLDRVEGKILANDINALETKLNDVIKVQQELLSFMTTITKQLSSKPPANNEKGT
STDDAIAEDKENFKDFLSINQKLEKVLVEQEKYREATKRHGQDGPQVDEIARKLMIWLLP
LIFIMLVMAYYTFRIRQEIIKTKLL*
SEQ ID NO: 107
ADE13 amino acid sequence; systematic name YLR359W
MPDYDNYTTPLSSRYASKEMSATFSLRNRFSTWRKLWLNLAIAEKELGLTVVTDEAIEQM
RKHVEITDDEIAKASAQEAIVRHDVMAHVHTFGETCPAAAGIIHLGATSCFVTDNADLIF
IRDAYDIIIPKLVNVINRLAKFAMEYKDLPVLGWTHFQPAQLTTLGKRATLWIQELLWDL
RNFERARNDIGLRGVKGTTGTQASFLALFHGNHDKVEALDERVTELLGFDKVYPVTGQTY
SRKIDIDVLAPLSSFAATAHKMATDIRLLANLKEVEEPFEKSQIGSSAMAYKRNPMRCER
VCSLARHLGSLFSDAVQTASVQWFERTLDDSAIRRISLPSAFLTADILLSTLLNISSGLV
VYPKVIERRIKGELPFMATENIIMAMVEKNASRQEVHERIRVLSHQAAAVVKEEGGENDL
IERVKRDEFFKPIWEELDSLLEPSTFVGRAPQQVEKFVQKDVNNALQPFQKYLNDEQVKL
NV*
SEQ ID NO: 108
FLC1 amino acid sequence; systematic name YPL221W
MQVLVTLWCLICTCLVLPVAAKKRTLTASSLVTCMENSQLSANSFDVSFSPDDRSLHYDL
DMTTQIDSYIYAYVDVYAYGFKIITENFDVCSMGWKQFCPVHPGNIQIDSIEYIAQKYVK
MIPGIAYQVPDIDAYVRLNIYNNVSENLACIQVFFSNGKTVSQIGVKWVTAVIAGIGLLT
SAVLSTFGNSTAASHISANTMSLFLYFQSVAVVAMQHVDSVPPIAAAWSENLAWSMGLIR
ITFMQKIFRWYVEATGGSASLYLTATTMSVLTQRGLDYLKNTSVYKRAENVLYGNSNTLI
FRGIKRMGYRMKIENTAIVCTGFTFFVLCGYFLAGFIMACKYSIELCIRCGWMRSDRFYQ
FRKNWRSVLKGSLLRYIYIGFTQLTILSFWEFTERDSAGVIVIACLFIVLSCGLMAWAAY
RTIFFASKSVEMYNNPAALLYGDEYVLNKYGFFYTMFNAKHYWWNALLTTYILVKALFVG
FAQASGKTQALAIFIIDLAYFVAIIRYKPYLDRPTNIVNIFICTVTLVNSFLFMFFSNLF
NQKYAVSAIMGWVFFIMNAAFSLLLLLMILAFTTIILFSKNPDSRFKPAKDDRASFQKHA
IPHEGALNKSVANELMALGNVAKDHTENWEYELKSQEGKSEDNLFGVEYDDEKTGTNSEN
AESSSKETTRPTFSEKVLRSLSIKRNKSKLGSFKRSAPDKITQQEVSPDRASSSPNSKSY
PGVSHTRQESEANNGLINAYEDEQFSLMEPSILEDAASSTQMHAMPARDLSLSSVANAQD
VTKKANILDPDYL*
SEQ ID NO: 109
AOS1 amino acid sequence; systematic name YPR180W
MDMKVEKLSEDEIALYDRQIRLWGMTAQANMRSAKVLLINLGAIGSEITKSIVLSGIGHL
TILDGHMVTEEDLGSQFFIGSEDVGQWKIDATKERIQDLNPRIELNFDKQDLQEKDEEFF
QQFDLVVATEMQIDEAIKINTLTRKLNIPLYVAGSNGLFAYVFIDLIEFISEDEKLQSVR
PTTVGPISSNRSIIEVTTRKDEEDEKKTYERIKTKNCYRPLNEVLSTATLKEKMTQRQLK
RVTSILPLTLSLLQYGLNQKGKAISFEQMKRDAAVWCENLGVPATVVKDDYIQQFIKQKG
IEFAPVAAIIGGAVAQDVINILGKRLSPLNNFIVFDGITLDMPLFEF*
SEQ ID NO: 110
YMC1 amino acid sequence; systematic name YPR058W
MSEEFPSPQLIDDLEEHPQHDNARVVKDLLAGTAGGIAQVLVGQPFDTTKVRLQTSSTPT
TAMEVVRKLLANEGPRGFYKGTLTPLIGVGACVSLQFGVNEAMKRFFHHRNADMSSTLSL
PQYYACGVTGGIVNSFLASPIEHVRIRLQTQTGSGTNAEFKGPLECIKKLRHNKALLRGL
TPTILREGHGCGTYFLVYEALIANQMNKRRGLERKDIPAWKLCIFGALSGTALWLMVYPL
DVIKSVMQTDNLQKPKFGNSISSVAKTLYANGGIGAFFKGFGPTMLRAAPANGATFATFE
LAMRLLG*
SEQ ID NO: 111
MRPL20 amino acid sequence; systematic name YKR085C
MIGRGVCCRSFHTAGSAWKQFGFPKTQVTTIYNKTKSASNYKGYLKHRDAPGMYYQPSES
IATGSVNSETIPRSFMAASDPRRGLDMPVQSTKAKQCPNVLVGKSTVNGKTYHLGPQEID
EIRKLRLDNPQKYTRKFLAAKYGISPLFVSMVSKPSEQHVQIMESRLQEIQSRWKEKRRI
AREDRKRRKLLWYQA*
SEQ ID NO: 112
EMC1 amino acid sequence; systematic name YCL045C
MKITCTDLVYVFILLFLNTSCVQAVFSDDAFITDWQLANLGPWEKVIPDSRDRNRVLILS
NPTETSCLVSSFNVSSGQILFRNVLPFTIDEIQLDSNDHNAMVCVNSSSNHWQKYDLHDW
FLLEEGVDNAPSTTILPQSSYLNDQVSIKNNELHILDEQSKLAEWKLELPQGFNKVEYFH
REDPLALVLNVNDTQYMGFSANGTELIPVWQRDEWLTNVVDYAVLDVFDSRDVELNKDMK
AELDSNSLWNAYWLRLTTNWNRLINLLKENQFSPGRVFTKLLALDAKDTTVSDLKFGFAK
ILIVLTHDGFIGGLDMVNKGQLIWKLDLEIDQGVKMFWTDKNHDELVVFSHDGHYLTIEV
TKDQPIIKSRSPLSERKTVDSVIRLNEHDHQYLIKFEDKDHLLFKLNPGKNTDVPIVANN
HSSSHIFVTEHDTNGIYGYIIENDTVKQTWKKAVNSKEKMVAYSKRETTNLNTLGITLGD
KSVLYKYLYPNLAAYLIANEEHHTITFNLIDTITGEILITQEHKDSPDFRFPMDIVFGEY
WVVYSYFSSEPVPEQKLVVVELYESLTPDERLSNSSDNFSYDPLTGHINKPQFQTKQFIF
PEIIKTMSISKTTDDITTKAIVMELENGQITYIPKLLLNARGKPAEEMAKDKKKEFMATP
YTPVIPINDNFIITHFRNLLPGSDSQLISIPTNLESTSIICDLGLDVFCTRITPSGQFDL
MSPTFEKGKLLITIFVLLVITYFIRPSVSNKKLKSQWLIK*
SEQ ID NO: 113
YMR155W amino acid sequence; systematic name YMR155W
MVKKHQNSKMGNTNHFGHLKSFVGGNVVALGAGTPYLFSFYAPQLLSKCHIPVSASSKLS
FSLTIGSSLMGILAGIVVDRSPKLSCLIGSMCVFIAYLILNLCYKHEWSSTFLISLSLVL
IGYGSVSGFYASVKCANTNFPQHRGTAGAFPVSLYGLSGMVFSYLCSKLFGENIEHVFIF
LMVACGCMILVGYFSLDIFSNAEGDDASIKEWELQKSRETDDNIVPLYENSNDYIGSPVR
SSSPATYETYALSDNFQETSEFFALEDRQLSNRPLLSPSSPHTKYDFEDENTSKNTVGEN
SAQKSMRLHVFQSLKSSTFIGYYIVLGILQGVGLMYIYSVGFMVQAQVSTPPLNQLPINA
EKIQSLQVTLLSLLSFCGRLSSGPISDFLVKKFKAQRLWNIVIASLLVFLASNKISHDFS
SIEDPSLRASKSFKNISVCSAIFGYSFGVLFGTFPSIVADRFGTNGYSTLWGVLTTGGVF
SVSVFTDILGRDFKANTGDDDGNCKKGVLCYSYTFMVTKYCAAFNLLFVLGIIGYTYYRR
RATANSL*
SEQ ID NO: 114
LCB2 nucleic acid sequence
ATGAGTACTCCTGCAAACTATACCCGTGTGCCCCTGTGCGAACCAGAGGAGCTGCCAGAC
GACATACAAAAAGAAAATGAATATGGTACACTAGATTCTCCGGGGCATTTGTATCAAGTC
AAGTCACGTCATGGGAAGCCACTACCTGAGCCCGTTGTCGACACCCCTCCTTATTACATT
TCTTTGTTAACATATCTAAATTATTTGATTCTGATTATATTAGGTCATGTTCACGACTTC
TTAGGTATGACCTTCCAAAAAAACAAACATCTGGATCTTTTAGAGCATGATGGGTTAGCA
CCTTGGTTTTCAAATTTCGAGAGTTTTTATGTCAGGAGAATTAAAATGAGAATTGATGAT
TGCTTTTCTAGACCAACTACTGGTGTTCCTGGTAGATTTATTCGTTGTATTGATAGAATT
TCTCATAATATAAATGAGTATTTTACCTACTCAGGCGCAGTGTATCCATGCATGAACTTA
TCATCATATAACTATTTAGGCTTCGCACAAAGTAAGGGTCAATGTACCGATGCCGCCTTG
GAATCTGTCGATAAATATTCTATTCAATCTGGTGGTCCAAGAGCTCAAATCGGTACCACA
GATTTGCACATTAAAGCAGAGAAATTAGTTGCTAGATTTATCGGTAAGGAGGATGCCCTC
GTTTTTTCGATGGGTTATGGTACAAATGCAAACTTGTTCAACGCTTTCCTCGATAAAAAG
TGTTTAGTTATCTCTGACGAATTGAACCACACCTCTATTAGAACAGGTGTTAGGCTTTCT
GGTGCTGCTGTGCGAACTTTCAAGCATGGTGATATGGTGGGTTTAGAAAAGCTTATCAGA
GAACAGATAGTACTTGGTCAACCAAAAACAAATCGTCCATGGAAGAAAATTTTAATTTGC
GCAGAAGGGTTGTTTTCCATGGAAGGTACTTTGTGTAACTTGCCAAAATTGGTTGAATTG
AAGAAGAAATATAAATGTTACTTGTTTATCGATGAAGCCCATTCTATAGGCGCTATGGGC
CCAACTGGTCGCGGTGTTTGTGAAATATTTGGCGTTGATCCCAAGGACGTCGACATTCTA
ATGGGTACTTTCACTAAGTCGTTTGGTGCTGCTGGTGGTTACATTGCTGCTGATCAATGG
ATTATCGATAGACTGAGGTTGGATTTAACCACTGTGAGTTATAGTGAGTCAATGCCGGCT
CCTGTTTTAGCTCAAACTATTTCCTCATTACAAACCATTAGTGGTGAAATATGTCCCGGA
CAAGGTACTGAAAGATTGCAACGTATAGCCTTTAATTCCCGTTATCTACGTTTAGCTTTG
CAAAGGTTAGGATTTATTGTCTACGGTGTGGCTGACTCACCAGTTATTCCCTTACTACTG
TATTGTCCCTCAAAGATGCCCGCATTTTCGAGAATGATGTTACAAAGACGGATTGCTGTT
GTTGTTGTTGCTTATCCTGCTACTCCGCTGATCGAATCAAGAGTAAGATTCTGTATGTCT
GCATCTTTAACAAAGGAAGATATCGATTATTTACTGCGTCATGTTAGTGAAGTTGGTGAC
AAATTGAATTTGAAATCAAATTCCGGCAAATCCAGTTACGACGGTAAACGTCAAAGATGG
GACATCGAGGAAGTTATCAGGAGAACACCTGAAGATTGTAAGGACGACAAGTATTTTGTT
AATTGA
SEQ ID NO: 115
CHA1 nucleic acid sequence
ATGTCGATAGTCTACAATAAAACACCATTATTACGTCAATTCTTCCCCGGAAAGGCTTCT
GCACAATTTTTCTTGAAATATGAATGCCTTCAACCAAGTGGCTCCTTCAAAAGTAGAGGA
ATCGGTAATCTCATCATGAAAAGTGCCATTCGAATTCAAAAGGACGGTAAAAGATCTCCT
CAGGTTTTCGCTAGTTCTGGCGGTAATGCCGGTTTTGCTGCTGCAACAGCATGTCAAAGA
CTGTCTCTACCATGTACAGTCGTGGTTCCTACAGCGACAAAGAAGAGAATGGTAGATAAA
ATCAGGAACACCGGTGCCCAGGTTATCGTGAGTGGTGCCTACTGGAAAGAAGCAGATACT
TTTTTAAAAACAAATGTCATGAATAAAATAGACTCTCAGGTCATTGAGCCCATTTATGTT
CATCCCTTCGATAATCCGGATATTTGGGAAGGACATTCATCTATGATAGATGAAATAGTA
CAAGATTTGAAATCGCAACATATTTCCGTGAATAAGGTTAAAGGCATAGTATGCAGCGTT
GGTGGAGGTGGTTTATACAATGGTATTATTCAAGGTTTGGAAAGGTATGGTTTAGCTGAT
AGGATCCCTATTGTGGGGGTGGAAACGAATGGATGTCATGTTTTCAATACTTCTTTGAAA
ATAGGCCAACCAGTTCAATTCAAGAAGATAACAAGTATTGCTACTTCTCTAGGAACGGCC
GTGATCTCTAATCAAACTTTCGAATACGCTCGCAAATACAACACCAGATCCGTTGTAATA
GAGGACAAAGATGTTATTGAAACCTGTCTTAAATATACACATCAATTCAATATGGTGATT
GAACCGGCATGTGGCGCCGCATTGCATTTGGGTTACAACACTAAGATCCTAGAAAATGCA
CTGGGCTCAAAATTAGCTGCGGATGACATTGTGATAATTATTGCTTGTGGCGGCTCCTCT
AATACTATAAAGGACTTGGAAGAAGCGTTGGATAGCATGAGAAAAAAAGACACTCCTGTA
ATAGAAGTCGCTGACAATTTCATATTTCCAGAAAAAAATATTGTGAATTTAAAAAGTGCT
TGA*
SEQ ID NO: 116
HXT5 nucleic acid sequence
ATGTCGGAACTTGAAAACGCTCATCAAGGCCCCTTGGAAGGGTCTGCTACTGTGAGCACA
AATTCTAACTCATACAACGAGAAGTCAGGAAACTCGACTGCTCCTGGTACCGCCGGTTAC
AACGATAATTTGGCACAAGCTAAACCCGTCTCAAGTTACATTTCCCATGAAGGCCCTCCC
AAAGACGAACTGGAAGAGCTTCAGAAGGAGGTTGACAAACAACTAGAGAAGAAATCGAAG
TCGGATTTACTATTTGTATCCGTCTGCTGTTTGATGGTTGCTTTTGGTGGGTTCGTGTTT
GGGTGGGATACTGGTACTATATCTGGTTTTGTCAGGCAAACAGACTTCATTAGGCGATTT
GGCAGCACCCGTGCAAACGGGACTACCTATCTTTCCGATGTCAGAACCGGTTTGATGGTT
TCTATTTTCAACATCGGCTGCGCTATCGGAGGTATAGTTTTGTCAAAGCTCGGTGATATG
TATGGACGTAAGATTGGTCTGATGACTGTTGTCGTCATTTACTCAATTGGGATCATCATC
CAAATCGCCTCCATTGACAAATGGTATCAATATTTCATTGGAAGAATCATCTCAGGACTG
GGCGTTGGTGGTATTACAGTTTTGGCGCCTATGCTAATTTCTGAAGTGTCGCCTAAGCAG
TTGCGTGGTACTCTGGTTTCATGTTACCAATTAATGATCACTTTCGGTATCTTTTTGGGA
TATTGTACTAATTTTGGTACCAAGAATTACTCAAACTCTGTCCAATGGAGGGTACCATTA
GGCTTATGCTTTGCATGGTCTATTTTTATGATTGTTGGTATGACGTTCGTTCCTGAATCC
CCACGTTATCTGGTAGAAGTGGGAAAAATTGAAGAGGCCAAGCGGTCCTTAGCAAGAGCT
AACAAAACCACTGAAGACTCTCCTTTAGTAACTTTAGAAATGGAGAACTATCAGTCTTCT
ATTGAAGCTGAGAGATTGGCGGGCTCTGCTTCTTGGGGGGAATTGGTTACTGGTAAGCCC
CAGATGTTCAGACGTACACTAATGGGTATGATGATTCAATCTTTACAACAGCTGACAGGT
GACAATTACTTCTTTTACTATGGTACTACAATTTTCCAGGCTGTTGGTTTGGAAGATTCA
TTTGAAACTGCTATTGTTTTGGGTGTTGTTAATTTTGTTTCGACTTTTTTCTCGCTATAT
ACCGTCGATCGTTTTGGTCGTCGTAATTGTTTGTTATGGGGCTGTGTAGGTATGATTTGT
TGCTATGTCGTCTATGCCTCTGTTGGTGTTACCAGATTATGGCCAAACGGTCAAGATCAA
CCATCTTCAAAGGGTGCTGGTAACTGTATGATTGTTTTCGCATGTTTCTACATTTTCTGT
TTCGCTACCACTTGGGCCCCCGTTGCCTATGTCCTTATCTCTGAGTCGTATCCCTTAAGA
GTACGTGGTAAAGCAATGTCGATTGCAAGTGCCTGTAACTGGATTTGGGGGTTCTTGATC
AGTTTTTTCACTCCATTTATTACTTCAGCAATCAATTTCTATTATGGCTATGTCTTTATG
GGTTGTATGGTGTTCGCATACTTTTATGTGTTCTTCTTTGTTCCAGAGACAAAGGGCTTA
ACATTAGAAGAAGTCAACGAAATGTATGAAGAAAATGTGCTACCTTGGAAGTCTACCAAA
TGGATCCCACCATCTAGGAGAACAACAGATTATGACCTAGACGCTACTAGAAATGATCCG
AGACCATTTTATAAAAGGATGTTCACTAAAGAAAAATAA
SEQ ID NO: 117
MTD1 nucleic acid sequence
ATGTCGAAGCCTGGTCGTACTATTTTAGCAAGCAAGGTCGCCGAAACTTTCAATACCGAA
ATAATTAACAACGTAGAGGAATACAAGAAGACACATAATGGTCAAGGTCCCCTTCTTGTG
GGATTCCTAGCTAATAATGATCCTGCTGCAAAGATGTATGCTACATGGACTCAAAAGACT
AGCGAGTCAATGGGGTTCCGCTATGACTTAAGGGTCATTGAAGATAAGGATTTTTTGGAA
GAAGCGATAATACAAGCTAACGGCGATGACTCTGTGAACGGTATCATGGTATACTTTCCT
GTTTTCGGTAATGCTCAAGATCAGTATTTGCAACAGGTTGTGTGCAAGGAAAAAGATGTA
GAAGGGTTAAATCATGTTTACTACCAAAACCTGTACCATAATGTCAGATACCTGGACAAA
GAAAACCGTTTGAAATCCATTCTACCTTGCACACCACTAGCTATCGTTAAGATATTGGAA
TTCTTGAAAATTTACAACAATTTGTTACCAGAAGGAAACAGACTGTATGGGAAGAAATGC
ATAGTAATTAACAGGTCAGAAATCGTCGGTAGACCACTGGCGGCGCTATTAGCCAATGAC
GGTGCCACAGTATACTCTGTGGACGTTAACAACATTCAAAAATTCACCCGTGGTGAAAGT
TTGAAATTAAACAAGCATCATGTGGAAGACCTTGGGGAGTACTCTGAAGATCTGTTGAAA
AAGTGTTCTCTTGATTCAGATGTGGTCATCACTGGTGTCCCTAGTGAAAATTACAAATTC
CCCACCGAATACATCAAAGAAGGTGCCGTCTGCATCAATTTTGCATGCACCAAAAATTTT
AGCGATGATGTCAAGGAAAAAGCTTCTCTTTACGTTCCAATGACTGGTAAAGTTACCATT
GCAATGTTGTTGAGAAACATGTTACGTTTAGTAAGGAACGTAGAACTGTCTAAAGAAAAA
TAG
SEQ ID NO: 118
MSC6 nucleic acid sequence
ATGCTTTCCCATAATGCTTTAAGGGCCTTTGATTGTTCAAAGGTGATTATTTCACGAAGA
TGTCTAACCTCTTCAACATCGATATACCAACAAAGCAGCGTTCACTTACAAGAAACAGAT
GATGGACATTCAGGAAATAGAGAAAAGCACGTCTCACCGTTTGAAAGGGTACAAAATTTG
GCTGCTGATTTGAAGAACGAGTTGAAAGCTCCAGATTCAGATATCAATGAAGTTTTTAAT
GACTTTAAAGATAAGATTGAATCGTTGAAACAGAAATTAAGGAACCCTTCACCTATGGAA
AGATCACACTTGTTAGCGAATTTTTCTTCGGATCTCCTACAGGAATTAAGTTACAGAAGC
AAAAATATGACGCTAGATCCTTATCAAGTATTAAACACATTGTGCCAATACAAATTGGCA
CGCTCACAACATTTCACGATTGTTTTAAAGTACCTTCTATATAATCAATCACCACAGGAC
GTTATTGCCTTATGGGTGAAGTACTTGGAAACCATTTCCGAAAACCCAGTGATCTTACTT
CAAAATAGTTCTTCTCGTGCACATATGCAAAATATTGCAATTACCACCATTGCTTACTTA
TCTTTACCAGAGAATACTGTGGATATCAATATTCTGTATAAGATTTTACAGATCGATCGT
AAAATGGGCCAGGTTTTACCTTTTAACATGATTAGAAGAATGTTAAGTACAGAATTTAGC
TCTCTTGAAAGAAGAGACGTGATTATCAAAAATCTAAACACTTTGTACTATCAATACACA
GTACAGGATAGTGATCATTTCTTAAGTCAAATTGAAAATGCTCCTAGATGGATAGATTTA
AGGGATCTTTATGGCCAATACAATAAACTTGAAGGTGAGAAAAATGTAGAGATCATAAGC
AAGTTCATGGACAAGTTTATTGATTTGGATAAACCCGACCAAGTTGTTACTATTTATAAC
CAGTATAGCAAGGTTTTCCCAAATAGTACGTCGCTGAAAGATTGTCTTTTAAGAGCTGTG
TCGCACTTACGAGCTAAATCGAGTAAAGAGAAGTTGGACAGAATTCTAGCAGTCTGGAAC
AGTGTTATCAAACCAGGAGATAATATTAAAAACACATCTTATGCGACGCTAGTTAACGCA
CTAACTGATTCTGGAAATTTCAACCATTTAAAGGAATTTTGGGAAGAAGAACTTCCTAAA
AAGTTCAAGAAAGATCCCATCGTGAAGGAAGCATTTCTCCTGGCCTTATGTCAAACTTCG
CCTCTAAAGTATGACCAAGTCAAAGGGGAGTTAGCAGAGACTGTTAAAACCAAGAAGTTG
TTCAATAAAGTTTTATTGCTAATGTTAGATGATGAAAAAGTGAGCGAAGAACAATTCAAC
ACATTTTACTATAACCATTATCCATCAGATGGTGTGTTACCCCCTACTTTGGATACTCTA
AGCATTAAAATGTACGCTAATTATAAATTTCAGGCAGAAGATACACGCCCACAATTCGAT
CTATTGCAAAGTGTTTCCATTAATCCCACCGATTATGAAAAGGTTGAAAAGATTACGAAA
GCCTTTATTTCAGTGTGCCCCACTGTCGAGCCGATTCGTCAACTTTACAAACAATTGGGA
ACTCACTTAAATGCTAGGAATTATGCAGACTTTATTTCCGCAGAGTTTAATAAGCCTGAC
GGCACAGTGGCCGAGGCAAAGAATTTGTTTTCTGATTTTCTCTCATATCAAAAGACTAGA
AAGAGAAACGTGGATAATACGCCTCTAAATGCTTTATTATTGGGGTTCTGTGATAAACTT
TACAAGAGTAAACATAGCGAGTACGTTCCCTACATCGAAAAGTACTACAATCTAGCTAAG
GATTCAAGTATCAGGGTGTCGAACTTGGCCGTTTCGAAAATTCTATTCAACTTGGCCACA
TTTGCACGCAATACTCAGCAGTTATCTGACAAAGAGGTTGCTTTTATTAACCAGTTTATG
CGAGATTTAGGCACTAATGAGGGTTTTCGTCCCAACCCTAAGGATATTCAAATTTTAAAA
GAATGTGATGGAATTACTGTTCCAGAAAAGTTGACTTAA
SEQ ID NO: 119
SCW10 nucleic acid sequence
ATGCGTTTTTCAAATTTCCTAACTGTATCTGCATTATTAACCGGAGCTCTAGGAGCTCCT
GCTGTTCGCCATAAACATGAAAAGCGTGACGTTGTTACTGCCACAGTCCATGCGCAGGTT
ACTGTTGTCGTTTCCGGTAACAGCGGCGAAACTATTGTTCCAGTGAACGAGAATGCTGTT
GTAGCTACTACCAGCAGTACTGCAGTTGCTTCTCAAGCAACTACATCCACTTTAGAACCA
ACAACTTCCGCTAATGTCGTCACTTCTCAACAACAAACCAGCACTCTTCAATCTTCCGAG
GCAGCATCTACGGTTGGTTCTTCGACTTCATCCTCACCCTCATCCTCATCCTCAACTTCA
TCTTCAGCTTCATCCTCCGCTTCATCTAGTATCTCAGCCTCCGGTGCTAAGGGTATTACT
TACAGTCCTTACAATGATGATGGGTCCTGTAAATCTACTGCTCAAGTCGCCTCAGATTTA
GAACAGTTGACTGGTTTTGACAACATCAGATTATATGGCGTTGACTGTAGTCAGGTTGAG
AATGTCTTGCAAGCTAAAACTTCAAGCCAGAAATTATTCTTAGGCATATATTACGTTGAC
AAAATTCAAGACGCCGTTGATACTATTAAATCTGCAGTTGAGTCTTATGGCTCCTGGGAT
GATATTACCACTGTTTCTGTCGGTAACGAACTGGTCAATGGCGGTTCTGCCACTACGACG
CAAGTCGGTGAATACGTTTCCACGGCCAAGTCAGCTTTAACCTCTGCTGGTTATACAGGC
TCAGTCGTTTCCGTTGATACCTTCATTGCTGTTATAAATAACCCTGACCTGTGTAATTAT
TCTGACTATATGGCTGTCAACGCCCATGCATACTTCGATGAAAATACTGCGGCCCAAGAT
GCAGGACCATGGGTACTAGAACAAATCGAAAGGGTTTACACTGCTTGTGGTGGGAAAAAG
GACGTCGTTATTACCGAAACTGGTTGGCCATCTAAGGGTGATACTTACGGCGAAGCTGTC
CCATCTAAAGCAAACCAAGAAGCCGCCATTTCTTCTATCAAAAGCTCCTGCGGCTCTTCA
GCTTACTTATTTACCGCCTTCAATGATCTATGGAAAGATGATGGGCAATACGGTGTTGAA
AAATACTGGGGTATTCTATCAAGTGATTAA
SEQ ID NO: 120
YAL065C nucleic acid sequence
ATGAACAGTGCTACCAGTGAGACAACAACCAATACTGGAGCTGCTGAGACAACTACCAGT
ACTGGAGCTGCTGAGACGAAAACAGTAGTCACCTCTTCAATTTCAAGATTCAATCATGCT
GAAACACAGACGGCTTCCGCGACCGATGTGATTGGTCACAGCAGTAGTGTTGTTTCTGTA
TCCGAAACTGGCAACACCAAGAGTCTAATAACTTCCGGGTTAAGTACTATGTCGCAACAG
CCTCGTAGCACACCAGCAAGTAGCATAATAGGATCTAGTACTGCCTCTTTAGAAATCTCA
ACCTACGTTGGTATTGCCAATGGTCTGTTGACCAATAATGGCATAAGTGTTTTTATTTCC
ACCGTATTGCTGGCAATCGTATGGTAA
SEQ ID NO: 121
YJL107C nucleic acid sequence
ATGGACGGTAGAAATGAAAAACCAACCACTCCTGTGTCAGATTTTCGGGTGGGAAGCTCC
GAGCAAAGTCAAGCGGGAGTGAATCTTGAAGATAGTAGTGACCATCGCACTTCCAATTCA
GCCGAGAGCAAAAAAGGCAATTTAAGTGGTAAAAGCATCAGTGATCTAGGTATTTCTAAT
AATGATAACAAAAATGTAAGATTCACTGCTGATACGGATGCTCTAGAAAATGATTTGTCT
TCAAGATCTACAGAAACCAGCGATAATTCTAAGGGCACAGATGGACAAGATGAAGAAGAT
AGGCCTGCTCGCCACAAGAGGAAGCCTAAAGTTTCTTTCACACATTTAAGGAACAATGGT
AAGGATGGAGACGATGAGACGTTCATCAAGAAGATAATAAATAACCTGACTGGAAATCAA
GGGGGTTTGGTCCCTGGCTTGGCACCAATACCTTCAGAAAATGAAAATGGGAAGAATGAT
ATAGAAAAAAATAACCGTAATGAAGAAATTCCCTTATCCGATCTAGCTGATGCGTCTAAA
ATCGTAGACGTTCATGAGGGCGACGATAAAGAAAAACTGGAGGCTCTCAAATTAGAAGGT
GACGTAAATTGTACGTCGGATGGCGAAACGTTAGGCTCAAGTTCAAAAAATTCATTTCTG
GCTCCTGCAGTGGATCATTTTGATGATTATGCAGAAAACAATTCATCCGACGATAACGAA
GGGTTTATTGAAACCTCCACATACGTACCCCCTCCATCTCAAGTGAAAAGTGGAGTACTA
GGGTCATTATTGAAACTTTACCAAAATGAAGATCAAAATTCAAGCTCAATCTTTTCAGAT
TCACAAGCTGTAACAACAGATGATGAAGGTATTTCTTCTACTGCTGGAAACAAAGACGTA
CCAGTTGCCAAGCGTAGCAGATTACAAAATTTAAAAGGCAAGGCTAAAAAAGGCAGAATG
CCTAGACTGAAGAAAAGACTAAAAACTGAAGCGAAAATTACGGTTCACATTGCAGACATT
TTACAAAGACACCGGTTCATCCTACGCATGTGTAGAGCTCTTATGATGTATGGTGCTCCG
ACGCATAGGCTTGAAGAATATATGGTTATGACTTCTAGAGTCCTTGAAATAGATGGTCAG
TTTTGTATCTTCCAGGTTGTATGA
SEQ ID NO: 122
CSM3 nucleic acid sequence
ATGGATCAAGATTTTGACAGTTTATTACTAGGTTTCAATGACTCCGATAGTGTCCAAAAA
GACCCAACTGTACCAAATGGCTTGGATGGTTCAGTAGTTGATCCTACCATTGCGGATCCA
ACCGCAATTACAGCTAGAAAGAGAAGGCCTCAAGTAAAATTAACAGCCGAAAAACTACTC
AGTGATAAAGGTTTACCATATGTTTTGAAAAATGCACATAAAAGGATACGAATTTCCTCA
AAAAAAAACTCATATGACAACTTATCAAATATTATTCAGTTTTACCAGCTTTGGGCACAT
GAATTGTTTCCCAAGGCAAAATTTAAGGATTTTATGAAGATCTGTCAAACAGTAGGTAAA
ACAGATCCAGTTCTTAGAGAATATAGAGTCAGCCTTTTTAGGGACGAGATGGGCATGAGT
TTCGATGTTGGCACACGGGAGACTGGGCAAGACCTGGAAAGACAATCACCTATGGTTGAA
GAACATGTCACTTCCGCGGAAGAGAGGCCTATTGTCGCAGATAGTTTTGCGCAAGACAAA
AGGAATGTAAACAATGTCGATTACGATAATGACGAAGATGACGATATCTATCACCTTTCT
TATCGCAACAGAAGAGGACGAGTTTTGGACGAACGTGGGAATAATGAAACGGTACTTAAC
AACGTTGTGCCGCCTAAGGAAGATTTGGATGCATTATTGAAGACATTCAGGGTACAAGGG
CCCGTTGGCCTTGAAGAAAATGAGAAGAAGCTCTTATTAGGATGGCTAGATGCGCATAGA
AAAATGGAAAAAGGCTCTATGACTGAAGAAGACGTTCAACTGATTCAAAGTTTGGAAGAG
TGGGAAATGAATGATATAGAGGGACAACATACTCATTATGATTTATTGCCAGGGGGAGAT
GAGTTTGGCGTAGATCAAGATGAGTTGGATGCTATGAAGGAAATGGGCTTTTAG
SEQ ID NO: 123
RGT2 nucleic acid sequence
ATGAACGATAGCCAAAACTGCCTACGACAGAGGGAAGAAAATAGTCATCTGAATCCTGGA
AATGACTTCGGCCACCACCAGGGTGCAGAATGTACGATAAATCATAACAACATGCCACAC
CGCAATGCATACACAGAATCTACGAATGACACGGAAGCAAAGTCCATAGTGATGTGCGAC
GATCCTAACGCATACCAAATTTCCTACACAAATAATGAGCCGGCGGGAGATGGAGCTATA
GAAACCACGTCCATTCTACTATCGCAACCGCTGCCGCTGCGATCGAATGTGATGTCTGTC
TTGGTAGGCATATTTGTTGCCGTGGGGGGCTTCTTGTTTGGGTATGACACTGGACTTATA
AACAGTATCACGGATATGCCGTATGTTAAAACCTACATTGCTCCGAACCATTCATATTTC
ACCACTAGCCAAATAGCCATACTCGTATCATTCCTCTCCCTAGGAACATTTTTCGGTGCG
TTAATCGCTCCCTATATTTCAGATTCATATGGTAGGAAGCCAACAATTATGTTTAGTACC
GCTGTTATCTTTTCCATCGGAAACTCATTACAGGTGGCATCCGGTGGCTTGGTGCTATTA
ATCGTCGGAAGAGTGATCTCAGGTATCGGGATCGGGATAATCTCTGCTGTGGTTCCTCTT
TATCAAGCTGAAGCTGCGCAGAAGAACCTTAGAGGTGCCATCATTTCCAGTTATCAGTGG
GCTATCACTATTGGGTTACTCGTGTCCAGTGCAGTATCGCAAGGAACTCATTCCAAAAAT
GGCCCGTCTTCATATAGAATACCAATTGGTTTGCAGTACGTTTGGTCAAGTATTTTAGCT
GTGGGCATGATATTCCTTCCAGAGAGTCCAAGATATTACGTCTTGAAGGATGAACTCAAT
AAAGCTGCAAAATCGTTATCCTTTTTAAGAGGCCTCCCGATCGAAGATCCAAGACTCTTA
GAGGAGCTTGTTGAAATAAAAGCCACTTACGATTATGAAGCATCGTTCGGCCCGTCAACA
CTTTTAGATTGTTTCAAAACAAGTGAAAATAGACCCAAACAGATTTTACGAATATTTACT
GGTATCGCCATACAAGCTTTTCAACAGGCATCTGGTATCAATTTTATATTCTACTATGGA
GTTAATTTTTTCAACAACACAGGGGTGGACAACTCTTACTTGGTTTCTTTTATCAGCTAT
GCCGTCAACGTCGCCTTCAGTATACCGGGTATGTATTTAGTGGATCGAATTGGTAGAAGA
CCAGTCCTTCTTGCTGGAGGTGTCATAATGGCAATAGCAAATTTAGTCATTGCCATCGTT
GGTGTTTCCGAGGGAAAAACTGTTGTTGCTAGTAAAATTATGATTGCTTTTATATGCCTT
TTCATTGCTGCATTTTCGGCGACATGGGGTGGTGTCGTGTGGGTGGTATCTGCTGAACTG
TACCCACTTGGTGTCAGATCGAAATGTACCGCCATATGCGCTGCCGCAAATTGGCTAGTT
AATTTCACCTGTGCCCTGATTACACCTTACATTGTTGATGTCGGATCACACACTTCTTCA
ATGGGGCCCAAAATATTCTTCATTTGGGGCGGCTTAAATGTCGTGGCCGTTATCGTTGTT
TATTTCGCTGTTTATGAAACGAGGGGATTGACTTTGGAAGAGATTGACGAGTTATTTAGA
AAGGCCCCAAATAGCGTCATTTCTAGCAAATGGAACAAAAAAATAAGGAAAAGGTGCTTA
GCCTTTCCCATTTCACAACAAATAGAGATGAAAACTAATATCAAGAACGCTGGAAAGTTG
GACAACAACAACAGTCCAATTGTACAGGATGACAGCCACAACATAATCGATGTGGATGGA
TTCTTGGAGAACCAAATACAGTCCAATGATCATATGATTGCGGCGGATAAAGGAAGTGGC
TCGTTAGTAAACATCATCGATACTGCCCCCCTAACATCTACAGAGTTTAAACCCGTGGAA
CATCCGCCAGTAAATTACGTCGACTTGGGGAATGGTTTGGGTCTGAATACATACAATAGA
GGTCCTCCTTCTATCATTTCTGACTCTACTGATGAGTTCTATGAGGAAAATGACTCTTCT
TATTACAATAACAACACTGAACGAAATGGAGCTAACAGCGTCAATACATATATGGCTCAA
CTAATCAATAGCTCATCTACTACAAGCAACGACACATCGTTCTCTCCATCACACAATAGC
AATGCAAGAACGTCCTCTAATTGGACGAGTGACCTCGCTAGTAAGCACAGCCAATACACT
TCCCCCCAATAA
SEQ ID NO: 124
CHS7 nucleic acid sequence
ATGGCATTTAGTGATTTTGCTGCCATATGCTCAAAGACCCCGTTGCCATTATGTTCGGTA
ATAAAGTCTAAAACCCATCTAATACTTTCGAACTCAACAATTATACATGATTTTGATCCT
TTAAATTTGAATGTCGGTGTACTGCCACGCTGTTATGCTCGGTCGATTGATCTTGCCAAT
ACAGTCATCTTTGATGTCGGGAACGCATTCATAAATATTGGTGCTCTAGGTGTCATTTTA
ATCATACTTTATAACATAAGACAGAAGTATACTGCTATTGGCAGGTCTGAATATCTCTAC
TTTTTCCAACTAACATTGCTATTGATAATATTTACCTTGGTGGTAGACTGTGGTGTATCT
CCCCCCGGCTCTGGGTCATATCCATACTTCGTGGCTATACAAATAGGACTGGCGGGTGCA
TGTTGCTGGGCCTTATTGATAATCGGGTTTTTAGGTTTCAATTTATGGGAAGATGGGACT
ACAAAGTCCATGCTGTTGGTCCGTGGAACGTCCATGCTAGGATTCATAGCCAATTTTTTA
GCCTCTATTTTAACCTTCAAAGCATGGATCACCGACCATAAAGTAGCAACAATGAACGCT
TCAGGGATGATTGTCGTCGTTTACATAATAAACGCCATTTTCTTATTCGTTTTCGTTATT
TGTCAATTACTGGTATCCCTATTGGTAGTTCGAAACTTATGGGTCACAGGAGCTATCTTT
TTGGGGCTATTTTTCTTTGTAGCAGGCCAGGTATTGGTTTATGCCTTCTCTACACAAATT
TGTGAAGGGTTCAAGCACTACTTAGATGGCCTCTTTTTTGGAAGCATCTGTAATGTGTTC
ACATTAATGATGGTTTACAAGACTTGGGATATGACTACCGACGACGACTTGGAATTTGGT
GTAAGTGTTAGCAAGGACGGTGACGTGGTGTATGATAATGGATTTATGTGA
SEQ ID NO: 125
BOP2 nucleic acid sequence
ATGGTTGCCGCTTTAACGTATTTGCCTACTGAGCTTATCCAAAGGATATTTGAGTTCACT
GTGGTGGAAACAGACTCTCAATATTGGTTGTACAATTTAGTGGCTCTAATTGATTTTTCT
GTCTCTTCGAGAGGTGGTGGCTCTATAACGGAAGACTTCTTGACAAATTACGTTAGGAAG
AATTTGATGGTTTTAGATCTGACCTGTGAGGCCACGCAAGACTCGATTTTACGAGCGGAG
TACGGGTTTCTGAAGAGATTGTTGCCATACATTGACATGGACGCACAATATATCAGAGTT
GTTGATTTGGAAACCAATGCTGACAAGGCCCAGAATTTAAAAGCAGAAAAACTTATTGTT
ATATTTGACGAATTCTCAGATTTGAAACTCATAGAAACCTTCTTCCCCTTGGCGAATTCC
AATTCAAACATAATCGAGTTCGTATTCTGTGTTCGCAATATAAAGAGTTCGTTTTATTCA
CCTTTGGAAAAATTACATATTGCGAACATAGTCGCAGATATTGATATTAACACATTGTAT
CTGGACTTCGTGGATTCAAATATCTATTCGGATCAAAATTTCTTTGGGATTTTTGATCCC
GATATTTTTCAGCTGATTAATAAAAACTATAGAAACTTCTTTTCTAAGACTAACGAAAAG
GGGAAGAAAAGACCCCCCATTTGCAAGAAAATCTGTTTTCCCTTTGTTGAAACATTGAAT
TTGGATTATATGGCCCTTGATTCATTCTTTAATTCGATACTGCATAAACTAACAACAAAG
ATAAAAACATTTGAAAGGAACAATGAGTTTGACGTGGATAAAAATTTAAATTTAAACTCG
ACAACGACAGTAGCAGCTTTAATTATCAAGTCGATCTTGCAACAATTCTTCAACAATTTT
CATATCAGCTTCCCTAATTTGGTTACCTTGAATTTTATTAAGATGTCTACCTACCCAAAC
AATAATGAGATTACCCAATGTTGTAACTTCATAGATTTATCTTCATATGTTCTAAACAAA
TGTTTAAGTGAGAATATCTCGATAAATTTCCTCTTTCAGTTGCACTCTTTGAAAAATTGG
TCAATGCCCAAGATTAAAGAATTCACTGGGCACAAATTCAAGTATGACGAAACAACATTT
TCAGGTTCACCAGAAAGGTACATCAAATCATTGAGGGGAAACATTAAAATTTTGCAAGAA
ATGGCAATTAACGAAACCAACGATGGTACTTGCTATTTCAGAGTCAAGTTGATACCTGAG
GGGGTAGAAAAAACTCAAATAATCAACTGGATCCCCTTTACTTCTTCATTTAGCGATGAT
ACCTTCAAACAAAGACACCATTTAAAGAGGCCAATGATTTGCTTGAAGAACAACTCTTTA
AGATCGCTCACTGTCAAAATCATACGTATTGAAAAATGTTCATCCATCCGAATCCAGGGA
TTTTACCTACCAAATCTGCAGGAACTGTTCATCAACAATACCCTTTGCGACACCACCCAA
CACCAAAAACAAGCGTCAAATGATATGAGTTGTATAGAGTTCACTTCATGGAATGAACTA
CCACAATGCAAGAAATTGGGATTTGCTCAATTAGAGGACGACTCTAACTACGTTCTTAAT
ATCAGTAACCTACAAGACCATTTACCAAATCTGGACCTGCGGGAGAGTTTCCCAACTTTC
TTCGATATAAGACAGAAGTTTGTCGTGGTTTGA
SEQ ID NO: 126
YDR271C nucleic acid sequence
ATGAATATTAATTATTATTATTGTTATAAATCTATATGCTCGTGGATTTTTTTAAATAAA
TTAGACTTACCTGTTATTTACAAGACTTCTTCGTTTGACATTAGTCCGGCCTGTGATTCT
ATGTCTTGCTCACCTGCAATAGCCCGCGTAGAAAAAAGCCTTGACCAAAAATTCCCAATC
GAAAATTTGGACTTGAAATCTGAAATCCCATGTGATTCAATATCCGGTGGAGTCCACTTC
TTCAACATCAATGAACTTAGAACGACACTGACCGAGCTGAATGCCATCGCTAAACCGGCG
AGCATTGGAGGAAGAGTTATGCCCCAAGGAATGAGCACACCCATAGCAATCGGAATCATG
AATATATTATAG
SEQ ID NO: 127
PAU7 nucleic acid sequence
ATGGTCAAATTAACTTCAATCGCTGCCGGTGTCGCCGCCATTGCTGCTGGTGCCTCCGCC
GCAGCAACCACTACATTATCTCAATCTGACGAAAGAGTTAATTTGGTTGAATTAGGTGTT
TATGTTTCCGATATCAGAGCTCATTTGGCTGAATACTACTCTTTCTAA
SEQ ID NO: 128
YGL258W-A nucleic acid sequence
ATGGCATTTGAAAGACAAGGAAAGATCGAAAAGAAGATATCGTATTCCTTATTTTTGAAT
GGACCTAATGTACACTTTGGGAGCATCTTATTCGGTGCAGTCGATAAAAGTAAGTACGCA
GAAGAGCTCTGCACACATCCTATGCGTCAAGCTTATAATACCCTTGATTCAAACTCAAGA
ATAATTATCACAGTACAGAGTGTTGCAATTTTGGACGGCAAACTTGTATGGTAA
SEQ ID NO: 129
SLU7 nucleic acid sequence
ATGAATAATAACAGCAGAAACAACGAAAATCGAAGCACTATTAACAGAAATAAAAGGCAA
CTACAACAAGCAAAAGAAAAAAATGAAAATATTCATATCCCCAGGTATATTAGAAATCAA
CCATGGTACTATAAGGATACCCCCAAAGAACAAGAAGGGAAGAAGCCCGGCAATGATGAT
ACGAGCACTGCAGAAGGAGGAGAAAAAAGCGACTACTTGGTGCATCATAGGCAAAAAGCA
AAAGGGGGTGCTTTAGATATTGACAATAATTCAGAACCAAAAATTGGTATGGGTATAAAG
GATGAGTTCAAACTAATCAGACCCCAGAAGATGTCCGTCCGAGATTCTCATTCGCTGTCA
TTTTGTAGGAATTGTGGGGAAGCAGGGCATAAGGAGAAAGACTGCATGGAAAAACCTCGT
AAGATGCAGAAGCTTGTTCCCGATTTAAATTCACAAAAAAATAATGGCACAGTTTTAGTA
CGAGCTACTGATGATGACTGGGACTCCAGAAAAGATAGATGGTACGGTTACTCAGGGAAA
GAATACAATGAACTGATAAGTAAGTGGGAGCGTGATAAAAGAAATAAAATAAAAGGAAAA
GACAAATCCCAAACTGATGAAACACTATGGGATACAGATGAAGAGATAGAACTAATGAAG
TTAGAACTTTACAAGGATTCCGTAGGTTCATTGAAGAAAGATGATGCTGATAATTCTCAG
TTGTATAGGACATCAACGAGATTGAGAGAAGATAAGGCTGCTTACTTGAACGACATAAAT
TCAACGGAGAGTAATTATGATCCTAAATCAAGATTGTACAAAACTGAAACACTGGGCGCA
GTTGATGAAAAATCAAAAATGTTCCGCAGACATTTGACAGGTGAAGGCCTAAAATTAAAC
GAATTGAACCAGTTTGCTAGATCTCACGCTAAGGAAATGGGTATACGTGATGAAATTGAG
GATAAGGAAAAAGTACAACATGTTTTAGTCGCCAATCCTACTAAATATGAATATCTGAAG
AAAAAACGGGAACAAGAAGAAACCAAGCAGCCCAAGATTGTCAGCATTGGAGATCTGGAA
GCTAGGAAAGTAGATGGTACAAAGCAATCTGAGGAACAACGGAACCACTTAAAAGATTTA
TATGGTTAA
SEQ ID NO: 130
ARP6 nucleic acid sequence
ATGGAAACACCACCCATTGTGATTGATAATGGCTCATACGAAATCAAGTTTGGTCCTTCC
ACGAATAAGAAACCGTTCCGAGCTTTAAATGCATTGGCCAAAGATAAATTTGGGACATCG
TATTTATCAAATCATATCAAAAACATCAAAGATATTTCATCTATCACCTTCAGGAGGCCA
CATGAACTAGGACAGCTCACATTATGGGAATTAGAGAGTTGTATATGGGATTATTGCCTT
TTCAATCCTTCAGAGTTTGATGGGTTTGATCTGAAAGAGGGAAAGGGTCATCATTTGGTT
GCTAGCGAGAGCTGTATGACTTTACCAGAATTAAGTAAGCATGCCGACCAGGTGATATTT
GAAGAATATGAATTCGACAGTCTTTTCAAGTCTCCTGTAGCAGTCTTTGTACCATTTACC
AAGTCATATAAGGGTGAAATGAGAACAATTTCAGGTAAGGACGAAGATATCGATATTGTC
CGTGGCAACTCAGACAGTACAAATTCCACATCAAGCGAGTCCAAGAATGCGCAGGATTCA
GGTAGCGATTATCATGATTTCCAATTAGTTATTGATTCCGGGTTTAATTGTACTTGGATA
ATTCCTGTCCTGAAGGGAATACCGTACTATAAAGCGGTAAAAAAATTGGACATTGGAGGC
CGTTTCCTAACTGGGCTACTAAAGGAAACTCTATCATTCAGACACTACAATATGATGGAT
GAAACCATACTTGTTAACAATATCAAGGAACAATGCTTGTTCGTTAGCCCGGTGTCTTAT
TTTGATAGTTTCAAAACGAAGGATAAGCATGCACTAGAATATGTACTTCCTGACTTCCAA
ACAAGCTTTCTTGGTTACGTAAGAAACCCCAGAAAAGAAAATGTACCGTTACCTGAAGAT
GCGCAGATCATAACACTGACAGATGAGCTTTTCACAATACCAGAAACTTTTTTCCATCCA
GAAATTTCGCAAATTACTAAACCAGGCATTGTGGAGGCCATCCTAGAGAGCCTTTCCATG
TTGCCCGAAATAGTGCGACCTCTTATGGTAGGAAACATTGTATGTACAGGAGGAAACTTT
AATCTGCCCAATTTCGCCCAACGGCTTGCGGCAGAACTACAAAGGCAATTACCCACAGAT
TGGACTTGTCATGTTTCGGTGCCCGAAGGTGACTGTGCTCTGTTTGGGTGGGAAGTGATG
TCACAGTTTGCAAAGACAGATTCCTACCGAAAAGCGAGGGTCACAAGAGAAGAATACTAT
GAGCATGGTCCCGATTGGTGTACGAAGCACAGGTTTGGTTACCAGAATTGGATATAA
SEQ ID NO: 131
MRP21 nucleic acid sequence
ATGTTGAAGAGCACGCTGAGGCTTTCAAGAATCTCTCTCAGAAGAGGTTTCACAACGATC
GACTGTTTACGCCAACAAAATTCGGATATCGATAAAATCATACTAAATCCAATCAAATTA
GCTCAGGGAAGCAACAGCGATCGTGGCCAAACCTCTAAAAGCAAAACTGATAATGCAGAT
ATTTTATCAATGGAAATTCCAGTAGATATGATGCAATCTGCTGGGAGAATAAACAAGAGG
GAGCTTCTATCCGAGGCGGAAATTGCTAGAAGTAGCGTGGAGAATGCACAAATGAGATTC
AATTCTGGAAAATCTATAATCGTGAATAAGAACAACCCTGCAGAATCATTTAAGAGATTA
AACAGGATCATGTTTGAGAACAATATTCCCGGAGATAAAAGAAGTCAACGGTTTTACATG
AAGCCGGGGAAAGTGGCTGAATTGAAGAGATCTCAAAGGCATAGGAAGGAATTCATGATG
GGCTTCAAGAGGTTGATTGAAATTGTTAAAGATGCCAAGAGGAAAGGATACTAA
SEQ ID NO: 132
nucleic acid sequence
ATGGATCAAGATTTTGACAGTTTATTACTAGGTTTCAATGACTCCGATAGTGTCCAAAAA
GACCCAACTGTACCAAATGGCTTGGATGGTTCAGTAGTTGATCCTACCATTGCGGATCCA
ACCGCAATTACAGCTAGAAAGAGAAGGCCTCAAGTAAAATTAACAGCCGAAAAACTACTC
AGTGATAAAGGTTTACCATATGTTTTGAAAAATGCACATAAAAGGATACGAATTTCCTCA
AAAAAAAACTCATATGACAACTTATCAAATATTATTCAGTTTTACCAGCTTTGGGCACAT
GAATTGTTTCCCAAGGCAAAATTTAAGGATTTTATGAAGATCTGTCAAACAGTAGGTAAA
ACAGATCCAGTTCTTAGAGAATATAGAGTCAGCCTTTTTAGGGACGAGATGGGCATGAGT
TTCGATGTTGGCACACGGGAGACTGGGCAAGACCTGGAAAGACAATCACCTATGGTTGAA
GAACATGTCACTTCCGCGGAAGAGAGGCCTATTGTCGCAGATAGTTTTGCGCAAGACAAA
AGGAATGTAAACAATGTCGATTACGATAATGACGAAGATGACGATATCTATCACCTTTCT
TATCGCAACAGAAGAGGACGAGTTTTGGACGAACGTGGGAATAATGAAACGGTACTTAAC
AACGTTGTGCCGCCTAAGGAAGATTTGGATGCATTATTGAAGACATTCAGGGTACAAGGG
CCCGTTGGCCTTGAAGAAAATGAGAAGAAGCTCTTATTAGGATGGCTAGATGCGCATAGA
AAAATGGAAAAAGGCTCTATGACTGAAGAAGACGTTCAACTGATTCAAAGTTTGGAAGAG
TGGGAAATGAATGATATAGAGGGACAACATACTCATTATGATTTATTGCCAGGGGGAGAT
GAGTTTGGCGTAGATCAAGATGAGTTGGATGCTATGAAGGAAATGGGCTTTTAG
SEQ ID NO: 133
AFG2 nucleic acid sequence
ATGGCTCCTAAATCTAGTTCTTCCGGTTCCAAAAAGAAATCATCGGCAAGTTCTAATAGT
GCTGATGCAAAAGCATCCAAATTTAAATTGCCTGCTGAATTTATTACCAGACCACATCCT
TCTAAAGATCATGGCAAGGAAACATGCACAGCATATATTCATCCTAACGTATTATCCTCG
CTTGAGATAAATCCGGGATCATTTTGTACTGTCGGTAAGATAGGCGAAAATGGTATTTTA
GTAATAGCTAGAGCGGGTGATGAAGAAGTACATCCTGTTAATGTTATCACCCTTTCCACA
ACTATACGATCTGTTGGGAACCTTATCCTTGGTGATCGTCTAGAATTAAAGAAAGCCCAG
GTGCAACCACCTTATGCCACTAAGGTTACCGTGGGGTCCTTACAAGGATATAATATTTTG
GAATGTATGGAGGAAAAAGTAATTCAAAAGCTACTGGATGATAGTGGCGTTATAATGCCT
GGAATGATTTTTCAAAACTTAAAAACAAAAGCAGGTGATGAAAGCATTGATGTCGTAATT
ACAGATGCGAGCGATGATTCGCTTCCCGACGTCAGCCAACTAGATCTTAACATGGACGAT
ATGTACGGTGGATTAGATAACCTGTTTTATCTATCTCCACCTTTTATATTCAGAAAAGGC
TCCACACATATAACTTTTTCGAAAGAAACCCAGGCAAATCGTAAATACAATCTTCCGGAG
CCCTTATCCTATGCAGCAGTGGGCGGCTTAGACAAGGAGATTGAATCACTGAAAAGTGCT
ATTGAAATACCTCTTCATCAACCGACGCTATTTAGTAGCTTTGGTGTTTCTCCCCCTCGA
GGTATACTTCTTCACGGACCCCCAGGTACTGGTAAAACTATGCTTTTGAGAGTTGTAGCA
AATACGTCCAACGCACACGTCCTAACCATTAATGGCCCCTCAATCGTCTCCAAATATCTT
GGTGAAACGGAAGCGGCATTAAGAGATATTTTTAATGAAGCAAGGAAGTACCAGCCTTCC
ATTATTTTCATTGACGAAATTGATTCAATAGCACCAAATAGAGCAAACGATGACTCCGGT
GAAGTTGAGAGCAGAGTCGTGGCTACATTGCTTACCCTAATGGATGGCATGGGCGCTGCA
GGTAAAGTGGTGGTAATTGCTGCTACAAACAGGCCTAATTCTGTCGACCCTGCTCTCAGG
AGACCTGGCAGGTTTGACCAAGAAGTAGAAATTGGTATACCAGACGTTGATGCCAGATTT
GACATTTTAACTAAGCAATTCTCAAGAATGTCCTCGGATCGTCACGTATTAGATTCTGAA
GCGATCAAGTACATTGCTTCTAAAACGCATGGCTATGTTGGTGCTGATTTAACTGCTCTC
TGCAGAGAATCAGTTATGAAGACGATACAACGAGGACTAGGAACAGACGCCAATATTGAC
AAGTTTTCCCTAAAAGTTACATTGAAAGATGTGGAGAGCGCCATGGTTGATATCAGACCC
AGCGCAATGAGAGAAATCTTCTTAGAAATGCCAAAAGTTTATTGGTCTGACATTGGCGGC
CAAGAAGAGCTTAAAACAAAGATGAAAGAAATGATACAGTTGCCTTTGGAGGCTTCGGAG
ACTTTTGCCAGGCTGGGAATTTCTGCACCAAAAGGTGTATTACTTTACGGGCCGCCAGGT
TGCTCCAAGACATTAACCGCAAAAGCTCTCGCTACAGAATCGGGTATCAACTTCTTAGCT
GTGAAAGGGCCTGAAATTTTTAACAAGTATGTAGGGGAATCCGAAAGAGCTATAAGAGAA
ATTTTCCGCAAAGCACGCTCTGCAGCTCCAAGTATTATCTTCTTTGATGAAATCGATGCA
TTATCTCCTGATAGAGACGGGAGTTCCACCTCTGCAGCTAATCACGTGCTCACATCTTTA
CTCAATGAGATTGATGGTGTTGAAGAGTTAAAGGGTGTAGTTATTGTAGCGGCGACGAAT
AGACCTGATGAAATAGATGCTGCTCTTCTAAGGCCTGGTAGGTTAGATAGACACATTTAC
GTTGGCCCTCCAGACGTAAACGCCCGCTTGGAAATCTTAAAGAAGTGCACAAAGAAATTT
AATACAGAAGAGTCTGGAGTCGATCTTCATGAATTGGCAGACCGTACAGAAGGTTATTCC
GGAGCTGAAGTTGTGCTGCTTTGTCAAGAAGCGGGCTTGGCTGCCATAATGGAAGATTTA
GATGTCGCAAAAGTGGAATTACGTCATTTTGAGAAAGCTTTTAAAGGAATTGCTAGGGGC
ATTACTCCAGAAATGCTCTCTTATTATGAAGAGTTTGCTCTAAGAAGCGGTTCATCTTCG
TAA
SEQ ID NO: 134
YJL152W nucleic acid sequence
ATGCCGCATTTAGCCGCCGAAGCGCATACTTGGCCTCCGCATATTTCACATTCAACACTT
TCGATTCCGCATCCAACCCCGGAACACCGGCACGTATTTCATAAAAAGGACGTGAAGAAC
AAAAGGAACGAAGAAAAAGGCAATAATTTACTCTATGTGTTATTTAGAACTACGGTGATA
AAGAGCTCGTTCCGATCACTAAGTACGGCCGGAAGAGAGCTGTTGTTTGTTGTCCATCAA
GGGCACATCGGCACCGGCCTCATCGTCTTCATCATATGCTGGAGGCTGTGCTTGAGATTC
CTCTGCAGGGTGAGCTTCCAGGTCACGGTCTACGGCGGGCGCAGTCGCATGTCTGCGTGA
SEQ ID NO: 135
PPT2 nucleic acid sequence
ATGAGTTTTGCATCGAGGAATATTGGACGTAAGATAGCAGGAGTGGGAGTTGACATTGTA
TACTTGCCAAGATTTGCACATATACTAGAGAAATATTCCCCATTCGACCCATGTGGCCGT
TCTACCTTGAATAAAATAACACGGAAGTTCATGCATGAAAAGGAAAGATTTCATTTCAGT
AATCTTCTCATCGAAGAAAACTGCTTAACTCCACGATTGCATGAATATATAGCGGGAGTT
TGGGCTTTGAAGGAATGCTCATTGAAAGCGTTGTGTTGCTGTGTTTCAAAGCATGATCTA
CCTCCTGCCCAAGTACTGTACGCTGGAATGCTATATAAAACACAAACCGATACAGGTGTA
CCTCAGTTAGAGTTTGATAAGATGTTTGGAAAAAAGTATCCAAAGTATCAACAGCTCTCG
AAAAACTACGATTCTCTCTTTTCCACTCATGAGTTTTTAGTTTCGCTATCCCATGATAAA
GATTATTTAATTGCAGTAACAAACTTGGTAGAAAGAGAGTAA
SEQ ID NO: 136
PGS1 nucleic acid sequence
ATGACGACTCGTTTGCTCCAACTCACTCGTCCTCATTACAGATTATTATCCCTACCTCTC
CAGAAACCCTTCAATATAAAAAGGCAGATGTCCGCTGCGAACCCTTCTCCATTTGGCAAT
TATTTGAACACGATCACTAAGTCCCTACAACAGAATTTACAAACATGCTTTCATTTCCAA
GCAAAAGAAATCGATATAATCGAATCTCCATCTCAGTTTTACGATCTCTTGAAGACAAAA
ATACTTAATTCACAAAATAGAATATTCATTGCGTCTCTGTATTTAGGCAAAAGCGAGACT
GAGTTGGTGGACTGCATATCCCAGGCATTGACCAAGAACCCCAAGTTGAAAGTTTCTTTT
CTACTTGATGGCCTTCGAGGAACAAGAGAATTGCCTTCCGCCTGTTCCGCCACTTTATTA
TCGTCTTTAGTAGCCAAATATGGGTCAGAGAGAGTGGATTGCCGATTGTACAAGACGCCT
GCTTATCATGGTTGGAAAAAAGTCTTGGTTCCCAAGAGATTTAATGAAGGTTTAGGCTTA
CAACATATGAAAATATATGGGTTTGATAACGAGGTCATTCTTTCGGGAGCCAACCTTTCG
AACGACTATTTCACCAACAGACAAGATAGATACTATCTCTTTAAATCTCGAAACTTCTCC
AACTATTATTTTAAATTACATCAACTCATAAGTTCCTTCAGTTATCAGATTATAAAGCCA
ATGGTGGATGGTAGCATCAACATCATTTGGCCAGATTCGAATCCTACTGTTGAACCGACG
AAAAATAAAAGGCTGTTTTTAAGGGAAGCATCTCAATTACTAGATGGCTTTTTAAAGAGT
TCTAAACAAAGCCTCCCGATTACTGCCGTGGGTCAATTCTCCACATTAGTTTACCCAATT
TCTCAATTCACTCCACTTTTTCCCAAATATAATGACAAATCGACCGAAAAAAGAACAATA
TTGTCATTGCTTTCCACTATAACAAGCAATGCCATTTCTTGGACGTTCACTGCAGGATAC
TTCAATATTTTGCCAGACATCAAAGCAAAACTGCTGGCAACGCCGGTTGCTGAGGCAAAT
GTAATAACAGCTTCCCCCTTTGCAAACGGCTTTTACCAATCAAAGGGCGTCTCATCAAAT
TTACCTGGTGCTTACTTGTACCTGTCAAAAAAATTTCTACAAGATGTATGTAGGTACAGA
CAAGATCATGCTATTACATTAAGAGAATGGCAAAGAGGCGTAGTAAATAAGCCGAATGGT
TGGTCATATCACGCAAAAGGTATTTGGCTTTCCGCTCGTGATAAAAATGATGCTAACAAT
TGGAAACCCTTTATCACGGTTATAGGATCTTCAAACTATACGAGAAGGGCGTATTCATTA
GATTTGGAATCGAATGCTCTCATTATTACAAGAGATGAAGAGCTAAGAAAAAAAATGAAA
GCAGAGTTAGATAATTTATTACAATATACAAAACCTGTAACTCTAGAAGACTTTCAATCA
GACCCAGAAAGACATGTTGGCACTGGTGTAAAGATAGCTACCTCCATTTTGGGTAAAAAA
CTTTAG
SEQ ID NO: 137
YHC1 nucleic acid sequence; systematic name YLR2989C
ATGACGAGATACTATTGTGAATACTGTCATTCGTATTTGACCCATGACACGTTGAGCGTT
CGTAAATCGCACTTGGTCGGTAAGAATCACCTTCGTATAACAGCTGACTATTATAGGAAC
AAAGCAAGAGACATTATTAATAAACATAATCATAAAAGACGCCACATTGGAAAAAGAGGC
AGGAAAGAAAGAGAAAACAGTAGTCAAAATGAGACGCTAAAAGTTACATGCCTTTCAAAT
AAGGAGAAAAGACACATCATGCATGTGAAGAAAATGAACCAAAAAGAACTGGCACAAACC
TCAATAGATACCTTGAAATTGTTATACGATGGCTCACCAGGATATTCCAAAGTATTTGTG
GATGCTAACAGGTTTGATATAGGAGATTTGGTTAAAGCCAGCAAATTACCCCAAAGAGCC
AATGAAAAATCTGCACACCATTCCTTCAAGCAAACTTCAAGATCCAGAGATGAGACGTGC
GAGAGCAATCCATTTCCTAGGTTGAATAACCCAAAGAAGCTAGAACCCCCAAAGATATTA
TCACAATGGAGTAACACCATTCCAAAAACTTCTATATTTTACAGTGTAGATATACTGCAA
ACCACGATCAAGGAGTCCAAGAAGCGGATGCATTCCGACGGCATACGGAAACCGTCGAGT
GCCAACGGATATAAAAGGAGGCGGTATGGAAATTAA
SEQ ID NO: 138
YJL045W nucleic acid sequence
ATGTTATCTTTGAAAAAAGGAATAACAAAATCATACATCTTGCAAAGAACTTTCACTTCT
TCCTCTGTTGTTCGTCAAATTGGGGAAGTGAAATCTGAATCGAAACCGCCGGCCAAATAT
CATATTATCGACCATGAATATGATTGTGTGGTGGTAGGCGCTGGCGGTGCAGGTTTAAGA
GCAGCTTTCGGTTTGGCTGAAGCTGGATACAAGACTGCTTGTTTATCCAAGTTGTTTCCA
ACAAGGTCACATACTGTGGCTGCTCAGGGTGGAATTAATGCTGCGCTGGGAAATATGCAT
CCAGATGATTGGAAATCGCACATGTACGACACTGTCAAGGGTTCTGACTGGCTCGGAGAC
CAAGATGCAATCCATTACATGACAAGAGAAGCACCTAAGTCTGTCATTGAACTAGAACAT
TACGGTATGCCCTTTTCGAGGACTGAAGATGGAAGGATTTACCAGAGAGCATTTGGGGGA
CAATCCAAAGATTTTGGTAAAGGTGGACAGGCCTATAGGACTTGTGCGGTGGCAGATAGA
ACAGGTCACGCAATGCTTCATACATTGTATGGACAAGCGCTGAAAAATAATACACACTTC
TTTATTGAATACTTTGCAATGGATTTGTTGACCCATAATGGCGAGGTTGTGGGTGTCATT
GCCTATAATCAGGAGGACGGTACAATTCACAGATTCAGAGCACATAAGACCGTCATCGCG
ACAGGCGGATACGGTAGAGCTTACTTCTCTTGCACTTCTGCTCACACTTGTACAGGTGAC
GGTAATGCTATGGTTTCTCGCGCTGGATTTCCACTAGAGGATTTAGAATTTGTTCAATTT
CATCCGTCAGGAATTTATGGGTCTGGCTGCCTAATCACTGAAGGTGCCCGTGGTGAGGGT
GGATTTTTATTGAATTCTGAAGGAGAAAGGTTTATGGAACGCTATGCTCCTACTGCCAAG
GACTTGGCAAGCAGGGATGTTGTTTCCAGAGCAATCACCATGGAAATCAGGGCTGGCAGA
GGTGTCGGGAAAAACAAGGATCATATCCTTTTACAATTAAGCCATCTACCACCTGAGGTA
CTAAAGGAAAGGCTACCGGGAATATCTGAAACAGCTGCTGTCTTTGCGGGTGTCGATGTC
ACCCAGGAGCCAATTCCTGTCTTGCCAACTGTCCATTATAATATGGGAGGCATTCCCACA
AAATGGACTGGTGAAGCATTGACCATTGACGAGGAAACTGGAGAGGATAAGGTCATCCCA
GGATTGATGGCGTGTGGTGAAGCTGCTTGCGTATCGGTTCATGGAGCGAACAGATTAGGC
GCTAACTCACTACTGGATTTAGTCGTTTTCGGTCGCGCCGTTGCAAATACCATTGCTGAC
ACATTACAGCCTGGCTTGCCTCATAAGCCATTGGCTTCAAACATCGGGCACGAGTCAATT
GCTAATTTGGATAAAGTAAGAAATGCTCGCGGCTCACTGAAAACCTCTCAAATCAGGTTG
AACATGCAAAGGACAATGCAAAAAGATGTTTCTGTTTTCAGGACGCAAGACACTCTAGAT
GAAGGTGTTAGAAATATTACTGAAGTGGACAAGACATTTGAGGATGTGCACGTTTCTGAT
AAGTCAATGATCTGGAATTCTGATCTCGTAGAAACTCTGGAATTGCAAAATTTACTTACT
TGTGCCACACAAACGGCTGTTTCTGCTTCCAAAAGAAAGGAGTCTCGTGGTGCTCATGCG
AGAGAGGACTATGCAAAAAGAGATGATGTGAATTGGAGAAAGCACACATTATCATGGCAA
AAGGGGACATCAACACCTGTAAAAATCAAGTACAGGAATGTAATCGCACATACTTTAGAT
GAGAATGAATGCGCCCCAGTCCCTCCAGCTGTCAGATCCTATTAA
SEQ ID NO: 139
NDD1 nucleic acid sequence
ATGGACAGAGATATAAGCTACCAGCAAAATTATACCTCAACTGGGGCAACTGCAACTTCC
TCAAGACAGCCCTCTACGGACAATAATGCAGATACAAATTTTTTGAAGGTAATGTCAGAA
TTCAAATATAATTTTAACAGTCCGTTACCTACAACGACTCAATTCCCCACGCCCTATTCT
TCTAATCAGTATCAACAGACTCAAGATCATTTTGCCAATACAGACGCTCACAACAGTTCG
AGCAACGAATCGTCGTTGGTAGAGAACAGTATATTACCGCATCATCAGCAGATACAACAG
CAACAACAACAACAACAACAACAACAACAACAACAGCAAGCTCTAGGTTCACTTGTACCT
CCTGCTGTCACAAGGACAGATACAAGTGAGACTTTGGACGATATCAACGTTCAACCTTCT
TCTGTTTTGCAGTTCGGCAACTCTTTACCCAGCGAATTTTTGGTTGCATCCCCAGAGCAA
TTCAAAGAATTTTTGTTGGACTCTCCGTCCACCAATTTCAATTTCTTTCACAAAACTCCG
GCAAAGACACCACTTCGATTTGTAACAGATTCTAACGGTGCTCAGCAAAGCACCACAGAG
AACCCAGGTCAACAACAGAATGTTTTTAGCAATGTCGATTTGAACAATCTTTTGAAGAGT
AATGGAAAAACACCCTCATCTTCATGCACCGGCGCATTTTCACGCACTCCTCTGAGTAAG
ATTGACATGAATCTCATGTTCAATCAACCGCTGCCGACATCTCCATCAAAAAGGTTCTCC
TCCCTGTCGTTGACACCATATGGAAGAAAAATTCTGAATGACGTCGGTACACCTTATGCA
AAAGCATTGATATCGTCTAACAGCGCGTTAGTGGATTTTCAGAAGGCAAGAAAGGATATT
ACCACTAATGCAACATCCATAGGGCTGGAAAATGCCAACAACATCTTACAGAGAACGCCG
CTAAGATCTAACAATAAAAAATTATTTATTAAAACCCCCCAGGATACCATCAATAGCACT
AGCACACTAACTAAGGACAACGAAAATAAACAGGACATATACGGCTCTTCACCGACTACC
ATCCAATTAAATTCATCAATAACTAAATCTATCTCCAAATTGGATAACTCTAGAATTCCC
TTGTTAGCTTCGAGATCAGATAACATTCTGGATTCCAATGTGGATGACCAATTGTTTGAT
TTGGGGTTGACAAGATTACCTTTATCACCAACACCAAATTGTAATTCTTTGCATAGTACA
ACCACAGGTACATCTGCCTTACAAATTCCTGAGCTACCCAAGATGGGGTCTTTTAGAAGT
GATACGGGAATCAATCCAATTTCAAGTTCAAACACAGTTTCTTTTAAGAGCAAATCAGGC
AATAATAATTCAAAGGGTCGAATCAAAAAAAATGGGAAGAAACCTTCCAAATTTCAAATT
ATTGTGGCAAATATTGATCAATTTAACCAGGATACATCATCGTCATCTTTATCATCATCA
TTGAATGCAAGTTCGAGTGCAGGGAATTCAAATTCAAACGTAACAAAGAAAAGAGCAAGT
AAACTCAAAAGATCACAGTCTTTACTTTCTGATTCCGGATCGAAATCACAAGCAAGGAAA
AGCTGTAATTCTAAATCTAATGGAAATTTATTCAATTCACAGTAA
SEQ ID NO: 140
KEX2 nucleic acid sequence
ATGAAAGTGAGGAAATATATTACTTTATGCTTTTGGTGGGCCTTTTCAACATCCGCTCTT
GTATCATCACAACAAATTCCATTGAAGGACCATACGTCACGACAGTATTTTGCTGTAGAA
AGCAATGAAACATTATCCCGCTTGGAGGAAATGCATCCAAATTGGAAATATGAACATGAT
GTTCGAGGGCTACCAAACCATTATGTTTTTTCAAAAGAGTTGCTAAAATTGGGCAAAAGA
TCATCATTAGAAGAGTTACAGGGGGATAACAACGACCACATATTATCTGTCCATGATTTA
TTCCCGCGTAACGACCTATTTAAGAGACTACCGGTGCCTGCTCCACCAATGGACTCAAGC
TTGTTACCGGTAAAAGAAGCTGAGGATAAACTCAGCATAAATGATCCGCTTTTTGAGAGG
CAGTGGCACTTGGTCAATCCAAGTTTTCCTGGCAGTGATATAAATGTTCTTGATCTGTGG
TACAATAATATTACAGGCGCAGGGGTCGTGGCTGCCATTGTTGATGATGGCCTTGACTAC
GAAAATGAAGACTTGAAGGATAATTTTTGCGCTGAAGGTTCTTGGGATTTCAACGACAAT
ACCAATTTACCTAAACCAAGATTATCTGATGACTACCATGGTACGAGATGTGCAGGTGAA
ATAGCTGCCAAAAAAGGTAACAATTTTTGCGGTGTCGGGGTAGGTTACAACGCTAAAATC
TCAGGCATAAGAATCTTATCCGGTGATATCACTACGGAAGATGAAGCTGCGTCCTTGATT
TATGGTCTAGACGTAAACGATATATATTCATGCTCATGGGGTCCCGCTGATGACGGAAGA
CATTTACAAGGCCCTAGTGACCTGGTGAAAAAGGCTTTAGTAAAAGGTGTTACTGAGGGA
AGAGATTCCAAAGGAGCGATTTACGTTTTTGCCAGTGGAAATGGTGGAACTCGTGGTGAT
AATTGCAATTACGACGGCTATACTAATTCCATATATTCTATTACTATTGGGGCTATTGAT
CACAAAGATCTACATCCTCCTTATTCCGAAGGTTGTTCCGCCGTCATGGCAGTCACGTAT
TCTTCAGGTTCAGGCGAATATATTCATTCGAGTGATATCAACGGCAGATGCAGTAATAGC
CACGGTGGAACGTCTGCGGCTGCTCCATTAGCTGCCGGTGTTTACACTTTGTTACTAGAA
GCCAACCCAAACCTAACTTGGAGAGACGTACAGTATTTATCAATCTTGTCTGCGGTAGGG
TTAGAAAAGAACGCTGACGGAGATTGGAGAGATAGCGCCATGGGGAAGAAATACTCTCAT
CGCTATGGCTTTGGTAAAATCGATGCCCATAAGTTAATTGAAATGTCCAAGACCTGGGAG
AATGTTAACGCACAAACCTGGTTTTACCTGCCAACATTGTATGTTTCCCAGTCCACAAAC
TCCACGGAAGAGACATTAGAATCCGTCATAACCATATCAGAAAAAAGTCTTCAAGATGCT
AACTTCAAGAGAATTGAGCACGTCACGGTAACTGTAGATATTGATACAGAAATTAGGGGA
ACTACGACTGTCGATTTAATATCACCAGCGGGGATAATTTCAAACCTTGGCGTTGTAAGA
CCAAGAGATGTTTCATCAGAGGGATTCAAAGACTGGACATTCATGTCTGTAGCACATTGG
GGTGAGAACGGCGTAGGTGATTGGAAAATCAAGGTTAAGACAACAGAAAATGGACACAGG
ATTGACTTCCACAGTTGGAGGCTGAAGCTCTTTGGGGAATCCATTGATTCATCTAAAACA
GAAACTTTCGTCTTTGGAAACGATAAAGAGGAGGTTGAACCAGCTGCTACAGAAAGTACC
GTATCACAATATTCTGCCAGTTCAACTTCTATTTCCATCAGCGCTACTTCTACATCTTCT
ATCTCAATTGGTGTGGAAACGTCGGCCATTCCCCAAACGACTACTGCGAGTACCGATCCT
GATTCTGATCCAAACACTCCTAAAAAACTTTCCTCTCCTAGGCAAGCCATGCATTATTTT
TTAACAATATTTTTGATTGGCGCCACATTTTTGGTGTTATACTTCATGTTTTTTATGAAA
TCAAGGAGAAGGATCAGAAGGTCAAGAGCGGAAACGTATGAATTCGATATCATTGATACA
GACTCTGAGTACGATTCTACTTTGGACAATGGAACTTCCGGAATTACTGAGCCCGAAGAG
GTTGAGGACTTCGATTTTGATTTGTCCGATGAAGACCATCTTGCAAGTTTGTCTTCATCA
GAAAACGGTGATGCTGAACATACAATTGATAGTGTACTAACAAACGAAAATCCATTTAGT
GACCCTATAAAGCAAAAGTTCCCAAATGACGCCAACGCAGAATCTGCTTCCAATAAATTA
CAAGAATTACAGCCTGATGTTCCTCCATCTTCCGGACGATCGTGA
SEQ ID NO: 141
COG7 nucleic acid sequence
ATGGTAGAGTTGACAATTACGGGTGATGATGATGATATATTGAGTATGTTTTTTGATGAG
GAGTTCGTTCCCCATGCATTCGTTGATATACTCTTATCAAATGCCTTAAACGAAGATCAG
ATTCAAACGCAATCAGTATCCTCATTGCTATTAACCAGGTTGGATTTTTACACAAAGAAC
CTTACAAAAGAGTTGGAAAGCACCATATGGAATTTGGATAAATTATCTCAAACGTTACCA
AGAACTTGGGCATCTTCTAGGTATCACAAAGAAGCAGAACAGAACGATTCCTCATTGTAT
TCTACTGAATCCTTAAAATCATCGAAGCTTGAATATTACTTAGATACGTTGGCAAGTGCT
GTAAGAGCATTAGAAACAGGAATGCATAATGTAACTGAGAAACTAAGCGATCTAGATAAC
GAAAATAATCGCAATACCAATGTGAGGCAACAACTGCAAAGTTTAATGTTGATTAAGGAG
AGAATTGAAAAAGTGGTATATTACCTGGAACAAGTTAGGACCGTTACGAATATTTCGACA
GTTAGAGAAAATAATACAACCAGCACGGGGACAGATCTTTCGATAACAGATTTTAGAACA
TCATTGAAAGCATTAGAGGATACAATCGATGAATCTTTAAGCTCTGCGATTGATAACGAG
GCTAAAGATGAAACAAACAAGGATTTGATTGGGAGAATTGATTCACTTTCTGAACTGAAA
TGTCTGTTTAAAGGCCTAGATAAGTTCTTTGCTGAGTATAGCAACTTTTCGGAGAGCATA
AAATCAAAAGCACAAAGTTATTTATCAACCAAGAATATTGACGATGGTATGATATCATAA
SEQ ID NO: 142
PRP45 nucleic acid sequence
ATGTTTAGTAACAGACTACCACCTCCAAAACATTCTCAAGGACGAGTTTCGACGGCTTTG
AGCTCAGATCGCGTTGAGCCGGCAATATTGACTGACCAAATCGCTAAAAACGTTAAGCTC
GATGATTTTATTCCAAAGAGACAGTCTAATTTCGAACTATCGGTTCCTTTGCCAACGAAA
GCAGAAATCCAAGAATGTACAGCAAGAACCAAGTCATACATTCAGCGGCTTGTGAATGCG
AAACTAGCCAACTCAAATAACAGGGCATCATCAAGGTACGTCACCGAAACACATCAGGCA
CCCGCGAATCTATTATTGAACAACAGCCACCATATTGAGGTAGTGTCCAAGCAAATGGAT
CCATTGTTGCCAAGGTTCGTTGGGAAGAAGGCGAGAAAGGTTGTAGCACCCACAGAAAAC
GACGAAGTCGTGCCTGTTCTCCATATGGATGGCAGCAATGATAGGGGAGAAGCTGATCCA
AATGAGTGGAAGATACCTGCAGCTGTGTCAAACTGGAAAAATCCAAATGGTTATACCGTG
GCCTTGGAAAGACGTGTAGGTAAAGCTCTTGACAACGAAAATAATACCATCAACGATGGG
TTTATGAAGCTCTCCGAAGCGTTAGAAAACGCTGACAAGAAGGCAAGACAAGAGATCAGG
TCCAAAATGGAATTGAAGCGGCTTGCTATGGAACAGGAAATGCTTGCTAAAGAATCTAAA
TTGAAAGAATTGAGCCAACGAGCCAGATACCACAACGGGACTCCGCAGACGGGAGCAATA
GTTAAGCCCAAAAAGCAAACGAGCACAGTGGCCAGACTAAAAGAGCTGGCGTACTCTCAA
GGAAGAGACGTATCCGAAAAGATAATTCTGGGCGCAGCAAAGCGTTCAGAACAACCGGAT
CTGCAGTACGATTCAAGATTTTTCACAAGAGGGGCAAATGCCTCCGCCAAAAGGCATGAA
GACCAGGTTTATGACAACCCACTGTTCGTCCAACAAGATATTGAAAGCATATACAAGACC
AACTACGAAAAGCTGGACGAAGCGGTCAATGTTAAGAGTGAAGGTGCCAGTGGTTCTCAC
GGCCCCATTCAGTTTACTAAAGCTGAATCCGATGATAAATCGGATAACTATGGCGCCTAG
SEQ ID NO: 143
MET16 nucleic acid sequence; systematic name YPR167C
ATGAAGACCTATCATTTGAATAATGATATAATTGTCACACAAGAACAGTTGGATCATTGG
AATGAACAACTAATCAAGCTGGAAACGCCACAGGAGATTATTGCATGGTCTATCGTAACG
TTTCCTCACCTTTTCCAAACCACTGCATTTGGTTTGACTGGCTTGGTTACTATCGATATG
TTGTCAAAGCTATCTGAAAAATACTACATGCCAGAACTATTATTTATAGACACTTTGCAC
CATTTCCCACAAACTTTAACACTAAAAAACGAGATTGAGAAAAAATACTACCAGCCTAAA
AATCAAACCATTCACGTATATAAGCCGGATGGATGTGAATCGGAGGCAGATTTTGCCTCG
AAATACGGGGATTTCTTATGGGAGAAAGATGATGACAAGTACGATTATCTGGCCAAAGTG
GAACCTGCACATCGTGCCTACAAAGAGCTACATATAAGTGCTGTGTTTACTGGTAGAAGA
AAATCACAAGGTTCTGCCCGCTCCCAACTGTCGATTATTGAAATAGACGAACTTAATGGA
ATCTTAAAAATAAATCCATTGATCAATTGGACGTTCGAGCAGGTTAAACAGTATATAGAT
GCAAACAATGTACCATACAACGAACTTTTGGACCTTGGATATAGATCCATTGGTGATTAC
CATTCCACACAACCCGTCAAGGAAGGTGAAGATGAGAGAGCAGGAAGATGGAAGGGCAAG
GCCAAGACCGAGTGTGGAATTCATGAAGCCAGCCGATTCGCGCAATTTTTAAAGCAAGAT
GCCTAG
SEQ ID NO: 144
YGR114C nucleic acid sequence
ATGTTTTCTTCTTTTTTTGGAAATACTTGTTCCTGGGTCTTCATTTTCATCATCATCGTT
GACAATGAAGCCTTCTTGCACTTTTCTTGCCTCATCTTCGTCTTCATCAATATCTTCGTC
TTCCTCAGAGGAGTCAAAGACATCTTCTCCTTCTTCTTCCTCACTAGGCGCTTTAGTTTC
ATCGTTGTCATTTACTATTTCTTCCTCGTCCCTAGGGACCAGCTTCGAATCTCCCGTCTC
TTCCATAAAAGGCAAATTCTATGTAAAGATTCTAGGCAATTAATGACCTGTTCGCTTGGG
TTATTCTTCAAAGCACAAATCAATATCTTTCTTCCTCCTTTTGCTTTAACCGTTGTCCAG
TTTCTTGTCAATTTGGTTTGCCATACATAA
SEQ ID NO: 145
RGI2 nucleic acid sequence
ATGACGAAAAAGGATAAGAAAGCAAAGGGTCCTAAGATGTCCACCATCACTACAAAAAGT
GGTGAGTCCTTAAAGGTTTTTGAGGATTTGCATGATTTTGAAACATATTTAAAGGGTGAG
ACGGAAGATCAAGAGTTCGACCATGTCCATTGCCAACTGAAGTACTATCCACCCTTTGTC
CTGCATGATGCGCATGATGATCCGGAAAAGATCAAAGAGACTGCCAATTCGCACTCTAAG
AAGTTTGTTCGCCATTTACACCAGCATGTTGAGAAGCACCTGCTAAAGGACATCAAAACC
GCTATCAACAAGCCAGAATTGAAATTCCACGATAAGAAAAAGCAGGAATCCTTTGACCGG
ATTGTTTGGAATTATGGCGAAGAAACGGAGTTGAACGCCAAGAAATTCAAGGTGTCTGTC
GAAGTTGTATGTAAACACGATGGCGCAATGGTAGATGTTGATTACAAGACAGAACCCTTG
CAGCCACTCATCTAA
SEQ ID NO: 146
YOR318C nucleic acid sequence
ATGTGTATGAACGTCACAATTTTCATAATTTTATTGAATGGGCAGGCAAATTATTCCAAG
TAAATAAATGGCGTAGTCCCTGAGTCCTCGATATTACTGTCTATTAGACTATGGTTAATC
CAACGGGAATATATTTCATCACATGATAGCGTACTGCTGCAAATTGATTAGTAAATTAGT
TCCCATCTGCCAGACACAATGTGTCAACTTCGCGTGCTCGTAAAAAAGTACTAACAACGG
AACACCTCAACAATCTTGATACTTAGTACTCTTACTACGTCATTTCTTTGTCCTAGTAAA
TTTGTTGAGAAAATTTACATAATCTGAGGAACTACAATTTCTAATCTCCAGGCACACCCA
CCACGTGCCTGTTGGCTGACCGAGACAAAAGTGGAGAAGACAGGCACGCAGAAACGAACG
TTTTGCAAGGAATGGATATGCTTCTGGAGCTTCTTCTTCCAGTCTATGCGAGATTGAATG
AGAGCGGCTGGTTGCTATGGTTTGTCTTCCATGATGTGTACGAAGCTGTGAAAATGAGTA
CTAAGGAGTCAGTGCACACCAGGGTAATCAATTTCCCTGATATTTTGTCAACTCAACAAA
TGAGACAGGGTCCATCTCAGATCAGGACACCTCTGGTAATGCTTCTTATGTGA
SEQ ID NO: 147
RAM2 nucleic acid sequence
ATGGAGGAGTACGATTATTCAGACGTTAAACCTTTGCCCATTGAGACAGACTTGCAGGAT
GAACTGTGCAGGATTATGTATACCGAGGATTATAAGCGGTTGATGGGACTCGCAAGGGCT
CTGATCAGCCTTAACGAACTGTCACCCAGGGCACTACAGCTAACAGCCGAAATTATCGAC
GTGGCGCCAGCCTTCTACACCATATGGAACTACCGATTCAATATCGTCAGGCACATGATG
AGTGAATCCGAAGACACTGTCTTGTACCTGAACAAGGAATTAGACTGGCTAGATGAAGTT
ACGCTGAATAATCCAAAGAACTATCAGATCTGGTCCTATAGACAGTCTCTTTTGAAGCTA
CATCCGTCTCCTTCCTTCAAAAGAGAGCTGCCTATCTTAAAACTGATGATTGATGATGAT
TCCAAGAATTATCACGTTTGGTCGTACAGAAAGTGGTGCTGTTTGTTCTTCAGTGACTTT
CAACATGAGCTCGCCTACGCCAGCGACCTCATCGAGACAGACATTTATAACAACAGCGCA
TGGACTCATAGGATGTTTTACTGGGTGAACGCTAAAGATGTCATTTCAAAAGTGGAATTG
GCCGACGAGCTCCAGTTCATTATGGACAAGATTCAATTGGTTCCGCAGAACATCAGTCCG
TGGACCTACCTCCGTGGTTTCCAAGAGCTATTCCATGATAGGCTACAGTGGGATAGCAAA
GTAGTCGACTTCGCCACAACCTTCATCGGTGACGTATTGTCACTTCCAATTGGCTCACCA
GAGGATTTGCCCGAGATCGAGTCCTCATATGCCCTGGAATTCCTGGCATATCACTGGGGG
GCAGACCCTTGTACCCGAGACAACGCTGTTAAGGCCTATAGCTTGCTAGCAATCAAATAC
GATCCTATTAGAAAAAACTTGTGGCACCACAAAATAAATAATCTGAACTGA
SEQ ID NO: 148
YPR027C nucleic acid sequence
ATGGTTGGTATTTACAGAATACTTGCTTCGTTCGTCCCACTCCTGGGTCTTCTTTTTGCA
TTCCATGATGATGACATGATAGATACTGTTACAATCATCAAAACTGTATATGAAACGGTG
ACATCAACTTCTACTGCACCTGCACCTGCCGCTACAAAATCTGTTAGTGAAAAGAAACTG
GATGACACTAAACTAACACTTCAAGTAATTCAAACTATGGTATCATGTTTTTCTGTAGGT
GAAAATCCGGCCAATATGATATCCTGTGGGCTAGGAGTTGTAATCTTAATGTTTTCATTA
ATCATCGAGCTTATCAACAAGCTCGAAAATGATGGTATCAATGAACCGCAAAGGTTATAT
GACCTAATTAAACCAAAATACGTCGAGCTACCTTCAAATTATGTGAATGAAAAAATCAAA
ACAACATTTGAACCTCTCGACCTATACTTAGGAGTAAATATGAATACTTCAGGAAGTGAA
CTAAACCAAAACTGTTTGATTCTCAAACTTGGCGAGAAGACGGCTCTGCCTTTCCCAGGC
TTGGCCCAGCAGATTTGTTATACAAAAGGCGCTTCAAATGAGTTCACAAATTATAAATTA
TCGGACATACAGGGCAATTTAAACGAAAACAGCCAAGGAATTGCTAATGGCGTTTTCCAG
AAAATATCAAACATTAGAAAAATATCAGGTAATTTTAAGTCTCAGCTTTATCAAATTTCA
GAAAAAATCACCGACGAAAATTGGGACGGTTCTGCTGTAGGCTTCACTGCTCATGGGAGA
GAAAAAGGCCCAAACAAATCTCAAATATCGGTTTCATTTTATAGGGATAATTAA
SEQ ID NO: 149
MGR3 nucleic acid sequence
ATGCTTTTACAAGGAATGCGTTTATCGCAAAGGTTACATAAGAGACATCTATTTGCTTCC
AAGATTTTAACGTGGACTACGAACCCTGCTCATATACGCCACCTACATGATATAAGGCCG
CCTGCATCAAACTTCAATACGCAAGAATCGGCCCCCATACCGGAGTCTCCAGCAAACTCA
CCAACTCGACCACAGATGGCACCTAAACCCAATTTGAAAAAAAAAAATCGTAGTTTAATG
TATTCTATTATTGGGGTTTCCATAGTAGGTTTATATTTTTGGTTTAAAAGTAACTCCAGG
AAACAAAAACTACCTCTTTCGGCGCAAAAAGTCTGGAAGGAAGCCATATGGCAAGAAAGT
GATAAAATGGATTTTAATTACAAAGAAGCGTTAAGGCGGTATATTGAGGCGTTGGATGAA
TGCGATCGCTCTCATGTCGATTTATTGTCAGATGATTATACCAGAATAGAGCTGAAAATT
GCTGAAATGTATGAAAAGCTCAATATGCTTGAAGAAGCCCAAAATTTGTACCAAGAATTA
TTAAGTCGGTTTTTCGAAGCGCTGAATGTTCCTGGCAAAGTTGATGAGAGTGAAAGAGGC
GAGGTTTTAAGAAAAGACTTGAGAATCTTGATTAAATCGTTAGAAATCAATAAGGACATA
GAAAGTGGCAAGAGAAAATTGCTACAACATTTACTTTTAGCTCAAGAGGAAATTTTAAGC
AAATCGCCAGAGTTGAAGGAATTTTTTGAAAACAGAAAAAAGAAGCTCTCGATGGTAAAA
GACATCAATAGAGACCCTAATGATGATTTTAAAACATTTGTTAGTGAGGAAAATATTAAG
TTTGATGAGCAAGGCTATATGATTTTGGATCTGGAAAAGAATAGCAGCGCTTGGGAACCC
TTTAAGGAAGAATTTTTTACTGCGAGAGATTTATATACAGCTTATTGTCTGTCATCAAAA
GACATAGCTGCAGCTCTAAGTTGCAAGATAACTAGTGTGGAATGGATGGTTATGGCAGAC
ATGCCACCAGGACAGATATTGCTATCACAGGCAAATTTGGGGTCATTGTTCTATCTTCAA
GCAGAAAAGCTAGAAGCTGACTTAAATCAATTAGAGCAAAAGAAAAGTAAAGAGTCCAAC
CAAGAGTTAGATATGGGAACATACATAAAAGCCGTTAGATTCGTACGCAAAAATCGTGAC
TTATGTCTGGAAAGAGCACAAAAATGTTACGACAGCGTTATTGCGTTTGCCAAAAGAAAC
AGAAAAATTAGGTTTCATGTGAAGGATCAACTGGATCCTTCAATTGCACAGTCAATTGCT
CTATCTACCTATGGAATGGGGGTTTTAAGCCTTCATGAAGGTGTTTTGGCTAAAGCTGAA
AAACTATTCAAAGATTCGATCACTATGGCCAAGGAGACTGAATTTAATGAACTCCTTGCA
GAAGCTGAAAAGGAACTAGAAAAGACGACAGTCTTGAAAGCGGCCAAAAAAGAGGGTTTA
AACTAA
SEQ ID NO: 150
FLO8 nucleic acid sequence
ATGAGTTATAAAGTGAATAGTTCGTATCCAGATTCAATTCCTCCCACGGAACAACCGTACATGGCAAGCC
AGTATAAACAAGATTTGCAGAGTAATATTGCAATGGCAACGAATAGTGAACAGCAGCGACAACAACAGCA
GCAGCAGCAACAGCAGCAACAGCAGTGGATAAATCAACCTACGGCGGAAAATTCGGATTTGAAGGAAAAA
ATGAACTGCAAGAATACGCTCAATGAGTACATATTTGACTTTCTTACGAAGTCGTCTTTGAAAAACACTG
CAGCAGCCTTTGCTCAAGATGCGCACCTAGATAGAGACAAAGGCCAAAACCCAGTCGACGGACCCAAATC
TAAAGAAAACAATGGTAACCAGAATACGTTCTCGAAGGTAGTAGATACACCTCAAGGCTTTTTGTATGAA
TGGTGGCAAATATTCTGGGACATCTTTAATACCAGTTCTTCCAGAGGTGGCTCAGAGTTCGCTCAGCAAT
ATTATCAACTAGTTCTTCAAGAACAAAGGCAGGAACAAATATATAGAAGCTTGGCTGTTCATGCGGCAAG
GCTACAACACGATGCAGAACGAAGAGGGGAATATAGTAACGAGGACATAGACCCCATGCACTTGGCTGCT
ATGATGCTAGGAAATCCTATGGCACCTGCGGTTCAAATGCGCAATGTTAATATGAACCCTATACCAATTC
CTATGGTTGGTAACCCTATCGTTAATAATTTTTCCATTCCACCATACAATAATGCAAACCCCACGACTGG
TGCAACTGCTGTTGCTCCCACAGCGCCGCCTTCCGGCGATTTTACAAATGTAGGGCCAACCCAGAATCGG
AGTCAAAACGTTACTGGCTGGCCAGTCTATAATTATCCAATGCAACCCACTACGGAAAATCCAGTGGGAA
ACCCGTGTAACAATAATACCACAAATAATACAACTAATAACAAATCTCCAGTGAACCAACCTAAAAGTTT
AAAAACTATGCATTCAACAGATAAACCAAATAATGTCCCGACGTCAAAATCTACAAGAAGTAGATCTGCA
ACCTCAAAAGCGAAGGGTAAAGTTAAAGCCGGTCTAGTGGCTAAGAGACGAAGAAAAAATAATACCGCTA
CAGTTTCCGCGGGATCGACGAACGCTTGTTCGCCAAATATTACCACACCAGGCTCAACAACAAGTGAACC
CGCTATGGTAGGTTCAAGAGTAAATAAGACTCCAAGATCAGATATTGCTACTAACTTCCGCAATCAAGCA
ATAATATTTGGCGAGGAAGATATTTATTCTAATTCCAAATCTAGCCCATCGTTGGATGGAGCATCACCTT
CCGCTTTAGCTTCTAAACAGCCCACAAAGGTAAGGAAAAATACAAAAAAGGCATCCACCTCAGCTTTTCC
AGTAGAGTCTACGAATAAACTCGGTGGCAACAGCGTGGTGACAGGTAAAAAGCGCAGTCCCCCTAACACT
AGAGTGTCGAGGAGGAAATCCACTCCTTCTGTTATTCTGAATGCTGATGCCACTAAGGATGAGAATAATA
TGTTAAGAACATTCTCGAATACTATTGCTCCGAATATTCATTCCGCTCCGCCCACTAAAACTGCGAATTC
TCTCCCTTTTCCAGGTATAAATTTGGGAAGTTTCAACAAGCCGGCTGTATCCAGTCCATTATCTTCAGTG
ACAGAGAGTTGCTTCGATCCAGAAAGTGGCAAGATTGCCGGAAAGAATGGACCCAAGCGAGCAGTAAACT
CAAAAGTTTCGGCATCATCCCCATTAAGCATAGCAACACCTCGGTCTGGTGACGCTCAGAAGCAAAGAAG
TTCTAAGGTACCAGGAAACGTGGTTATAAAGCCGCCACATGGGTTTTCAACCACCAATTTGAATATTACT
TTAAAGAACTCTAAAATAATCACTTCACAGAATAATACAGTATCCCAAGAATTGCCGAATGGGGGAAACA
TACTGGAGGCGCAAGTAGGCAATGATTCAAGAAGTAGTAAAGGCAATCGTAACACATTATCTACTCCAGA
GGAAAAAAAGCCGAGTAGTAATAATCAAGGATATGATTTTGACGCCCTCAAAAATTCAAGTTCTTTGTTG
TTTCCTAATCAAGCTTATGCTTCTAACAATAGAACACCAAACGAGAATTCAAATGTTGCTGATGAAACCT
CTGCATCTACAAATAGTGGCGATAATGATAACACATTAATTCAGCCCTCATCCAATGTGGGTACAACTTT
GGGTCCTCAGCAAACCAGTACTAATGAAAATCAGAATGTACACTCTCAGAACTTGAAGTTTGGGAATATT
GGTATGGTTGAAGACCAAGGACCGGATTACGATCTCAATTTACTGGATACAAATGAAAATGATTTCAATT
TTATTAATTGGGAAGGCTGA
SEQ ID NO: 151
BRE2 nucleic acid sequence
ATGAAGTTGGGTATTATACCTTACCAGGAAGGTACTGATATTGTTTACAAGAATGCTCTC
CAGGGTCAGCAAGAAGGGAAGAGACCTAATTTACCACAGATGGAAGCAACGCACCAAATC
AAGTCATCGGTTCAGGGTACAAGTTATGAGTTTGTCCGCACAGAAGATATTCCATTGAAT
CGAAGACATTTTGTGTACAGACCGTGTTCCGCAAATCCCTTTTTCACTATTTTGGGGTAT
GGCTGTACAGAATACCCATTTGACCACTCTGGAATGAGCGTCATGGACAGATCTGAAGGG
TTGTCAATTAGTCGAGATGGAAATGATCTGGTAAGTGTCCCGGATCAATACGGTTGGAGA
ACTGCAAGAAGCGATGTGTGTATTAAAGAAGGAATGACGTATTGGGAAGTGGAGGTAATT
CGTGGAGGAAACAAGAAATTCGCAGACGGTGTTAATAATAAGGAAAATGCTGATGATTCA
GTAGACGAAGTACAAAGTGGCATATACGAAAAAATGCACAAACAAGTGAATGACACCCCG
CATCTACGATTTGGAGTTTGCAGAAGAGAGGCCAGTTTAGAGGCTCCCGTAGGGTTTGAT
GTGTACGGGTATGGTATTAGAGACATTTCGTTAGAATCTATCCACGAAGGAAAATTGAAT
TGCGTCCTAGAAAATGGTTCGCCATTGAAAGAGGGTGATAAAATCGGATTTCTACTGAGT
CTTCCTAGCATTCATACACAAATCAAACAAGCTAAGGAGTTTACCAAAAGAAGAATTTTT
GCACTGAACTCCCATATGGATACGATGAATGAACCATGGAGAGAAGATGCTGAGAATGGG
CCTTCAAGGAAAAAATTAAAACAAGAGACAACGAACAAAGAATTTCAAAGGGCGCTATTA
GAAGATATTGAATATAACGACGTCGTCCGCGATCAAATCGCCATCAGGTATAAGAACCAG
TTGTTCTTTGAGGCAACGGACTATGTAAAGACAACGAAACCGGAATATTATTCTTCTGAT
AAGAGGGAAAGGCAAGACTATTACCAGTTAGAGGATTCATATCTTGCTATCTTTCAAAAT
GGTAAGTACCTAGGCAAAGCATTTGAAAATTTAAAGCCGTTGTTACCACCGTTCAGTGAG
TTACAATACAATGAAAAGTTCTATCTTGGATATTGGCAACATGGTGAAGCTCGTGATGAG
TCCAATGATAAAAACACAACCAGTGCCAAAAAGAAAAAGCAGCAACAAAAGAAAAAGAAG
GGATTGATACTCAGAAACAAATACGTAAATAATAACAAACTGGGTTACTATCCAACAATC
AGCTGTTTTAACGGTGGAACAGCGAGGATAATTAGTGAAGAAGATAAATTGGAGTACCTC
GATCAAATCCGATCAGCTTACTGTGTTGACGGGAATTCAAAAGTTAACACACTGGATACA
TTGTACAAAGAACAGATAGCTGAAGACATAGTATGGGATATAATCGATGAGTTGGAGCAA
ATTGCCCTACAGCAATAA
SEQ ID NO: 152
REC102 nucleic acid sequence
ATGGCAAGAGATATCACATTTTTGACCGTATTTTTAGAAAGTTGTGGCGCTGTAAATAAT
GATGAGGCAGGAAAATTGTTATCTGCTTGGACTTCAACCGTACGCATTGAGGGACCGGAA
TCAACCGACTCTAATTCATTATATATTCCACTGCTACCACCTGGAATGTTGAAAGTATGT
TTCTCCTAGCAAAATTAAAACCCATCCGTGAATGAAGCGTTACTAACTATAATAACTGGT
AGCTTTGTCACTCGTACCAGGAAAAGTGAAGATTAAACTGAATTTTAAAATGAACGATCG
ATTAGTTACGGAAGAGCAAGAGTTGTTTACAAAATTGCGCGAGATTGTAGGTTCAAGTAT
TCGCTTTTGGGAGGAACAACTGTTTTATCAAGTTCAAGATGTAAGCACCATAGAAAACCA
CGTCATTCTCAGTTTAAAATGTACAATTTTAACGGATGCTCAGATAAGTACGTTCATAAG
CAAACCCAGAGAGCTTCATACGCATGCCAAAGGATATCCTGAAATCTATTACCTTTCCGA
GTTATCAACAACTGTCAATTTTTTTTCTAAAGAGGGAAACTATGTCGAAATAAGCCAGGT
TATTCCTCATTTTAATGAATATTTTTCCTCTTTAATAGTGTCTCAATTGGAATTTGAATA
CCCGATGGTCTTCTCCATGATTTCAAGGCTCCGATTGAAGTGGCAACAAAGTTCGCTCGC
TCCGATATCCTACGCCCTAACGAGCAATTCAGTACTTCTTCCAATAATGCTTAACATGAT
TGCCCAAGACAAATCTTCAACAACCGCGTATCAAATTCTGTGTCGAAGAAGAGGTCCTCC
AATTCAGAATTTTCAAATTTTTTCCTTACCGGCTGTAACGTACAATAAGTAG
SEQ ID NO: 153
IDP3 nucleic acid sequence
ATGAGTAAAATTAAAGTTGTTCATCCCATCGTGGAAATGGACGGTGATGAGCAGACAAGA
GTTATTTGGAAACTTATCAAAGAAAAATTGATATTGCCATATTTAGATGTGGATTTAAAA
TACTATGACCTTTCAATCCAAGAGCGTGATAGGACTAATGATCAAGTAACAAAGGATTCT
TCTTATGCTACCCTAAAATATGGGGTTGCTGTCAAATGTGCCACTATAACACCCGATGAG
GCAAGAATGAAAGAATTTAACCTTAAAGAAATGTGGAAATCTCCAAATGGAACAATCAGA
AACATCCTAGGTGGAACTGTATTTAGAGAACCCATCATTATTCCAAAAATACCTCGTCTA
GTCCCTCACTGGGAGAAACCTATAATTATAGGCCGTCATGCTTTTGGTGACCAATATAGG
GCTACTGACATCAAGATTAAAAAAGCAGGCAAACTAAGGTTACAGTTTAGCTCAGATGAC
GGTAAAGAAAACATCGATTTAAAGGTTTATGAATTTCCTAAAAGTGGTGGGATCGCAATG
GCAATGTTTAATACAAATGATTCCATTAAAGGGTTCGCAAAGGCATCCTTCGAATTAGCT
CTCAAAAGAAAACTACCGTTATTCTTTACAACCAAAAACACTATTCTGAAAAATTATGAT
AATCAGTTCAAACAAATTTTCGATAATTTGTTCGATAAAGAATATAAGGAAAAGTTTCAG
GCTTTAAAAATAACGTACGAGCATCGTTTGATTGATGATATGGTAGCACAGATGCTAAAA
TCAAAGGGCGGGTTTATAATCGCCATGAAGAATTATGATGGCGATGTCCAGTCTGACATT
GTGGCACAAGGATTTGGGTCTCTTGGTTTAATGACGTCCATATTGATTACACCTGATGGT
AAAACGTTTGAAAGCGAGGCTGCCCATGGTACGGTGACCAGACATTTTAGAAAACATCAA
AGAGGCGAAGAAACATCAACAAATTCAATAGCCTCAATATTTGCCTGGACAAGGGCAATT
ATACAAAGAGGAAAATTAGACAATACAGATGATGTTATAAAATTTGGAAACTTACTAGAA
AAGGCTACTTTGGACACAGTTCAAGTGGGCGGAAAAATGACCAAGGATTTAGCATTGATG
CTTGGAAAGACTAATAGATCATCATATGTAACCACAGAAGAGTTTATTGATGAAGTTGCC
AAGAGGCTTCAAAACATGATGCTCAGCTCCAATGAAGACAAGAAAGGTATGTGCAAACTA
TAA
SEQ ID NO: 154
PEX18 nucleic acid sequence
ATGAATAGTAACCGATGCCAAACGAATGAGGTGAATAAATTTATTAGTAGTACAGAAAAG
GGGCCTTTTACGGGCAGGGACAATACGCTCTCTTTTAACAAAATCGGGAGCAGACTGAAT
TCACCACCGATTCTGAAGGATAAAATTGAGCTGAAATTTCTACAACACTCAGAAGATTTG
AATCAATCACGGTCCTACGTAAATATTCGTCCTAGAACCTTAGAGGATCAAAGTTACAAA
TTTGAAGCGCCAAATCTAAATGACAATGAAACTTCTTGGGCCAAGGATTTTAGATATAAC
TTCCCTAAGAATGTTGAACCGCCCATCGAAAATCAAATCGCGAATCTTAATATAAACAAC
GGGCTACGGACATCTCAGACAGATTTTCCCTTAGGCTTTTATTCACAGAAAAACTTTAAC
ATTGCTTCCTTCCCTGTGGTTGACCATCAGATATTCAAGACAACAGGTTTAGAACATCCT
ATCAACAGCCACATTGATTCTTTAATTAATGCTGAATTTTCGGAACTGGAAGCCAGTAGT
TTGGAAGAAGATGTCCATACAGAAGAGGAAAATTCAGGTACGAGTCTGGAAGATGAAGAA
ACTGCCATGAAAGGTTTGGCTTCCGATATAATTGAGTTTTGCGATAATAATAGTGCCAAT
AAAGATGTAAAAGAAAGACTAAACAGTTCAAAGTTTATGGGGCTGATGGGCAGCATTAGT
GATGGTTCTATAGTTTTAAAGAAGGATAACGGTACAGAAAGAAACCTTCAAAAACACGTA
GGTTTTTGTTTTCAGAATTCAGGAAACTGGGCTGGTCTTGAGTTCCATGATGTTGAAGAC
AGAATTGCTTAA
SEQ ID NO: 155
APS2 nucleic acid sequence
ATGGCAGTACAGTTTATACTGTGCTTTAATAAGCAGGGTGTGGTGCGGTTGGTGAGATGG
TTCGATGTACACAGTTCGGATCCTCAGCGTAGCCAGGATGCCATTGCGCAGATTTATAGA
CTCATATCTTCCAGAGATCATAAGCATCAGAGTAACTTCGTAGAGTTTTCCGATTCGACG
AAACTCATATACAGGAGGTATGCGGGTCTGTATTTTGTCATGGGTGTGGACTTACTTGAC
GATGAACCCATATATTTGTGCCACATCCATCTGTTTGTGGAGGTGCTAGATGCATTTTTC
GGCAATGTCTGTGAACTGGATATCGTATTCAACTTTTACAAAGTCTATATGATAATGGAC
GAGATGTTTATTGGAGGGGAAATACAAGAAATTTCAAAGGATATGCTGTTAGAAAGACTA
AGTATTTTAGATAGACTAGACTAG
SEQ ID NO: 156
HUG1 nucleic acid sequence
ATGACCATGGACCAAGGCCTTAACCCAAAGCAATTCTTCCTTGACGATGTCGTCCTACAA
GACACTTTGTGCTCAATGAGCAACCGTGTCAACAAGAGTGTCAAGACCGGCTACTTATTC
CCCAAGGATCACGTTCCTTCTGCCAACATCATTGCCGTCGAACGTCGCGGCGGTCTTTCT
GACATTGGTAAGAATACTTCCAACTAA
SEQ ID NO: 157
OSH7 nucleic acid sequence
ATGGCTCTCAATAAACTAAAGAATATACCTTCTTTAACAAACAGTTCTCATAGCTCAATT
AACGGCATTGCATCCAATGCTGCAAATTCCAAACCAAGCGGAGCAGACACGGATGATATC
GATGAGAATGATGAATCTGGGCAAAGTATTCTATTAAATATTATTTCCCAGCTGAAGCCA
GGTTGTGATTTATCTAGAATCACACTTCCGACATTTATTCTGGAAAAAAAATCGATGTTG
GAGAGAATCACTAATCAATTACAATTCCCAGATGTTCTTTTAGAAGCACACTCCAATAAA
GACGGGCTGCAAAGGTTCGTTAAAGTGGTAGCATGGTACCTAGCAGGTTGGCACATTGGG
CCCAGGGCTGTGAAGAAGCCCCTAAATCCCATTCTTGGAGAACACTTTACAGCTTATTGG
GATTTGCCTAACAAGCAACAAGCCTTTTACATTGCAGAACAAACGAGTCACCATCCTCCT
GAATCTGCGTATTTTTACATGATTCCAGAATCGAATATTAGAGTTGATGGAGTTGTTGTG
CCAAAATCGAAATTTTTAGGAAACTCAAGTGCTGCAATGATGGAGGGGTTAACTGTATTG
CAATTCCTTGATATCAAGGATGCAAATGGTAAACCAGAGAAATATACTCTATCGCAACCA
AATGTTTACGCAAGGGGAATTCTGTTTGGCAAGATGAGGATTGAATTGGGAGATCACATG
GTCATTATGGGTCCTAAGTATCAAGTGGATATTGAGTTCAAAACAAAGGGCTTTATTTCT
GGTACCTATGATGCAATTGAAGGTACAATTAAGGATTACGATGGTAAGGAATACTACCAA
ATTAGTGGTAAGTGGAATGATATTATGTATATCAAAGATTTGAGGGAAAAAAGCTCTAAA
AAGACTGTTCTCTTCGATACTCATCAGCATTTTCCTCTAGCTCCTAAAGTCCGCCCATTG
GAGGAACAGGGAGAATACGAATCGAGAAGGCTTTGGAAGAAGGTTACGGATGCGCTGGCT
GTACGTGACCATGAAGTAGCTACAGAAGAAAAGTTTCAGATAGAAAACCGCCAAAGAGAG
CTGGCCAAAAAGAGGGCCGAAGACGGCGTTGAATTTCATTCAAAACTATTTAGAAGGGCA
GAGCCAGGTGAGGATTTAGATTATTATATTTACAAGCACATCCCTGAAGGGACCGACAAG
CATGAAGAACAGATCAGGAGCATTTTGGAAACTGCCCCGATTTTACCAGGACAGACATTC
ACTGAAAAATTTTCTATTCCGGCTTATAAAAAGCATGGAATCCAAAAGAATTAG
SEQ ID NO: 158
KSS1 nucleic acid sequence
ATGGCTAGAACCATAACTTTTGATATCCCTTCCCAATATAAACTCGTAGATTTAATAGGT
GAGGGAGCGTACGGAACAGTATGTTCAGCAATTCATAAGCCTTCCGGCATAAAGGTAGCT
ATCAAGAAAATACAACCGTTTAGCAAAAAATTGTTTGTTACAAGAACTATACGTGAGATC
AAGCTTTTACGGTATTTCCATGAACACGAAAACATAATAAGTATATTGGATAAAGTAAGG
CCAGTATCCATAGACAAACTAAACGCTGTTTATTTAGTCGAAGAGTTGATGGAAACCGAT
TTACAAAAAGTAATTAATAATCAGAATAGCGGGTTTTCCACTTTAAGTGATGACCATGTT
CAATACTTTACATACCAAATCCTCAGAGCCTTAAAGTCTATTCACAGTGCACAAGTTATC
CATAGAGACATAAAGCCATCAAACCTGTTACTAAATTCCAATTGTGATCTCAAAGTCTGC
GATTTTGGACTAGCGAGGTGTTTAGCTAGCAGTAGCGATTCAAGAGAAACATTGGTAGGA
TTCATGACGGAGTACGTCGCAACGCGATGGTACAGGGCACCCGAGATAATGCTAACTTTT
CAAGAGTACACAACTGCGATGGATATATGGTCATGCGGATGCATTTTGGCTGAAATGGTC
TCCGGGAAGCCTTTGTTCCCAGGCAGAGACTATCATCATCAATTATGGCTAATTCTAGAA
GTCTTGGGAACTCCATCTTTCGAAGACTTTAATCAGATCAAATCCAAGAGGGCTAAAGAG
TATATAGCAAACTTACCTATGAGGCCACCCTTGCCATGGGAGACCGTCTGGTCAAAGACC
GATCTGAATCCAGATATGATAGATTTACTAGACAAAATGCTTCAATTCAATCCTGACAAA
AGAATAAGCGCAGCAGAAGCTTTAAGACACCCTTACCTGGCAATGTACCATGACCCAAGT
GATGAGCCGGAATATCCTCCACTTAATTTGGATGATGAATTTTGGAAACTGGATAACAAG
ATAATGCGTCCGGAAGAGGAGGAAGAAGTGCCCATAGAAATGCTCAAAGACATGCTTTAC
GATGAACTAATGAAGACCATGGAATAG
SEQ ID NO: 159
PTA1 nucleic acid sequence
ATGTCATCTGCAGAGATGGAACAATTGTTACAGGCCAAGACACTGGCCATGCACAACAAT
CCAACGGAGATGCTGCCCAAGGTGCTCGAAACTACGGCATCCATGTACCACAACGGTAAT
CTCAGCAAGCTGAAGTTGCCTTTGGCCAAGTTTTTTACACAGTTAGTTCTAGACGTGGTG
TCGATGGACTCTCCAATTGCGAATACTGAGAGACCGTTTATTGCTGCTCAATATCTGCCA
CTACTTCTTGCTATGGCGCAATCCACCGCGGACGTACTAGTGTACAAGAATATCGTGCTT
ATTATGTGCGCTTCATACCCGCTGGTGTTGGATCTGGTTGCTAAGACATCAAACCAGGAA
ATGTTTGATCAGTTGTGTATGCTGAAGAAGTTCGTGCTCTCGCACTGGAGAACTGCATAT
CCTTTGCGTGCCACCGTTGACGATGAAACGGATGTCGAACAATGGCTGGCGCAGATTGAC
CAAAATATCGGCGTGAAATTAGCGACCATCAAGTTCATATCTGAGGTCGTGCTGTCGCAA
ACTAAATCACCCAGCGGCAACGAGATTAATTCATCTACCATCCCGGATAACCACCCTGTG
TTGAACAAACCGGCTTTGGAGAGCGAGGCTAAGAGGCTTCTTGATATGTTGCTAAACTAC
CTAATTGAGGAACAGTACATGGTCTCGTCCGTTTTCATTGGTATCATCAATTCTTTATCC
TTCGTCATCAAAAGAAGGCCGCAGACAACAATAAGAATTCTTTCCGGGCTGTTGCGTTTC
AACGTCGACGCCAAGTTTCCCCTAGAGGGCAAGTCTGACTTGAACTACAAACTATCCAAG
AGATTTGTTGAAAGGGCGTACAAGAACTTTGTGCAATTTGGGCTAAAAAATCAAATCATT
ACAAAATCCCTCTCATCCGGATCAGGGTCATCGATCTACTCCAAGCTGACCAAGATTTCT
CAAACTTTACACGTTATTGGCGAAGAGACCAAGAGCAAGGGAATTTTGAACTTCGACCCT
TCCAAGGGCAATAGCAAGAAAACGTTGTCCAGGCAGGACAAACTAAAATACATCTCACTA
TGGAAAAGGCAATTATCCGCGTTATTGTCTACTCTAGGGGTGTCCACAAAGACCCCCACG
CCTGTGTCCGCACCTGCAACGGGCTCTTCAACCGAAAACATGCTTGATCAACTGAAGATA
TTGCAAAAATACACCCTCAACAAGGCTTCACACCAGGGCAATACTTTTTTCAACAACTCA
CCCAAACCAATCAGCAACACCTACTCATCTGTGTACTCATTGATGAACAGTTCGAACTCC
AACCAGGATGTGACCCAGCTACCCAATGACATACTTATCAAGCTGTCCACAGAGGCCATC
TTGCAAATGGACAGCACGAAACTGATCACCGGATTGTCTATCGTTGCTTCGAGGTACACG
GATTTAATGAATACGTACATCAATTCTGTACCGTCCTCGTCATCATCAAAGAGGAAATCC
GACGATGATGACGACGGCAACGACAATGAAGAAGTTGGAAACGATGGCCCAACGGCTAAT
AGCAAGAAAATCAAAATGGAAACAGAACCACTAGCGGAGGAACCAGAGGAGCCCGAAGAC
GATGACCGAATGCAGAAGATGCTTCAAGAAGAGGAAAGCGCCCAAGAAATCTCAGGAGAT
GCCAACAAATCAACTTCTGCCATTAAGGAGATCGCACCCCCCTTTGAACCTGACTCATTG
ACGCAGGATGAAAAACTAAAGTACCTCTCAAAGCTGACCAAGAAACTGTTTGAATTATCC
GGTCGCCAGGATACTACCCGGGCCAAATCTTCGTCTTCCTCCTCCATATTACTGGACGAT
GACGACTCCTCGTCATGGTTACACGTCTTAATCAGATTGGTTACGAGAGGAATCGAAGCA
CAAGAGGCCAGTGACCTGATTCGTGAAGAACTGCTTGGCTTCTTCATCCAGGATTTCGAG
CAACGTGTCAGTCTGATCATTGAATGGCTCAATGAAGAATGGTTCTTCCAAACCTCGCTG
CATCAAGATCCCTCTAACTACAAAAAATGGTCCTTAAGAGTTCTCGAGTCTCTGGGTCCA
TTCCTTGAAAACAAACACAGACGATTCTTCATCAGACTTATGAGCGAACTGCCCAGTCTT
CAAAGCGATCATCTTGAGGCACTGAAGCCTATCTGCCTGGATCCGGCAAGAAGTTCCCTT
GGTTTCCAAACGCTAAAGTTTCTCATTATGTTTAGACCCCCAGTGCAGGACACTGTTCGC
GACCTGCTGCATCAGCTAAAGCAAGAAGATGAAGGCTTACACAAGCAGTGCGATTCACTG
CTTGACAGGCTAAAATGA
SEQ ID NO: 160
YHR138C nucleic acid sequence
ATGAAGGCCAGTTACTTAGTTTTGATTTTCATTAGCATATTCTCCATGGCACAGGCATCT
TCCTTATCATCATACATCGTAACTTTCCCCAAGACGGATAATATGGCTACGGACCAGAAT
AGCATTATTGAAGATGTCAAAAAATATGTGGTGGACATAGGGGGTAAAATAACACACGAA
TATAGCTTGATAAAGGGCTTTACAGTGGACTTACCTGATAGCGACCAAATTTTGGACGGT
CTGAAAGAACGTTTGAGCTATATTGAAAGCGAGTACGGTGCTAAATGCAATTTGGAAAAG
GATTCAGAAGTTCATGCTCTAAACCGTGACCATTTAGTTGCTTAG
SEQ ID NO: 161
TSR3 nucleic acid sequence
ATGGGAAAAGGTAAAAATAAGATGCACGAACCCAAAAATGGAAGACCACAGAGAGGCGCT
AATGGGCACAGTTCCAGGCAAAACCATAGGCGCATGGAAATGAAGTACGATAATTCAGAA
AAAATGAAGTTTCCTGTTAAACTGGCTATGTGGGATTTTGATCATTGCGATCCAAAGAGA
TGCAGTGGTAAAAAACTTGAAAGGTTGGGCTTAATTAAATCATTGAGAGTTGGACAGAAA
TTTCAAGGTATTGTCGTTTCGCCAAACGGCAAAGGTGTTGTTTGCCCTGATGATCTAGAA
ATTGTCGAACAACACGGCGCTTCGGTGGTCGAGTGTTCTTGGGCACGTTTAGAAGAGGTA
CCCTTCAATAAAATAGGCGGTAAGCACGAAAGGCTGCTGCCGTATTTAGTGGCCGCTAAT
CAAGTAAATTACGGGAGACCATGGAGGCTCAATTGCGTTGAGGCATTAGCGGCTTGTTTT
GCTATCGTCGGGAGAATGGATTGGGCCAGTGAATTGTTATCACACTTCTCGTGGGGAATG
GGATTTTTAGAATTAAACAAAGAATTGCTTGAAATCTATCAGCAGTGCACTGACTGCGAC
TCTGTAAAGAGGGCTGAAGAGGAATGGTTGCAAAAATTAGAAAAGGAAACTCAAGAACGA
AAATCCCGAGCTAAAGAAGAAGATATATGGATGATGGGTAACATAAATAGAAGGGGTAAT
GGTTCGCAATCTGACACATCAGAGAGTGAGGAAAACTCAGAACAATCTGATTTGGAAGGC
AATAATCAATGTATTGAATATGACTCTTTAGGTAATGCTATTCGTATAGATAACATGAAA
AGCAGGGAAGCGCAATCTGAGGAATCAGAAGACGAGGAAAGTGGTTCAAAAGAAAATGGA
GAGCCTTTAAGTTATGACCCCTTAGGCAATTTAATTCGATAG
SEQ ID NO: 162
ECI1 nucleic acid sequence
ATGTCGCAAGAAATTAGGCAAAATGAGAAAATCAGTTATCGTATTGAAGGACCATTCTTC
ATTATTCACTTAATGAACCCTGACAATTTGAATGCACTAGAAGGTGAAGACTATATTTAT
TTAGGAGAGTTACTAGAACTAGCGGACAGAAATCGTGATGTATATTTTACAATTATACAA
AGCAGTGGTAGATTTTTTTCCAGTGGTGCTGATTTCAAGGGTATTGCAAAAGCCCAAGGG
GATGATACCAATAAATATCCTTCGGAAACAAGCAAGTGGGTGTCAAATTTTGTCGCTAGA
AATGTTTATGTCACTGATGCCTTCATCAAGCATTCCAAAGTTTTAATTTGCTGTTTGAAT
GGACCAGCAATAGGGTTGAGCGCGGCACTGGTAGCGTTATGTGACATTGTGTACAGTATA
AATGACAAGGTTTATTTGCTATACCCCTTTGCTAACTTAGGACTAATTACCGAAGGTGGT
ACAACGGTCTCTTTGCCATTGAAGTTTGGCACAAATACGACGTATGAATGCCTCATGTTC
AACAAACCATTCAAGTACGATATAATGTGCGAGAACGGATTTATAAGCAAGAATTTTAAC
ATGCCATCTTCAAACGCTGAAGCGTTCAATGCAAAGGTCTTAGAAGAATTGAGGGAGAAA
GTGAAAGGGCTATACCTGCCCAGTTGCTTAGGGATGAAAAAATTGCTGAAATCGAACCAC
ATCGATGCATTCAATAAGGCTAACTCAGTGGAAGTAAATGAATCTCTCAAGTATTGGGTA
GATGGAGAGCCCTTAAAAAGATTTAGGCAGCTGGGCTCGAAACAAAGGAAGCATCGTTTA
TGA
SEQ ID NO: 163
RDL2 (AIM42) nucleic acid sequence
ATGTTCAAGCATAGTACAGGTATTCTCTCGAGGACAGTTTCTGCAAGATCGCCTACATTG
GTCCTGAGAACATTTACAACGAAGGCTCCAAAGATCTATACTTTTGACCAGGTCAGGAAC
CTAGTCGAACACCCCAATGATAAAAAACTATTGGTAGATGTAAGGGAACCCAAGGAAGTA
AAGGATTACAAGATGCCAACTACAATAAATATTCCGGTGAATAGTGCCCCTGGCGCTCTT
GGATTGCCCGAAAAGGAGTTTCACAAAGTTTTCCAATTTGCTAAACCACCTCACGATAAA
GAATTGATTTTTCTTTGTGCGAAAGGAGTAAGAGCCAAAACTGCCGAAGAGTTGGCTCGA
TCTTATGGGTACGAAAACACTGGTATCTATCCTGGTTCTATTACTGAGTGGTTAGCTAAA
GGTGGTGCTGACGTTAAGCCCAAAAAATAA
SEQ ID NO: 164
SWD2 nucleic acid sequence
ATGACCACCGTGTCCATCAATAAGCCCAACCTGCTGAAATTCAAGCATGTTAAAAGCTTT
CAACCTCAAGAAAAAGACTGCGGACCCGTAACCTCATTGAATTTCGACGATAATGGCCAG
TTTCTACTGACCTCTTCTTCCAACGATACAATGCAATTGTACAGTGCCACGAACTGCAAA
TTCTTGGACACTATAGCCTCTAAGAAATATGGCTGTCACTCCGCTATCTTTACGCACGCA
CAAAACGAATGTATCTATTCCTCTACAATGAAAAATTTTGACATTAAATACCTTAATCTG
GAAACAAACCAATATCTAAGATATTTTTCCGGTCATGGCGCCCTAGTGAATGATTTGAAG
ATGAACCCCGTGAACGATACGTTTCTATCGTCGTCATACGATGAATCCGTTAGGCTTTGG
GATTTGAAGATCTCTAAACCGCAAGTTATTATACCAAGTCTCGTACCAAATTGTATCGCA
TATGATCCAAGTGGCCTTGTATTCGCATTGGGGAACCCAGAGAATTTCGAAATAGGGCTA
TATAATCTGAAAAAAATTCAGGAGGGTCCTTTCTTGATAATTAAAATTAATGATGCGACT
TTCAGTCAATGGAATAAATTAGAATTTTCTAACAATGGAAAGTATTTATTAGTTGGCTCC
TCGATAGGAAAGCATTTAATTTTTGACGCATTCACAGGTCAACAATTATTCGAACTAATA
GGAACAAGGGCCTTCCCGATGAGAGAATTTCTAGATTCTGGATCTGCTTGTTTCACACCA
GATGGTGAATTCGTCCTTGGAACCGATTATGACGGTAGGATTGCCATTTGGAATCATTCT
GATTCAATAAGTAACAAAGTATTAAGGCCGCAAGGGTTCATTCCCTGTGTTTCTCATGAG
ACCTGCCCCAGGTCAATTGCATTCAACCCTAAATATTCGATGTTTGTTACCGCAGACGAA
ACAGTAGATTTTTACGTGTACGATGAATGA
SEQ ID NO: 165
VPS71 nucleic acid sequence
ATGAAGGCGCTAGTTGAAGAGATTGATAAGAAAACTTACAATCCTGACATATATTTCACG
TCATTGGATCCTCAAGCACGTCGATATACTTCAAAGAAGATTAATAAGCAAGGCACAATA
TCCACCTCTAGGCCCGTAAAGCGCATAAACTACTCACTGGCAGATTTAGAGGCCAGGTTA
TATACTTCGAGATCTGAGGGAGATGGCAATAGTATAAGCAGACAGGATGACCGAAATAGT
AAGAATTCCCATTCATTTGAAGAAAGGTACACACAACAAGAGATTCTCCAGTCGGACAGG
AGGTTTATGGAACTTAACACAGAAAATTTCTCGGATTTACCAAATGTACCGACTTTATTA
AGTGATCTCACAGGCGTACCACGAGATAGAATTGAATCAACAACCAAACCGATCTCACAG
ACCTCGGATGGTCTCTCTGCATTAATGGGTGGTTCTTCTTTTGTAAAAGAGCATTCCAAG
TATGGTCATGGTTGGGTGCTTAAACCAGAAACCTTACGGGAAATACAATTATCGTATAAA
TCTACAAAACTACCTAAACCAAAGAGGAAGAATACCAATCGTATTGTGGCGTTAAAGAAG
GTTTTAAGTTCAAAAAGAAATTTACATTCGTTTTTAGATTCTGCGCTGCTAAACTTGATG
GATAAGAATGTCATTTACCACAATGTTTACAATAAACGATACTTCAAAGTGTTACCCCTA
ATTACGACATGCTCTATTTGCGGTGGCTACGATAGTATTTCAAGTTGCGTTAATTGTGGA
AATAAGATTTGTTCTGTAAGTTGTTTTAAATTGCATAATGAAACTAGGTGCAGAAATAGA
TAG
SEQ ID NO: 166
EMP47 nucleic acid sequence
ATGATGATGTTAATTACTATGAAAAGTACAGTACTGTTGAGTGTTTTTACCGTCTTAGCG
ACATGGGCTGGATTGCTAGAAGCTCATCCATTGGGTGACACTTCAGATGCATCCAAATTA
AGCTCAGACTACTCGCTCCCTGATCTCATTAATGCACGTAAAGTGCCCAATAACTGGCAA
ACTGGAGAACAAGCTAGTCTAGAGGAAGGGAGAATTGTATTGACTTCTAAGCAAAATTCC
AAGGGTTCACTTTGGTTGAAGCAAGGATTCGATTTGAAGGATTCTTTTACTATGGAGTGG
ACATTTAGGAGTGTTGGTTATTCTGGCCAAACCGACGGTGGCATATCATTTTGGTTTGTT
CAAGATTCTAACGTACCACGCGATAAGCAGTTATACAATGGGCCAGTGAACTATGATGGT
TTACAATTATTAGTGGATAACAATGGTCCATTGGGCCCAACACTTCGTGGTCAACTAAAT
GATGGTCAAAAGCCTGTAGATAAGACGAAAATCTATGATCAGAGTTTTGCATCTTGTTTG
ATGGGTTATCAGGATTCCTCCGTTCCTTCCACGATCAGAGTAACTTATGATTTGGAAGAC
GACAACTTATTAAAAGTTCAGGTGGACAATAAAGTCTGTTTCCAAACTAGGAAGGTTCGC
TTTCCCTCTGGGTCTTACCGTATTGGTGTCACCGCTCAAAATGGAGCAGTGAATAATAAT
GCAGAGTCTTTTGAAATATTCAAAATGCAATTTTTTAATGGCGTGATTGAAGATTCTTTG
ATCCCTAATGTGAATGCAATGGGTCAGCCAAAACTGATCACCAAATACATTGACCAACAA
ACCGGCAAAGAAAAATTGATTGAAAAAACAGCATTTGACGCTGACAAAGACAAAATTACA
AACTATGAATTGTATAAGAAACTGGATAGAGTTGAAGGTAAAATTCTTGCGAACGATATC
AATGCTTTAGAAACAAAGCTAAATGATGTCATTAAGGTCCAACAAGAGCTATTATCATTC
ATGACTACGATAACTAAACAGCTCTCTTCTAAGCCACCAGCTAATAATGAAAAGGGAACG
TCCACCGATGATGCAATCGCTGAGGATAAAGAAAATTTCAAAGACTTCTTATCAATCAAT
CAGAAATTGGAGAAAGTCCTGGTTGAACAAGAAAAGTATAGGGAAGCTACCAAACGTCAT
GGACAAGATGGTCCTCAGGTCGACGAAATTGCCAGAAAACTAATGATTTGGTTACTTCCA
TTAATTTTCATTATGTTGGTTATGGCATATTACACATTCAGAATCAGACAAGAGATCATA
AAGACCAAACTGCTATGA
SEQ ID NO: 167
ADE13 nucleic acid sequence
ATGCCTGACTATGACAATTACACTACGCCATTGTCTTCTAGATATGCCTCCAAGGAAATG
TCAGCAACGTTTTCTTTGAGAAACAGATTTTCCACATGGAGAAAACTATGGTTAAATTTG
GCTATTGCTGAGAAGGAATTGGGCTTAACTGTTGTTACAGATGAAGCAATTGAGCAAATG
CGCAAACACGTCGAAATCACTGATGATGAAATCGCAAAAGCTTCTGCTCAAGAAGCCATT
GTAAGACATGATGTTATGGCACATGTTCATACATTTGGTGAAACTTGTCCGGCTGCTGCG
GGTATCATTCACTTAGGTGCTACTTCCTGTTTCGTTACAGACAATGCTGATCTAATCTTT
ATTAGGGACGCCTACGATATTATTATTCCAAAACTTGTTAACGTCATCAACAGATTGGCT
AAGTTTGCTATGGAATACAAGGATTTGCCTGTATTGGGTTGGACTCACTTTCAACCAGCA
CAATTAACGACCTTGGGTAAGAGAGCTACTTTATGGATACAAGAGCTATTGTGGGATTTG
AGAAACTTTGAAAGAGCTAGAAACGATATCGGTCTACGTGGTGTTAAGGGTACTACTGGT
ACTCAGGCATCATTCTTGGCCTTATTCCATGGTAATCATGATAAAGTTGAAGCCCTTGAC
GAAAGAGTAACTGAATTATTAGGTTTCGATAAGGTATATCCAGTCACTGGTCAAACCTAC
TCAAGAAAAATTGACATTGACGTGTTGGCTCCTTTGTCTTCTTTTGCTGCTACTGCACAC
AAAATGGCTACTGACATAAGATTATTAGCCAACCTGAAGGAAGTTGAGGAACCTTTTGAG
AAATCACAAATCGGATCCTCTGCTATGGCTTACAAGAGAAACCCAATGCGTTGTGAGAGA
GTGTGCTCCTTGGCTAGACACTTAGGTTCCTTGTTTAGTGACGCCGTTCAAACTGCATCC
GTTCAATGGTTCGAAAGAACTCTGGATGATTCTGCTATTAGAAGAATTTCTTTACCAAGT
GCATTTTTAACCGCAGATATTCTATTATCTACTTTGTTGAACATCTCATCCGGTTTAGTT
GTGTATCCAAAGGTTATCGAAAGGAGAATTAAGGGTGAACTACCTTTTATGGCTACTGAA
AATATCATCATGGCTATGGTAGAAAAGAATGCCTCCAGACAAGAAGTACATGAGCGTATT
AGAGTGCTCTCTCATCAAGCCGCAGCAGTAGTCAAGGAAGAAGGTGGGGAAAATGATTTA
ATTGAACGAGTAAAGAGGGATGAATTTTTCAAGCCTATCTGGGAAGAATTAGATTCTTTA
CTGGAACCATCCACTTTTGTTGGTAGAGCTCCACAACAAGTTGAGAAATTTGTTCAAAAA
GACGTTAACAATGCTTTACAACCTTTCCAAAAGTACCTAAACGATGAACAAGTCAAGTTA
AATGTTTAG
SEQ ID NO: 168
FLC1 nucleic acid sequence
ATGCAAGTACTAGTGACGCTCTGGTGTCTAATATGCACATGCCTGGTACTACCAGTGGCC
GCCAAGAAAAGGACACTGACAGCGAGTTCACTGGTCACGTGCATGGAGAACTCACAGCTT
TCAGCCAATAGTTTCGATGTGTCGTTTTCTCCAGACGATCGATCGCTACATTACGATCTG
GATATGACCACGCAGATCGACTCTTACATCTACGCTTATGTGGACGTGTATGCCTACGGG
TTCAAGATTATTACGGAGAACTTCGACGTGTGTTCAATGGGTTGGAAGCAGTTTTGCCCT
GTGCACCCAGGTAACATACAAATCGACTCCATTGAATACATTGCCCAGAAGTACGTGAAA
ATGATTCCGGGAATTGCCTACCAAGTGCCCGATATTGATGCGTACGTAAGATTGAACATT
TATAACAACGTAAGTGAAAATTTGGCTTGTATCCAGGTTTTCTTTTCCAATGGGAAAACT
GTATCACAAATTGGGGTTAAATGGGTGACAGCTGTTATCGCCGGTATTGGTTTATTAACT
TCCGCTGTCTTGTCCACCTTCGGGAACTCCACAGCAGCATCTCACATTTCTGCAAACACC
ATGTCACTGTTCTTATATTTCCAATCTGTCGCTGTGGTCGCAATGCAACATGTAGACAGT
GTTCCACCCATTGCTGCTGCCTGGTCTGAAAACCTTGCCTGGTCGATGGGCTTGATCCGT
ATTACATTTATGCAGAAAATCTTCCGTTGGTATGTAGAGGCGACTGGAGGCTCCGCATCT
CTATATTTGACCGCGACAACAATGTCAGTGCTCACTCAACGAGGTCTGGATTACCTTAAA
AATACTTCGGTTTACAAGAGGGCGGAAAATGTCTTGTACGGTAACTCAAACACTTTAATC
TTTCGAGGAATTAAAAGAATGGGATACCGTATGAAGATTGAAAATACGGCCATCGTTTGT
ACTGGGTTCACATTCTTTGTGCTGTGCGGTTATTTTTTGGCCGGGTTTATCATGGCCTGC
AAATACAGTATCGAGTTATGTATAAGATGTGGTTGGATGCGGAGTGATAGGTTTTACCAA
TTTAGGAAAAACTGGAGGTCAGTTCTGAAAGGATCGTTGTTAAGATACATCTATATTGGG
TTCACGCAACTGACAATTTTAAGTTTTTGGGAGTTCACTGAACGGGATTCCGCCGGTGTT
ATTGTTATTGCATGCCTATTCATTGTATTGTCATGCGGGTTGATGGCGTGGGCTGCGTAC
AGAACCATTTTTTTCGCAAGTAAATCTGTGGAAATGTACAATAACCCAGCTGCTTTATTG
TATGGTGATGAGTACGTCTTAAACAAGTACGGGTTTTTCTACACCATGTTCAACGCAAAA
CATTATTGGTGGAATGCTCTTTTAACGACGTATATTCTTGTAAAAGCTTTATTTGTCGGA
TTCGCACAGGCATCAGGTAAAACGCAAGCATTGGCTATTTTCATTATTGACTTGGCGTAT
TTTGTTGCCATCATCCGTTATAAACCATATTTGGACCGTCCAACGAATATTGTCAACATT
TTTATTTGCACTGTCACCTTGGTCAACTCTTTCCTTTTCATGTTTTTCTCAAACTTGTTT
AACCAAAAGTATGCTGTCTCTGCCATCATGGGCTGGGTGTTTTTCATTATGAATGCTGCG
TTTTCTTTGCTTCTACTGTTGATGATTCTGGCCTTTACCACAATCATTCTGTTTTCTAAG
AATCCTGACTCCAGGTTCAAGCCAGCAAAGGATGACAGAGCATCTTTCCAAAAGCATGCT
ATTCCTCATGAAGGTGCCTTGAATAAGTCAGTGGCCAACGAATTAATGGCCCTAGGTAAT
GTGGCAAAGGATCATACCGAAAATTGGGAATACGAACTGAAGAGTCAAGAAGGTAAAAGT
GAAGATAATCTTTTCGGAGTTGAATACGATGACGAGAAAACAGGAACTAATTCAGAGAAT
GCTGAAAGTAGCAGTAAGGAAACCACCCGTCCAACCTTTTCTGAAAAGGTTTTACGTTCA
TTATCAATCAAAAGGAATAAGAGTAAACTGGGCAGTTTCAAGCGCAGCGCTCCGGATAAG
ATAACACAACAAGAGGTTTCTCCTGACCGCGCCAGCTCTTCGCCTAACAGCAAGTCATAC
CCCGGTGTCTCGCACACCAGGCAAGAATCTGAAGCGAATAATGGGCTAATCAATGCATAT
GAAGATGAGCAATTCAGTCTGATGGAACCAAGCATACTGGAAGACGCTGCTAGTTCCACC
CAAATGCATGCTATGCCAGCCCGAGATTTGAGCTTGAGCAGTGTTGCAAACGCCCAAGAT
GTTACTAAAAAAGCAAACATCCTGGATCCTGATTATTTGTAA
SEQ ID NO: 169
AOS1 nucleic acid sequence
ATGGATATGAAAGTAGAAAAATTAAGTGAAGATGAAATTGCACTGTATGATAGACAGATT
CGTCTATGGGGAATGACAGCACAGGCCAATATGAGATCAGCAAAAGTATTGCTGATCAAT
CTTGGAGCAATTGGTTCTGAAATTACCAAAAGTATCGTCCTTAGTGGTATAGGGCATTTA
ACCATATTGGATGGACACATGGTGACTGAAGAAGATTTAGGATCCCAGTTCTTCATAGGC
TCTGAAGATGTTGGCCAATGGAAGATTGATGCAACAAAAGAGAGAATTCAAGACTTGAAC
CCCCGTATAGAGTTGAACTTTGATAAGCAGGATTTGCAAGAAAAGGACGAAGAGTTCTTT
CAGCAGTTTGATTTAGTCGTGGCTACAGAAATGCAGATTGATGAAGCAATCAAAATTAAC
ACATTAACTCGAAAACTAAACATTCCATTATATGTTGCTGGTTCTAATGGATTGTTCGCT
TATGTTTTTATTGATTTGATTGAATTCATTTCAGAGGATGAAAAATTGCAAAGTGTAAGA
CCTACCACCGTTGGTCCCATTTCAAGCAATAGGAGCATTATAGAAGTTACTACTAGAAAA
GATGAAGAAGATGAAAAAAAAACATATGAACGAATCAAGACCAAGAACTGCTATAGGCCA
CTGAACGAAGTTTTAAGCACAGCAACATTAAAGGAAAAAATGACGCAAAGACAGTTAAAA
AGAGTCACTAGTATCTTGCCGTTAACCCTGTCCTTATTGCAATATGGTCTCAACCAAAAG
GGCAAGGCCATAAGCTTTGAACAAATGAAAAGAGATGCGGCCGTATGGTGTGAAAATCTG
GGTGTACCAGCAACAGTGGTAAAGGACGATTACATACAACAGTTTATCAAACAGAAAGGT
ATCGAGTTTGCTCCTGTCGCGGCCATTATAGGGGGTGCTGTAGCGCAGGATGTCATTAAC
ATTCTAGGTAAAAGGCTATCTCCATTAAATAATTTCATTGTTTTTGATGGTATTACATTA
GACATGCCACTTTTTGAGTTTTAG
SEQ ID NO: 170
YMC1 nucleic acid sequence
ATGTCTGAAGAATTTCCATCTCCTCAACTAATCGATGATTTGGAAGAACATCCACAGCAT
GATAATGCTCGAGTCGTGAAAGATTTGCTTGCAGGTACAGCGGGTGGTATTGCGCAAGTG
CTAGTGGGCCAGCCCTTTGATACGACAAAAGTTAGGTTACAAACATCGAGCACCCCAACA
ACAGCCATGGAAGTCGTCAGAAAGCTGCTTGCCAATGAAGGGCCTCGCGGGTTTTACAAA
GGAACTCTGACGCCATTAATTGGTGTTGGTGCATGTGTTTCATTACAATTTGGTGTTAAT
GAAGCTATGAAGAGATTTTTTCATCATCGCAATGCTGATATGTCATCGACTTTGTCATTG
CCACAGTATTACGCATGTGGTGTCACAGGCGGTATAGTAAACTCATTCTTGGCGTCCCCA
ATTGAGCATGTCAGGATTCGCTTGCAAACACAGACTGGCTCAGGCACCAACGCAGAATTC
AAGGGTCCTTTGGAATGCATCAAAAAATTAAGACATAACAAGGCCTTGCTACGTGGTTTA
ACACCTACAATATTGAGAGAAGGTCATGGATGTGGCACATATTTCTTAGTGTATGAAGCG
TTGATTGCTAACCAAATGAACAAAAGACGTGGACTAGAGAGAAAGGACATTCCTGCATGG
AAACTTTGTATTTTTGGAGCATTGTCTGGCACTGCCTTATGGTTGATGGTATATCCATTA
GATGTCATCAAGTCTGTCATGCAAACGGATAATTTACAAAAGCCTAAATTTGGTAATTCT
ATTTCCAGTGTAGCCAAGACTTTATATGCCAATGGAGGGATAGGCGCTTTTTTCAAAGGG
TTTGGTCCTACCATGCTAAGAGCTGCTCCCGCCAATGGTGCCACTTTTGCTACTTTTGAA
TTAGCGATGAGGTTATTGGGTTGA
SEQ ID NO: 171
MRPL20 nucleic acid sequence
ATGATTGGCAGAGGTGTGTGCTGCAGATCGTTCCACACTGCTGGATCTGCCTGGAAGCAA
TTTGGATTTCCCAAAACACAAGTGACAACGATTTACAACAAGACTAAGAGCGCATCTAAC
TATAAAGGGTATTTAAAGCACAGAGATGCTCCAGGAATGTACTATCAACCATCAGAATCC
ATCGCAACCGGATCTGTTAACAGTGAGACCATTCCACGTAGCTTTATGGCAGCCAGTGAC
CCTCGTAGAGGGCTTGACATGCCTGTTCAAAGCACTAAAGCGAAGCAGTGTCCAAATGTT
CTCGTAGGTAAGAGCACAGTGAACGGCAAAACCTATCATCTGGGACCTCAAGAAATTGAT
GAGATCCGGAAGTTACGTCTTGACAATCCTCAAAAGTATACACGCAAATTTTTGGCTGCA
AAATATGGCATTTCGCCATTATTTGTATCCATGGTCTCGAAACCTAGTGAACAACATGTA
CAAATTATGGAAAGTAGATTGCAAGAAATCCAATCACGCTGGAAGGAGAAGAGGCGTATA
GCCAGAGAGGACCGTAAGCGTAGAAAACTCCTGTGGTACCAGGCGTGA
SEQ ID NO: 172
EMC1 nucleic acid sequence
ATGAAGATAACGTGTACAGACTTGGTGTACGTCTTCATTTTACTCTTCCTAAACACGAGT
TGTGTCCAAGCCGTTTTTTCAGATGATGCATTTATCACTGATTGGCAACTGGCTAACTTA
GGTCCTTGGGAGAAAGTCATCCCTGATTCTCGAGACCGCAACAGGGTTCTCATCTTATCG
AACCCTACCGAAACTTCCTGCTTAGTTTCTTCGTTTAACGTTTCTTCCGGACAGATTCTT
TTCAGAAACGTTTTACCCTTTACCATTGATGAGATTCAACTGGATAGTAATGACCATAAC
GCAATGGTTTGTGTGAACTCTTCAAGCAACCATTGGCAGAAATATGATTTACACGATTGG
TTTTTACTAGAGGAAGGCGTAGATAATGCCCCTTCTACGACCATTTTACCTCAATCCTCA
TATTTAAACGATCAAGTATCTATTAAGAACAATGAACTACATATTCTCGATGAGCAGTCA
AAACTGGCAGAATGGAAATTGGAGTTACCTCAAGGGTTCAATAAAGTGGAATATTTTCAT
CGTGAAGATCCCCTGGCGTTAGTGTTGAACGTTAATGATACCCAATATATGGGATTCTCT
GCCAATGGCACAGAATTGATCCCCGTTTGGCAAAGAGATGAATGGTTGACTAACGTGGTA
GACTATGCTGTATTGGACGTCTTCGATTCTAGGGATGTGGAGTTGAACAAAGATATGAAA
GCGGAACTTGATTCAAATTCGCTTTGGAATGCTTACTGGCTTAGATTGACAACTAATTGG
AATCGCCTTATCAACTTATTGAAAGAAAACCAATTCTCACCAGGACGTGTCTTCACTAAA
CTCCTAGCTCTAGACGCTAAGGATACCACGGTATCAGATTTGAAGTTCGGATTCGCCAAA
ATCTTAATTGTTTTGACGCATGATGGCTTTATCGGCGGCCTTGATATGGTCAATAAGGGC
CAACTTATCTGGAAACTCGATTTAGAAATTGATCAGGGCGTCAAAATGTTCTGGACGGAT
AAAAACCATGACGAACTTGTTGTTTTTTCGCATGATGGGCATTATTTGACAATTGAAGTT
ACTAAAGATCAACCGATTATCAAATCAAGATCCCCCCTATCTGAAAGGAAAACTGTTGAT
TCCGTTATTAGGCTGAATGAACATGATCACCAGTATCTGATTAAGTTTGAGGATAAGGAT
CATTTACTGTTCAAATTGAATCCCGGCAAGAATACGGATGTACCAATAGTTGCCAACAAC
CATTCTAGTTCCCACATATTCGTCACAGAGCATGACACGAATGGCATTTATGGCTACATA
ATCGAAAACGATACGGTAAAACAAACTTGGAAAAAAGCCGTAAATTCGAAAGAGAAAATG
GTGGCATATAGCAAGAGGGAAACAACAAACCTAAACACTCTTGGTATTACACTAGGTGAC
AAATCGGTTCTTTATAAATATTTGTACCCCAACCTAGCGGCTTATCTGATCGCTAATGAA
GAACATCATACAATCACTTTTAACTTAATTGATACCATTACAGGAGAAATCCTCATTACC
CAAGAGCACAAGGATTCTCCGGATTTTAGGTTTCCAATGGATATTGTTTTCGGTGAATAT
TGGGTCGTTTATTCCTATTTCAGTTCTGAACCTGTTCCAGAACAAAAGTTAGTAGTGGTG
GAATTATATGAGTCACTAACCCCAGATGAGCGTTTGTCTAACTCAAGCGACAATTTTTCT
TATGATCCATTGACTGGACACATTAACAAACCTCAATTTCAAACTAAACAATTCATTTTT
CCCGAGATTATCAAAACAATGTCCATTTCCAAGACAACGGATGATATTACCACAAAGGCA
ATCGTTATGGAATTAGAAAATGGACAAATCACCTACATACCAAAGCTTTTATTGAATGCA
AGAGGTAAACCAGCAGAAGAAATGGCCAAGGATAAGAAAAAAGAGTTTATGGCTACCCCA
TACACGCCAGTTATCCCAATTAATGATAATTTCATTATCACTCATTTCAGAAATCTATTG
CCAGGATCCGATTCGCAGTTGATCTCCATCCCAACCAATCTGGAATCCACAAGCATTATA
TGTGATCTAGGCCTTGATGTATTTTGTACAAGGATCACACCTTCGGGCCAATTTGATTTA
ATGAGTCCTACTTTCGAAAAGGGTAAATTGCTTATTACTATATTCGTCTTGTTGGTGATC
ACGTATTTTATCCGTCCTTCTGTTTCAAACAAGAAGTTGAAATCCCAATGGCTAATTAAA
TAG
SEQ ID NO: 173
YMR155W nucleic acid sequence
ATGGTAAAGAAACACCAAAATAGTAAAATGGGTAATACAAATCACTTTGGACATCTCAAA
AGTTTTGTGGGAGGTAACGTGGTTGCCCTTGGTGCTGGAACACCTTATCTTTTCTCATTT
TATGCTCCTCAGCTACTGAGCAAGTGCCACATACCTGTTTCTGCCTCAAGTAAGCTATCC
TTCTCTTTAACAATAGGAAGCTCACTGATGGGAATTTTAGCAGGAATAGTTGTCGATCGA
AGTCCTAAACTGTCCTGTCTAATTGGTTCAATGTGTGTTTTCATCGCGTATTTGATTTTG
AATTTATGCTATAAGCACGAATGGTCTAGTACTTTTCTCATATCGTTAAGTTTGGTACTC
ATTGGATATGGTTCTGTCTCAGGTTTTTACGCTTCTGTGAAATGTGCAAATACAAATTTT
CCTCAACACAGGGGTACAGCTGGGGCATTTCCTGTGTCCCTATACGGTTTATCGGGCATG
GTGTTCTCATATCTTTGCTCAAAGCTTTTTGGTGAGAACATCGAGCATGTCTTCATTTTC
TTGATGGTTGCGTGTGGTTGCATGATTTTAGTAGGCTATTTCTCATTAGATATATTCTCT
AATGCAGAAGGAGATGATGCTAGCATTAAGGAATGGGAGCTTCAAAAAAGCAGGGAAACA
GACGATAATATAGTACCGTTATATGAGAACAGTAATGACTATATAGGTTCACCTGTGCGT
TCATCATCCCCTGCTACCTATGAAACTTATGCATTGTCAGACAATTTTCAGGAAACGTCA
GAATTTTTTGCACTTGAGGATAGACAGTTATCAAATCGACCATTGTTATCACCTTCTTCC
CCACACACAAAGTATGATTTCGAGGATGAGAATACCAGCAAAAATACAGTGGGCGAGAAT
AGCGCACAGAAAAGTATGAGATTACATGTATTCCAAAGCTTAAAATCTTCAACATTTATT
GGTTACTACATAGTATTGGGTATACTACAAGGCGTGGGTTTAATGTACATATATTCTGTG
GGGTTTATGGTACAAGCCCAGGTTTCTACTCCACCCTTAAATCAATTACCAATTAATGCA
GAAAAAATTCAATCATTACAAGTAACTCTCCTGTCTCTTCTTTCATTTTGCGGCAGATTA
TCATCTGGGCCTATATCAGATTTTTTGGTCAAGAAATTCAAAGCTCAAAGACTATGGAAT
ATTGTCATAGCATCGCTTTTGGTATTTCTTGCATCGAATAAAATATCCCATGACTTCAGC
AGCATTGAAGATCCTTCTTTAAGAGCCTCCAAATCATTCAAGAATATTTCGGTATGCTCA
GCGATCTTCGGTTATTCTTTTGGCGTTCTATTTGGTACTTTCCCCTCCATAGTAGCAGAT
AGATTTGGCACAAATGGGTATAGTACGCTGTGGGGTGTTTTAACGACTGGTGGTGTATTT
TCAGTGAGTGTTTTTACCGATATATTAGGTAGAGATTTCAAGGCAAATACAGGAGATGAT
GATGGGAACTGTAAAAAGGGAGTGCTTTGCTACAGCTATACTTTTATGGTTACGAAATAT
TGTGCCGCTTTTAATCTTTTGTTCGTTTTGGGGATAATTGGATATACGTACTATCGAAGA
AGAGCAACTGCAAATTCCCTGTAG

1. A recombinant yeast cell genetically modified to overexpress at least one protein, wherein the protein comprises at least 70% identity to an ERR3 (SEQ ID NO:1), FOX2 (SEQ ID NO:2), LYS1 (SEQ ID NO:3), MET1 (SEQ ID NO:4), MIG2 (SEQ ID NO:5), RMD6 (SEQ ID NO:6), RME1 (SEQ ID NO:7), SIP1 (SEQ ID NO:8), SNP1 (SEQ ID NO:9), TDH1 (SEQ ID NO:10), GPD1 (SEQ ID NO:11), RSF2 (SEQ ID NO:12), GND2 (SEQ ID NO:13), TRK1 (SEQ ID NO:14), HSP31 (SEQ ID NO:15), HSP33 (SEQ ID NO:16), HSP30 (SEQ ID NO:17), HSP32 (SEQ ID NO:18), ADH6 (SEQ ID NO:19), UFD4 (SEQ ID NO:20), PRO1 (SEQ ID NO:21), SIA1 (SEQ ID NO:22), ARIL (SEQ ID NO:23), LPP1 (SEQ ID NO:24), PMA2 (SEQ ID NO:25), PDR12 (SEQ ID NO:26), LCB2 (SEQ ID NO:55), CHA1 (SEQ ID NO:56), MTD1 (SEQ ID NO:58), MSC6 (SEQ ID NO:59), SCW10 (SEQ ID NO:60), YAL065C (SEQ ID NO:61), YJL107C (SEQ ID NO:62), CSM3 (SEQ ID NO:63), RGT2 (SEQ ID NO:64), CHS7 (SEQ ID NO:65), BOP2 (SEQ ID NO:66), YDR271C (SEQ ID NO:67), PAU7 (SEQ ID NO:68), YGL258W-A (SEQ ID NO:69), SLUT (SEQ ID NO:70), ARP6 (SEQ ID NO:71), MRP21 (SEQ ID NO:72), AFG2 (SEQ ID NO:73), YJL152W (SEQ ID NO:74), PPT2 (SEQ ID NO:75), PGS1 (SEQ ID NO:76), YHC1 (SEQ ID NO:77), YJL045W (SEQ ID NO:78), NDD1 (SEQ ID NO:79), KEX2 (SEQ ID NO:80), COG7 (SEQ ID NO:81), PRP45 (SEQ ID NO:82), MET16 (SEQ ID NO:83), YGR114C (SEQ ID NO:84), RGI2 (SEQ ID NO:85), YOR318C (SEQ ID NO:86), RAM2 (SEQ ID NO:87), YPR027C (SEQ ID NO:88), MGR3 (SEQ ID NO:89), FLO8 (SEQ ID NO:90), BRE2 (SEQ ID NO:91), REC102 (SEQ ID NO:92), IDP3 (SEQ ID NO:93), PEX18 (SEQ ID NO:94), APS2 (SEQ ID NO:95), HUG1 (SEQ ID NO:96), OSH7 (SEQ ID NO:97), KSS1 (SEQ ID NO:98), PTA1 (SEQ ID NO:99), YHR138C (SEQ ID NO:100), TSR3 (SEQ ID NO:101), ECI1 (SEQ ID NO:102), RDL2 (SEQ ID NO:103), SWD2 (SEQ ID NO:104), VPS71 (SEQ ID NO:105), EMP47 (SEQ ID NO:106), ADE13 (SEQ ID NO:107), FLC1 (SEQ ID NO:108), AOS1 (SEQ ID NO:109), YMC1 (SEQ ID NO:110), MRPL20 (SEQ ID NO:111), EMC1 (SEQ ID NO:1112), or YMR155W (SEQ ID NO:113) amino acid sequence.

2. The recombinant yeast cell of claim 1, wherein the protein has at least 70% identity to an amino acid sequence selected from SEQ ID NOS:1-26.

3. The recombinant yeast cell of claim 1, wherein the yeast cell comprises a recombinant expression construct comprising a promoter operably linked to a nucleic acid that encodes the protein.

4. The recombinant yeast cell of claim 3, wherein the promoter is a heterologous promoter.

5. The recombinant yeast cell of claim 3, wherein the recombinant expression construct is integrated into a yeast chromosome or is episomal.

6-8. (canceled)

9. The recombinant yeast cell of claim 1, wherein the protein is endogenous or is exogenous to the yeast cell.

10. (canceled)

11. The recombinant yeast cell of claim 1, wherein the protein has at least 80%, at least 90% identity, or at least 95% identity to an amino acid sequence of SEQ ID NO:1-26, 55, 56, or 58-113.

12-16. (canceled)

17. The recombinant yeast cell of claim 1, wherein the protein has at least 70%, at least 80% identity, or at least 90% identity, or at least 95% identity to an amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

18. (canceled)

19. (canceled)

20. The recombinant yeast cell of claim 1, wherein the protein has at least 70%, at least 80% identity, or at least 90% identity, or at least 95% identity to SEQ ID NO:2, SEQ ID NO:8, SEQ ID NO:10, SEQ ID NO:64, or SEQ ID NO:73.

21. (canceled)

22. (canceled)

23. The recombinant yeast cell of claim 1, wherein the yeast cell is genetically modified to overexpress at least a second protein, wherein the second protein comprises at least 70% identity to an ERR3 (SEQ ID NO:1), FOX2 (SEQ ID NO:2), LYS1 (SEQ ID NO:3), MET1 (SEQ ID NO:4), MIG2 (SEQ ID NO:5), RMD6 (SEQ ID NO:6), RME1 (SEQ ID NO:7), SIP1 (SEQ ID NO:8), SNP1 (SEQ ID NO:9), TDH1 (SEQ ID NO:10), GPD1 (SEQ ID NO:11), RSF2 (SEQ ID NO:12), GND2 (SEQ ID NO:13), TRK1 (SEQ ID NO:14), HSP31 (SEQ ID NO:15), HSP33 (SEQ ID NO:16), HSP30 (SEQ ID NO:17), HSP32 (SEQ ID NO:18), ADH6 (SEQ ID NO:19), UFD4 (SEQ ID NO:20), PRO1 (SEQ ID NO:21), SIA1 (SEQ ID NO:22), ARIL (SEQ ID NO:23), LPP1 (SEQ ID NO:24), PMA2 (SEQ ID NO:25), PDR12 (SEQ ID NO:26), LCB2 (SEQ ID NO:55), CHA1 (SEQ ID NO:56), MTD1 (SEQ ID NO:58), MSC6 (SEQ ID NO:59), SCW10 (SEQ ID NO:60), YAL065C (SEQ ID NO:61), YJL107C (SEQ ID NO:62), CSM3 (SEQ ID NO:63), RGT2 (SEQ ID NO:64), CHS7 (SEQ ID NO:65), BOP2 (SEQ ID NO:66), YDR271C (SEQ ID NO:67), PAU7 (SEQ ID NO:68), YGL258W-A (SEQ ID NO:69), SLUT (SEQ ID NO:70), ARP6 (SEQ ID NO:71), MRP21 (SEQ ID NO:72), AFG2 (SEQ ID NO:73), YJL152W (SEQ ID NO:74), PPT2 (SEQ ID NO:75), PGS1 (SEQ ID NO:76), YHC1 (SEQ ID NO:77), YJL045W (SEQ ID NO:78), NDD1 (SEQ ID NO:79), KEX2 (SEQ ID NO:80), COG7 (SEQ ID NO:81), PRP45 (SEQ ID NO:82), MET16 (SEQ ID NO:83), YGR114C (SEQ ID NO:84), RGI2 (SEQ ID NO:85), YOR318C (SEQ ID NO:86), RAM2 (SEQ ID NO:87), YPR027C (SEQ ID NO:88), MGR3 (SEQ ID NO:89), FLO8 (SEQ ID NO:90), BRE2 (SEQ ID NO:91), REC102 (SEQ ID NO:92), IDP3 (SEQ ID NO:93), PEX18 (SEQ ID NO:94), APS2 (SEQ ID NO:95), HUG1 (SEQ ID NO:96), OSH7 (SEQ ID NO:97), KSS1 (SEQ ID NO:98), PTA1 (SEQ ID NO:99), YHR138C (SEQ ID NO:100), TSR3 (SEQ ID NO:101), ECI1 (SEQ ID NO:102), RDL2 (SEQ ID NO:103), SWD2 (SEQ ID NO:104), VPS71 (SEQ ID NO:105), EMP47 (SEQ ID NO:106), ADE13 (SEQ ID NO:107), FLC1 (SEQ ID NO:108), AOS1 (SEQ ID NO:109), YMC1 (SEQ ID NO:110), MRPL20 (SEQ ID NO:111), EMC1 (SEQ ID NO:1112), or YMR155W (SEQ ID NO:113) amino acid sequence.

24. The recombinant yeast cell of claim 23, wherein the second protein has at least 70% identity, at least 80% identity, or at least 85% identity, or at least 90% identity, or at least 95% identity, to an amino acid sequence of SEQ ID NO:1-26, 55, 56, or 58-113.

25. (canceled)

26. The recombinant yeast cell of claim 1, wherein the recombinant yeast cell is a Candida sp., a Saccharomyces sp., or a Pichia sp.

27-29. (canceled)

30. The recombinant yeast cell of claim 1, wherein the yeast cell is capable of utilizing at least one fermentable sugar.

31. The recombinant yeast cell of claim 30, wherein the fermentable sugar is present in a cellulosic hydrolysate.

32. The recombinant yeast cell of claim 30, wherein the fermentable sugar comprises at least one pentose sugar and/or at least one hexose sugar.

33. (canceled)

34. (canceled)

35. The recombinant yeast cell of claim 30, wherein the yeast cell expresses at least one xylose utilization enzyme selected from xylose isomerase, xylose reductase, xylitol dehydrogenase, xylulokinase, xylitol isomerase and xylose transporter.

36. A fermentation composition comprising a yeast cell of claim 1 and at least one fermentable sugar.

37. The fermentation composition of claim 36, comprising a cellulosic hydrolysate and/or a lignocellulosic hydrosylate.

38. (canceled)

39. The fermentation composition of claim 36, wherein the at least one fermentable sugar comprises at least one hexose and/or at least one pentose sugar.

40. The fermentation composition of claim 36, wherein the fermentable sugar comprises glucose and/or xylose.

41. A method for producing at least one fermentation product, the method comprising maintaining the fermentation composition of claim 36, under conditions in which at least one fermentation product is produced.

42. The method of claim 4, wherein at least one fermentation product is an alcohol.

43. (canceled)

44. The method of claim 41, further comprising recovering at least one fermentation product from the fermentation composition.
